(19) United States
(12) Patent Application Publication
(10) Pub. No.: US 2014/0066369 A1
(43) Pub. Date: Mar. 6, 2014

(54) Modulators Of The GPR119 Receptor And The Treatment Of Disorders Related Thereto

(71) Applicants: Robert M. Jones, San Diego, CA (US); Daniel J. Buzard, San Diego, CA (US); Sangdon Han, San Diego, CA (US); Sun Hee Kim, San Diego, CA (US); Juerg Lehmann, San Diego, CA (US); Dawei Yue, San Diego, CA (US); Xiuwen Zhu, San Diego, CA (US)

(72) Inventors: Robert M. Jones, San Diego, CA (US); Daniel J. Buzard, San Diego, CA (US); Sangdon Han, San Diego, CA (US); Sun Hee Kim, San Diego, CA (US); Juerg Lehmann, San Diego, CA (US); Dawei Yue, San Diego, CA (US); Xiuwen Zhu, San Diego, CA (US)

(73) Assignee: ARENA PHARMACEUTICALS, INC., San Diego, CA (US)

(21) Appl. No.: 14/112,384

(22) PCT Filed: Apr. 18, 2012
(86) PCT No.: PCT/US12/34022

§ 371 Date: Oct. 17, 2013

Related U.S. Application Data
(60) Provisional application No. 61/476,995, filed on Apr. 19, 2011.

(51) Int. Cl. C07D 295/205; A61K 45/06; C07D 239/42; A61K 31/506; A61K 31/496; A61K 31/499; C07D 249/08; C07D 403/12; C07D 257/04; C07D 487/08; A61K 31/4995; C07D 241/38; A61K 31/495; C07D 271/07

(52) U.S. Cl. CPC C07D 295/205; A61K 31/495; A61K 45/06; C07D 239/42; A61K 31/506; A61K 31/496; C07D 271/07; C07D 249/08; C07D 403/12; C07D 257/04; C07D 487/08; A61K 31/4995; C07D 241/38; A61K 31/499
USPC 514/6.5; 544/389; 514/255.01; 544/295; 514/252.14; 514/254.03; 544/367; 544/366; 514/254.05; 544/349; 514/249; 544/231

(57) ABSTRACT

The present invention relates to compounds of Formula I and pharmaceutically acceptable salts, solvates, and hydrates thereof, that are useful as single pharmaceutical agents or in combination with one or more additional pharmaceutical agents, such as, a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, in the treatment of for example, a disorder selected from: a GPR119-receptor-related disorder; a condition ameliorated by increasing a blood incretin level; a condition characterized by low bone mass; a neurological disorder; a metabolic-related disorder; type 2 diabetes; obesity; and complications related thereto.

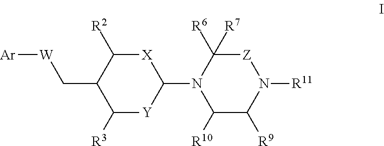

### FIELD OF THE INVENTION

The present invention relates to compounds of Formula I and pharmaceutically acceptable salts, solvates, and hydrates thereof, that are useful as a single agent or in combination with one or more pharmaceutical agents, such as, a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, in the treatment of, for example, a disorder selected from: a GPR119-receptor-related disorder; a condition ameliorated by increasing a blood incretin level; a condition characterized by low bone mass; a neurological disorder; a metabolic-related disorder; type 2 diabetes; obesity; and complications related thereto.

### BACKGROUND OF THE INVENTION

### A. Diabetes Mellitus

Diabetes mellitus is a serious disease afflicting over 100 million people worldwide. In the United States, there are more than 12 million diabetics, with 600,000 new cases diagnosed each year.

Diabetes mellitus is a diagnostic term for a group of disorders characterized by abnormal glucose homeostasis resulting in elevated blood sugar. There are many types of diabetes, but the two most common are type 1 (also referred to as insulin-dependent diabetes mellitus or IDDM) and type 2 (also referred to as non-insulin-dependent diabetes mellitus or NIDDM).

The etiology of the different types of diabetes is not the same; however, everyone with diabetes has two things in common: overproduction of glucose by the liver and little or no ability to move glucose out of the blood into the cells where it becomes the body's primary fuel.

People who do not have diabetes rely on insulin, a hormone made in the pancreas, to move glucose from the blood into the cells of the body. However, people who have diabetes either don't produce insulin or can't efficiently use the insulin they produce; therefore, they can't move glucose into their cells. Glucose accumulates in the blood creating a condition called hyperglycemia, and over time, can cause serious health problems.

Diabetes is a syndrome with interrelated metabolic, vascular, and neuropathic components. The metabolic syndrome, generally characterized by hyperglycemia, comprises alterations in carbohydrate, fat and protein metabolism caused by absent or markedly reduced insulin secretion and/or ineffective insulin action. The vascular syndrome consists of abnormalities in the blood vessels leading to cardiovascular, retinal and renal complications. Abnormalities in the peripheral and autonomic nervous systems are also part of the diabetic syndrome.

About 5% to 10% of the people who have diabetes have IDDM. These individuals don't produce insulin and therefore must inject insulin to keep their blood glucose levels normal. IDDM is characterized by low or undetectable levels of endogenous insulin production caused by destruction of the insulin-producing β cells of the pancreas, the characteristic that most readily distinguishes IDDM from NIDDM. IDDM, once termed juvenile-onset diabetes, strikes young and older adults alike.

Approximately 90 to 95% of people with diabetes have type 2 (or NIDDM). NIDDM subjects produce insulin, but the cells in their bodies are insulin resistant: the cells don't respond properly to the hormone, so glucose accumulates in their blood. NIDDM is characterized by a relative disparity between endogenous insulin production and insulin requirements, leading to elevated blood glucose levels. In contrast to IDDM, there is always some endogenous insulin production in NIDDM; many NIDDM patients have normal or even elevated blood insulin levels, while other NIDDM patients have inadequate insulin production (Rotwein, R. et al. N. Engl. J. Med. 308, 65-71 (1983)). Most people diagnosed with NIDDM are age 30 or older, and half of all new cases are age 55 and older. Compared with whites and Asians, NIDDM is more common among Native Americans, African-Americans, Latinos, and Hispanics. In addition, the onset can be insidious or even clinically unapparent, making diagnosis difficult.

The primary pathogenic lesion on NIDDM has remained elusive. Many have suggested that primary insulin resistance of the peripheral tissues is the initial event. Genetic epidemiological studies have supported this view. Similarly, insulin secretion abnormalities have been argued as the primary defect in NIDDM. It is likely that both phenomena are important contributors to the disease process (Rimoin, D. L., et. al. Emery and Rimoin's Principles and Practice of Medical Genetics 3^{rd} Ed. 1:1401-1402 (1996)).

Many people with NIDDM have sedentary lifestyles and are obese: they weigh approximately 20% more than the recommended weight for their height and build. Furthermore, obesity is characterized by hyperinsulinemia and insulin resistance, a feature shared with NIDDM, hypertension and atherosclerosis.

The patient with diabetes faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions (Perry, I. J., et al., BMJ 310, 560-564 (1995)).

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina at the back of the eye and increases risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

### B. Obesity

Obesity and diabetes are among the most common human health problems in industrialized societies. In industrialized countries a third of the population is at least 20% overweight. In the United States, the percentage of obese people has increased from 25% at the end of the 1970's, to 33% at the beginning the 1990's. Obesity is one of the most important risk factors for NIDDM. Definitions of obesity differ, but in general, a subject weighing at least 20% more than the recommended weight for his/her height and build is considered obese. The risk of developing NIDDM is tripled in subjects 30% overweight, and three-quarters with NIDDM are overweight.

Obesity, which is the result of an imbalance between caloric intake and energy expenditure, is highly correlated with insulin resistance and diabetes in experimental animals and human. However, the molecular mechanisms that are involved in obesity-diabetes syndromes are not clear. During early development of obesity, increased insulin secretion balances insulin resistance and protects patients from hyperglycemia (Le Stunff, et al. Diabetes 43, 696-702 (1989)). However, after several decades, β cell function deteriorates and non-insulin-dependent diabetes develops in about 20% of the obese population (Pederson, P. Diab. Metab. Rev. 5, 505-509 (1989)) and (Brancati, F. L., et al., Arch. Intern. Med. 159, 957-963 (1999)). Given its high prevalence in modern societies, obesity has thus become the leading risk factor for NIDDM (Hill, J. O., et al., Science 280, 1371-1374 (1998)). However, the factors which predispose a fraction of patients to alteration of insulin secretion in response to fat accumulation remain unknown.

Whether someone is classified as overweight or obese can be determined by a number of different methods, such as, on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see table below). There are problems with this definition in that it does not take into account the proportion of body mass that is muscle in relation to fat (adipose tissue). To account for this, alternately, obesity can be defined on the basis of body fat content: greater than 25% and 30% in males and females, respectively.

CLASSIFICATION OF WEIGHT BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Obesity considerably increases the risk of developing cardiovascular diseases as well. Coronary insufficiency, atheromatous disease, and cardiac insufficiency are at the forefront of the cardiovascular complication induced by obesity. It is estimated that if the entire population had an ideal weight, the risk of coronary insufficiency would decrease by 25% and the risk of cardiac insufficiency and of cerebral vascular accidents by 35%. The incidence of coronary diseases is doubled in subjects less than 50 years of age who are 30% overweight.

### C. Atherosclerosis

Atherosclerosis is a complex disease characterized by inflammation, lipid accumulation, cell death and fibrosis. Atherosclerosis is characterized by cholesterol deposition and monocyte infiltration into the subendothelial space, resulting in foam cell formation. Thrombosis subsequent to atherosclerosis leads to myocardial infarction and stroke. Atherosclerosis is the leading cause of mortality in many countries, including the United States. (See, e.g., Ruggeri, Nat Med (2002) 8:1227-1234; Arehart et al, Circ Res, Circ. Res. (2008) 102:986-993.)

### D. Osteoporosis

Osteoporosis is a disabling disease characterized by the loss of bone mass and microarchitectural deterioration of skeletal structure leading to compromised bone strength, which predisposes a patient to increased risk of fragility fractures. Osteoporosis affects more than 75 million people in Europe, Japan and the United States, and causes more than 2.3 million fractures in Europe and the United States alone. In the United States, osteoporosis affects at least 25% of all post-menopausal white women, and the proportion rises to 70% in women older than 80 years. One in three women older than 50 years will have an osteoporotic fracture that causes a considerable social and financial burden on society. The disease is not limited to women; older men also can be affected. By 2050, the worldwide incidence of hip fracture in men is projected to increase by 310% and 240% in women. The combined lifetime risk for hip, forearm, and vertebral fractures presenting clinically is around 40%, equivalent to the risk for cardiovascular disease. Osteoporotic fractures therefore cause substantial mortality, morbidity, and economic cost. With an ageing population, the number of osteoporotic fractures and their costs will at least double in the next 50 years unless effective preventive strategies are developed. (See, e.g., Atik et al., Clin Orthop Relat Res (2006) 443:19-24; Raisz, J Clin Invest (2005) 115:3318-3325; and World Health Organization Technical Report Series 921 (2003), Prevention and Management of Osteoporosis.)

### E. Inflammatory Bowel Disease (IBD)

Inflammatory bowel disease (IBD) is the general name for diseases that cause inflammation in the intestines and includes, e.g. Crohn's disease, ulcerative colitis, ulcerative proctitis. U.S. medical costs of inflammatory bowel disease for 1990 have been estimated to be $1.4 to $1.8 billion. Lost productivity has been estimated to have added an additional $0.4 to $0.8 billion, making the estimated cost of inflammatory bowel disease $1.8 to $2.6 billion. (See, e.g., Pearson, Nursing Times (2004) 100:86-90; Hay et al, J Clin Gastroenterol (1992) 14:309-317; Keighley et al, Ailment Pharmacol Ther (2003) 18:66-70.)

Enteritis refers to inflammation of the intestine, especially the small intestine, a general condition that can have any of numerous different causes. Enterocolitis refers to inflammation of the small intestine and colon.

Crohn's disease (CD) is an inflammatory process that can affect any portion of the digestive tract, but is most commonly seen in the last part of the small intestine otherwise called the (terminal) ileum and cecum. Altogether this area is also known as the ileocecal region. Other cases may affect one or more of: the colon only, the small bowel only (duodenum, jejunum and/or ileum), the anus, stomach or esophagus. In contrast with ulcerative colitis, CD usually does not affect the rectum, but frequently affects the anus instead. The inflammation extends deep into the lining of the affected organ. The inflammation can cause pain and can make the intestines empty frequently, resulting in diarrhea. Crohn's disease may also be called enteritis. Granulomatous colitis is another name for Crohn's disease that affects the colon. Ileitis is CD of the ileum which is the third part of the small intestine. Crohn's colitis is CD affecting part or all of the colon.

Ulcerative colitis (UC) is an inflammatory disease of the large intestine, commonly called the colon. UC causes inflammation and ulceration of the inner lining of the colon and rectum. The inflammation of UC is usually most severe in the rectal area with severity diminishing (at a rate that varies from patient to patient) toward the cecum, where the large and small intestine join. Inflammation of the rectum is called proctitis. Inflammation of the sigmoid colon (located just above the rectum) is called sigmoiditis. Inflammation involving the entire colon is termed pancolitis. The inflammation causes the colon to empty frequently resulting in diarrhea. As the lining of the colon is destroyed ulcers form releasing mucus, pus and blood. Ulcerative proctitis is a form of UC that affects only the rectum.

### F. GPR119

GPR119 is a G protein-coupled receptor (GPR119; e.g., human GPR119, GenBank® Accession No. AAP72125 and alleles thereof; e.g., mouse GPR119, GenBank® Accession No. AY288423 and alleles thereof) and is selectively expressed on pancreatic beta cells. GPR119 activation leads to elevation of a level of intracellular cAMP, consistent with GPR119 being coupled to Gs. Agonists to GPR119 stimulate glucose-dependent insulin secretion in vitro and lower an elevated blood glucose level in vivo; see, e.g., International Applications WO 04/065380 and WO 04/076413, and EP 1338651, the disclosure of each of which is herein incorporated by reference in its entirety. In the literature, GPR119 has also been referred to as RUP3 (see, International Application WO 00/31258) and as Glucose-Dependent Insulinotropic Receptor GDIR (see, Jones, et. al. Expert Opin. Ther. Patents (2009), 19(10): 1339-1359).

GPR119 agonists also stimulate the release of Glucose-dependent Insulinotropic Polypeptide (GIP), Glucagon-Like Peptide-1 (GLP-1), and at least one other L-cell peptide, Peptide YY (PYY) (Jones, et. al. Expert Opin. Ther. Patents (2009), 19(10): 1339-1359); for specific references related to GPR119 agonists and the release of:

GIP, see Shah, Current Opinion in Drug Discovery & Development, (2009) 12:519-532; Jones, et al., Ann. Rep. Med. Chem., (2009) 44:149-170; WO 2007/120689; and WO 2007/120702; GLP-1, see Shah, Current Opinion in Drug Discovery & Development, (2009) 12:519-532; Jones, et al., Ann. Rep. Med. Chem., (2009) 44:149-170; Schwartz et. al., Cell Metabolism, 2010, 11:445-447; and WO 2006/076231; and

PYY, see Schwartz et. al., Cell Metabolism, 2010, 11:445-447; and WO 2009/126245.

As mentioned above, GPR119 agonists enhance incretin release and therefore can be used in treatment of disorders related to the incretins, such as, GIP, GLP-1, and PYY. However, a number of the incretins, such as, GIP and GLP-1, are substrates for the enzyme DPP-IV. Jones and co-workers (Jones, et al., Ann. Rep. Med. Chem., (2009) 44:149-170) have demonstrated that a combined administration of a GPR119 agonist, (2-Fluoro-4-methanesulfonyl-phenyl)-{6-[4-(3-isopropyl-[1,2,4]oxadiazol-5-yl)-piperidin-1-yl]-5-nitro-pyrimidin-4-yl}-amine (see, compound B111 in WO 2004/065380) and a DPP-IV inhibitor acutely increased plasma GLP-1 levels and improved glucose tolerance to a significantly greater degree than either agent alone.

### G. Glucose-dependent Insulinotropic Polypeptide (GIP)

Glucose-dependent insulinotropic polypeptide (GIP, also known as gastric inhibitory polypeptide) is a peptide incretin hormone of 42 amino acids that is released from duodenal endocrine K cells after meal ingestion. The amount of GIP released is largely dependent on the amount of glucose consumed. GIP has been shown to stimulate glucose-dependent insulin secretion in pancreatic beta cells. GIP mediates its actions through a specific G protein-coupled receptor, namely GIPR.

As GIP contains an alanine at position 2, it is an excellent substrate for dipeptidyl peptidase-4 (DPP-IV), an enzyme regulating the degradation of GIP. Full-length GIP(1-42) is rapidly converted to bioinactive GIP(3-42) within minutes of secretion from the gut K cell.

Inhibition of DPP-IV has been shown to augment GIP bioactivity. (See, e.g., Drucker, Cell Metab (2006) 3:153-165; McIntosh et al., Regul Pept (2005) 128:159-165; Deacon, Regul Pept (2005) 128:117-124; and Ahren et al., Endocrinology (2005) 146:2055-2059.). Analysis of full length bioactive GIP, for example in blood, can be carried out using N-terminal-specific assays (see, e.g., Deacon et al, J Clin Endocrinol Metab (2000) 85:3575-3581).

Recently, GIP has been shown to promote bone formation. GIP has been shown to activate osteoblastic receptors, resulting in increases in collagen type I synthesis and alkaline phosphatase activity, both associated with bone formation. GIP has been shown to inhibit osteoclast activity and differentiation in vitro. GIP administration has been shown to prevent the bone loss due to ovariectomy. GIP receptor (GIPR) knockout mice evidence a decreased bone size, lower bone mass, altered bone microarchitecture and biochemical properties, and altered parameters for bone turnover, especially in bone formation. (See, e.g., Thong et al, Am J Physiol Endocrinol Metab (2007) 292:E543-E548; Bollag et al., Endocrinology (2000) 141:1228-1235; Bollag et al., Mol Cell Endocrinol (2001) 177:35-41; Xie et al., Bone (2005) 37:759-769; and Tsukiyama et al., Mol Endocrinol (2006) 20:1644-1651.)

The usefulness of GIP for maintaining or increasing bone density or formation has been acknowledged by the United State Trademark and Patent Office by issuance of U.S. Pat. No. 6,410,508 for the treatment of reduced bone mineralization by administration of GIP peptide. However, current GIP peptide agonists suffer from a lack of oral bioavailability, negatively impacting patient compliance. An attractive alternative approach is to develop an orally active composition for increasing an endogenous level of GIP activity.

### H. Glucagon-Like Peptide-1 (GLP-1)

Glucagon-like peptide-1 (GLP-1) is an incretin hormone derived from the posttranslaltional modification of proglucagon and secreted by gut endocrine cells. GLP-1 mediates its actions through a specific G protein-coupled receptor (GPCR), namely GLP-1R. GLP-1 is best characterized as a hormone that regulates glucose homeostasis. GLP-1 has been shown to stimulate glucose-dependent insulin secretion and to increase pancreatic beta cell mass. GLP-1 has also been shown to reduce the rate of gastric emptying and to promote satiety. The efficacy of GLP-1 peptide agonists in controlling blood glucose in Type 2 diabetics has been demonstrated in several clinical studies [see, e.g., Nauck et al., Drug News Perspect (2003) 16:413-422], as has its efficacy in reducing body mass [Zander et al., Lancet (2002) 359:824-830].

GLP-1 receptor agonists are additionally useful in protecting against myocardial infarction and against cognitive and neurodegenerative disorders. GLP-1 has been shown to be cardioprotective in a rat model of myocardial infarction [Bose et al., Diabetes (2005) 54:146-151], and GLP-1R has been shown in rodent models to be involved in learning and neuroprotection [During et al., Nat. Med. (2003) 9:1173-1179; and Greig et al., Ann N Y Acad Sci (2004) 1035:290-315].

Certain disorders such as Type 2 diabetes are characterized by a deficiency in GLP-1 [see, e.g., Nauck et al., Diabetes (2004) 53 Suppl 3:S190-196].

Current GLP-1 peptide agonists suffer from a lack of oral bioavailability, negatively impacting patient compliance. Efforts to develop orally bioavailable non-peptidergic, small-molecule agonists of GLP-1R have so far been unsuccessful [Mentlein, Expert Opin Investig Drugs (2005) 14:57-64]. An attractive alternative approach is to develop an orally active composition for increasing an endogenous level of GLP-1 in the blood.

### I. Peptide YY (PYY)

Peptide YY (PYY) is a 36 amino acid peptide originally isolated in 1980 from porcine intestine (Tatemoto et al, Nature (1980) 285:417-418). PYY is secreted from enteroendocrine L-cells within both the large and small intestine. It has been shown that in rat and human gut concentrations of immunoreactive PYY are low in duodenum and jejunum, high in ileum and colon, and highest in rectum (Lundberg et al, PNAS USA (1982) 79:4471-4475; Adrian et al, Gastroenterol. (1985) 89:1070-1077; Ekblad et al, Peptides (2002) 23:251-261; Ueno et al, Regul Pept (2008) 145:12-16). (PYY expression in rat been reported to also extend to alpha cells of the islets of Langerhans and to cells in the medulla oblongata (Ekblad et al, Peptides (2002) 23:251-261; PYY is released into the circulation as PYY₁₋₃₆ and PYY₃₋₃₆ (Eberlein et al, Peptides (1989) 10:797-803). PYY₃₋₃₆ is generated from PYY₁₋₃₆ by cleavage of the N-terminal Tyr and Pro residues by dipeptidyl peptidase IV. PYY₃₋₃₆ is the predominant form of PYY in human postprandial plasma (Grandt et al, Regul. Pept. (1994) 51:151-159). PYY₁₋₃₆ and PYY₃₋₃₆ have been reported to have comparable agonist activity at NPY Y2 receptor (Y2R), a G protein-coupled receptor (Parker et al, Br. J. Pharmacol. (2008) 153:420-431); however, PYY₃₋₃₆ has been reported to be a high-affinity Y2R selective agonist (Keire et al, Am. J. Physiol. Gastrointest. Liver Physiol. (2000) 279:G126-G131). PYY was subsequently reported to reduce high-fat food intake in rats after peripheral administration (Okada et al, Endocrinology Supplement (1993) 180) and to cause weight loss in mice after peripheral administration (Morley et al, Life Sciences (1987) 41:2157-2165).

Peripheral administration of PYY₃₋₃₆ has been reported to markedly reduce food intake and weight gain in rats, to decrease appetite and food intake in humans, and to decrease food intake in mice, but not in Y2R-null mice, which was said to suggest that the food intake effect requires the Y2R. In human studies, infusion of PYY₃₋₃₆ was found to significantly decrease appetite and reduce food intake by 33% over 24 hours. Infusion of PYY₃₋₃₆ to reach the normal postprandial circulatory concentrations of the peptide led to peak serum levels of PYY₃₋₃₆ within 15 minutes, followed by a rapid decline to basal levels within 30 minutes. It was reported that there was significant inhibition of food intake in the 12-hour period following the PYY₃₋₃₆ infusion, but was essentially no effect on food intake in the 12-hour to 24-hour period. In a rat study, repeated administration of PYY₃₋₃₆ intraperitoneally (injections twice daily for 7 days) reduced cumulative food intake (Batterham et al, Nature (2002) 418:650-654; Renshaw et al, Current Drug Targets (2005) 6:171-179).

Peripheral administration of PYY₃₋₃₆ has been reported to reduce food intake, body weight gain and glycemic indices in diverse rodent models of metabolic diseases of both sexes (Pittner et al, Int. J. Obes. Relat. Metab. Disord. (2004) 28:963-971). It has been reported that blockade of Y2R with the specific antagonist BIIE-246 attenuates the effect of peripherally administered endogenous and exogenous PYY₃₋₃₆ for reducing food intake (Abbott et al, Brain Res (2005) 1043:139-144). It has been reported that peripheral administration of a novel long-acting selective Y2R polyethylene glycol-conjugated peptide agonist reduces food intake and improves glucose metabolism (glucose disposal, plasma insulin and plasma glucose) in rodents (Ortiz et al, JPET (2007) 323:692-700; Lamb et al, J. Med. Chem. (2007) 50:2264-2268). It has been reported that PYY ablation in mice leads to the development of hyperinsulinemia and obesity (Boey et al, Diabetologia (2006) 49:1360-1370). It has been reported that peripheral administration of a long-acting, potent and highly selective Y2R agonist inhibits food intake and promotes fat metabolism in mice (Balasubramaniam et al, Peptides (2007) 28:235-240).

There is evidence that agents which stimulate PYY synthesis in vivo can confer protection against diet-induced and genetic obesity and can improve glucose tolerance (Boey et al, Neuropeptides (2008) 42:19-30).

It has been reported that Y2R agonists such as PYY₁₋₃₆ and PYY₃₋₃₆ can confer protection against epileptic seizures, such as against kainate seizures (El Bahh et al, Eur. J. Neurosci. (2005) 22:1417-1430; Woldbye et al, Neurobiology of Disease (2005) 20:760-772).

It has been reported that Y2R agonists such as PYY₁₋₃₆ and PYY₃₋₃₆ act as proabsorbtive (or anti-secretory) hormones, increasing upon intravenous administration the absorption of both water and sodium in various parts of the bowel (Bilchik et al, Gastroenterol. (1993) 105:1441-1448; Liu et al, J. Surg. Res. (1995) 58:6-11; Nightingale et al, Gut (1996) 39:267-272; Liu et al, Am Surg (1996) 62:232-236; Balasubramaniam et al, J. Med. Chem. (2000) 43:3420-3427). It has been reported that Y2R agonists such as PYY analogues inhibit secretion and promote absorption and growth in the intestinal epithelium (Balasubramaniam et al, J. Med. Chem. (2000) 43:3420-3427). It has been reported that PYY promotes intestinal growth in normal rats (Gomez et al, Am. J. Physiol. (1995) 268:G71-G81). It has been reported that Y2R agonists such as PYY₁₋₃₆ and PYY₃₋₃₆ inhibit bowel motility and work to prevent diarrhea (EP1902730; also see Cox, Peptides (2007) 28:345-351).

It has been reported that Y2R agonists such as PYY₁₋₃₆ and PYY₃₋₃₆ can confer protection against inflammatory bowel disease such as ulcerative colitis and Crohn's disease (WO 03/105763). It has been reported that PYY-deficient mice exhibit an osteopenic phenotype, i.e. that PYY can increase bone mass and/or can confer protection against loss of bone mass (e.g., decreases loss of bone mass) (Wortley et al, Gastroenterol. (2007) 133:1534-1543). It has been reported that PYY₃₋₃₆ can confer protection in rodent models of pancreatitis (Vona-Davis et al, Peptides (2007) 28:334-338).

It has been reported that angiogenesis is impaired in Y2R-deficient mice (Lee et al, Peptides (2003) 24:99-106), i.e. that agonists of Y2R such as PYY₁₋₃₆ and PYY₃₋₃₆ promote angiogenesis. It has been reported that would healing is impaired in Y2R-deficient mice (Ekstrand et al, PNAS USA (2003) 100:6033-6038), i.e. that agonists of Y2R such as PYY₁₋₃₆ and PYY₃₋₃₆ promote wound healing. It has been reported that ischemic angiogenesis is impaired in Y2R-deficient mice (Lee et al, J. Clin. Invest. (2003) 111:1853-1862), i.e. that agonists of Y2R such as PYY₁₋₃₆ and PYY₃₋₃₆ promotes revascularization and restoration of function of ischemic tissue. It has been reported that agonists of Y2R such as PYY₁₋₃₆ and PYY₃₋₃₆ mediate increases in collateral-dependent blood flow in a rat model of peripheral arterial disease (Cruze et al, Peptides (2007) 28:269-280).

It has been reported that PYY and Y2R agonists such as PYY₃₋₃₆ can suppress tumor growth in the cases of, e.g., pancreatic cancer such as pancreatic ductal adenocarcinoma, breast cancer such as breast infiltrative ductal adenocarcinoma, colon cancer such as colon adenocarcinoma and Barrett's adenocarcinoma (Liu et al, Surgery (1995) 118:229-236; Liu et al, J. Surg. Res. (1995) 58:707-712; Grise et al, J. Surg. Res. (1999) 82:151-155; Tseng et al, Peptides (2002) 23:389-395; McFadden et al, Am. J. Surg. (2004) 188:516-519).

It has been reported that stimulation of Y2R such as by PYY₃₋₃₆ leads to an increase in plasma adiponectin (Ortiz et al, JPET (2007) 323:692-700). Adiponectin is an adipokine with potent anti-inflammatory properties (Ouchi et al, Clin Chim Acta (2007) 380:24-30; Tilg et al, Nat. Rev. Immunol. (2006) 6:772-783). Adiponectin exerts anti-atherogenic effects by targeting vascular endothelial cells and macrophages and insulin-sensitizing effects, predominantly in muscle and liver (Kubota et al, J. Biol. Chem. (2002) 277:25863-25866; Maeda et al, Nat. Med. (2002) 8:731-737). Low adiponectin levels have been reported to be associated with atherogenic lipoproteins in dyslipidemia (elevated triglycerides, small dense LDL cholesterol, low HDL cholesterol) (Marso et al, Diabetes Care (2008) February 5 Epub ahead of print). Adiponectin has been implicated in high density lipoprotein (HDL) assembly (Oku et al, FEBS Letters (2007) 581:5029-5033). Adiponectin has been found to ameliorate the abnormalities of metabolic syndrome, including insulin resistance, hyperglycemia, and dyslipidemia, in a mouse model of obesity-linked metabolic syndrome associated with decreased adiponectin levels (Hara et al, Diabetes Care (2006) 29:1357-1362). Adiponectin has been reported to stimulate angiogenesis in response to tissue ischemia (Shibata et al, J. Biol. Chem. (2004) 279:28670-28674). Adiponectin has been reported to prevent cerebral ischemic injury through endothelial nitric oxide synthase-dependent mechanisms (Nishimura et al, Circulation (2008) 117:216-223). Adiponectin has been reported to confer protection against myocardial ischemia-reperfusion injury (Shibata et al, Nat Med (2005) 11:1096-1103; Tao et al, Circulation (2007) 115:1408-1416). Adiponectin has been reported to confer protection against myocardial ischemia-reperfusion injury via AMP-activated protein kinase, Akt, and nitric oxide (Gonon et al, Cardiovasc Res. (2008) 78:116-122). Adiponectin has been reported to confer protection against the development of systolic dysfunction following myocardial infarction, through its abilities to suppress cardiac hypertrophy and interstitial fibrosis, and protect against myocyte and capillary loss (Shibata et al, J. Mol. Cell. Cardiol. (2007) 42:1065-1074). Adiponectin has been reported to confer protection against inflammatory lung disease; adiponectin-deficient mice exhibit an emphysema-like phenotype (Summer et al, Am J. Physiol. Lung Cell Mol. Physiol. (Mar. 7, 2008)). Adiponectin has been reported to confer protection against allergic airway inflammation and airway hyperresponsiveness such as may be associated with asthma (Shore et al, J. Allergy Clin. Immunol (2006) 118:389-395). Adiponectin has been suggested to confer protection against pulmonary arterial hypertension by virtue of its insulin-sensitizing effects (Hansmann et al, Circulation (2007) 115:1275-1284). Adiponectin has been reported to ameliorate obesity-related hypertension, with said amelioration of hypertension being associated in part with upregulated prostacyclin expression (Ohashi et al, Hypertension (2006) 47:1108-1116). Adiponectin has been reported to decrease tumor necrosis factor (TNF)-α-induced expression of the adhesion molecules VCAM-1, E-selectin and ICAM-1 in human aortic endothelial cells (HAECs) (Ouchi et al, Circulation (1999) 100:2473-2476) and to inhibit production of TNF-α in macrophages (Yokota et al, Blood (2000) 96:1723-1732). Adiponectin has been reported to confer protection against restenosis after vascular intervention (Matsuda et al, J Biol Chem (2002) 277:37487-37491). The central role of TNF-α in inflammation has been demonstrated by the ability of agents that block the action of TNF-α to treat a range of inflammatory conditions. TNF-α-mediated inflammatory conditions encompass rheumatoid arthritis, inflammatory bowel disease such as Crohn's disease, ankylosing spondylitis, psoriasis, ischemic brain injury, cardiac allograft rejection, asthma, and the like (Bradley, J Pathol (2008) 214:149-160). See, e.g., Yamamoto et al, Clinical Science (2002) 103:137-142; Behre, Scand J Clin Lab Invest (2007) 67:449-458; Guerre-Millo, Diabetes & Metabolism (2008) 34:12-18; Parker et al, Br. J. Pharmacol. (2008) 153:420-431.

### SUMMARY OF THE INVENTION

One aspect of the present invention is directed to compounds, as described herein, and pharmaceutically acceptable salts, solvates, and hydrates thereof, which bind to and modulate the activity of a GPCR, referred to herein as GPR119, and uses thereof.

One aspect of the present invention encompasses, inter alia, certain heterocyclyl derivatives selected from compounds of Formula I and pharmaceutically acceptable salts, solvates, and hydrates thereof:

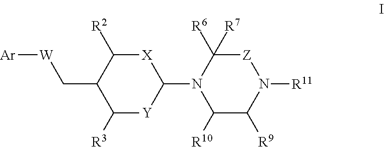

wherein:

Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl;

W is selected from: O and NR¹; or W is absent;

R¹ is selected from: H and C₁-C₆ alkyl;

R² and R³ are each H; or R² and R³ together form —CH₂—CH₂—;

X is selected from: —CH₂—CH₂— and CHR⁴; or X is absent;

Y is CHR⁵; or Y is absent;

R⁴ and R⁵ are each H; or R⁴ and R⁵ together form —CH₂—CH₂—;

Z is selected from: —CH₂—CH₂— and CHR⁸;

R⁶, R⁹ and R¹⁰ are each H, and R⁷ and R⁸ are each independently selected from: H and C₁-C₆ alkyl; or R⁸, R⁹ and R¹⁰ are each H, and R⁶ and R⁷ together form —CH₂—CH₂—; or R⁶, R⁸ and R⁹ are each H, and R⁷ and R¹⁰ together form —CH₂—CH₂; or R⁶, R⁷ and R¹⁰ are each H, and R⁸ and R⁹ together form —CH₂—CH₂; or R⁶, R⁷ and R⁹ are each H, and R⁸ and R¹⁰ together form —CH₂—CH₂—; and

R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

One aspect of the present invention pertains to compounds of Formula Ia and pharmaceutically acceptable salts, solvates, and hydrates thereof:

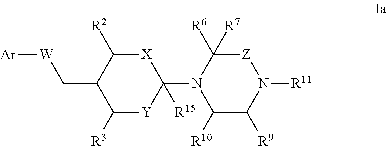

wherein:

Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl;

W is selected from: O and NR¹; or W is absent;

R¹ is selected from: H and C₁-C₆ alkyl;

R² and R³ are each H; or R² and R³ together form —CH₂—CH₂—;

X is selected from: —CH₂—CH₂—, CHR⁴, C(O), CHF, and CF₂; or X is absent;

Y is CHR⁵; or Y is absent;

R⁴ and R⁵ are each H; or R⁴ and R⁵ together form —CH₂—CH₂—;

Z is selected from: —CH₂—CH₂—, CHR⁸, and C(O);

R⁶, R⁹ and R¹⁰ are each H, and R⁷ and R⁸ are each independently selected from: H and C₁-C₆ alkyl; or R⁸, R⁹ and R¹⁰ are each H, and R⁶ and R⁷ together form —CH₂—CH₂—; or R⁶, R⁸ and R⁹ are each H, and R⁷ and R¹⁰ together form —CH₂—CH₂, or a bond; or R⁶, R⁷ and R¹⁰ are each H, and R⁸ and R⁹ together form —CH₂—CH₂; or R⁶, R⁷ and R⁹ are each H, and R⁸ and R¹⁰ together form —CH₂—CH₂—; or R⁶, R⁷ and R⁸ are each H, and R⁹ and R¹⁰ together form —CH₂—;

R¹¹ is selected from: C₁-C₆ alkoxycarbonyl, C₁-C₆ alkylsulfonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, heteroaryl-C₁-C₆ alkylene, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen; and

R¹⁵ is selected from: H and cyano.

One aspect of the present invention pertains to compositions comprising a compound of the present invention.

One aspect of the present invention pertains to compositions comprising a compound of the present invention and a pharmaceutically acceptable carrier.

One aspect of the present invention pertains to methods for preparing a composition comprising the step of admixing a compound of the present invention and a pharmaceutically acceptable carrier.

One aspect of the present invention pertains to compositions obtained by a method of the present invention.

One aspect of the present invention pertains to pharmaceutical products selected from: a pharmaceutical composition, a formulation, a dosage form, a combined preparation, a twin pack, and a kit; comprising a compound of the present invention.

One aspect of the present invention pertains to compositions comprising: a first pharmaceutical agent selected from a compound of the present invention; and a second pharmaceutical agent.

One aspect of the present invention pertains to compositions comprising a compound of the present invention and a second pharmaceutical agent.

One aspect of the present invention pertains to methods for preparing a composition comprising the step of admixing a compound of the present invention and a second pharmaceutical agent.

One aspect of the present invention pertains to pharmaceutical product selected from: a pharmaceutical composition, a formulation, a dosage form, a combined preparation, a twin pack, and a kit; comprising a compound of the present invention and a second pharmaceutical agent.

One aspect of the present invention pertains to methods for preparing a composition comprising: a first pharmaceutical agent selected from a compound of the present invention; and a second pharmaceutical agent.

One aspect of the present invention pertains to compositions comprising: a first pharmaceutical agent selected from a compound of the present invention; a second pharmaceutical agent; and a pharmaceutically acceptable carrier.

One aspect of the present invention pertains to methods for preparing a composition comprising the step of admixing a first pharmaceutical agent selected from a compound of the present invention; a second pharmaceutical agent; and a pharmaceutically acceptable carrier.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention; and a second pharmaceutical agent.

One aspect of the present invention pertains to methods for modulating the activity of a GPR119 receptor, comprising administering to an individual in need thereof, a therapeutically effective amount of: a compound, a composition, or a pharmaceutical product of the present invention.

One aspect of the present invention pertains to methods for modulating the activity of a GPR119 receptor, comprising prescribing to an individual in need thereof, a therapeutically effective amount of: a compound, a composition, or a pharmaceutical product of the present invention.

One aspect of the present invention pertains to methods for agonizing a GPR119 receptor, comprising administering to an individual in need thereof, a therapeutically effective amount of: a compound, a composition, or a pharmaceutical product of the present invention.

One aspect of the present invention pertains to methods for agonizing a GPR119 receptor, comprising prescribing to an individual in need thereof, a therapeutically effective amount of: a compound, a composition, or a pharmaceutical product of the present invention.

One aspect of the present invention pertains to methods for increasing the secretion of an incretin in an individual, comprising administering to the individual in need thereof, a therapeutically effective amount of: a compound, a composition, or a pharmaceutical product of the present invention.

One aspect of the present invention pertains to methods for increasing the secretion of an incretin in an individual, comprising prescribing to the individual in need thereof, a therapeutically effective amount of: a compound, a composition, or a pharmaceutical product of the present invention.

One aspect of the present invention pertains to methods for increasing a blood incretin level in an individual, comprising administering to the individual in need thereof, a therapeutically effective amount of: a compound, a composition, or a pharmaceutical product of the present invention.

One aspect of the present invention pertains to methods for increasing a blood incretin level in an individual, comprising prescribing to the individual in need thereof, a therapeutically effective amount of: a compound, a composition, or a pharmaceutical product of the present invention.

One aspect of the present invention pertains to methods increasing the secretion of an incretin in an individual, or for increasing a blood incretin level in an individual, comprising administering to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual; comprising administering to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual, comprising prescribing to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity; in an individual; comprising administering to the individual in need thereof, a therapeutically effective amount of: a compound, a composition, or a pharmaceutical product of the present invention.

One aspect of the present invention pertains to uses of a compound, a composition, or a pharmaceutical product of the present invention; in the manufacture of a medicament for modulating the activity of a GPR119 receptor in an individual.

One aspect of the present invention pertains to uses of a compound, a composition, or a pharmaceutical product of the present invention; in the manufacture of a medicament for agonizing a GPR119 receptor in an individual.

One aspect of the present invention pertains to uses of a compound, a composition, or a pharmaceutical product of the present invention; in the manufacture of a medicament for increasing the secretion of an incretin in an individual.

One aspect of the present invention pertains to uses of a compound, a composition, or a pharmaceutical product of the present invention; in the manufacture of a medicament for increasing a blood incretin level in an individual.

One aspect of the present invention pertains to uses of a compound, a composition, or a pharmaceutical product of the present invention; in the manufacture of a medicament for increasing the secretion of an incretin in an individual, or for increasing a blood incretin level in an individual.

One aspect of the present invention pertains to uses of a compound, a composition, or a pharmaceutical product of the present invention; in the manufacture of a medicament for treating a disorder in an individual, wherein the disorder is selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity.

One aspect of the present invention pertains to uses of a compound, a composition, or a pharmaceutical product of the present invention; in the manufacture of a medicament for the treating a disorder in an individual, wherein the disorder is selected from: a GPR119-receptor-related disorder; a condition ameliorated by increasing a blood incretin level; a condition characterized by low bone mass; a neurological disorder; a metabolic-related disorder; and obesity.

One aspect of the present invention pertains to compounds, compositions, and pharmaceutical products of the present invention for use in a method of treatment of the human or animal body by therapy.

One aspect of the present invention pertains to compounds, compositions, and pharmaceutical products of the present invention for use in a method of modulating the activity of a GPR119 receptor in an individual.

One aspect of the present invention pertains to compounds, compositions, and pharmaceutical products of the present invention for use in a method of agonizing a GPR119 receptor in an individual.

One aspect of the present invention pertains to compounds, compositions, and pharmaceutical products of the present invention for use in a method of increasing the secretion of an incretin in an individual.

One aspect of the present invention pertains to compounds, compositions, and pharmaceutical products of the present invention for use in a method of increasing a blood incretin level in an individual.

One aspect of the present invention pertains to compounds, compositions, and pharmaceutical products of the present invention for use in a method of increasing the secretion of an incretin in an individual or increasing a blood incretin level in an individual.

One aspect of the present invention pertains to compounds, compositions, and pharmaceutical products of the present invention for use in a method of treating a disorder in an individual, wherein the disorder is selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity.

One aspect of the present invention pertains to compounds, compositions, and pharmaceutical products of the present invention for use in a method of treating a disorder in an individual, wherein the disorder is selected from: a GPR119-receptor-related disorder; a condition ameliorated by increasing a blood incretin level; a condition characterized by low bone mass; a neurological disorder; a metabolic-related disorder; and obesity.

One aspect of the present invention pertains to pharmaceutical products selected from: a pharmaceutical composition, a formulation, a dosage form, a combined preparation, a twin pack, and a kit; comprising a compound of the present invention; for use in a method of treatment of the human or animal by therapy.

One aspect of the present invention pertains to pharmaceutical products selected from: a pharmaceutical composition, a formulation, a dosage form, a combined preparation, a twin pack, and a kit; comprising a compound of the present invention; for modulating the activity of a GPR119 receptor in an individual.

One aspect of the present invention pertains to pharmaceutical products selected from: a pharmaceutical composition, a formulation, a dosage form, a combined preparation, a twin pack, and a kit; comprising a compound of the present invention; for agonizing a GPR119 receptor in an individual.

One aspect of the present invention pertains to pharmaceutical products selected from: a pharmaceutical composition, a formulation, a dosage form, a combined preparation, a twin pack, and a kit; comprising a compound of the present invention; for increasing the secretion of an incretin in an individual.

One aspect of the present invention pertains to pharmaceutical products selected from: a pharmaceutical composition, a formulation, a dosage form, a combined preparation, a twin pack, and a kit; comprising a compound of the present invention; for increasing a blood incretin level in an individual.

One aspect of the present invention pertains to pharmaceutical products selected from: a pharmaceutical composition, a formulation, a dosage form, a combined preparation, a twin pack, and a kit; comprising a compound of the present invention for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual.

One aspect of the present invention pertains to methods for modulating the activity of a GPR119 receptor, comprising prescribing to an individual in need thereof, a therapeutically effective amount of a first pharmaceutical agent selected from a compound of the present invention, in combination with a therapeutically effective amount of a second pharmaceutical agent.

One aspect of the present invention pertains to methods for agonizing a GPR119 receptor, comprising prescribing to an individual in need thereof, a therapeutically effective amount of a first pharmaceutical agent selected from a compound of the present invention, in combination with a therapeutically effective amount of a second pharmaceutical agent.

One aspect of the present invention pertains to methods for increasing the secretion of an incretin in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for increasing a blood incretin level in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for modulating the activity of a GPR119 receptor, comprising administering to an individual in need thereof, a therapeutically effective amount of a first pharmaceutical agent selected from a compound of the present invention, in combination with a therapeutically effective amount of a second pharmaceutical agent.

One aspect of the present invention pertains to methods for agonizing a GPR119 receptor, comprising administering to an individual in need thereof, a therapeutically effective amount of a first pharmaceutical agent selected from a compound of the present invention, in combination with a therapeutically effective amount of a second pharmaceutical agent.

One aspect of the present invention pertains to methods for increasing the secretion of an incretin in an individual, comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to methods for increasing a blood incretin level in an individual, comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to methods for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual; comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to uses of a first pharmaceutical agent selected from a compound of the present invention, in combination with a second pharmaceutical agent in the manufacture of a medicament for modulating the activity of a GPR119 receptor in an individual.

One aspect of the present invention pertains to uses of a first pharmaceutical agent in combination with a second pharmaceutical agent selected from a compound of the present invention, in the manufacture of a medicament for modulating the activity of a GPR119 receptor in an individual.

One aspect of the present invention pertains to uses of a first pharmaceutical agent selected from a compound of the present invention, in combination with a second pharmaceutical agent in the manufacture of a medicament for agonizing a GPR119 receptor in an individual.

One aspect of the present invention pertains to uses of a first pharmaceutical agent in combination with a second pharmaceutical agent selected from a compound of the present invention, in the manufacture of a medicament for agonizing a GPR119 receptor in an individual.

One aspect of the present invention pertains to uses of a first pharmaceutical agent selected from a compound of the present invention, in combination with a second pharmaceutical agent in the manufacture of a medicament for increasing the secretion of an incretin in an individual.

One aspect of the present invention pertains to uses of a first pharmaceutical agent in combination with a second pharmaceutical agent selected from a compound of the present invention, in the manufacture of a medicament for increasing the secretion of an incretin in an individual.

One aspect of the present invention pertains to uses of a first pharmaceutical agent selected from a compound of the present invention, in combination with a second pharmaceutical agent in the manufacture of a medicament for increasing a blood incretin level in an individual.

One aspect of the present invention pertains to uses of a first pharmaceutical agent in combination with a second pharmaceutical agent selected from a compound of the present invention, in the manufacture of a medicament for increasing a blood incretin level in an individual.

One aspect of the present invention pertains to uses of a first pharmaceutical agent selected from a compound of the present invention, in combination with a second pharmaceutical agent, in the manufacture of a medicament for the treatment of a disorder selected from: a GPR119-receptor-related disorder; a condition ameliorated by increasing a blood incretin level; a condition characterized by low bone mass; a neurological disorder; a metabolic-related disorder; and obesity.

One aspect of the present invention pertains to uses of a first pharmaceutical agent in combination with a second pharmaceutical agent selected from a compound of the present invention, in the manufacture of a medicament for the treatment of a disorder selected from: a GPR119-receptor-related disorder; a condition ameliorated by increasing a blood incretin level; a condition characterized by low bone mass; a neurological disorder; a metabolic-related disorder; and obesity.

One aspect of the present invention pertains to uses of a pharmaceutical agent in combination with a compound of the present invention, in the manufacture of a medicament for increasing the secretion of an incretin in an individual, or for increasing a blood incretin level in an individual.

One aspect of the present invention pertains to uses of a pharmaceutical agent in combination with a compound of the present invention, in the manufacture of a medicament for the treatment of a disorder selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity.

One aspect of the present invention pertains to first pharmaceutical agents selected from a compound according of the present invention, for use in combination with a second pharmaceutical agent for use in a method of treatment of the human or animal body by therapy.

One aspect of the present invention pertains to first pharmaceutical agents selected from a compound according of the present invention, for use in combination with a second pharmaceutical agent for use in a method of modulating the activity of a GPR119 receptor in an individual.

One aspect of the present invention pertains to first pharmaceutical agents selected from a compound according of the present invention, for use in combination with a second pharmaceutical agent for use in a method of agonizing a GPR119 receptor in an individual.

One aspect of the present invention pertains to first pharmaceutical agents selected from a compound according of the present invention, for use in combination with a second pharmaceutical agent for use in a method of increasing the secretion of an incretin in an individual.

One aspect of the present invention pertains to first pharmaceutical agents selected from a compound according of the present invention, for use in combination with a second pharmaceutical agent for use in a method of increasing a blood incretin level in an individual.

One aspect of the present invention pertains to first pharmaceutical agents selected from a compound according of the present invention, for use in combination with a second pharmaceutical agent for use in a method of treatment of a disorder selected from: a GPR119-receptor-related disorder; a condition ameliorated by increasing a blood incretin level; a condition characterized by low bone mass; a neurological disorder; a metabolic-related disorder; and obesity; in an individual.

One aspect of the present invention pertains to first pharmaceutical agents for use in combination with a second pharmaceutical agent selected from a compound of the present invention, for use in a method of treatment of the human or animal body by therapy.

One aspect of the present invention pertains to first pharmaceutical agents for use in combination with a second pharmaceutical agent selected from a compound of the present invention, for use in a method of modulating the activity of a GPR119 receptor in an individual.

One aspect of the present invention pertains to first pharmaceutical agents for use in combination with a second pharmaceutical agent selected from a compound of the present invention, for use in a method of agonizing a GPR119 receptor in an individual.

One aspect of the present invention pertains to first pharmaceutical agents for use in combination with a second pharmaceutical agent selected from a compound of the present invention, for use in a method of increasing the secretion of an incretin in an individual.

One aspect of the present invention pertains to first pharmaceutical agents for use in combination with a second pharmaceutical agent selected from a compound of the present invention, for use in a method of increasing a blood incretin level in an individual.

One aspect of the present invention pertains to first pharmaceutical agents for use in combination with a second pharmaceutical agent selected from a compound of the present invention, for use in a method of treatment of a disorder selected from: a GPR119-receptor-related disorder; a condition ameliorated by increasing a blood incretin level; a condition characterized by low bone mass; a neurological disorder; a metabolic-related disorder; and obesity; in an individual.

One aspect of the present invention pertains to pharmaceutical agents for use in combination with a compound or a composition of the present invention in a method of treating the human or animal by therapy.

One aspect of the present invention pertains to pharmaceutical agents for use in combination with a compound or a composition of the present invention for increasing the secretion of an incretin in an individual, or for increasing a blood incretin level in an individual.

One aspect of the present invention pertains to pharmaceutical agents for use in combination with a compound or a composition of the present invention for the treatment of a disorder selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity; in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of treatment of the human or animal body by therapy.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of modulating the activity of a GPR119 receptor in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of agonizing a GPR119 receptor in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of increasing the secretion of an incretin in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of increasing a blood incretin level in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of treatment of a disorder selected from: a GPR119-receptor-related disorder; a condition ameliorated by increasing a blood incretin level; a condition characterized by low bone mass; a neurological disorder; a metabolic-related disorder; and obesity; in an individual.

One aspect of the present invention pertains to methods for preparing a pharmaceutical product of the present invention comprising: mixing the compound with a first pharmaceutically acceptable carrier to prepare a compound dosage form, mixing the second pharmaceutical agent with a second pharmaceutically acceptable carrier to prepare a second pharmaceutical agent dosage form, and providing the compound dosage form and the second pharmaceutical agent dosage form in a combined dosage form for simultaneous, separate, or sequential use.

These and other aspects of the invention disclosed herein will be set forth in greater detail as the patent disclosure proceeds.

### BRIEF DESCRIPTION OF THE DRAWINGS

FIG. 1 shows the effects of Compound 17 on glucose excursion in male 129SVE mice.

FIG. 2 shows the effects of Compound 17 on glucose excursion reduction in male 129SVE mice.

FIG. 3 shows the effects of Compound 17 on GIP release in male 129SVE mice.

FIG. 4 shows a general synthetic method for the preparation of compounds of Formula I, wherein W is O. FIG. 4 also shows a general synthetic method for the preparation of compounds of Formula I, wherein W is O, and R¹¹ is C₁-C₆ alkoxycarbonyl, C₁-C₆ alkylthiocarbonyl, C₃-C₇ cycloalkoxycarbonyl, C₃-C₇ cycloalkylcarbonyl, C₃-C₇ cycloalkythiocarbonyl, or heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

FIG. 5 shows a general synthetic method for the preparation of compounds of Formula I, wherein W is O. FIG. 5 also shows a general synthetic method for the preparation of compounds of Formula I, wherein W is O, and R¹¹ is optionally substituted 1,2,4-oxadiazol-5-yl. FIG. 5 also shows a general synthetic method for the preparation of compounds of Formula I, wherein W is O, and R¹¹ is optionally substituted 1,2,4-oxadiazol-3-yl.

FIG. 6 shows general synthetic methods for the preparation of compounds of Formula I, wherein W is O. FIG. 6 also shows general synthetic methods for the preparation of compounds of Formula I, wherein W is O, and Ar is substituted with C₁-C₆ alkylsulfonyl, or cyano.

FIG. 7 shows a general synthetic method for the preparation of compounds of Formula I, wherein W is O.

FIG. 8 shows a general synthetic methods for the preparation of compounds of Formula I, wherein W is O, and R¹¹ is C₃-C₇ cycloalkylalkyl or C₃-C₇ cycloalkylcarbonyl, each optionally substituted with one or more substituents selected from: C₁-C₆ alkoxy, C₁-C₆ alkyl, C₃-C₇ cycloalkyl, C₁-C₆ haloalkyl, and halogen. FIG. 8 also shows a general synthetic method for the preparation of compounds of Formula I, wherein W is NR¹.

FIG. 9 shows general synthetic methods for the preparation of compounds of Formula I wherein W is O and R¹¹ is Boc. FIG. 9 also shows general synthetic methods for the preparation of compounds of Formula I, wherein, R¹¹ is optionally substituted 1,2,4-oxadiazol-5-ylmethyl.

FIG. 10 shows general synthetic methods for the preparation of compounds of Formula I, wherein Z is CF₂. FIG. 10 also shows general synthetic methods for the preparation of compounds of Formula Ia, wherein R¹⁵ is CN. FIG. 10 also shows general synthetic methods for the preparation of compounds of Formula I, wherein Z is CHF.

FIG. 11 shows general synthetic methods for the preparation of compounds of Formula I wherein W is O and R¹¹ is Boc. FIG. 11 also shows general synthetic methods for the preparation of compounds of Formula I, wherein R⁷ and le° together form a bond. FIG. 11 also shows general synthetic methods for the preparation of compounds of Formula I, wherein R¹¹ is C₁-C₆ alkoxycarbonyl, C₁-C₆ alkylthiocarbonyl, C₃-C₇ cycloalkoxycarbonyl, C₃-C₇ cycloalkylcarbonyl, C₃-C₇ cycloalkythiocarbonyl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

FIG. 12 shows a synthetic method for the preparation of perfluorophenyl 3-(trifluoromethyl)oxetan-3-yl carbonate, an intermediate useful in the synthesis of compounds of Formula I. FIG. 12 also shows general synthetic methods for the preparation of compounds of Formula I, wherein C₁-C₆ alkoxycarbonyl, C₁-C₆ alkylthiocarbonyl, C₃-C₇ cycloalkoxycarbonyl, C₃-C₇ cycloalkylcarbonyl, C₃-C₇ cycloalkythiocarbonyl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen. FIG. 12 also shows general synthetic methods for the preparation of compounds of Formula I, wherein R¹¹ is C₁-C₆ alkylsulfonyl optionally substituted with one or more substituents selected from: C₁-C₆ alkoxy, C₁-C₆ alkyl, C₃-C₇ cycloalkyl, C₁-C₆ haloalkyl, and halogen.

### DETAILED DESCRIPTION OF THE INVENTION

### Definitions

For clarity and consistency, the following definitions will be used throughout this patent document.

The term “agonist” as used herein refers to a moiety that interacts with and activates a G-protein-coupled receptor, for instance a GPR119-receptor, and can thereby initiate a physiological or pharmacological response characteristic of that receptor. For example, an agonist may activate an intracellular response upon binding to a receptor, or enhance GTP binding to a membrane.

The term “antagonist” as used herein refers to a moiety that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

The term “GPR119” as used herein includes the human amino acid sequences found in GeneBank accession number AY288416, and naturally-occurring allelic variants thereof, and mammalian orthologs thereof. A preferred human GPR119 for use in screening and testing of the compounds of the invention is provided in the nucleotide sequence of Seq. ID.No:1 and the corresponding amino acid sequence in Seq. ID.No:2 found in PCT Application No. WO2005/007647.

The term “in need of treatment” and the term “in need thereof” when referring to treatment are used interchangeably and refer to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” refers to any animal, including mammals, preferably mice, rats, other rodents, rabbits, dogs, cats, swine, cattle, sheep, horses, or primates, and most preferably humans.

The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist, or decreases GTP binding to a membrane. Preferably, the baseline intracellular response is inhibited in the presence of the inverse agonist by at least 30%, more preferably by at least 50% and most preferably by at least 75%, as compared with the baseline response in the absence of the inverse agonist.

The term “modulate or modulating” refers to an increase or decrease in the amount, quality, response or effect of a particular activity, function or molecule.

The term “composition” refers to a compound, including but not limited to, salts, solvates, and hydrates of a compound of the present invention, in combination with at least one additional component.

The term “pharmaceutical composition” refers to a composition comprising at least one active ingredient, such as a compound as described herein; including but not limited to, salts, solvates, and hydrates of compounds of the present invention, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “therapeutically effective amount” refers to the amount of active compound or pharmaceutical agent that elicits the biological or medicinal response in a tissue, system, animal, individual or human that is being sought by a researcher, veterinarian, medical doctor or other clinician or caregiver or by an individual, which includes one or more of the following:

(1) preventing the disease, for example, preventing a disease, condition or disorder in an individual that may be predisposed to the disease, condition or disorder but does not yet experience or display the pathology or symptomatology of the disease;

(2) inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) ameliorating the disease, for example, ameliorating a disease, condition or disorder in an individual that is experiencing or displaying the pathology or symptomatology of the disease, condition or disorder (i.e., reversing the pathology and/or symptomatology).

### Chemical Group, Moiety or Radical

The term “C₁-C₆ alkoxy” refers to a radical comprising a C₁-C₆ alkyl group attached to an oxygen atom, wherein C₁-C₆ alkyl has the same definition as found herein. Some embodiments contain 1 to 5 carbons. Some embodiments contain 1 to 4 carbons. Some embodiments contain 1 to 3 carbons. Some embodiments contain 1 or 2 carbons. Examples include, but are not limited to methoxy, ethoxy, n-propoxy, isopropoxy, n-butoxy, tert-butoxy, isobutoxy, and sec-butoxy.

The term “aryl” refers to an aromatic ring radical containing 6 to 10 ring carbons. Examples include, but are not limited to, phenyl and naphthyl.

The term “C₁-C₄ alkylcarboxamide” refers to a radical comprising a C₁-C₄ alkyl group attached to the nitrogen of an amide group, wherein C₁-C₄ alkyl has the same definition as found herein. The C₁-C₆ alkylcarboxamido may be represented by the following:

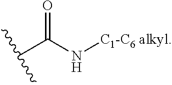

Examples include, but are not limited to, N-methylcarboxamide, N-ethylcarboxamide, N-n-propylcarboxamide, N-isopropylcarboxamide, N-n-butylcarboxamide, N-sec-butylcarboxamide, N-isobutylcarboxamide, N-tert-butylcarboxamide.

The term “C₁-C₆ alkoxycarbonyl” refers to a radical comprising a C₁-C₆ alkoxy group attached to a carbonyl, wherein C₁-C₆ alkoxy has the same definition as found herein. Examples include, but are not limited to, methoxycarbonyl, ethoxycarbonyl, propoxycarbonyl, isopropoxycarbonyl, butoxycarbonyl, butoxycarbonyl, isobutoxycarbonyl, tert-butoxycarbonyl.

The term “C₁-C₆ alkyl” refers to a straight or branched carbon radical containing 1 to 6 carbons. Some embodiments contain 1 to 5 carbons. Some embodiments contain 1 to 4 carbons. Some embodiments contain 1 to 3 carbons. Some embodiments contain 1 or 2 carbons. Examples of an alkyl group include, but are not limited to, methyl, ethyl, n-propyl, isopropyl, n-butyl, sec-butyl, isobutyl, tert-butyl, pentyl, isopentyl, t-pentyl, neopentyl, 1-methylbutyl [i.e., —CH(CH₃)CH₂CH₂CH₃], 2-methylbutyl [i.e., —CH₂CH(CH₃)CH₂CH₃], and n-hexyl.

The term “C₁-C₆ alkylheteroaryl” refers to a radical comprising a C₁-C₆ alkyl group attached to a heteroaryl group, wherein C₁-C₆ alkyl has the same definition as described herein. Examples include, but are not limited to, 4-methyl-1H-pyrazol-yl.

The term “C₁-C₆ alkylsulfinyl” refers to a radical comprising a C₁-C₆ alkyl group attached to the sulfur of a sulfinyl group, wherein C₁-C₆ alkyl has the same definition as described herein. Examples include, but are not limited to, methylsulfinyl, ethylsulfinyl, n-propylsulfinyl, isopropylsulfinyl, n-butylsulfinyl, sec-butylsulfinyl, isobutylsulfinyl, and tert-butylsulfinyl.

The term “C₁-C₆ alkylsulfonyl” refers to a radical comprising a C₁-C₆ alkyl group attached to the sulfur of a sulfonyl group, wherein C₁-C₆ alkyl has the same definition as described herein. Examples include, but are not limited to, methylsulfonyl, ethylsulfonyl, n-propylsulfonyl, isopropylsulfonyl, n-butylsulfonyl, sec-butylsulfonyl, isobutylsulfonyl, and tert-butylsulfonyl.

The term “C₁-C₆ alkylthio” refers to a radical comprising a C₁-C₆ alkyl group attached to a sulfur atom, wherein C₁-C₆ alkyl has the same definition as described herein. Examples include, but are not limited to, methylthio, ethylthio, n-propylthio, isopropylthio, n-butylthio, sec-butylthio, isobutylthio, and tert-butylthio.

The term “C₁-C₆ alkylthiocarbonyl” refers to a radical comprising a C₁-C₆ alkylthio group attached to a carbonyl group, wherein C₁-C₆ alkylthio has the same definition as described herein. The C₁-C₆ alkylthiocarbonyl group may be represented by the following:

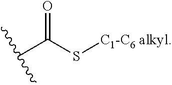

Examples include, but are not limited to, methylthiocarbonyl, ethylthiocarbonyl, n-propylthiocarbonyl, isopropylthiocarbonyl, n-butylthiocarbonyl, sec-butylthiocarbonyl, isobutylthiocarbonyl, and tert-butylthiocarbonyl.

The term “C₁-C₆ haloalkylthio” refers to a radical comprising a C₁-C₆ haloalkyl group attached to a sulfur atom, wherein C₁-C₆ haloalkyl has the same definition as described herein. Examples include, but are not limited to, fluoromethylthio, difluoromethylthio, trifluoromethylthio, 2,2,2-trifluoroethylthio, pentafluoroethylthio, 2-fluoropropan-2-ylthio, 1,1-difluoropropylthio, 1,3-difluoropropan-2-ylthio, (S)-1-fluoropropan-2-ylthio, (R)-1-fluoropropan-2-ylthio, 1,1,1-trifluoropropan-2-ylthio, and 1,1,1,3,3,3-hexafluoropropan-2-ylthio.

The term “C₂-C₈ dialkylcarboxamide” refers to a radical comprising two C₁-C₄ alkyl groups, that are the same or different, attached to an amide group, wherein C₁-C₄ alkyl has the same definition as described herein. A C₂-C₈ dialkylcarboxamido may be represented by the following groups:

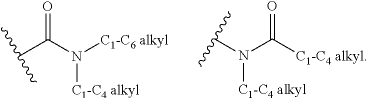

Examples include, but are not limited to, N,N-dimethylcarboxamide, N-methyl-N-ethylcarboxamide, N,N-diethylcarboxamide, N-methyl-N-isopropylcarboxamide.

The term “C₃-C₇ cycloalkoxy” refers to a radical comprising a C₃-C₇ cycloalkyl group attached to an oxygen atom, wherein C₃-C₇ cycloalkyl has the same definition as described herein. Examples include, but are not limited to, cyclopropyloxy, cyclobutyloxy, cyclopentyloxy, and cyclohexyloxy.

The term “C₃-C₇ cycloalkoxycarbonyl” refers to a radical comprising a C₃-C₇ cycloalkoxy group attached to a carbonyl group, wherein C₃-C₇ cycloalkoxy has the same definition as described herein. Examples include, but are not limited to, cyclopropyl, cyclobutyl, cyclopentyl, and cyclohexyl.

The term “C₃-C₇ cycloalkyl” refers to a saturated ring radical containing 3 to 7 carbons. Some embodiments contain 3 to 4 carbons. Some embodiments contain 3 to 5 carbons. Some embodiments contain 4 to 6 carbons. Some embodiments contain 5 to 6 carbons. Examples include cyclopropyl, cyclobutyl, cyclopentyl, and cyclohexyl.

The term “C₃-C₇ cycloalkylcarbonyl” refers to a radical comprising a C₃-C₇ cycloalkyl group attached to a carbonyl group, wherein C₃-C₇ cycloalkyl has the same definition as described herein. Examples include, but are not limited to, cyclopropylcarbonyl, cyclobutylcarbonyl, cyclopentylcarbonyl, and cyclohexylcarbonyl.

The term “C₃-C₇ cycloalkylsulfinyl” refers to a radical comprising a C₃-C₇ cycloalkyl group attached to the sulfur of a sulfinyl group, wherein C₃-C₇ cycloalkyl has the same definition as described herein. Examples include, but are not limited to, cyclopropylsulfinyl, cyclobutylsulfinyl, cyclopentylsulfinyl, and cyclohexylsulfinyl.

The term “C₃-C₇ cycloalkylsulfonyl” refers to a radical comprising a C₃-C₇ cycloalkyl group attached to the sulfur of a sulfonyl group, wherein C₃-C₇ cycloalkyl has the same definition as described herein. Examples include, but are not limited to, cyclopropylsulfonyl, cyclobutylsulfonyl, cyclopentylsulfonyl, and cyclohexylsulfonyl.

The term “C₃-C₇ cycloalkylthio” refers to a radical comprising a C₃-C₇ cycloalkyl group attached to a sulfur atom, wherein C₃-C₇ cycloalkyl has the same definition as described herein. Examples include, but are not limited to, cyclopropylthio, cyclobutylthio, cyclopentylthio, and cyclohexylthio.

The term “C₃-C₇ cycloalkythiocarbonyl” refers to a radical comprising a C₃-C₇ cycloalkylthio group attached to a carbonyl group, wherein the C₃-C₇ cycloalkythio radical has the same definition as described herein. The C₁-C₆ alkylthiocarbonyl group may be represented by the following:

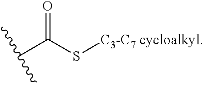

Examples include, but are not limited to, cyclopropylthiocarbonyl, cyclobutylthiocarbonyl, cyclopentylthiocarbonyl, and cyclohexylthiocarbonyl.

The term “C₄-C₁₃ cycloalkylalkyl” refers to a radical comprising a C₃-C₇ cycloalkyl group attached to a C₁-C₆ alkyl group, wherein the C₃-C₇ cycloalkyl and C₁-C₆ alkyl groups have the same definitions as described herein. Examples include, but are not limited to cyclopropylmethyl, cyclobutylmethyl, cyclopentylmethyl, cyclohexylmethyl, cyclopropylethyl, etc.

The term “carboxamide” refers to the group —CONH₂.

The term “cyano” refers to the group —CN.

The term “C₁-C₆ haloalkyl” refers to a radical comprising a C₁-C₆ alkyl group substituted with one or more halogens, wherein C₁-C₆ alkyl has the same definition as found herein. The C₁-C₆ haloalkyl may be fully substituted in which case it can be represented by the formula C_{q}L_{2q+1}, wherein L is a halogen and “q” is 1, 2, 3, 4, 5 or 6. When more than one halogen is present then they may be the same or different and selected from: fluorine, chlorine, bromine, and iodine. In some embodiments, haloalkyl contains 1 to 5 carbons. In some embodiments, haloalkyl contains 1 to 4 carbons. In some embodiments, haloalkyl contains 1 to 3 carbons. In some embodiments, haloalkyl contains 1 or 2 carbons. Examples of haloalkyl groups include, but are not limited to, fluoromethyl, difluoromethyl, trifluoromethyl, 2,2,2-trifluoroethyl, pentafluoroethyl, 2-fluoropropan-2-yl, 1,1-difluoropropyl, 1,3-difluoropropan-2-yl, (S)-1-fluoropropan-2-yl, (R)-1-fluoropropan-2-yl, 1,1,1-trifluoropropan-2-yl, and 1,1,1,3,3,3-hexafluoropropan-2-yl.

The term “halogen” refers to a fluoro, chloro, bromo or iodo group.

The term “heteroaryl” refers to a ring system containing 5 to 10 ring atoms, that may contain a single ring or two fused rings, and wherein at least one ring is aromatic and at least one ring atom of the aromatic ring is a heteroatom selected from, for example: O, S and N, wherein N is optionally substituted with H, C₁-C₄ acyl, C₁-C₄ alkyl, or O (i.e., forming an N-oxide) and S is optionally substituted with one or two oxygens. In some embodiments, the aromatic ring contains one heteroatom. In some embodiments, the aromatic ring contains two heteroatoms. In some embodiments, the aromatic ring contains three heteroatoms. Some embodiments are directed to 5-membered heteroaryl rings. Examples of a 5-membered heteroaryl ring include, but are not limited to, furanyl, thienyl, pyrrolyl, imidazolyl, oxazolyl, thiazolyl, isoxazolyl, pyrazolyl, isothiazolyl, oxadiazolyl, triazolyl, tetrazolyl, and thiadiazolyl. Some embodiments are directed to 6-membered heteroaryl rings. Examples of a 6-membered heteroaryl ring include, but are not limited to, pyridinyl, pyrazinyl, pyrimidinyl, pyridazinyl, and triazinyl.

The term “heteroaryl-C₁-C₆ alkyl” refers to a radical comprising a heteroaryl group attached to a C₁-C₆ alkyl group, wherein the heteroaryl and C₁-C₆ alkyl groups have the same definitions as described herein. Examples include, but are not limited to oxadiazolylmethyl, furanylmethyl, thienylmethyl, pyrrolylmethyl, imidazolylmethyl, oxazolylmethyl, thiazolylmethyl, isoxazolylmethyl, pyrazolylmethyl, isothiazolylmethyl, triazolylmethyl, tetrazolylmethyl, thiadiazolylmethyl, pyridinylmethyl, pyrazinylmethyl, pyrimidinylmethyl, pyridazinylmethyl, triazinylmethyl, oxadiazolylethyl, furanylethyl, thienylethyl, pyrrolylethyl, imidazolylethyl, oxazolylethyl, thiazolylethyl, isoxazolylethyl, pyrazolylethyl, isothiazolylethyl, triazolylethyl, tetrazolylethyl, thiadiazolylethyl, pyridinylethyl, pyrazinylethyl, pyrimidinylethyl, pyridazinylethyl, triazinylethyl, etc.

The term “heterocyclyl” refers to a non-aromatic ring radical containing 3 to 10 ring atoms, wherein one, two or three ring atoms are heteroatoms selected independently from, for example: O, S, and N, wherein when heterocyclyl is other than Ring A then N is optionally substituted with H, C₁-C₄ acyl or C₁-C₄ alkyl; and S is optionally substituted with one or two oxygens. Examples of a heterocyclyl group include, but are not limited to, aziridinyl, azetidinyl, piperidinyl, morpholinyl, piperazinyl, pyrrolidinyl, [1,3]-dioxolanyl, thiomorpholinyl, [1,4]oxazepanyl, 1,1-dioxothiomorpholinyl, azepanyl, tetrahydrofuranyl, tetrahydropyranyl, tetrahydrothiopyranyl, 1-oxo-hexahydro-1λ⁴-thiopyranyl, 1,1-dioxo-hexahydro-1λ⁶-thiopyranyl, and azabicyclo[3.2.1]octanyl. In some embodiments “heterocyclyl” refers to piperidin-4-yl, 3-azabicyclo[3.2.1]octan-8-yl, and 8-azabicyclo[3.2.1]octan-3-yl.

The term “heterocyclyloxy” refers to a radical comprising a heterocyclyl group attached to an oxygen radical, wherein heterocyclyl has the same definition as described herein.

The term “heterocyclyloxycarbonyl” refers to a radical comprising a heterocyclyloxy group attached to a carbonyl group, wherein heterocyclyloxy has the same definition as described herein.

The term “phenyl” refers to the group —C₆H₅.

### Compounds

One aspect of the present invention encompasses, inter alia, certain heterocyclyl derivatives selected from compounds of Formula I and pharmaceutically acceptable salts, solvates, and hydrates thereof:

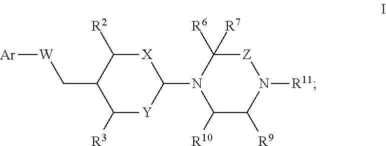

wherein Ar, W, X, Y, Z, R², R³, R⁶, R⁷, R⁹, R¹⁰, and R¹¹, have the same definitions as described herein, supra and infra. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables (e.g., Ar, W, X, Y, Z, R¹, R², R³, R⁴, R⁵, R⁶, R⁷, R⁸, R⁹, R¹⁰, and R¹¹) contained within the generic chemical formulae described herein, for example, Formulae I-XX, are specifically embraced by the present invention just as if each and every combination was individually and explicitly recited, to the extent that such combinations embrace compounds that result in stable compounds (i.e., compounds that can be isolated, characterized and tested for biological activity). In addition, all subcombinations of the chemical groups listed in the embodiments describing such variables, as well as all subcombinations of uses and medical indications described herein, are also specifically embraced by the present invention just as if each and every subcombination of chemical groups and subcombination of uses and medical indications was individually and explicitly recited herein. In addition, some embodiments include every combination of one or more pharmaceutical agents, such as a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, and the like, either specifically disclosed herein or specifically disclosed in any reference recited herein just as if each and every combination was individually and explicitly recited. Still further, some embodiments of the present invention include every combination of one or more embodiments pertaining to the chemical groups represented by the variables and generic chemical formulae as described herein or every combination of one or more compounds of Formula I together/in combination with every combination of one or more pharmaceutical agents, such as a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, and the like, either specifically disclosed herein or specifically disclosed in any reference recited herein just as if each and every combination was individually and explicitly recited.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents, and the like. Likewise, “substituted with one or more substituents” refers to the substitution of a group with one substituent up to the total number of substituents physically allowed by the group. Further, when a group is substituted with more than one group they can be identical or they can be different.

Compounds of the invention can also include tautomeric forms, such as keto-enol tautomers and the like. Tautomeric forms can be in equilibrium or sterically locked into one form by appropriate substitution. It is understood that the various tautomeric forms are within the scope of the compounds of the present invention.

It is understood and appreciated that compounds of Formula I and formulae related thereto may have one or more chiral centers and therefore can exist as enantiomers and/or diastereoisomers. The invention is understood to extend to and embrace all such enantiomers, diastereoisomers and mixtures thereof, including but not limited to racemates. It is understood that compounds of Formula I and formulae used throughout this disclosure represent all individual enantiomers and mixtures thereof, unless stated or shown otherwise.

It is understood and appreciated that certain compounds of Formula I and formulae related thereto exist as meso isomers. Such meso isomers may be referred to as cis and trans isomers. The cis meso isomers of compounds of Formula I are named herein using the designation (1s,4s) and the trans meso isomers of compounds of Formula I are named herein using the designation (1r,4r) as shown below:

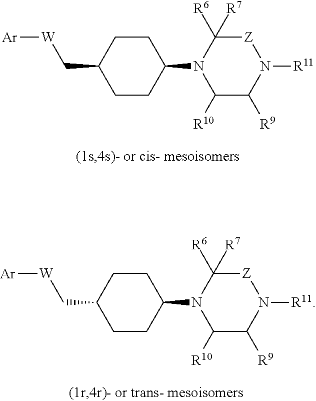

One aspect of the present invention pertains to compounds of the present invention wherein the mesoisomer stereochemistry is (1r,4r).

One aspect of the present invention pertains to compounds of the present invention wherein the mesoisomer stereochemistry is (1s,4s).

One aspect of the present invention pertains to compounds of Formula I and pharmaceutically acceptable salts, solvates, and hydrates thereof:

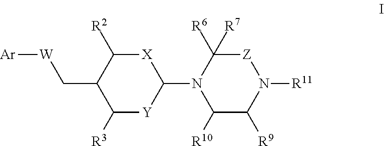

wherein:

Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl;

W is selected from: O and NR¹; or W is absent;

One aspect of the present invention pertains to compounds of Formula II and pharmaceutically acceptable salts, solvates, and hydrates thereof:

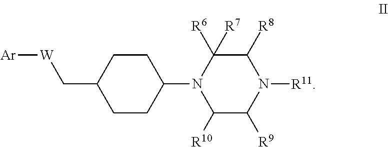

One aspect of the present invention pertains to compounds of Formula III and pharmaceutically acceptable salts, solvates, and hydrates thereof:

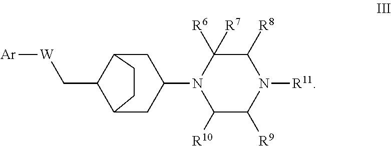

One aspect of the present invention pertains to compounds of Formula IV and pharmaceutically acceptable salts, solvates, and hydrates thereof:

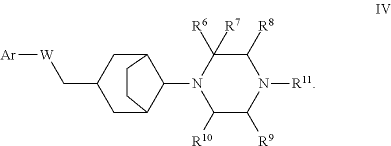

One aspect of the present invention pertains to compounds of Formula V and pharmaceutically acceptable salts, solvates, and hydrates thereof:

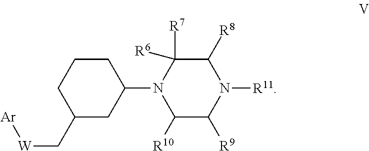

One aspect of the present invention pertains to compounds of Formula VI and pharmaceutically acceptable salts, solvates, and hydrates thereof:

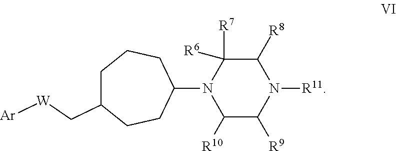

One aspect of the present invention pertains to compounds of Formula VII and pharmaceutically acceptable salts, solvates, and hydrates thereof:

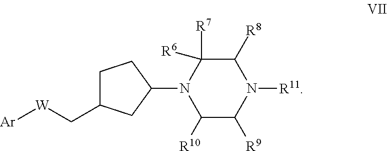

One aspect of the present invention pertains to compounds of Formula VIII and pharmaceutically acceptable salts, solvates, and hydrates thereof:

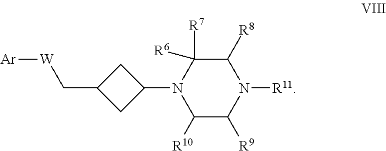

One aspect of the present invention pertains to compounds of Formula IX and pharmaceutically acceptable salts, solvates, and hydrates thereof:

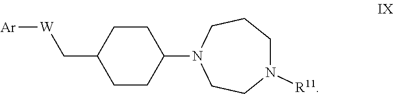

One aspect of the present invention pertains to compounds of Formula X and pharmaceutically acceptable salts, solvates, and hydrates thereof:

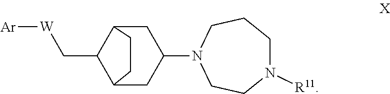

One aspect of the present invention pertains to compounds of Formula XI and pharmaceutically acceptable salts, solvates, and hydrates thereof:

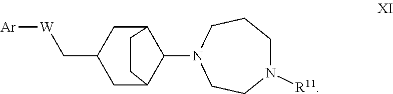

One aspect of the present invention pertains to compounds of Formula XII and pharmaceutically acceptable salts, solvates, and hydrates thereof:

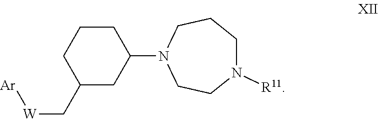

One aspect of the present invention pertains to compounds of Formula XIII and pharmaceutically acceptable salts, solvates, and hydrates thereof:

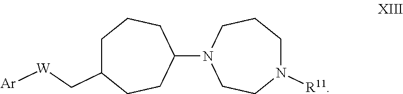

One aspect of the present invention pertains to compounds of Formula XIV and pharmaceutically acceptable salts, solvates, and hydrates thereof:

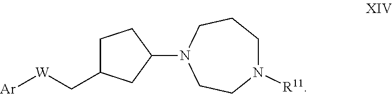

One aspect of the present invention pertains to compounds of Formula XV and pharmaceutically acceptable salts, solvates, and hydrates thereof:

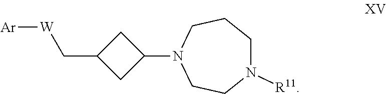

### The Group Ar

In some embodiments, Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl

In some embodiments, Ar is selected from: aryl and heteroaryl; each optionally substituted with one or more substituents selected from: chloro, cyano, dimethylcarbamoyl, cyclopropylsulfonyl, fluoro, imidazolyl, methyl, methylpyrazolyl, methylsulfonyl, pyrrolyl, tetrazolyl, triazolyl, and trifluoromethylthio.

In some embodiments, Ar is selected from: phenyl, pyrazinyl, pyridinyl, and pyrimidinyl; each optionally substituted with one or more substituents selected from: chloro, cyano, dimethylcarbamoyl, cyclopropylsulfonyl, fluoro, imidazolyl, methyl, methylpyrazolyl, methylsulfonyl, pyrrolyl, tetrazolyl, triazolyl, and trifluoromethylthio.

In some embodiments, Ar is selected from: 1-methylpyrrolo[3,4-c]pyrazol-5(1H,4H,6H)-yl, 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(dimethylcarbamoyl)-3-methylphenyl, 4-(methylsulfonyl)phenyl, 4-(trifluoromethylthio)phenyl, 4-cyanophenyl, 5-(methylsulfonyl)-1H-indol-1-yl, 5-(methylsulfonyl)indolin-1-yl, 5-(methylsulfonyl)pyrazin-2-yl, 5-(methylsulfonyl)pyridin-2-yl, and 6-cyano-2-methylpyrimidin-4-yl.

In some embodiments, Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl.

In some embodiments, Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, 6-cyano-2-methylpyrimidin-4-yl, and pyridin-4-yl.

In some embodiments, Ar is 1-methylpyrrolo[3,4-c]pyrazol-5(1H,4H,6H)-yl.

In some embodiments, Ar is 2-chloro-4-(methylsulfonyl)phenyl

In some embodiments, Ar is 2-fluoro-4-(1H-1,2,3-triazol-1-yl)phenyl.

In some embodiments, Ar is 2-fluoro-4-(1H-1,2,4-triazol-1-yl)phenyl.

In some embodiments, Ar is 2-fluoro-4-(1H-imidazol-1-yl)phenyl.

In some embodiments, Ar is 2-fluoro-4-(1H-pyrrol-1-yl)phenyl.

In some embodiments, Ar is 2-fluoro-4-(1H-tetrazol-1-yl)phenyl.

In some embodiments, Ar is 2-fluoro-4-(2H-1,2,3-triazol-2-yl)phenyl.

In some embodiments, Ar is 2-fluoro-4-(4H-1,2,4-triazol-4-yl)phenyl.

In some embodiments, Ar is 2-fluoro-4-(4-methyl-1H-pyrazol-1-yl)phenyl.

In some embodiments, Ar is 2-fluoro-4-(methylsulfonyl)phenyl.

In some embodiments, Ar is 2-methyl-6-(methylsulfonyl)pyridin-3-yl.

In some embodiments, Ar is 3-chloro-4-(methylsulfonyl)phenyl.

In some embodiments, Ar is 3-cyanopyridin-4-yl.

In some embodiments, Ar is 4-(1H-1,2,4-triazol-1-yl)phenyl.

In some embodiments, Ar is 4-(1H-tetrazol-1-yl)phenyl.

In some embodiments, Ar is 4-(cyclopropylsulfonyl)-2-fluorophenyl.

In some embodiments, Ar is 4-(dimethylcarbamoyl)-2-fluorophenyl.

In some embodiments, Ar is 4-(dimethylcarbamoyl)-3-methylphenyl.

In some embodiments, Ar is 4-(methylsulfonyl)phenyl.

In some embodiments, Ar is 4-(trifluoromethylthio)phenyl.

In some embodiments, Ar is 4-cyanophenyl.

In some embodiments, Ar is 5-(methylsulfonyl)-1H-indol-1-yl.

In some embodiments, Ar is 5-(methylsulfonyl)indolin-1-yl.

In some embodiments, Ar is 5-(methylsulfonyl)pyrazin-2-yl.

In some embodiments, Ar is 5-(methylsulfonyl)pyridin-2-yl.

In some embodiments, Ar is 6-cyano-2-methylpyrimidin-4-yl.

In some embodiments, Ar is 3-cyanopyridin-4-yl.

In some embodiments, Ar is pyridin-4-yl.

### The Group W

In some embodiments, W is selected from: O and NR¹; or W is absent.

In some embodiments, W is O.

In some embodiments, W is NR¹.

In some embodiments, W is NH.

In some embodiments, W is absent.

### The Group R¹

In some embodiments, R¹ is selected from: H and C₁-C₆ alkyl.

In some embodiments, R¹ is H.

In some embodiments, R¹ is C₁-C₆ alkyl.

In some embodiments, R¹ is C₁-C₆ methyl.

### The Groups R² and R³

In some embodiments, R² and R³ are each H; or R² and R³ together form —CH₂—CH₂—.

In some embodiments, R² and R³ are each H.

In some embodiments, R² is H.

In some embodiments, R³ is H.

In some embodiments, R² and R³ together form —CH₂—CH₂—.

### The Group X

In some embodiments, X is selected from: —CH₂—CH₂— and CHR⁴; or X is absent.

In some embodiments, X is —CH₂—CH₂—.

In some embodiments, X is CHR⁴.

In some embodiments, X is CH₂.

In some embodiments, X is absent.

In some embodiments, X is selected from: —CH₂—CH₂—, CHR⁴, C(O), CHF, and CF₂; or X is absent

In some embodiments, X is CHF or CF₂.

In some embodiments, X is CHF.

In some embodiments, X is CF₂.

In some embodiments, X is C(O).

### The Group Y

In some embodiments, Y is CHR⁵; or Y is absent.

In some embodiments, Y is CHR⁵.

In some embodiments, Y is CH₂.

In some embodiments, Y is absent.

### The Groups R⁴ and R⁵

In some embodiments, R⁴ and R⁵ are each H; or R⁴ and R⁵ together form —CH₂—CH₂—.

In some embodiments, R⁴ and R⁵ are each H.

In some embodiments, R⁴ is H.

In some embodiments, R⁵ is H.

In some embodiments, X is CHR⁴; Y is CHR⁵; and R⁴ and R⁵ together form —CH₂—CH₂—.

### The Group Z

In some embodiments, Z is selected from: —CH₂—CH₂— and CHR⁸.

In some embodiments, Z is —CH₂—CH₂—.

In some embodiments, Z is CHR⁸.

In some embodiments, Z is CH₂.

In some embodiments, Z is selected from: —CH₂—CH₂—, CHR⁸, and C(O).

In some embodiments, Z is C(O).

### The Groups R⁶, R⁷, R⁸, R⁹, and R¹⁰

In some embodiments, R⁶, R⁹, and R¹⁰ are each H, and R⁷ and R⁸ are each independently selected from: H and C₁-C₆ alkyl; or R⁸, R⁹, and R¹⁰ are each H, and R⁶ and R⁷ together form —CH₂—CH₂—; or R⁶, R⁸, and R⁹ are each H, and R⁷ and R¹⁰ together form —CH₂—CH₂; or R⁶, R⁷, and R¹⁰ are each H, and R⁸ and R⁹ together form —CH₂—CH₂; or R⁶, R⁷, and R⁹ are each H, and R⁸ and R¹⁰ together form —CH₂—CH₂—.

In some embodiments, R⁶, R⁹ and R¹⁰ are each H, and R⁷ and R⁸ are each independently selected from: H and C₁-C₆ alkyl; or R⁸, R⁹ and R¹⁰ are each H, and R⁶ and R⁷ together form —CH₂—CH₂—; or R⁶, R⁸ and R⁹ are each H, and R⁷ and R¹⁰ together form —CH₂—CH₂, or a bond; or R⁶, R⁷ and R¹⁰ are each H, and R⁸ and R⁹ together form —CH₂—CH₂; or R⁶, R⁷ and R⁹ are each H, and R⁸ and R¹⁰ together form —CH₂—CH₂—; or R⁶, R⁷ and R⁸ are each H, and R⁹ and R¹⁰ together form —CH₂—.

In some embodiments, R⁶, R⁸, R⁹ and R¹⁰ are each H; and R⁷ is methyl; or R⁶, R⁷, R⁹ and R¹⁰ are each H; and R⁸ is methyl; or R⁸, R⁹ and R¹⁰ are each H; and R⁶ and R⁷ together form —CH₂—CH₂—; or R⁶, R⁸ and R⁹ are each H; and R⁷ and R¹⁰ together form —CH₂—CH₂—; or R⁶, R⁷ and R¹⁰ are each H; and R⁸ and R⁹ together form —CH₂—CH₂—.

In some embodiments, R⁶, R⁷ and R⁸ are each H; and R⁹ and R¹⁰ together form —CH₂—; or R⁶, R⁸ and R⁹ are each H; and R⁷ and R¹⁰ together form a bond.

In some embodiments, R⁶, R⁹, and R¹⁰ are each H, and R⁷ and R⁸ are each independently selected from: H and C₁-C₆ alkyl.

In some embodiments, R⁶, R⁷, R⁸, R⁹, and R¹⁰ are each H.

In some embodiments, R⁶, R⁸, R⁹, and R¹⁰ are each H; and R⁷ is C₁-C₆ alkyl.

In some embodiments, R⁶, R⁷, R⁹, and R¹⁰ are each H; and R⁸ is C₁-C₆ alkyl.

In some embodiments, R⁶, R⁸, R⁹, and R¹⁰ are each H; and R⁷ is methyl.

In some embodiments, R⁶, R⁷, R⁹, and R¹⁰ are each H; and R⁸ is methyl.

In some embodiments, R⁸, R⁹, and R¹⁰ are each H, and R⁶ and R⁷ together form —CH₂—CH₂—.

In some embodiments, R⁶, R⁸ and R⁹ are each H, and R⁷ and R¹⁰ together form —CH₂—CH₂, or a bond.

In some embodiments, R⁶, R⁸, and R⁹ are each H, and R⁷ and R¹⁰ together form —CH₂—CH₂.

In some embodiments, R⁶, R⁸ and R⁹ are each H, and R⁷ and R¹⁰ together form a bond.

In some embodiments, R⁶, R⁷, and R¹⁰ are each H, and R⁸ and R⁹ together form —CH₂—CH₂.

In some embodiments, R⁶, R⁷, and R⁹ are each H, and R⁸ and R¹⁰ together form —CH₂—CH₂.

In some embodiments, R⁶, R⁷ and R⁸ are each H, and R⁹ and R¹⁰ together form —CH₂

### The Group R¹¹

In some embodiments, R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

In some embodiments, R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: chloro, cyclopropyl, difluoromethyl, ethyl, fluoro, 2-fluoropropan-2-yl, isopropyl, methoxy, methyl, and trifluoromethyl.

In some embodiments, R¹¹ is selected from: sec-butoxycarbonyl, tert-butoxycarbonyl, cyclobutoxycarbonyl, cyclobutylmethyl, cyclobutylthiocarbonyl, cyclopropoxycarbonyl, cyclopropylcarbonyl, cyclopropylmethyl, cyclopropylthiocarbonyl, ethoxycarbonyl, isopropoxycarbonyl, isopropylthiocarbonyl, 1,2,4-oxadiazolyl, (oxetan-3-yloxy)carbonyl, n-propoxycarbonyl, and pyrimidin-2-yl; each optionally substituted with one or more substituents selected from: chloro, cyclopropyl, difluoromethyl, ethyl, fluoro, 2-fluoropropan-2-yl, isopropyl, methoxy, methyl, and trifluoromethyl.

In some embodiments, R¹¹ is selected from: (1-(trifluoromethyl)cyclobutoxy)carbonyl, (1-(trifluoromethyl)cyclobutyl)methyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (3-(trifluoromethyl)oxetan-3-yloxy)carbonyl, (3,3,4,4,4-pentafluorobutan-2-yloxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(difluoromethyl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, 5-isopropyl-1,2,4-oxadiazol-3-yl, 5-methoxypyrimidin-2-yl, cyclobutylthiocarbonyl, cyclopropylthiocarbonyl, isopropylthiocarbonyl, and tert-butoxycarbonyl.

In some embodiments, wherein R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, and heteroaryl; each optionally substituted with one or more substituents selected from: chloro, cyclopropyl, ethyl, fluoro, 2-fluoropropan-2-yl, isopropyl, methyl, and trifluoromethyl.

In some embodiments, R¹¹ is selected from: tert-butoxycarbonyl, cyclobutoxycarbonyl, cyclobutylthiocarbonyl, cyclopropoxycarbonyl, cyclopropylcarbonyl, cyclopropylmethyl, cyclopropylthiocarbonyl, ethoxycarbonyl, isopropoxycarbonyl, 1,2,4-oxadiazolyl, n-propoxycarbonyl, and pyrimidin-2-yl; each optionally substituted with one or more substituents selected from: chloro, cyclopropyl, difluoromethyl, ethyl, fluoro, 2-fluoropropan-2-yl, isopropyl, methoxy, methyl, and trifluoromethyl.

In some embodiments, R¹¹ is selected from: (1-(trifluoromethyl)cyclobutoxy)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, cyclobutylthiocarbonyl, cyclopropylthiocarbonyl, and tert-butoxycarbonyl.

In some embodiments, R¹¹ is selected from: C₁-C₆ alkoxycarbonyl, C₁-C₆ alkylsulfonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, heteroaryl-C₁-C₆ alkylene, and heterocyclyloxycarbonyl; each optionally substituted with one or more substituents selected from: chloro, cyclopropyl, ethyl, fluoro, 2-fluoropropan-2-yl, isopropyl, methyl, and trifluoromethyl.

In some embodiments, R¹¹ is selected from: (1-(trifluoromethyl)cyclobutoxy)carbonyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (2,2,2-trifluoroethyl)sulfonyl, (3-(trifluoromethyl)oxetan-3-yloxy)carbonyl, 2,2,3,3,3-pentafluoropropoxy)carbonyl, 3-(2-fluoropropan-2-yl)-1,2,4-oxadiazol-5-yl, 3-(trifluoromethyl)-1,2,4-oxadiazol-5-yl, 3-(trifluoromethyl)-1,2,4-oxadiazol-5-ylmethyl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, cyclobutylthiocarbonyl, cyclopropylthiocarbonyl, and tert-butoxycarbonyl.

In some embodiments, R¹¹ is (1-(trifluoromethyl)cyclobutoxy)carbonyl.

In some embodiments, R¹¹ is (1-(trifluoromethyl)cyclobutyl)methyl.

In some embodiments, R¹¹ is (1-(trifluoromethyl)cyclopropyl)carbonyl.

In some embodiments, R¹¹ is (1-(trifluoromethyl)cyclopropyl)methyl.

In some embodiments, R¹¹ is (1,1,1,3,3,3-hexafluoropropan-2-yloxy)carbonyl.

In some embodiments, R¹¹ is (1,1,1-trifluoro-2-methylpropan-2-yloxy)carbonyl.

In some embodiments, R¹¹ is (1,1,1-trifluoropropan-2-yloxy)carbonyl.

In some embodiments, R¹¹ is (1-fluoro-2-methylpropan-2-yloxy)carbonyl.

In some embodiments, R¹¹ is (1-fluoropropan-2-yloxy)carbonyl.

In some embodiments, R¹¹ is (1-methylcyclobutoxy)carbonyl.

In some embodiments, R¹¹ is (1-methylcyclopropoxy)carbonyl.

In some embodiments, R¹¹ is (2,2,2-trifluoroethoxy)carbonyl.

In some embodiments, R¹¹ is (2,2,2-trifluoroethyl)sulfonyl.

In some embodiments, R¹¹ is (3-(trifluoromethyl)oxetan-3-yloxy)carbonyl.

In some embodiments, R¹¹ is (3,3,4,4,4-pentafluorobutan-2-yloxy)carbonyl.

In some embodiments, R¹¹ is (trifluoromethyl)cyclopropanecarbonyl.

In some embodiments, R¹¹ is 2,2,3,3,3-pentafluoropropoxy)carbonyl.

In some embodiments, R¹¹ is 3-(2-fluoropropan-2-yl)-1,2,4-oxadiazol-5-yl.

In some embodiments, R¹¹ is 3-(difluoromethyl)-1,2,4-oxadiazol-5-yl.

In some embodiments, R¹¹ is 3-(trifluoromethyl)-1,2,4-oxadiazol-5-yl.

In some embodiments, R¹¹ is 3-(trifluoromethyl)-1,2,4-oxadiazol-5-ylmethyl.

In some embodiments, R¹¹ is 3-cyclopropyl-1,2,4-oxadiazol-5-yl.

In some embodiments, R¹¹ is 3-isopropyl-1,2,4-oxadiazol-5-yl.

In some embodiments, R¹¹ is 5-(2-fluoropropan-2-yl)-1,2,4-oxadiazol-3-yl.

In some embodiments, R¹¹ is 5-(trifluoromethyl)-1,2,4-oxadiazol-3-yl.

In some embodiments, R¹¹ is 5-(trifluoromethyl)pyrimidin-2-yl.

In some embodiments, R¹¹ is 5-chloropyrimidin-2-yl.

In some embodiments, R¹¹ is 5-ethylpyrimidin-2-yl.

In some embodiments, R¹¹ is 5-isopropyl-1,2,4-oxadiazol-3-yl.

In some embodiments, R¹¹ is 5-methoxypyrimidin-2-yl.

In some embodiments, R¹¹ is cyclobutylthiocarbonyl.

In some embodiments, R¹¹ is cyclopropylthiocarbonyl.

In some embodiments, R¹¹ is isopropylthiocarbonyl.

In some embodiments, R¹¹ is tert-butoxycarbonyl.

### The Group R¹⁵

In some embodiments, R¹⁵ is selected from: H and cyano.

In some embodiments, R¹⁵ is H.

In some embodiments, R¹⁵ is cyano.

### Certain Combinations

In some embodiments, Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(dimethylcarbamoyl)-3-methylphenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl; and W is selected from: O and NR¹; or Ar is selected from: 1-methylpyrrolo[3,4-c]pyrazol-5(1H,4H,6H)-yl, 5-(methylsulfonyl)-1H-indol-1-yl, and 5-(methylsulfonyl)indolin-1-yl; and W is absent.

In some embodiments, Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(dimethylcarbamoyl)-3-methylphenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl; and W is selected from: O and NR¹.

In some embodiments, Ar is selected from: 1-methylpyrrolo[3,4-c]pyrazol-5(1H,4H,6H)-yl, 5-(methylsulfonyl)-1H-indol-1-yl, and 5-(methylsulfonyl)indolin-1-yl; and W is absent.

One aspect of the present invention pertains to compounds of Formula XVI and pharmaceutically acceptable salts, solvates, and hydrates thereof:

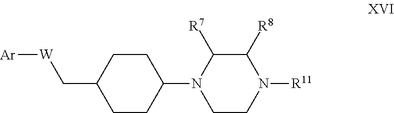

wherein:

Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl;

W is selected from: O and NR¹; or W is absent;

R¹ is selected from: H and C₁-C₆ alkyl;

R⁷ and R⁸ are each independently selected from: H and C₁-C₆ alkyl; and

R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

One aspect of the present invention pertains to compounds of Formula XVI and pharmaceutically acceptable salts, solvates, and hydrates thereof:

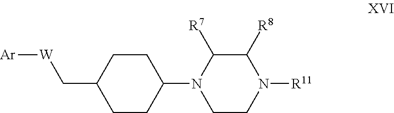

wherein:

Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(dimethylcarbamoyl)-3-methylphenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl; and

W is selected from: O and NR¹;

or

Ar is selected from: 1-methylpyrrolo[3,4-c]pyrazol-5(1H,4H,6H)-yl, 5-(methylsulfonyl)-1H-indol-1-yl, and 5-(methylsulfonyl)indolin-1-yl; and

W is absent;

R¹ is selected from: H and methyl;

R⁷ and R⁸ are each independently selected from: H and methyl; and

R¹¹ is selected from: (1-(trifluoromethyl)cyclobutoxy)carbonyl, (1-(trifluoromethyl)cyclobutyl)methyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (3-(trifluoromethyl)oxetan-3-yloxy)carbonyl, (3,3,4,4,4-pentafluorobutan-2-yloxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(difluoromethyl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, 5-isopropyl-1,2,4-oxadiazol-3-yl, 5-methoxypyrimidin-2-yl, cyclobutylthiocarbonyl, cyclopropylthiocarbonyl, isopropylthiocarbonyl, and tert-butoxycarbonyl.

One aspect of the present invention pertains to compounds of Formula XVI and pharmaceutically acceptable salts, solvates, and hydrates thereof:

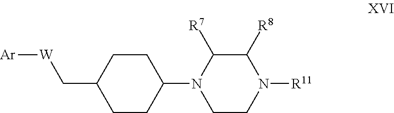

wherein:

Ar is selected from: aryl and heteroaryl; each optionally substituted with one or more substituents selected from: C₁-C₆ alkyl, C₁-C₆ alkylsulfonyl, cyano, C₃-C₇ cycloalkylsulfonyl, C₂-C₈ dialkylcarboxamide, C₁-C₆ haloalkylthio, halogen, heteroaryl, and C₁-C₆ alkylheteroaryl;

W is selected from: O and NH;

R⁷ and R⁸ are each independently selected from: H and C₁-C₆ alkyl; and

R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, and heteroaryl; each optionally substituted with one or more substituents selected from: C₁-C₆ alkyl, C₃-C₇ cycloalkyl, C₁-C₆ haloalkyl, and halogen.

One aspect of the present invention pertains to compounds of Formula XVI and pharmaceutically acceptable salts, solvates, and hydrates thereof:

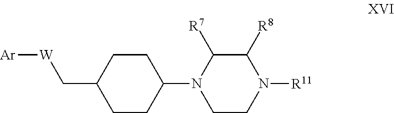

wherein:

Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl;

W is selected from: O and NH;

R⁷ and R⁸ are each independently selected from: H and methyl; and

R¹¹ is selected from: (1-(trifluoromethyl)cyclobutoxy)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, cyclobutylthiocarbonyl, cyclopropylthiocarbonyl, and tert-butoxycarbonyl.

One aspect of the present invention pertains to compounds of Formula XVII and pharmaceutically acceptable salts, solvates, and hydrates thereof:

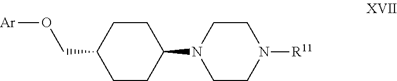

wherein:

Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl; and

R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

One aspect of the present invention pertains to compounds of Formula XVII and pharmaceutically acceptable salts, solvates, and hydrates thereof:

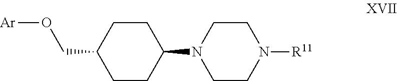

wherein:

Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(dimethylcarbamoyl)-3-methylphenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl; and

R¹¹ is selected from: (1-(trifluoromethyl)cyclobutoxy)carbonyl, (1-(trifluoromethyl)cyclobutyl)methyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (3-(trifluoromethyl)oxetan-3-yloxy)carbonyl, (3,3,4,4,4-pentafluorobutan-2-yloxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(difluoromethyl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, 5-isopropyl-1,2,4-oxadiazol-3-yl, 5-methoxypyrimidin-2-yl, cyclobutylthiocarbonyl, cyclopropylthiocarbonyl, isopropylthiocarbonyl, and tert-butoxycarbonyl.

One aspect of the present invention pertains to compounds of Formula XVII and pharmaceutically acceptable salts, solvates, and hydrates thereof:

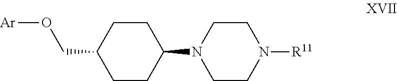

wherein:

Ar is selected from: aryl and heteroaryl; each optionally substituted with one or more substituents selected from: C₁-C₆ alkyl, C₁-C₆ alkylsulfonyl, cyano, C₃-C₇ cycloalkylsulfonyl, C₂-C₈ dialkylcarboxamide, C₁-C₆ haloalkylthio, halogen, heteroaryl, and C₁-C₆ alkylheteroaryl; and

R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, and heteroaryl; each optionally substituted with one or more substituents selected from: C₁-C₆ alkyl, C₃-C₇ cycloalkyl, C₁-C₆ haloalkyl, and halogen.

One aspect of the present invention pertains to compounds of Formula XVII and pharmaceutically acceptable salts, solvates, and hydrates thereof:

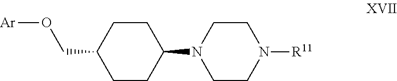

wherein:

Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl; and

R¹¹ is selected from: (1-(trifluoromethyl)cyclobutoxy)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, cyclobutylthiocarbonyl, cyclopropylthiocarbonyl, and tert-butoxycarbonyl.

One aspect of the present invention pertains to compounds of Formula XVIII and pharmaceutically acceptable salts, solvates, and hydrates thereof:

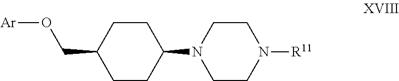

wherein:

Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl; and

R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

One aspect of the present invention pertains to compounds of Formula XVIII and pharmaceutically acceptable salts, solvates, and hydrates thereof:

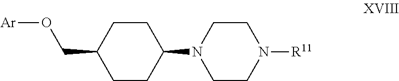

wherein:

Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(dimethylcarbamoyl)-3-methylphenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl; and

R¹¹ is selected from: (1-(trifluoromethyl)cyclobutoxy)carbonyl, (1-(trifluoromethyl)cyclobutyl)methyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (3-(trifluoromethyl)oxetan-3-yloxy)carbonyl, (3,3,4,4,4-pentafluorobutan-2-yloxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(difluoromethyl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, 5-isopropyl-1,2,4-oxadiazol-3-yl, 5-methoxypyrimidin-2-yl, cyclobutylthiocarbonyl, cyclopropylthiocarbonyl, isopropylthiocarbonyl, and tert-butoxycarbonyl.

One aspect of the present invention pertains to compounds of Formula XVIII and pharmaceutically acceptable salts, solvates, and hydrates thereof:

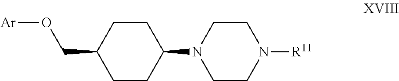

wherein:

Ar is selected from: aryl and heteroaryl; each optionally substituted with one or more substituents selected from: C₁-C₆ alkyl, C₁-C₆ alkylsulfonyl, cyano, C₃-C₇ cycloalkylsulfonyl, C₂-C₈ dialkylcarboxamide, C₁-C₆ haloalkylthio, halogen, heteroaryl, and C₁-C₆ alkylheteroaryl; and

R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, and heteroaryl; each optionally substituted with one or more substituents selected from: C₁-C₆ alkyl, C₃-C₇ cycloalkyl, C₁-C₆ haloalkyl, and halogen.

One aspect of the present invention pertains to compounds of Formula XVIII and pharmaceutically acceptable salts, solvates, and hydrates thereof:

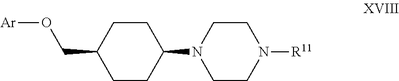

wherein:

Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl; and

R¹¹ is selected from: (1-(trifluoromethyl)cyclobutoxy)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, cyclobutylthiocarbonyl, cyclopropylthiocarbonyl, and tert-butoxycarbonyl.

One aspect of the present invention pertains to compounds of Formula Ia and pharmaceutically acceptable salts, solvates, and hydrates thereof:

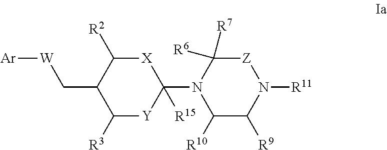

wherein:

Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl;

W is selected from: O and NR¹; or W is absent;

R¹ is selected from: H and C₁-C₆ alkyl;

R² and R³ are each H; or R² and R³ together form —CH₂—CH₂—;

X is selected from: —CH₂—CH₂—, CHR⁴, C(O), CHF, and CF₂; or X is absent;

Y is CHR⁵; or Y is absent;

R⁴ and R⁵ are each H; or R⁴ and R⁵ together form —CH₂—CH₂—;

Z is selected from: —CH₂—CH₂—, CHR⁸, and C(O);

R⁶, R⁹ and R¹⁰ are each H, and R⁷ and R⁸ are each independently selected from: H and C₁-C₆ alkyl; or R⁸, R⁹ and R¹⁰ are each H, and R⁶ and R⁷ together form —CH₂—CH₂—; or R⁶, R⁸ and R⁹ are each H, and R⁷ and R¹⁰ together form —CH₂—CH₂, or a bond; or R⁶, R⁷ and R¹⁰ are each H, and R⁸ and R⁹ together form —CH₂—CH₂; or R⁶, R⁷ and R⁹ are each H, and R⁸ and R¹⁰ together form —CH₂—CH₂—; or R⁶, R⁷ and R⁸ are each H, and R⁹ and R¹⁰ together form —CH₂—;

R¹¹ is selected from: C₁-C₆ alkoxycarbonyl, C₁-C₆ alkylsulfonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, heteroaryl-C₁-C₆ alkylene, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen; and

R¹⁵ is selected from: H and cyano.

One aspect of the present invention pertains to compounds of Formula XIX and pharmaceutically acceptable salts, solvates, and hydrates thereof:

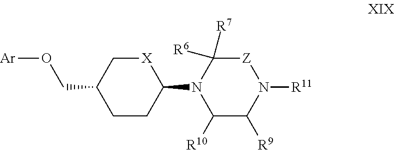

wherein:

Ar is selected from: aryl and heteroaryl; each optionally substituted with one or more substituents selected from: C₁-C₆ alkyl, C₁-C₆ alkylsulfonyl, cyano, C₃-C₇ cycloalkylsulfonyl, C₂-C₈ dialkylcarboxamide, C₁-C₆ haloalkylthio, halogen, heteroaryl, and C₁-C₆ alkylheteroaryl;

X is selected from: —CH₂—, C(O), CHF, and CF₂

R⁶, R⁸, R⁹ and R¹⁰ are each H; and R⁷ is C₁-C₆ alkyl; or R⁶, R⁷, R⁹ and R¹⁰ are each H; and R⁸ is C₁-C₆ alkyl; or R⁸, R⁹ and R¹⁰ are each H; and R⁶ and R⁷ together form —CH₂—CH₂—; or R⁶, R⁸ and R⁹ are each H; and R⁷ and R¹⁰ together form —CH₂—CH₂—; or R⁶, R⁷ and R¹⁰ are each H; and R⁸ and R⁹ together form —CH₂—CH₂—; or R⁶, R⁷ and R⁸ are each H; and R⁹ and R¹⁰ together form —CH₂—; or R⁶, R⁸ and R⁹ are each H; and R⁷ and R¹⁰ together form a bond; and

R¹¹ is selected from: C₁-C₆ alkoxycarbonyl, C₁-C₆ alkylsulfonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, heteroaryl-C₁-C₆ alkylene, and heterocyclyloxycarbonyl; each optionally substituted with one or more substituents selected from: chloro, cyclopropyl, ethyl, fluoro, 2-fluoropropan-2-yl, isopropyl, methyl, and trifluoromethyl

One aspect of the present invention pertains to compounds of Formula XIX and pharmaceutically acceptable salts, solvates, and hydrates thereof:

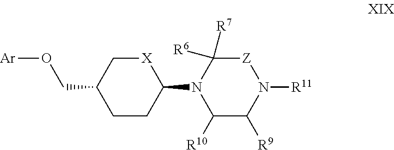

wherein:

Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, 6-cyano-2-methylpyrimidin-4-yl, and pyridin-4-yl;

X is selected from: —CH₂—, C(O), CHF, and CF₂

R⁶, R⁸, R⁹ and R¹⁰ are each H; and R⁷ is methyl; or R⁶, R⁷, R⁹ and R¹⁰ are each H; and R⁸ is methyl; or R⁸, R⁹ and R¹⁰ are each H; and R⁶ and R⁷ together form —CH₂—CH₂—; or R⁶, R⁸ and

R⁹ are each H; and R⁷ and R¹⁰ together form —CH₂—CH₂—; or R⁶, R⁷ and R¹⁰ are each H; and R⁸ and R⁹ together form —CH₂—CH₂—; or R⁶, R⁷ and R⁸ are each H; and R⁹ and R¹⁰ together form —CH₂—; or R⁶, R⁸ and R⁹ are each H; and R⁷ and R¹⁰ together form a bond; and

R¹¹ is selected from: (1-(trifluoromethyl)cyclobutoxy)carbonyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (2,2,2-trifluoroethyl)sulfonyl, (3-(trifluoromethyl)oxetan-3-yloxy)carbonyl, 2,2,3,3,3-pentafluoropropoxy)carbonyl, 3-(2-fluoropropan-2-yl)-1,2,4-oxadiazol-5-yl, 3-(trifluoromethyl)-1,2,4-oxadiazol-5-yl, 3-(trifluoromethyl)-1,2,4-oxadiazol-5-ylmethyl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, cyclobutylthiocarbonyl, cyclopropylthiocarbonyl, and tert-butoxycarbonyl.

One aspect of the present invention pertains to compounds of Formula XX and pharmaceutically acceptable salts, solvates, and hydrates thereof:

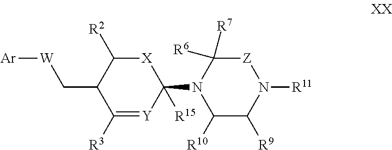

wherein:

Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl;

W is selected from: O and NR¹; or W is absent;

R¹ is selected from: H and C₁-C₆ alkyl;

R² and R³ are each H; or R² and R³ together form —CH₂—CH₂—;

X is selected from: —CH₂—CH₂—, CHR⁴, C(O), CHF, and CF₂; or X is absent;

Y is CR⁵;

R⁴ and R⁵ are each H; or R⁴ and R⁵ together form —CH₂—CH₂—;

Z is selected from: —CH₂—CH₂—, CHR⁸, and C(O);

R⁶, R⁹ and R¹⁰ are each H, and R⁷ and R⁸ are each independently selected from: H and C₁-C₆ alkyl; or R⁸, R⁹ and R¹⁰ are each H, and R⁶ and R⁷ together form —CH₂—CH₂—; or R⁶, R⁸ and R⁹ are each H, and R⁷ and R¹⁰ together form —CH₂—CH₂, or a bond; or R⁶, R⁷ and R¹⁰ are each H, and R⁸ and R⁹ together form —CH₂—CH₂; or R⁶, R⁷ and R⁹ are each H, and R⁸ and R¹⁰ together form —CH₂—CH₂—; or R⁶, R⁷ and R⁸ are each H, and R⁹ and R¹⁰ together form —CH₂—;

R¹¹ is selected from: C₁-C₆ alkoxycarbonyl, C₁-C₆ alkylsulfonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, heteroaryl-C₁-C₆ alkylene, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen; and

R¹⁵ is selected from: H and cyano

Some embodiments of the present invention include every combination of one or more compound and pharmaceutically acceptable salts, solvates, and hydrates thereof selected from the following group shown in Table A.

TABLE A

| # | Chemical Structure | Name |
|---|---|---|
| 1 | 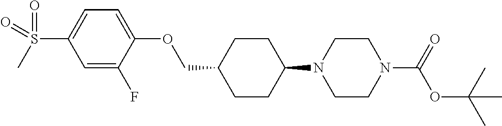 [SMILES: CC(C)(C)OC(=O)N1CCN([C@H]2CC[C@H](COc3ccc(S(C)(=O)=O)cc3F)CC2)CC1] | tert-Butyl 4-((1r,4r)-4-((2- fluoro-4- (methylsulfonyl)phenoxy) methyl)cyclohexyl) piperazine-1-carboxylate |
| 2 | 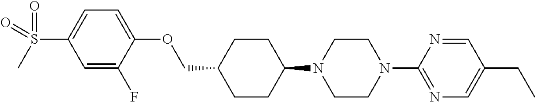 [SMILES: CCc1cnc(N2CCN([C@H]3CC[C@H](COc4ccc(S(C)(=O)=O)cc4F)CC3)CC2)nc1] | 5-Ethyl-2-(4-((1r,4r)-4-((2- fluoro-4- (methylsulfonyl)phenoxy) methyl)cyclohexyl)piperazin- 1-yl)pyrimidine |
| 3 | 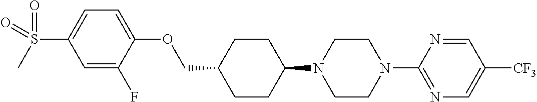 [SMILES: CS(=O)(=O)c1ccc(OC[C@H]2CC[C@H](N3CCN(c4ncc(C(F)(F)F)cn4)CC3)CC2)c(F)c1] | 2-(4-((1r,4r)-4-((2-Fluoro-4- (methylsulfonyl)phenoxy) methyl)cyclohexyl) piperazin-1-yl)-5- (trifluoromethyl)pyrimidine |
| 4 | 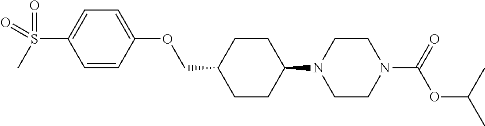 [SMILES: CC(C)OC(=O)N1CCN([C@H]2CC[C@H](COc3ccc(S(C)(=O)=O)cc3)CC2)CC1] | Isopropyl 4-((1r,4r)-4-((4- (methylsulfonyl)phenoxy) methyl)cyclohexyl) piperazine-1-carboxylate |
| 5 | 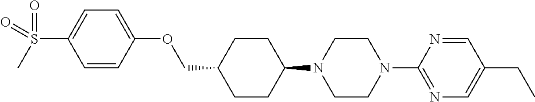 [SMILES: CCc1cnc(N2CCN([C@H]3CC[C@H](COc4ccc(S(C)(=O)=O)cc4)CC3)CC2)nc1] | 5-Ethyl-2-(4-((1r,4r)-4-((4- (methylsulfonyl)phenoxy) methyl)cyclohexyl)piperazin- 1-yl)pyrimidine |
| 6 | 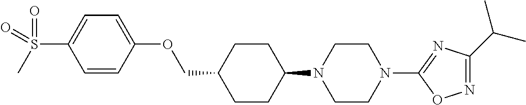 [SMILES: CC(C)c1noc(N2CCN([C@H]3CC[C@H](COc4ccc(S(C)(=O)=O)cc4)CC3)CC2)n1] | 3-Isopropyl-5-(4-((1r,4r)-4- ((4-(methylsulfonyl) phenoxy)methyl)cyclohexyl) piperazin-1-yl)-1,2,4- oxadiazole |
| 7 | 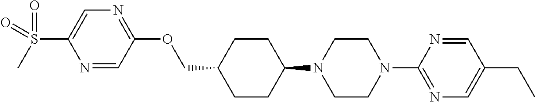 [SMILES: CCc1cnc(N2CCN([C@H]3CC[C@H](COc4cnc(S(C)(=O)=O)cn4)CC3)CC2)nc1] | 5-Ethyl-2-(4-((1r,4r)-4-((5- (methylsulfonyl)pyrazin-2- yloxy)methyl)cyclohexyl) piperazin-1-yl)pyrimidine |
| 8 | 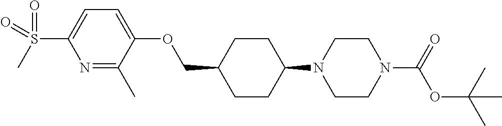 [SMILES: Cc1nc(S(C)(=O)=O)ccc1OC[C@H]1CC[C@@H](N2CCN(C(=O)OC(C)(C)C)CC2)CC1] | tert-Butyl 4-((1s,4s)-4-((2- methyl-6- (methylsulfonyl)pyridin-3- yloxy)methyl)cyclohexyl) piperazine-1-carboxylate |
| 9 | 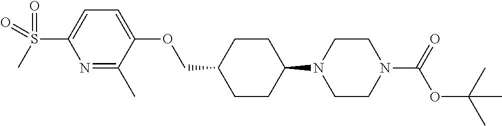 [SMILES: Cc1nc(S(C)(=O)=O)ccc1OC[C@H]1CC[C@H](N2CCN(C(=O)OC(C)(C)C)CC2)CC1] | tert-Butyl 4-((1r,4r)-4-((2- methyl-6-(methylsulfonyl) pyridin-3-yloxy)methyl) cyclohexyl)piperazine-1- carboxylate |
| 10 | 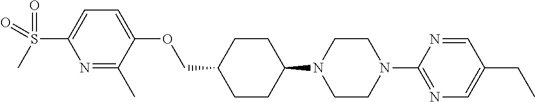 [SMILES: CCc1cnc(N2CCN([C@H]3CC[C@H](COc4ccc(S(C)(=O)=O)nc4C)CC3)CC2)nc1] | 5-Ethyl-2-(4-((1r,4r)-4-((2- methyl-6-(methylsulfonyl) pyridin-3-yloxy)methyl) cyclohexyl)piperazin-1- yl)pyrimidine |
| 11 | 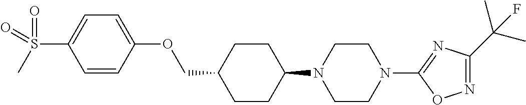 [SMILES: CC(C)(F)c1noc(N2CCN([C@H]3CC[C@H](COc4ccc(S(C)(=O)=O)cc4)CC3)CC2)n1] | 3-(2-Fluoropropan-2-yl)-5- (4-((1r,4r)-4-((4- (methylsulfonyl)phenoxy) methyl)cyclohexyl)piperazin- 1-yl)-1,2,4-oxadiazole |
| 12 | 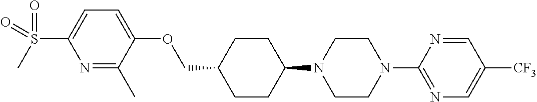 [SMILES: Cc1nc(S(C)(=O)=O)ccc1OC[C@H]1CC[C@H](N2CCN(c3ncc(C(F)(F)F)cn3)CC2)CC1] | 2-(4-((1r,4r)-4-((2-Methyl- 6-(methylsulfonyl)pyridin- 3-yloxy)methyl)cyclohexyl) piperazin-1-yl)-5- (trifluoromethyl)pyrimidine |
| 13 | 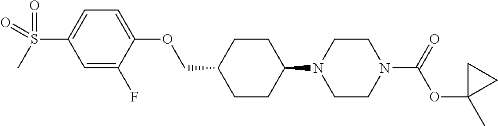 [SMILES: CC1(OC(=O)N2CCN([C@H]3CC[C@H](COc4ccc(S(C)(=O)=O)cc4F)CC3)CC2)CC1] | 1-Methylcyclopropyl 4- ((1r,4r)-4-((2-fluoro-4- (methylsulfonyl)phenoxy) methyl)cyclohexyl) piperazine-1-carboxylate |
| 14 | 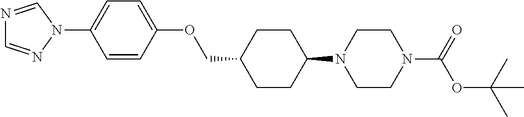 [SMILES: CC(C)(C)OC(=O)N1CCN([C@H]2CC[C@H](COc3ccc(-n4cncn4)cc3)CC2)CC1] | tert-Butyl 4-((1r,4r)-4-((4- (1H-1,2,4-triazol-1-yl) phenoxy)methyl) cyclohexyl)piperazine-1- carboxylate |
| 15 | 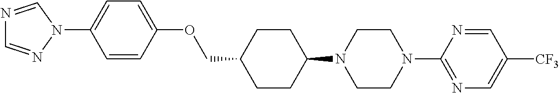 [SMILES: FC(F)(F)c1cnc(N2CCN([C@H]3CC[C@H](COc4ccc(-n5cncn5)cc4)CC3)CC2)nc1] | 2-(4-((1r,4r)-4-((4-(1H- 1,2,4-Triazol-1-yl)phenoxy) methyl)cyclohexyl) piperazin-1-yl)-5- (trifluoromethyl)pyrimidine |
| 16 | 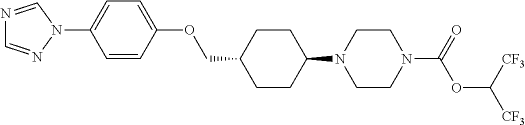 [SMILES: O=C(OC(C(F)(F)F)C(F)(F)F)N1CCN([C@H]2CC[C@H](COc3ccc(-n4cncn4)cc3)CC2)CC1] | 1,1,1,3,3,3- Hexafluoropropan-2-yl 4- ((1r,4r)-4-((4-(1H-1,2,4- triazol-1- yl)phenoxy)methyl) cyclohexyl)piperazine-1- carboxylate |
| 17 | 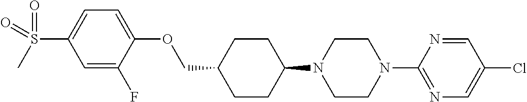 [SMILES: CS(=O)(=O)c1ccc(OC[C@H]2CC[C@H](N3CCN(c4ncc(Cl)cn4)CC3)CC2)c(F)c1] | 5-Chloro-2-(4-((1r,4r)-4- ((2-fluoro-4-(methylsulfonyl) phenoxy)methyl) cyclohexyl)piperazin-1- yl)pyrimidine |
| 18 | 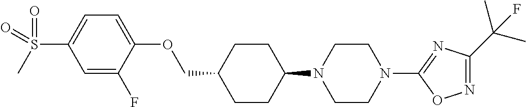 [SMILES: CC(C)(F)c1noc(N2CCN([C@H]3CC[C@H](COc4ccc(S(C)(=O)=O)cc4F)CC3)CC2)n1] | 5-(4-((1r,4r)-4-((2-Fluoro-4- (methylsulfonyl)phenoxy) methyl)cyclohexyl)piperazin- 1-yl)-3-(2-fluoropropan-2- yl)-1,2,4-oxadiazole |
| 19 | 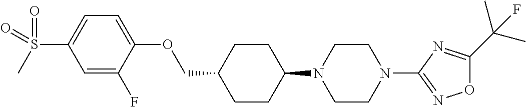 [SMILES: CC(C)(F)c1nc(N2CCN([C@H]3CC[C@H](COc4ccc(S(C)(=O)=O)cc4F)CC3)CC2)no1] | 3-(4-((1r,4r)-4-((2-Fluoro-4- (methylsulfonyl)phenoxy) methyl)cyclohexyl)piperazin- 1-yl)-5-(2-fluoropropan-2- yl)-1,2,4-oxadiazole |
| 20 | 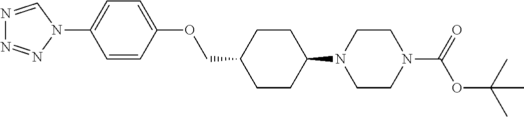 [SMILES: CC(C)(C)OC(=O)N1CCN([C@H]2CC[C@H](COc3ccc(-n4cnnn4)cc3)CC2)CC1] | tert-Butyl 4-((1r,4r)-4-((4- (1H-tetrazol-1-yl)phenoxy) methyl)cyclohexyl) piperazine-1-carboxylate |
| 21 | 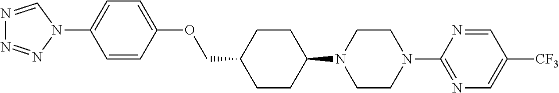 [SMILES: FC(F)(F)c1cnc(N2CCN([C@H]3CC[C@H](COc4ccc(-n5cnnn5)cc4)CC3)CC2)nc1] | 2-(4-((1r,4r)-4-((4-(1H- Tetrazol-1- yl)phenoxy)methyl) cyclohexyl)piperazin-1-yl)- 5-(trifluoromethyl) pyrimidine |
| 22 | 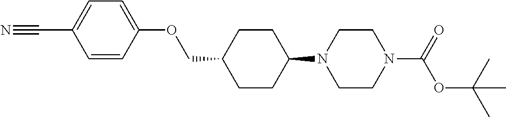 [SMILES: CC(C)(C)OC(=O)N1CCN([C@H]2CC[C@H](COc3ccc(C#N)cc3)CC2)CC1] | tert-Butyl 4-((1r,4r)-4-((4- cyanophenoxy)methyl) cyclohexyl)piperazine-1- carboxylate |
| 23 | 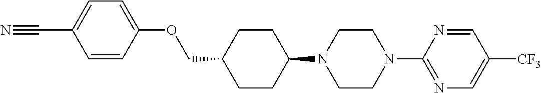 [SMILES: N#Cc1ccc(OC[C@H]2CC[C@H](N3CCN(c4ncc(C(F)(F)F)cn4)CC3)CC2)cc1] | 4-(((1r,4r)-4-(4-(5- (Trifluoromethyl)pyrimidin- 2-yl)piperazin-1- yl)cyclohexyl)methoxy) benzonitrile |
| 24 | 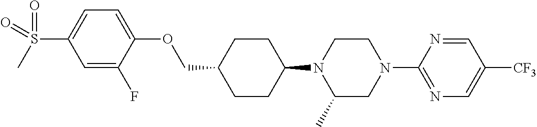 [SMILES: C[C@H]1CN(c2ncc(C(F)(F)F)cn2)CCN1[C@H]1CC[C@H](COc2ccc(S(C)(=O)=O)cc2F)CC1] | 2-((S)-4-((1r,4S)-4-((2- Fluoro-4- (methylsulfonyl)phenoxy) methyl)cyclohexyl)-3 methylpiperazin-1-yl)-5- (trifluoromethyl)pyrimidine |
| 25 | 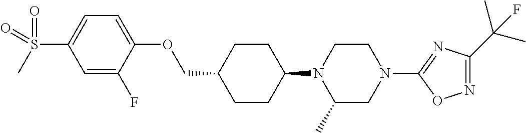 [SMILES: C[C@H]1CN(c2nc(C(C)(C)F)no2)CCN1[C@H]1CC[C@H](COc2ccc(S(C)(=O)=O)cc2F)CC1] | 5-((S)-4-((1r,4S)-4-((2- Fluoro-4- (methylsulfonyl)phenoxy) methyl)cyclohexyl)-3- methylpiperazin-1-yl)-3-(2- fluoropropan-2-yl)-1,2,4- oxadiazole |
| 26 | 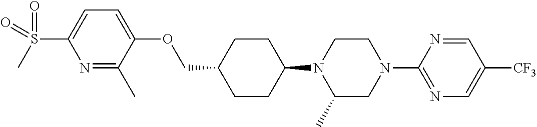 [SMILES: Cc1nc(S(C)(=O)=O)ccc1OC[C@H]1CC[C@H](N2CCN(c3ncc(C(F)(F)F)cn3)C[C@@H]2C)CC1] | 2-((S)-3-Methyl-4-((1r,4S)- 4-((2-methyl-6- (methylsulfonyl)pyridin-3- yloxy)methyl)cyclohexyl) piperazin-1-yl)-5- (trifluoromethyl)pyrimidine |
| 27 | 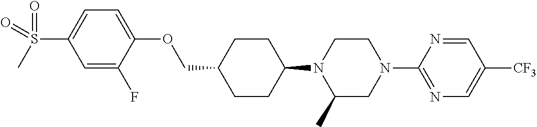 [SMILES: C[C@@H]1CN(c2ncc(C(F)(F)F)cn2)CCN1[C@H]1CC[C@H](COc2ccc(S(C)(=O)=O)cc2F)CC1] | 2-((R)-4-((1r,4R)-4-((2- Fluoro-4- (methylsulfonyl)phenoxy) methyl)cyclohexyl)-3- methylpiperazin-1-yl)-5- (trifluoromethyl)pyrimidine |
| 28 | 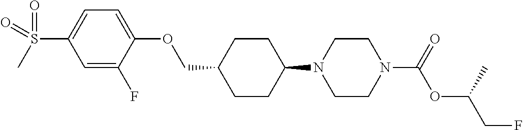 [SMILES: C[C@H](CF)OC(=O)N1CCN([C@H]2CC[C@H](COc3ccc(S(C)(=O)=O)cc3F)CC2)CC1] | (R)-1-Fluoropropan-2-yl 4- ((1r,4R)-4-((2-fluoro-4- (methylsulfonyl)phenoxy) methyl)cyclohexyl) piperazine-1-carboxylate |
| 29 | 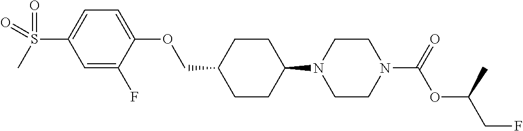 [SMILES: C[C@@H](CF)OC(=O)N1CCN([C@H]2CC[C@H](COc3ccc(S(C)(=O)=O)cc3F)CC2)CC1] | (S)-1-Fluoropropan-2-yl 4- ((1r,4S)-4-((2-fluoro-4- (methylsulfonyl)phenoxy) methyl)cyclohexyl) piperazine-1-carboxylate |
| 30 | 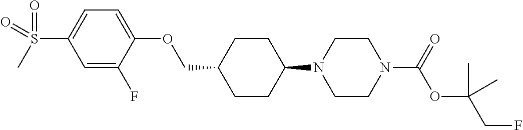 [SMILES: CC(C)(CF)OC(=O)N1CCN([C@H]2CC[C@H](COc3ccc(S(C)(=O)=O)cc3F)CC2)CC1] | 1-Fluoro-2-methylpropan-2- yl 4-((1r,4r)-4-((2-fluoro-4- (methylsulfonyl)phenoxy) methyl)cyclohexyl) piperazine-1-carboxylate |
| 31 | 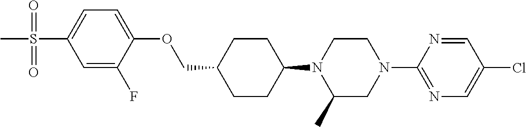 [SMILES: C[C@@H]1CN(c2ncc(Cl)cn2)CCN1[C@H]1CC[C@H](COc2ccc(S(C)(=O)=O)cc2F)CC1] | 5-Chloro-2-((R)-4-((1r,4R)- 4-((2-fluoro-4- (methylsulfonyl)phenoxy) methyl)cyclohexyl)-3- methylpiperazin-1- yl)pyrimidine |
| 32 | 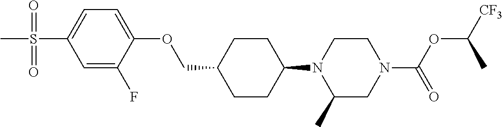 [SMILES: C[C@@H]1CN(C(=O)O[C@H](C)C(F)(F)F)CCN1[C@H]1CC[C@H](COc2ccc(S(C)(=O)=O)cc2F)CC1] | (R)-((R)-1,1,1- Trifluoropropan-2-yl) 4- ((1r,4R)-4-((2-fluoro-4- (methylsulfonyl)phenoxy) methyl)cyclohexyl)-3- methylpiperazine-1- carboxylate |
| 33 | 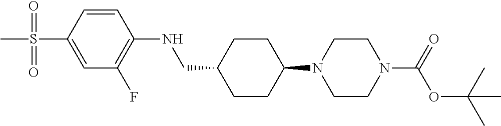 [SMILES: CC(C)(C)OC(=O)N1CCN([C@H]2CC[C@H](CNc3ccc(S(C)(=O)=O)cc3F)CC2)CC1] | tert-Butyl 4-((1r,4r)-4-((2- fluoro-4-(methylsulfonyl) phenylamino)methyl) cyclohexyl)piperazine-1- carboxylate |
| 34 | 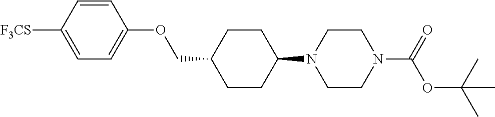 | tert-Butyl 4-((1r,4r)-4-((4- (trifluoromethylthio) phenoxy)methyl) cyclohexyl)piperazine-1- carboxylate |
| 35 | 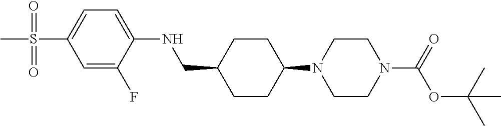 [SMILES: CC(C)(C)OC(=O)N1CCN([C@H]2CC[C@@H](CNc3ccc(S(C)(=O)=O)cc3F)CC2)CC1] | tert-Butyl 4-((1s,4s)-4-((2- fluoro-4-(methylsulfonyl) phenylamino)methyl) cyclohexyl)piperazine-1- carboxylate |
| 36 | 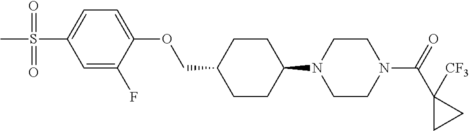 [SMILES: CS(=O)(=O)c1ccc(OC[C@H]2CC[C@H](N3CCN(C(=O)C4(C(F)(F)F)CC4)CC3)CC2)c(F)c1] | (4-((1r,4r)-4-((2-Fluoro-4- (methylsulfonyl)phenoxy) methyl)cyclohexyl) piperazin-1-yl)(1- (trifluoromethyl) cyclopropyl)methanone |
| 37 | 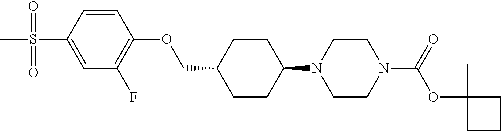 [SMILES: CC1(OC(=O)N2CCN([C@H]3CC[C@H](COc4ccc(S(C)(=O)=O)cc4F)CC3)CC2)CCC1] | 1-Methylcyclobutyl 4- ((1r,4r)-4-((2-fluoro-4- (methylsulfonyl)phenoxy) methyl)cyclohexyl) piperazine-1-carboxylate |
| 38 | 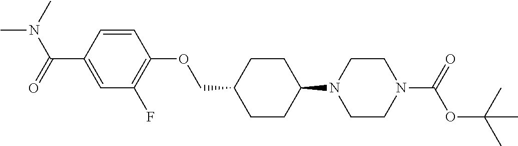 [SMILES: CN(C)C(=O)c1ccc(OC[C@H]2CC[C@H](N3CCN(C(=O)OC(C)(C)C)CC3)CC2)c(F)c1] | tert-Butyl 4-((1r,4r)-4-((4- (dimethylcarbamoyl)-2- fluorophenoxy)methyl) cyclohexyl)piperazine-1- carboxylate |
| 39 | 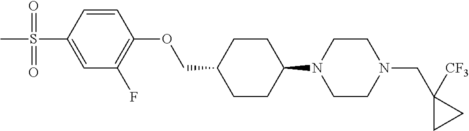 [SMILES: CS(=O)(=O)c1ccc(OC[C@H]2CC[C@H](N3CCN(CC4(C(F)(F)F)CC4)CC3)CC2)c(F)c1] | 1-((1r,4r)-4-((2-Fluoro-4- (methylsulfonyl)phenoxy) methyl)cyclohexyl)-4-((1- (trifluoromethyl)cyclopropyl) methyl)piperazine |
| 40 | 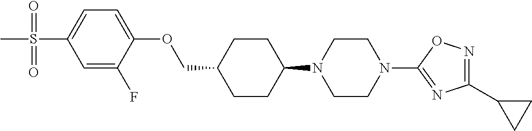 [SMILES: CS(=O)(=O)c1ccc(OC[C@H]2CC[C@H](N3CCN(c4nc(C5CC5)no4)CC3)CC2)c(F)c1] | 3-Cyclopropyl-5-(4-((1r,4r)- 4-((2-fluoro-4- (methylsulfonyl)phenoxy) methyl)cyclohexyl)piperazin- 1-yl)-1,2,4-oxadiazole |
| 41 | 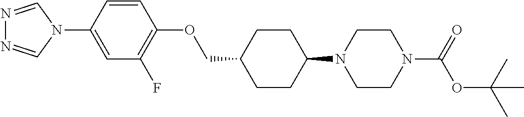 [SMILES: CC(C)(C)OC(=O)N1CCN([C@H]2CC[C@H](COc3ccc(-n4cnnc4)cc3F)CC2)CC1] | tert-Butyl 4-((1r,4r)-4-((2- fluoro-4-(4H-1,2,4-triazol-4- yl)phenoxy)methyl) cyclohexyl)piperazine-1- carboxylate |
| 42 | 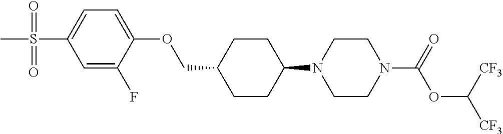 [SMILES: CS(=O)(=O)c1ccc(OC[C@H]2CC[C@H](N3CCN(C(=O)OC(C(F)(F)F)C(F)(F)F)CC3)CC2)c(F)c1] | 1,1,1,3,3,3- Hexafluoropropan-2-yl 4- ((1r,4r)-4-((2-fluoro-4- (methylsulfonyl)phenoxy) methyl)cyclohexyl) piperazine-1-carboxylate |
| 43 | 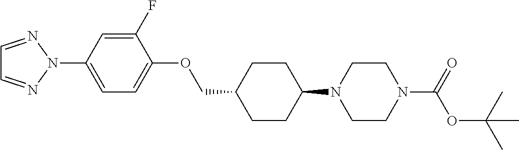 [SMILES: CC(C)(C)OC(=O)N1CCN([C@H]2CC[C@H](COc3ccc(-n4nccn4)cc3F)CC2)CC1] | tert-Butyl 4-((1r,4r)-4-((2- fluoro-4-(2H-1,2,3-triazol-2- yl)phenoxy)methyl) cyclohexyl)piperazine-1- carboxylate |
| 44 | 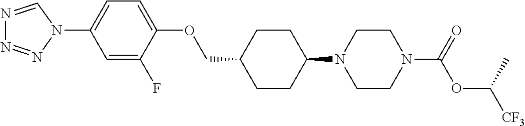 [SMILES: C[C@@H](OC(=O)N1CCN([C@H]2CC[C@H](COc3ccc(-n4cnnn4)cc3F)CC2)CC1)C(F)(F)F] | (R)-1,1,1-Trifluoropropan-2- yl 4-((1r,4R)-4-((2-fluoro-4- (1H-tetrazol-1-yl)phenoxy) methyl)cyclohexyl) piperazine-1-carboxylate |
| 45 | 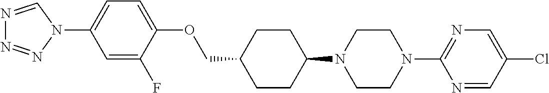 [SMILES: Fc1cc(-n2cnnn2)ccc1OC[C@H]1CC[C@H](N2CCN(c3ncc(Cl)cn3)CC2)CC1] | 5-chloro-2-(4-((1r,4r)-4-((2- fluoro-4-(1H-tetrazol-1- yl)phenoxy)methyl) cyclohexyl)piperazin-1- yl)pyrimidine |
| 46 | 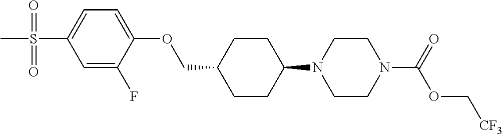 [SMILES: CS(=O)(=O)c1ccc(OC[C@H]2CC[C@H](N3CCN(C(=O)OCC(F)(F)F)CC3)CC2)c(F)c1] | 2,2,2-Trifluoroethyl 4- ((1r,4r)-4-((2-fluoro-4- (methylsulfonyl)phenoxy) methyl)cyclohexyl) piperazine-1-carboxylate |
| 47 | 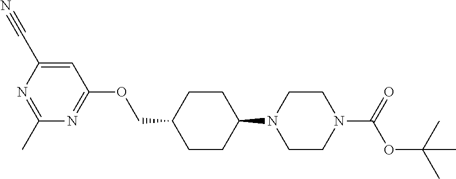 [SMILES: Cc1nc(C#N)cc(OC[C@H]2CC[C@H](N3CCN(C(=O)OC(C)(C)C)CC3)CC2)n1] | tert-Butyl 4-((1r,4r)-4-((6- cyano-2-methylpyrimidin-4- yloxy)methyl)cyclohexyl) piperazine-1-carboxylate |
| 48 | 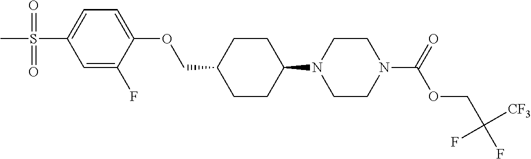 [SMILES: CS(=O)(=O)c1ccc(OC[C@H]2CC[C@H](N3CCN(C(=O)OCC(F)(F)C(F)(F)F)CC3)CC2)c(F)c1] | 2,2,3,3,3-Pentafluoropropyl 4-((1r,4r)-4-((2-fluoro-4- (methylsulfonyl)phenoxy) methyl)cyclohexyl) piperazine-1-carboxylate |
| 49 | 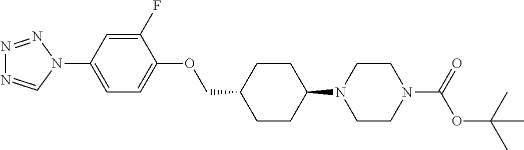 [SMILES: CC(C)(C)OC(=O)N1CCN([C@H]2CC[C@H](COc3ccc(-n4cnnn4)cc3F)CC2)CC1] | tert-Butyl 4-((1r,4r)-4-((2- fluoro-4-(1H-tetrazol-1- yl)phenoxy)methyl) cyclohexyl)piperazine-1- carboxylate |
| 50 | 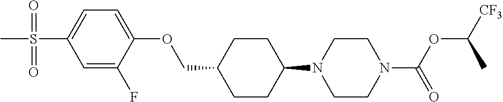 [SMILES: C[C@@H](OC(=O)N1CCN([C@H]2CC[C@H](COc3ccc(S(C)(=O)=O)cc3F)CC2)CC1)C(F)(F)F] | (R)-1,1,1-Trifluoropropan-2- yl 4-((1r,4R)-4-((2-fluoro-4- (methylsulfonyl)phenoxy) methyl)cyclohexyl) piperazine-1-carboxylate |
| 51 | 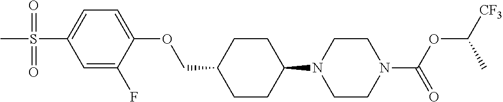 [SMILES: C[C@H](OC(=O)N1CCN([C@H]2CC[C@H](COc3ccc(S(C)(=O)=O)cc3F)CC2)CC1)C(F)(F)F] | (S)-1,1,1-Trifluoropropan-2- yl 4-((1r,4S)-4-((2-fluoro-4- (methylsulfonyl)phenoxy) methyl)cyclohexyl) piperazine-1-carboxylate |
| 52 | 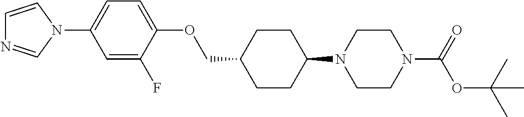 [SMILES: CC(C)(C)OC(=O)N1CCN([C@H]2CC[C@H](COc3ccc(-n4ccnc4)cc3F)CC2)CC1] | tert-Butyl 4-((1r,4r)-4-((2- fluoro-4-(1H-imidazol-1- yl)phenoxy)methyl) cyclohexyl)piperazine-1- carboxylate |
| 53 | 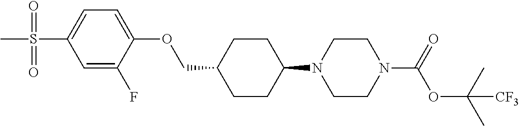 [SMILES: CC(C)(OC(=O)N1CCN([C@H]2CC[C@H](COc3ccc(S(C)(=O)=O)cc3F)CC2)CC1)C(F)(F)F] | 1,1,1-Trifluoro-2- methylpropan-2-yl 4- ((1r,4r)-4-((2-fluoro-4- (methylsulfonyl)phenoxy) methyl)cyclohexyl) piperazine-1-carboxylate |
| 54 | 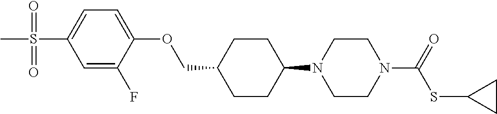 [SMILES: CS(=O)(=O)c1ccc(OC[C@H]2CC[C@H](N3CCN(C(=O)SC4CC4)CC3)CC2)c(F)c1] | S-Cyclopropyl 4-((1r,4r)-4- ((2-fluoro-4- (methylsulfonyl)phenoxy) methyl)cyclohexyl) piperazine-1-carbothioate |
| 55 | 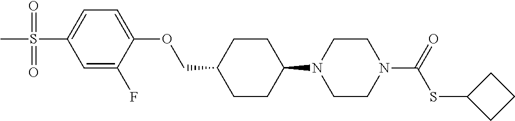 [SMILES: CS(=O)(=O)c1ccc(OC[C@H]2CC[C@H](N3CCN(C(=O)SC4CCC4)CC3)CC2)c(F)c1] | S-Cyclobutyl 4-((1r,4r)-4- ((2-fluoro-4- (methylsulfonyl)phenoxy) methyl)cyclohexyl) piperazine-1-carbothioate |
| 56 | 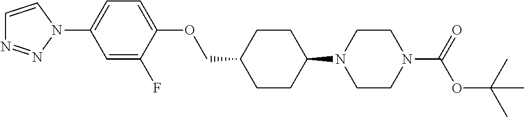 [SMILES: CC(C)(C)OC(=O)N1CCN([C@H]2CC[C@H](COc3ccc(-n4ccnn4)cc3F)CC2)CC1] | tert-Butyl 4-((1r,4r)-4-((2- fluoro-4-(1H-1,2,3-triazol-1- yl)phenoxy)methyl) cyclohexyl)piperazine-1- carboxylate |
| 57 | 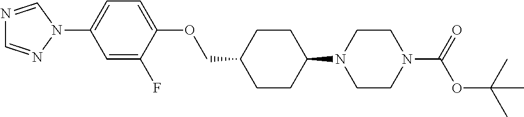 [SMILES: CC(C)(C)OC(=O)N1CCN([C@H]2CC[C@H](COc3ccc(-n4cncn4)cc3F)CC2)CC1] | tert-Butyl 4-((1r,4r)-4-((2- fluoro-4-(1H-1,2,4-triazol-1- yl)phenoxy)methyl) cyclohexyl)piperazine-1- carboxylate |
| 58 | 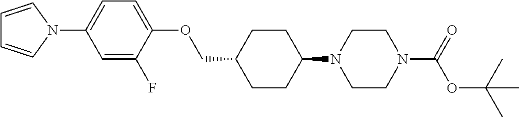 [SMILES: CC(C)(C)OC(=O)N1CCN([C@H]2CC[C@H](COc3ccc(-n4cccc4)cc3F)CC2)CC1] | tert-Butyl 4-((1r,4r)-4-((2- fluoro-4-(1H-pyrrol-1-yl) phenoxy)methyl) cyclohexyl)piperazine-1- carboxylate |
| 59 | 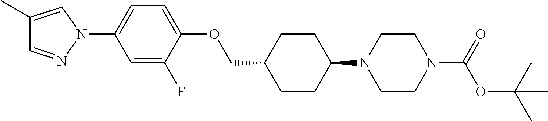 [SMILES: Cc1cnn(-c2ccc(OC[C@H]3CC[C@H](N4CCN(C(=O)OC(C)(C)C)CC4)CC3)c(F)c2)c1] | tert-Butyl 4-((1r,4r)-4-((2- fluoro-4-(4-methyl-1H- pyrazol-1-yl)phenoxy) methyl)cyclohexyl) piperazine-1-carboxylate |
| 60 | 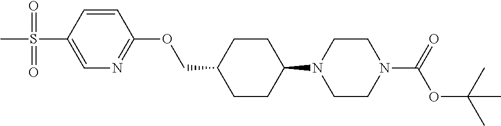 [SMILES: CC(C)(C)OC(=O)N1CCN([C@H]2CC[C@H](COc3ccc(S(C)(=O)=O)cn3)CC2)CC1] | tert-Butyl 4-((1r,4r)-4-((5- (methylsulfonyl)pyridin-2- yloxy)methyl)cyclohexyl) piperazine-1-carboxylate |
| 61 | 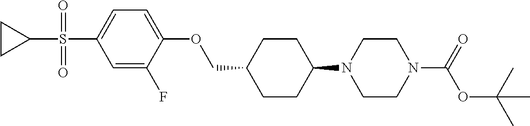 [SMILES: CC(C)(C)OC(=O)N1CCN([C@H]2CC[C@H](COc3ccc(S(=O)(=O)C4CC4)cc3F)CC2)CC1] | tert-Butyl 4-((1r,4r)-4-((4- (cyclopropylsulfonyl)-2- fluorophenoxy)methyl) cyclohexyl)piperazine-1- carboxylate |
| 62 | 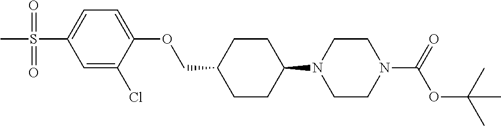 [SMILES: CC(C)(C)OC(=O)N1CCN([C@H]2CC[C@H](COc3ccc(S(C)(=O)=O)cc3Cl)CC2)CC1] | tert-Butyl 4-((1r,4r)-4-((2- chloro-4-(methylsulfonyl) phenoxy)methyl) cyclohexyl)piperazine-1- carboxylate |
| 63 | 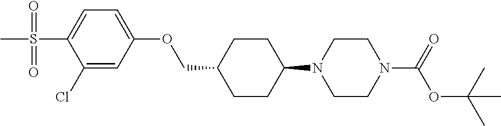 [SMILES: CC(C)(C)OC(=O)N1CCN([C@H]2CC[C@H](COc3ccc(S(C)(=O)=O)c(Cl)c3)CC2)CC1] | tert-Butyl 4-((1r,4r)-4-((3- chloro-4-(methylsulfonyl) phenoxy)methyl) cyclohexyl)piperazine-1- carboxylate |
| 64 | 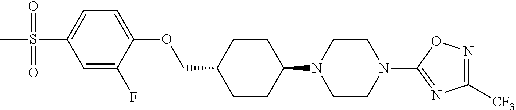 [SMILES: CS(=O)(=O)c1ccc(OC[C@H]2CC[C@H](N3CCN(c4nc(C(F)(F)F)no4)CC3)CC2)c(F)c1] | 5-(4-((1r,4r)-4-((2-Fluoro-4- (methylsulfonyl)phenoxy) methyl)cyclohexyl)piperazin- 1-yl)-3-(trifluoromethyl)- 1,2,4-oxadiazole |
| 65 | 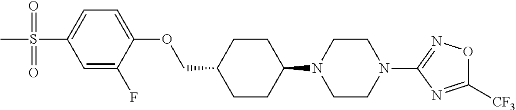 [SMILES: CS(=O)(=O)c1ccc(OC[C@H]2CC[C@H](N3CCN(c4noc(C(F)(F)F)n4)CC3)CC2)c(F)c1] | 3-(4-((1r,4r)-4-((2-Fluoro-4- (methylsulfonyl)phenoxy) methyl)cyclohexyl)piperazin- 1-yl)-5-(trifluoromethyl)- 1,2,4-oxadiazole |
| 66 | 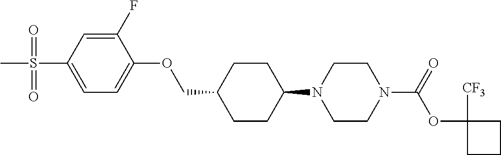 [SMILES: CS(=O)(=O)c1ccc(OC[C@H]2CC[C@H](N3CCN(C(=O)OC4(C(F)(F)F)CCC4)CC3)CC2)c(F)c1] | 1-(Trifluoromethyl) cyclobutyl 4-((1r,4r)-4-((2- fluoro-4-(methylsulfonyl) phenoxy)methyl) cyclohexyl)piperazine-1- carboxylate |
| 67 | 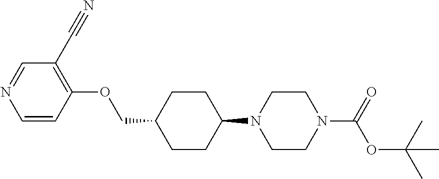 [SMILES: CC(C)(C)OC(=O)N1CCN([C@H]2CC[C@H](COc3ccncc3C#N)CC2)CC1] | tert-Butyl 4-((1r,4r)-4-((3- cyanopyridin-4- yloxy)methyl)cyclohexyl) piperazine-1-carboxylate |
| 68 | 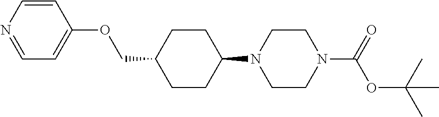 [SMILES: CC(C)(C)OC(=O)N1CCN([C@H]2CC[C@H](COc3ccncc3)CC2)CC1] | tert-Butyl 4-((1r,4r)-4- ((pyridin-4-yloxy) methyl)cyclohexyl) piperazine-1-carboxylate |
| 69 | 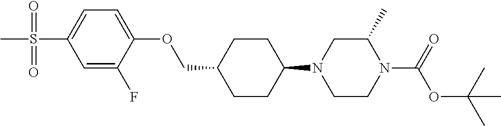 [SMILES: C[C@H]1CN([C@H]2CC[C@H](COc3ccc(S(C)(=O)=O)cc3F)CC2)CCN1C(=O)OC(C)(C)C] | (S)-tert-Butyl 4-((1r,4S)-4- ((2-fluoro-4- (methylsulfonyl)phenoxy) methyl)cyclohexyl)-2- methyl piperazine-1- carboxylate |
| 70 | 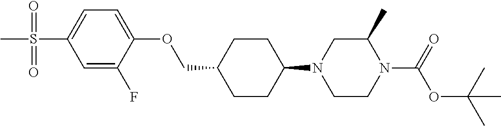 [SMILES: C[C@@H]1CN([C@H]2CC[C@H](COc3ccc(S(C)(=O)=O)cc3F)CC2)CCN1C(=O)OC(C)(C)C] | (R)-tert-Butyl 4-((1r,4R)-4- ((2-fluoro-4- (methylsulfonyl)phenoxy) methyl)cyclohexyl)-2- methylpiperazine-1- carboxylate |
| 71 | 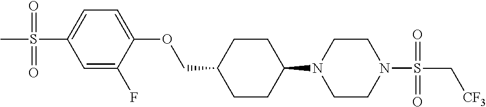 [SMILES: CS(=O)(=O)c1ccc(OC[C@H]2CC[C@H](N3CCN(S(=O)(=O)CC(F)(F)F)CC3)CC2)c(F)c1] | 1-((1r,4r)-4-((2-Fluoro-4- (methylsulfonyl)phenoxy) methyl)cyclohexyl)-4- (2,2,2-trifluoroethylsulfonyl) piperazine |
| 72 | 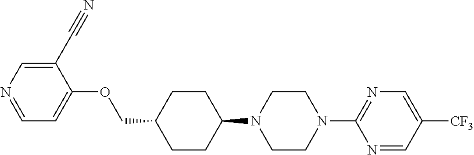 [SMILES: N#Cc1cnccc1OC[C@H]1CC[C@H](N2CCN(c3ncc(C(F)(F)F)cn3)CC2)CC1] | 4-(((1r,4r)-4-(4-(5- (Trifluoromethyl)pyrimidin- 2-yl)piperazin-1- yl)cyclohexyl)methoxy) nicotinonitrile |
| 73 | 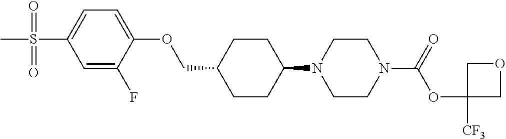 [SMILES: CS(=O)(=O)c1ccc(OC[C@H]2CC[C@H](N3CCN(C(=O)OC4(C(F)(F)F)COC4)CC3)CC2)c(F)c1] | 3-(Trifluoromethyl)oxetan- 3-yl 4-((1r,4r)-4-((2-fluoro- 4-(methylsulfonyl)phenoxy) methyl)cyclohexyl) piperazine-1-carboxylate |
| 74 | 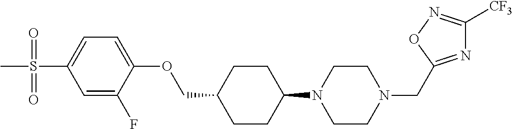 [SMILES: CS(=O)(=O)c1ccc(OC[C@H]2CC[C@H](N3CCN(Cc4nc(C(F)(F)F)no4)CC3)CC2)c(F)c1] | 5-((4-((1r,4r)-4-((2-Fluoro- 4-(methylsulfonyl)phenoxy) methyl)cyclohexyl)piperazin- 1-yl)methyl)-3-(trifluoromethyl)- 1,2,4-oxadiazole |
| 75 | 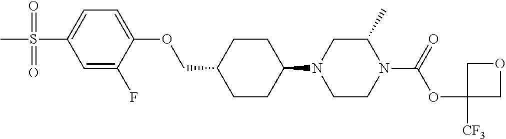 [SMILES: C[C@H]1CN([C@H]2CC[C@H](COc3ccc(S(C)(=O)=O)cc3F)CC2)CCN1C(=O)OC1(C(F)(F)F)COC1] | (S)-3-(Trifluoromethyl) oxetan-3-yl 4-((1r,4S)-4-((2- fluoro-4-(methylsulfonyl) phenoxy)methyl)cyclohexyl)- 2-methylpiperazine- 1-carboxylate |
| 76 | 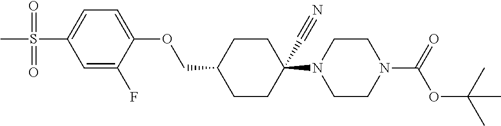 [SMILES: CC(C)(C)OC(=O)N1CCN([C@]2(C#N)CC[C@@H](COc3ccc(S(C)(=O)=O)cc3F)CC2)CC1] | tert-Butyl 4-((1r,4r)-1- cyano-4-((2-fluoro-4- (methylsulfonyl)phenoxy) methyl)cyclohexyl) piperazine-1-carboxylate |
| 77 | 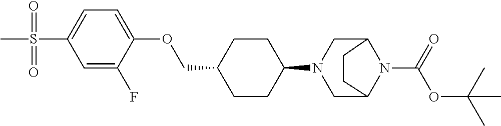 [SMILES: CC(C)(C)OC(=O)N1C2CCC1CN([C@H]1CC[C@H](COc3ccc(S(C)(=O)=O)cc3F)CC1)C2] | tert-Butyl 3-((1r,4r)-4-((2- fluoro-4-(methylsulfonyl) phenoxy)methyl)cyclohexyl)- 3,8-diazabicyclo [3.2.1]octane-8-carboxylate |
| 78 | 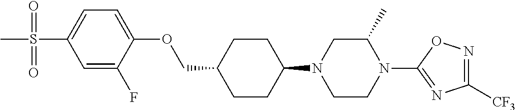 [SMILES: C[C@H]1CN([C@H]2CC[C@H](COc3ccc(S(C)(=O)=O)cc3F)CC2)CCN1c1nc(C(F)(F)F)no1] | 5-((S)-4-((1r,4S)-4-((2- Fluoro-4-(methylsulfonyl) phenoxy)methyl)cyclohexyl)- 2-methylpiperazin-1- yl)-3-(trifluoromethyl)- 1,2,4-oxadiazole |
| 79 | 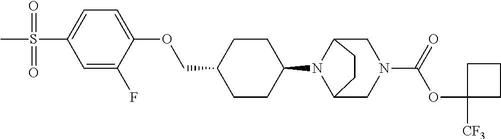 [SMILES: CS(=O)(=O)c1ccc(OC[C@H]2CC[C@H](N3C4CCC3CN(C(=O)OC3(C(F)(F)F)CCC3)C4)CC2)c(F)c1] | 1-(Trifluoromethyl) cyclobutyl 8-((1r,4r)-4-((2- fluoro-4-(methylsulfonyl) phenoxy)methyl)cyclohexyl)- 3,8-diazabicyclo [3.2.1]octane-3-carboxylate |
| 80 | 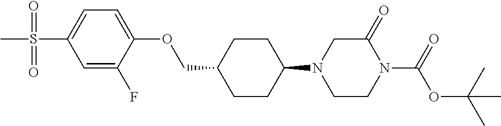 [SMILES: CC(C)(C)OC(=O)N1CCN([C@H]2CC[C@H](COc3ccc(S(C)(=O)=O)cc3F)CC2)CC1=O] | tert-Butyl 4-((1r,4r)-4-((2- fluoro-4-(methylsulfonyl) phenoxy)methyl)cyclohexyl)- 2-oxopiperazine-1- carboxylate |
| 81 | 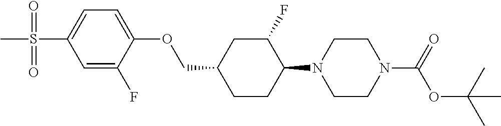 [SMILES: CC(C)(C)OC(=O)N1CCN([C@H]2CC[C@H](COc3ccc(S(C)(=O)=O)cc3F)C[C@@H]2F)CC1] | tert-Butyl 4-((1R,2S,4R)-2- fluoro-4-((2-fluoro-4- (methylsulfonyl)phenoxy) methyl)cyclohexyl) piperazine-1-carboxylate |
| 82 | 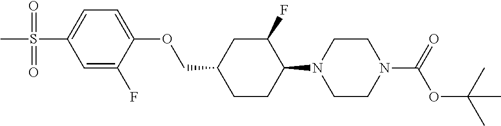 [SMILES: CC(C)(C)OC(=O)N1CCN([C@H]2CC[C@H](COc3ccc(S(C)(=O)=O)cc3F)C[C@H]2F)CC1] | tert-Butyl 4-((1R,2R,4R)-2- fluoro-4-((2-fluoro-4- (methylsulfonyl)phenoxy) methyl)cyclohexyl) piperazine-1-carboxylate |
| 83 | 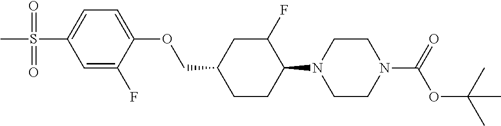 [SMILES: CC(C)(C)OC(=O)N1CCN([C@H]2CC[C@H](COc3ccc(S(C)(=O)=O)cc3F)CC2F)CC1] | tert-Butyl 4-((1R,4R)-2- fluoro-4-((2-fluoro-4- (methylsulfonyl)phenoxy) methyl)cyclohexyl) piperazine-1-carboxylate |
| 84 | 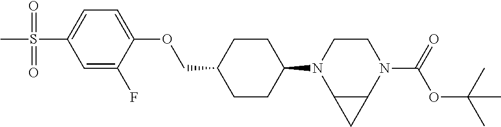 [SMILES: CC(C)(C)OC(=O)N1CCN([C@H]2CC[C@H](COc3ccc(S(C)(=O)=O)cc3F)CC2)C2CC21] | tert-Butyl 5-((1r,4r)-4-((2- fluoro-4- (methylsulfonyl)phenoxy) methyl)cyclohexyl)-2,5- diazabicyclo[4.1.0]heptane- 2-carboxylate |
| 85 | 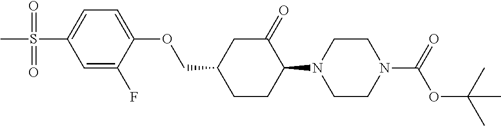 [SMILES: CC(C)(C)OC(=O)N1CCN([C@H]2CC[C@H](COc3ccc(S(C)(=O)=O)cc3F)CC2=O)CC1] | tert-Butyl 4-(1R,4R)-4-((2- fluoro-4-(methylsulfonyl) phenoxy)methyl)-2- oxocyclohexyl)piperazine-1- carboxylate |
| 86 | 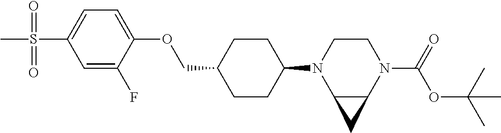 [SMILES: CC(C)(C)OC(=O)N1CCN([C@H]2CC[C@H](COc3ccc(S(C)(=O)=O)cc3F)CC2)[C@@H]2C[C@@H]21] | (1S,6R)-tert-Butyl 5- ((1r,4R)-4-((2-fluoro-4- (methylsulfonyl)phenoxy) methyl)cyclohexyl)-2,5- diazabicyclo[4.1.0]heptane- 2-carboxylate |
| 87 | 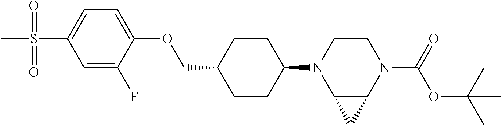 [SMILES: CC(C)(C)OC(=O)N1CCN([C@H]2CC[C@H](COc3ccc(S(C)(=O)=O)cc3F)CC2)[C@H]2C[C@H]21] | (1R,6S)-tert-Butyl 5- ((1r,4S)-4-((2-fluoro-4- (methylsulfonyl)phenoxy) methyl)cyclohexyl)-2,5- diazabicyclo[4.1.0]heptane- 2-carboxylate |
| 88 | 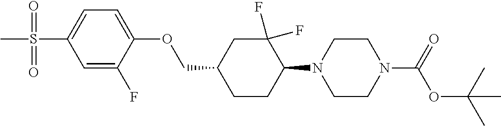 [SMILES: CC(C)(C)OC(=O)N1CCN([C@H]2CC[C@H](COc3ccc(S(C)(=O)=O)cc3F)CC2(F)F)CC1] | tert-Butyl 4-((1R,4R)-2,2- difluoro-4-((2-fluoro-4- (methylsulfonyl)phenoxy) methyl)cyclohexyl) piperazine-1-carboxylate |
| 89 | 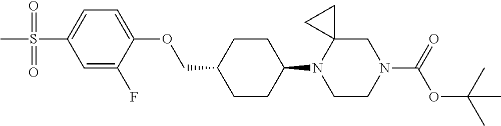 [SMILES: CC(C)(C)OC(=O)N1CCN([C@H]2CC[C@H](COc3ccc(S(C)(=O)=O)cc3F)CC2)C2(CC2)C1] | tert-Butyl 4-((1r,4r)-4-((2- fluoro-4-(methylsulfonyl) phenoxy)methyl) cyclohexyl)-4,7-diazaspiro [2.5]octane-7-carboxylate |
| 90 | 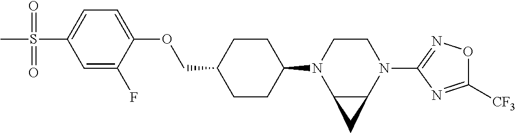 [SMILES: CS(=O)(=O)c1ccc(OC[C@H]2CC[C@H](N3CCN(c4noc(C(F)(F)F)n4)[C@H]4C[C@H]43)CC2)c(F)c1] | 3-((1S,6R)-5-((1r,4R)-4-((2- Fluoro-4- (methylsulfonyl)phenoxy) methyl)cyclohexyl)-2,5- diazabicyclo[4.1.0]heptan-2- yl)-5-(trifluoromethyl)- 1,2,4-oxadiazole |
| 91 | 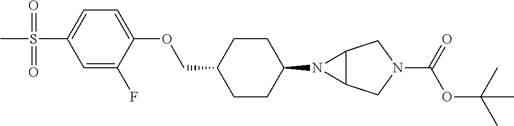 [SMILES: CC(C)(C)OC(=O)N1CC2C(C1)N2[C@H]1CC[C@H](COc2ccc(S(C)(=O)=O)cc2F)CC1] | tert-Butyl 6-((1r,4r)-4-((2- fluoro-4-(methylsulfonyl) phenoxy)methyl)cyclohexyl)- 3,6-diazabicyclo [3.1.0]hexane-3-carboxylate |

Additionally, individual compounds and chemical genera of the present invention, for example those compounds found in Table A including, isomers, diastereoisomers and enantiomers thereof, encompass all pharmaceutically acceptable salts, solvates, and hydrates, thereof. Further, mesoisomers of individual compounds and chemical genera of the present invention, for example those compounds found in Table A, encompass all pharmaceutically acceptable salts, solvates and particularly hydrates, thereof.

The compounds of the Formula I of the present invention may be prepared according to relevant published literature procedures that are used by one skilled in the art. Exemplary reagents and procedures for these reactions appear hereinafter in the working Examples.

Protection and deprotection may be carried out by procedures generally known in the art (see, for example, Greene, T. W. and Wuts, P. G. M., Protecting Groups in Organic Synthesis, 3^{rd} Edition, 1999 [Wiley]).

It is understood that the present invention embraces, each isomer, each diastereoisomer, each enantiomer and mixtures thereof of each compound and generic formulae disclosed herein just as if they were each individually disclosed with the specific stereochemical designation for each chiral carbon. Separation of the individual isomers and enantiomers (such as, by chiral HPLC, recrystallization of diastereoisomeric mixtures and the like) or selective synthesis (such as, by enantiomeric selective syntheses and the like) of the individual isomers can be accomplished by application of various methods which are well known to practitioners in the art.

One aspect of the present invention pertains to compounds, compositions, and pharmaceutical products of the present invention for use in a method of treatment of the human or animal body by therapy.

One aspect of the present invention pertains to compounds, compositions, and pharmaceutical products of the present invention for use in a method of modulating the activity of a GPR119 receptor in an individual.

One aspect of the present invention pertains to compounds, compositions, and pharmaceutical products of the present invention for use in a method of agonizing a GPR119 receptor in an individual.

One aspect of the present invention pertains to compounds, compositions, and pharmaceutical products of the present invention for use in a method of increasing the secretion of an incretin in an individual.

One aspect of the present invention pertains to compounds, compositions, and pharmaceutical products of the present invention for use in a method of increasing a blood incretin level in an individual.

One aspect of the present invention pertains to compounds, compositions, and pharmaceutical products of the present invention for use in a method of treating a disorder in an individual, wherein the disorder is selected from: a GPR119-receptor-related disorder; a condition ameliorated by increasing a blood incretin level; a condition characterized by low bone mass; a neurological disorder; a metabolic-related disorder; and obesity.

### Pharmaceutical Products, Methods, and Uses

One aspect of the present invention pertains to pharmaceutical products selected from: a pharmaceutical composition, a formulation, a dosage form, a combined preparation, a twin pack, and a kit; comprising a compound of the present invention.

One aspect of the present invention pertains to methods for preparing a composition comprising the step of admixing a compound of the present invention and a pharmaceutically acceptable carrier.

One aspect of the present invention pertains to methods for preparing a composition comprising: a first pharmaceutical agent selected from a compound of the present invention; and a second pharmaceutical agent.

One aspect of the present invention pertains to methods for preparing a composition comprising the step of admixing a first pharmaceutical agent selected from a compound of the present invention; a second pharmaceutical agent; and a pharmaceutically acceptable carrier.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention; and a second pharmaceutical agent.

One aspect of the present invention pertains to methods for modulating the activity of a GPR119 receptor, comprising administering to an individual in need thereof, a therapeutically effective amount of: a compound, a composition, or a pharmaceutical product of the present invention.

One aspect of the present invention pertains to methods for modulating the activity of a GPR119 receptor, comprising prescribing to an individual in need thereof, a therapeutically

effective amount of: a compound, a composition, or a pharmaceutical product of the present invention.

One aspect of the present invention pertains to methods for agonizing a GPR119 receptor, comprising administering to an individual in need thereof, a therapeutically effective amount of: a compound, a composition, or a pharmaceutical product of the present invention.

One aspect of the present invention pertains to methods for agonizing a GPR119 receptor, comprising prescribing to an individual in need thereof, a therapeutically effective amount of: a compound, a composition, or a pharmaceutical product of the present invention.

One aspect of the present invention pertains to methods for increasing the secretion of an incretin in an individual, comprising administering to the individual in need thereof, a therapeutically effective amount of: a compound, a composition, or a pharmaceutical product of the present invention.

One aspect of the present invention pertains to methods for increasing the secretion of an incretin in an individual, comprising prescribing to the individual in need thereof, a therapeutically effective amount of: a compound, a composition, or a pharmaceutical product of the present invention.

One aspect of the present invention pertains to methods for increasing a blood incretin level in an individual, comprising administering to the individual in need thereof, a therapeutically effective amount of: a compound, a composition, or a pharmaceutical product of the present invention.

One aspect of the present invention pertains to methods for increasing a blood incretin level in an individual, comprising prescribing to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual; comprising administering to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual, comprising prescribing to the individual in need thereof, a therapeutically effective amount of: a compound, a composition, or a pharmaceutical product of the present invention.

One aspect of the present invention pertains to uses of a compound, a composition, or a pharmaceutical product of the present invention; in the manufacture of a medicament for modulating the activity of a GPR119 receptor in an individual.

One aspect of the present invention pertains to uses of a compound, a composition, or a pharmaceutical product of the present invention; in the manufacture of a medicament for agonizing a GPR119 receptor in an individual.

One aspect of the present invention pertains to uses of a compound, a composition, or a pharmaceutical product of the present invention; in the manufacture of a medicament for increasing the secretion of an incretin in an individual.

One aspect of the present invention pertains to uses of a compound, a composition, or a pharmaceutical product of the present invention; in the manufacture of a medicament for increasing a blood incretin level in an individual.

One aspect of the present invention pertains to uses of a compound, a composition, or a pharmaceutical product of the present invention; in the manufacture of a medicament for the treating a disorder in an individual, wherein the disorder is selected from: a GPR119-receptor-related disorder; a condition ameliorated by increasing a blood incretin level; a condition characterized by low bone mass; a neurological disorder; a metabolic-related disorder; and obesity.

One aspect of the present invention pertains to pharmaceutical products selected from: a pharmaceutical composition, a formulation, a dosage form, a combined preparation, a twin pack, and a kit; comprising a compound of the present invention; for use in a method of treatment of the human or animal by therapy.

One aspect of the present invention pertains to pharmaceutical products selected from: a pharmaceutical composition, a formulation, a dosage form, a combined preparation, a twin pack, and a kit; comprising a compound of the present invention; for modulating the activity of a GPR119 receptor in an individual.

One aspect of the present invention pertains to pharmaceutical products selected from: a pharmaceutical composition, a formulation, a dosage form, a combined preparation, a twin pack, and a kit; comprising a compound of the present invention; for agonizing a GPR119 receptor in an individual.

One aspect of the present invention pertains to pharmaceutical products selected from: a pharmaceutical composition, a formulation, a dosage form, a combined preparation, a twin pack, and a kit; comprising a compound of the present invention; for increasing the secretion of an incretin in an individual.

One aspect of the present invention pertains to pharmaceutical products selected from: a pharmaceutical composition, a formulation, a dosage form, a combined preparation, a twin pack, and a kit; comprising a compound of the present invention; for increasing a blood incretin level in an individual.

One aspect of the present invention pertains to pharmaceutical products selected from: a pharmaceutical composition, a formulation, a dosage form, a combined preparation, a twin pack, and a kit; comprising a compound of the present invention for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual.

One aspect of the present invention pertains to methods for modulating the activity of a GPR119 receptor, comprising prescribing to an individual in need thereof, a therapeutically effective amount of a first pharmaceutical agent selected from a compound of the present invention, in combination with a therapeutically effective amount of a second pharmaceutical agent.

One aspect of the present invention pertains to methods for agonizing a GPR119 receptor, comprising prescribing to an individual in need thereof, a therapeutically effective amount of a first pharmaceutical agent selected from a compound of the present invention, in combination with a therapeutically effective amount of a second pharmaceutical agent.

One aspect of the present invention pertains to methods for increasing the secretion of an incretin in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for increasing a blood incretin level in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for modulating the activity of a GPR119 receptor, comprising administering to an individual in need thereof, a therapeutically effective amount of a first pharmaceutical agent selected from a compound of the present invention, in combination with a therapeutically effective amount of a second pharmaceutical agent.

One aspect of the present invention pertains to methods for agonizing a GPR119 receptor, comprising administering to an individual in need thereof, a therapeutically effective amount of a first pharmaceutical agent selected from a compound of the present invention, in combination with a therapeutically effective amount of a second pharmaceutical agent.

One aspect of the present invention pertains to methods for increasing the secretion of an incretin in an individual, comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to methods for increasing a blood incretin level in an individual, comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to methods for the treatment of a disorder selected from: a GPR119-receptor-related disorder; a condition ameliorated by increasing a blood incretin level; a condition characterized by low bone mass; a neurological disorder; a metabolic-related disorder; and obesity; in an individual; comprising administering to the individual in need thereof, a therapeutically effective amount of a first pharmaceutical agent selected from a compound of the present invention, in combination with a therapeutically effective amount of a second pharmaceutical agent.

In some embodiments, the first pharmaceutical agent and the second pharmaceutical agent are administered simultaneously, separately, or sequentially.

In some embodiments, the first pharmaceutical agent and the second pharmaceutical agent are administered simultaneously.

In some embodiments, the first pharmaceutical agent and the second pharmaceutical agent are administered separately.

In some embodiments, the first pharmaceutical agent and the second pharmaceutical agent are administered sequentially.

One aspect of the present invention pertains to uses of a first pharmaceutical agent selected from a compound of the present invention, in combination with a second pharmaceutical agent in the manufacture of a medicament for modulating the activity of a GPR119 receptor in an individual.

One aspect of the present invention pertains to uses of a first pharmaceutical agent in combination with a second pharmaceutical agent selected from a compound of the present invention, in the manufacture of a medicament for modulating the activity of a GPR119 receptor in an individual.

One aspect of the present invention pertains to uses of a first pharmaceutical agent selected from a compound of the present invention, in combination with a second pharmaceutical agent in the manufacture of a medicament for agonizing a GPR119 receptor in an individual.

One aspect of the present invention pertains to uses of a first pharmaceutical agent in combination with a second pharmaceutical agent selected from a compound of the present invention, in the manufacture of a medicament for agonizing a GPR119 receptor in an individual.

One aspect of the present invention pertains to uses of a first pharmaceutical agent selected from a compound of the present invention, in combination with a second pharmaceutical agent in the manufacture of a medicament for increasing the secretion of an incretin in an individual.

One aspect of the present invention pertains to uses of a first pharmaceutical agent in combination with a second pharmaceutical agent selected from a compound of the present invention, in the manufacture of a medicament for increasing the secretion of an incretin in an individual.

One aspect of the present invention pertains to uses of a first pharmaceutical agent selected from a compound of the present invention, in combination with a second pharmaceutical agent in the manufacture of a medicament for increasing a blood incretin level in an individual.

One aspect of the present invention pertains to uses of a first pharmaceutical agent in combination with a second pharmaceutical agent selected from a compound of the present invention, in the manufacture of a medicament for increasing a blood incretin level in an individual.

One aspect of the present invention pertains to uses of a first pharmaceutical agent selected from a compound of the present invention, in combination with a second pharmaceutical agent, in the manufacture of a medicament for the treatment of a disorder selected from: a GPR119-receptor-related disorder; a condition ameliorated by increasing a blood incretin level; a condition characterized by low bone mass; a neurological disorder; a metabolic-related disorder; and obesity.

One aspect of the present invention pertains to uses of a first pharmaceutical agent in combination with a second pharmaceutical agent selected from a compound of the present invention, in the manufacture of a medicament for the treatment of a disorder selected from: a GPR119-receptor-related disorder; a condition ameliorated by increasing a blood incretin level; a condition characterized by low bone mass; a neurological disorder; a metabolic-related disorder; and obesity.

In some embodiments, the treatment comprises administering the first pharmaceutical agent and the second pharmaceutical agent simultaneously, separately, or sequentially.

In some embodiments, the treatment comprises administering the first pharmaceutical agent and the second pharmaceutical agent simultaneously.

In some embodiments, the treatment comprises administering the first pharmaceutical agent and the second pharmaceutical agent separately.

In some embodiments, the treatment comprises administering the first pharmaceutical agent and the second pharmaceutical agent sequentially.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of treatment of the human or animal body by therapy.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of modulating the activity of a GPR119 receptor in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of agonizing a GPR119 receptor in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of increasing the secretion of an incretin in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of increasing a blood incretin level in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of treatment of a disorder selected from: a GPR119-receptor-related disorder; a condition ameliorated by increasing a blood incretin level; a condition characterized by low bone mass; a neurological disorder; a metabolic-related disorder; and obesity; in an individual.

In some embodiments, the method comprises administering the first pharmaceutical agent and the second pharmaceutical agent simultaneously, separately, or sequentially.

In some embodiments, the method comprises administering the first pharmaceutical agent and the second pharmaceutical agent simultaneously.

In some embodiments, the method comprises administering the first pharmaceutical agent and the second pharmaceutical agent separately.

In some embodiments, the method comprises administering the first pharmaceutical agent and the second pharmaceutical agent sequentially.

In some embodiments, the pharmaceutical product comprises a pharmaceutical composition.

In some embodiments, the pharmaceutical product comprises a formulation.

In some embodiments, the pharmaceutical product comprises a dosage form.

In some embodiments, the pharmaceutical product comprises a combined preparation,

In some embodiments, the pharmaceutical product comprises a twin pack.

In some embodiments, the pharmaceutical product comprises a kit.

One aspect of the present invention pertains to methods for preparing a pharmaceutical product of the present invention comprising: mixing the compound with a first pharmaceutically acceptable carrier to prepare a compound dosage form, mixing the second pharmaceutical agent with a second pharmaceutically acceptable carrier to prepare a second pharmaceutical agent dosage form, and providing the compound dosage form and the second pharmaceutical agent dosage form in a combined dosage form for simultaneous, separate, or sequential use.

In some embodiments, the first pharmaceutically acceptable carrier and the second pharmaceutically acceptable carrier are different pharmaceutically acceptable carriers.

In some embodiments, the different pharmaceutically acceptable carriers are suitable for administration by the same route or different routes.

In some embodiments, the first pharmaceutically acceptable carrier and the second pharmaceutically acceptable carrier are substantially the same pharmaceutically acceptable carriers.

In some embodiments, the substantially the same pharmaceutically acceptable carriers are suitable for administration by the same route.

In some embodiments, the substantially the same pharmaceutically acceptable carriers are suitable for oral administration.

In some embodiments, the incretin is GLP-1.

In some embodiments, the incretin is GIP.

In some embodiments, the incretin is PYY.

In some embodiments, the disorder is a GPR119-receptor-related disorder.

In some embodiments, the disorder is a condition ameliorated by increasing a blood incretin level; and the incretin is GLP-1.

In some embodiments, the disorder is a condition ameliorated by increasing a blood incretin level; and the incretin is GIP.

In some embodiments, the disorder is a condition ameliorated by increasing a blood incretin level; and the incretin is PYY.

In some embodiments, the disorder is a condition characterized by low bone mass.

In some embodiments, the disorder is a condition characterized by low bone mass selected from: osteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine, and loss of height.

In some embodiments, the disorder is osteoporosis.

In some embodiments, the disorder is a neurological disorder.

In some embodiments, the disorder is a neurological disorder selected from: stroke and Parkinsonism.

In some embodiments, the disorder is a metabolic-related disorder.

In some embodiments, the disorder is a metabolic-related disorder selected from: diabetes, type 1 diabetes, type 2 diabetes, inadequate glucose tolerance, impaired glucose tolerance, insulin resistance, hyperglycemia, hyperlipidemia, hypertriglyceridemia, hypercholesterolemia, dyslipidemia, atherosclerosis, stroke, syndrome X, hypertension, pancreatic beta-cell insufficiency, enteroendocrine cell insufficiency, glucosuria, metabolic acidosis, cataracts, diabetic nephropathy, diabetic neuropathy, peripheral neuropathy, diabetic coronary artery disease, diabetic cerebrovascular disease, diabetic peripheral vascular disease, diabetic retinopathy, metabolic syndrome, a condition related to diabetes, myocardial infarction, learning impairment, memory impairment, a neurodegenerative disorder, a condition ameliorated by increasing a blood GLP-1 level in an individual with a neurodegenerative disorder, excitotoxic brain damage caused by severe epileptic seizures, Alzheimer's disease, Parkinson's disease, Huntington's disease, prion-associated disease, stroke, motor-neuron disease, traumatic brain injury, spinal cord injury, and obesity.

In some embodiments, the disorder is type 2 diabetes.

In some embodiments, the disorder is hyperglycemia.

In some embodiments, the disorder is hyperlipidemia.

In some embodiments, the disorder is hypertriglyceridemia.

In some embodiments, the disorder is type 1 diabetes.

In some embodiments, the disorder is dyslipidemia.

In some embodiments, the disorder is syndrome X.

In some embodiments, the disorder is obesity.

In some embodiments, the first pharmaceutical agent and the second pharmaceutical agent are provided in amounts which give a synergistic effect in treating the disorder.

In some embodiments, the amount of the first pharmaceutical agent alone is substantially therapeutically ineffective at treating the disorder.

In some embodiments, the amount of the second pharmaceutical agent alone is substantially therapeutically ineffective at treating the disorder.

In some embodiments, the first pharmaceutical agent is selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue.

In some embodiments, the first pharmaceutical agent is selected from: a DPP-IV inhibitor, a biguanide, an alpha-glucosidase inhibitor, a sulfonylurea, an SGLT2 inhibitor, and a meglitinide.

In some embodiments, the first pharmaceutical agent is a DPP-IV inhibitor.

In some embodiments, the first pharmaceutical agent is a biguanide.

In some embodiments, the first pharmaceutical agent is an alpha-glucosidase inhibitor.

In some embodiments, the first pharmaceutical agent is a sulfonylurea.

In some embodiments, the first pharmaceutical agent is an SGLT2 inhibitor.

In some embodiments, the first pharmaceutical agent is a meglitinide.

In some embodiments, the first pharmaceutical agent is a DPP-IV inhibitor selected from the following DPP-IV inhibitors and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-ylboronic acid;
4(S)-fluoro-1-[2-[(1R,3S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5-difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine

In some embodiments, the first pharmaceutical agent is a biguanide selected from the following biguanides and pharmaceutically acceptable salts, solvates, and hydrates thereof:

(phenylethyl)biguanide;

dimethylbiguanide;

butylbiguanide; and

1-(p-chlorophenyl)-5-isopropylbiguanide.

In some embodiments, the first pharmaceutical agent is an alpha-glucosidase inhibitor selected from the following alpha-glucosidase inhibitors and pharmaceutically acceptable salts, solvates, and hydrates thereof:

(2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;

(2R,3R,4R,5S)-1-(2-hydroxyethyl)-2-(hydroxymethyl)piperidine-3,4,5-triol; and

(1S,2S,3R,4S,5S)-5-(1,3-dihydroxypropan-2-ylamino)-1-(hydroxymethyl)cyclohexane-1,2,3,4-tetraol.

In some embodiments, the first pharmaceutical agent is a sulfonylurea selected from the following sulfonylureas and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the first pharmaceutical agent is an SGLT2 inhibitor selected from the following SGLT2 inhibitors and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the first pharmaceutical agent is a meglitinide selected from the following meglitinides and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-((3aR,7aS)-1H-isoindol-2(3H,3aH,4H,5H,6H,7H,7aH)-yl)-4-oxobutanoic acid.

In some embodiments, the second pharmaceutical agent is selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue.

In some embodiments, the second pharmaceutical agent is selected from: a DPP-IV inhibitor, a biguanide, an alpha-glucosidase inhibitor, a sulfonylurea, an SGLT2 inhibitor, and a meglitinide.

In some embodiments, the second pharmaceutical agent is a DPP-IV inhibitor.

In some embodiments, the second pharmaceutical agent is a biguanide.

In some embodiments, the second pharmaceutical agent is an alpha-glucosidase inhibitor.

In some embodiments, the second pharmaceutical agent is a sulfonylurea.

In some embodiments, the second pharmaceutical agent is an SGLT2 inhibitor.

In some embodiments, the second pharmaceutical agent is a meglitinide.

In some embodiments, the second pharmaceutical agent is a DPP-IV inhibitor selected from the following DPP-IV inhibitors and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-yl boronic acid;
4(S)-fluoro-1-[2-[(1R,3S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5-difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine

In some embodiments, the second pharmaceutical agent is a biguanide selected from the following biguanides and pharmaceutically acceptable salts, solvates, and hydrates thereof:

(phenylethyl)biguanide;

dimethylbiguanide;

butylbiguanide; and

1-(p-chlorophenyl)-5-isopropylbiguanide.

In some embodiments, the second pharmaceutical agent is an alpha-glucosidase inhibitor selected from the following alpha-glucosidase inhibitors and pharmaceutically acceptable salts, solvates, and hydrates thereof:

(2R,3R,4R,5R)-4-(2R,3R,4R,5S,6R)-5-(2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;

(2R,3R,4R,5S)-1-(2-hydroxyethyl)-2-(hydroxymethyl)piperidine-3,4,5-triol; and
(1S,2S,3R,4S,5S)-5-(1,3-dihydroxypropan-2-ylamino)-1-(hydroxymethyl)cyclohexane-1,2,3,4-tetraol.

In some embodiments, the second pharmaceutical agent is a sulfonylurea selected from the following sulfonylureas and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the second pharmaceutical agent is an SGLT2 inhibitor selected from the following SGLT2 inhibitors and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the second pharmaceutical agent is a meglitinide selected from the following meglitinides and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-(3aR,7aS)-1H-isoindol-2(3H,3aH,4H,5H,6H,7H,7aH)-yl)-4-oxobutanoic acid.

One aspect of the present invention pertains to methods for weight management, comprising administering to an individual in need thereof, a therapeutically effective amount of a compound of the present invention in combination with a therapeutically effective amount of a pharmaceutical agent, such as any agent described herein; wherein the compound and the pharmaceutical agent.

In some embodiments, the weight management comprises weight loss. In some embodiments, the weight management comprises maintenance of weight loss. In some embodiments, the weight management further comprises a reduced-calorie diet. In some embodiments, the weight management further comprises a program of regular exercise. In some embodiments, the weight management further comprises both a reduced-calorie diet and a program of regular exercise.

In some embodiments, the individual in need of weight management is a patient with an initial body mass of index ≧40 kg/m²; ≧39 kg/m²; ≧38 kg/m²; ≧37 kg/m²; ≧36 kg/m²; ≧35 kg/m²; ≧34 kg/m²; ≧33 kg/m²; ≧32 kg/m²; ≧31 kg/m²; ≧30 kg/m²; ≧29 kg/m²; ≧28 kg/m²; ≧27 kg/m²; ≧26 kg/m²; ≧25 kg/m²; ≧24 kg/m²; ≧23 kg/m²; ≧22 kg/m²; ≧21 kg/m²; or ≧20 kg/m²; and the patient optionally has at least one or at least two weight related comorbid condition(s).

In some embodiments, the comorbid condition(s) when present are selected from: hypertension, dyslipidemia, cardiovascular disease, glucose intolerance, and sleep apnea.

### Indications

In the context of the present invention, a compound as described herein, or a composition or pharmaceutical composition thereof, can be utilized for modulating the activity of GPR119-receptor-related diseases, conditions and/or disorders as described herein.

In some embodiments, modulating the activity includes the treatment of a GPR119-receptor-related disorder. In some embodiments, the GPR119-receptor-related disorder is a condition ameliorated by increasing a blood incretin level. In some embodiments, the GPR119-receptor-related disorder is a condition characterized by low bone mass. In some embodiments, the GPR119-receptor-related disorder is a neurological disorder. In some embodiments, the GPR119-receptor-related disorder is a metabolic-related disorder. In some embodiments, the GPR119-receptor-related disorder is type 2 diabetes. In some embodiments, the GPR119-receptor-related disorder is obesity.

Some embodiments of the present invention include every combination of one or more conditions characterized by low bone mass selected from: osteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine, and loss of height.

In some embodiments, the neurological disorder selected from: stroke and Parkinsonism.

Some embodiments of the present invention include every combination of one or more metabolic-related disorders selected from: type 1 diabetes, type 2 diabetes mellitus, and conditions associated therewith, such as, but not limited to, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertriglyceridemia, insulin resistance, impaired glucose metabolism, erectile dysfunction, skin and connective tissue disorders, foot ulcerations and ulcerative colitis, endothelial dysfunction and impaired vascular compliance.

Some embodiments of the present invention include every combination of one or more metabolic-related disorders selected from: diabetes, type 1 diabetes, type 2 diabetes, inadequate glucose tolerance, impaired glucose tolerance, insulin resistance, hyperglycemia, hyperlipidemia, hypertriglyceridemia, hypercholesterolemia, dyslipidemia, atherosclerosis, stroke, syndrome X, hypertension, pancreatic beta-cell insufficiency, enteroendocrine cell insufficiency, glucosuria, metabolic acidosis, cataracts, diabetic nephropathy, diabetic neuropathy, peripheral neuropathy, diabetic coronary artery disease, diabetic cerebrovascular disease, diabetic peripheral vascular disease, diabetic retinopathy, metabolic syndrome, a condition related to diabetes, myocardial infarction, learning impairment, memory impairment, a neurodegenerative disorder, a condition ameliorated by increasing a blood GLP-1 level in an individual with a neurodegenerative disorder, excitotoxic brain damage caused by severe epileptic seizures, Alzheimer's disease, Parkinson's disease, Huntington's disease, prion-associated disease, stroke, motor-neuron disease, traumatic brain injury, spinal cord injury, and obesity.

In some embodiments, the disorder is type 2 diabetes. In some embodiments, the disorder is hyperglycemia. In some embodiments, the disorder is hyperlipidemia. In some embodiments, the disorder is hypertriglyceridemia. In some embodiments, the disorder is type 1 diabetes. In some embodiments, the disorder is dyslipidemia. In some embodiments, the disorder is syndrome X. In some embodiments, the disorder is obesity.

### Compositions and Formulations

In any of the embodiments that recites the terms “first pharmaceutical agent selected from a compound of the present invention” and “second pharmaceutical agent”, it is appreciated that the term “second pharmaceutical agent” may in some aspects be further limited to a pharmaceutical agent that is not a compound of Formula I, and may refer to a pharmaceutical agent that is not detectable or has an EC₅₀ that is greater than a value selected from: 50 μM, 10 μM, 1 μM, and 0.1 μM in a GPR119 receptor activity assay as described in Example 4.

In any of the embodiments that recites the terms “first pharmaceutical agent” and “second pharmaceutical agent selected from a compound of the present invention”, it is appreciated that the term “first pharmaceutical agent” may in some aspects be further limited to a pharmaceutical agent that is not a compound of Formula I, and may refer to a pharmaceutical agent that is not detectable or has an EC₅₀ that is greater than a value selected from: 50 μM, 10 μM, 1 μM, and 0.1 μM in a GPR119 receptor activity assay as described in Example 4.

One aspect of the present invention pertains to compositions comprising a compound of the present invention.

One aspect of the present invention pertains to compositions comprising a compound of the present invention and a pharmaceutically acceptable carrier.

One aspect of the present invention pertains to compositions obtained by a method of the present invention.

One aspect of the present invention pertains to compositions comprising: a first pharmaceutical agent selected from a compound of the present invention; and a second pharmaceutical agent.

One aspect of the present invention pertains to compositions comprising: a first pharmaceutical agent selected from a compound of the present invention; a second pharmaceutical agent; and a pharmaceutically acceptable carrier.

In some embodiments, the first pharmaceutical agent is selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue.

In some embodiments, the first pharmaceutical agent is selected from: a DPP-IV inhibitor, a biguanide, an alpha-glucosidase inhibitor, a sulfonylurea, an SGLT2 inhibitor, and a meglitinide.

In some embodiments, the first pharmaceutical agent is a DPP-IV inhibitor.

In some embodiments, the first pharmaceutical agent is a biguanide.

In some embodiments, the first pharmaceutical agent is an alpha-glucosidase inhibitor.

In some embodiments, the first pharmaceutical agent is a sulfonylurea.

In some embodiments, the first pharmaceutical agent is an SGLT2 inhibitor.

In some embodiments, the first pharmaceutical agent is a meglitinide.

In some embodiments, the first pharmaceutical agent is a DPP-IV inhibitor selected from the following DPP-IV inhibitors and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-ylboronic acid;
4(S)-fluoro-1-[2-[(1R,3S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5-difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine

In some embodiments, the first pharmaceutical agent is a biguanide selected from the following biguanides and pharmaceutically acceptable salts, solvates, and hydrates thereof:

(phenylethyl)biguanide;

dimethylbiguanide;

butylbiguanide; and

1-(p-chlorophenyl)-5-isopropylbiguanide.

In some embodiments, the first pharmaceutical agent is an alpha-glucosidase inhibitor selected from the following alpha-glucosidase inhibitors and pharmaceutically acceptable salts, solvates, and hydrates thereof:
(2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;
R,3R,4R,5S)-1-(2-hydroxyethyl)-2-(hydroxymethyl)piperidine-3,4,5-triol; and
(1S,2S,3R,4S,5S)-5-(1,3-dihydroxypropan-2-ylamino)-1-(hydroxymethyl)cyclohexane-1,2,3,4-tetraol.

In some embodiments, the first pharmaceutical agent is a sulfonylurea selected from the following sulfonylureas and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the first pharmaceutical agent is an SGLT2 inhibitor selected from the following SGLT2 inhibitors and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the first pharmaceutical agent is a meglitinide selected from the following meglitinides and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-((3aR,7aS)-1H-isoindol-2(3H,3aH,4H,5H,6H,7H,7aH)-yl)-4-oxobutanoic acid.

In some embodiments, the second pharmaceutical agent is selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue.

In some embodiments, the second pharmaceutical agent is selected from: a DPP-IV inhibitor, a biguanide, an alpha-glucosidase inhibitor, a sulfonylurea, an SGLT2 inhibitor, and a meglitinide.

In some embodiments, the second pharmaceutical agent is a DPP-IV inhibitor.

In some embodiments, the second pharmaceutical agent is a biguanide.

In some embodiments, the second pharmaceutical agent is an alpha-glucosidase inhibitor.

In some embodiments, the second pharmaceutical agent is a sulfonylurea.

In some embodiments, the second pharmaceutical agent is an SGLT2 inhibitor.

In some embodiments, the second pharmaceutical agent is a meglitinide.

In some embodiments, the second pharmaceutical agent is a DPP-IV inhibitor selected from the following DPP-IV inhibitors and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-yl boronic acid;
4(S)-fluoro-1-[2-[(1R,3S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5-difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-([6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl]methyl)-4-fluorobenzonitrile;
S)-1-{[2-(5-methyl-2-phenyl-oxazol-4-yl)-ethylamino]-acetyl}-pyrrolidine-2-carbonitrile;
(2S)-1-{[1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro-[1,2,4]-triazolo[4,3-a]pyrazin-7(8H)-yl]carbonyl}-6-(2,4,5-trifluorophenyl)cyclohex-3-en-1-amine

In some embodiments, the second pharmaceutical agent is a biguanide selected from the following biguanides and pharmaceutically acceptable salts, solvates, and hydrates thereof: (phenylethyl)biguanide;

dimethylbiguanide;

butylbiguanide; and

1-(p-chlorophenyl)-5-isopropylbiguanide.

In some embodiments, the second pharmaceutical agent is an alpha-glucosidase inhibitor selected from the following alpha-glucosidase inhibitors and pharmaceutically acceptable salts, solvates, and hydrates thereof:
(2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;
(2R,3R,4R,5S)-1-(2-hydroxyethyl)-2-(hydroxymethyl)piperidine-3,4,5-triol; and
(1S,2S,3R,4S,5S)-5-(1,3-dihydroxypropan-2-ylamino)-1-(hydroxymethyl)cyclohexane-1,2,3,4-tetraol.

In some embodiments, the second pharmaceutical agent is a sulfonylurea selected from the following sulfonylureas and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the second pharmaceutical agent is an SGLT2 inhibitor selected from the following SGLT2 inhibitors and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the second pharmaceutical agent is a meglitinide selected from the following meglitinides and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-((3aR,7aS)-1H-isoindol-2(3H,3aH,4H,5H,6H,7H,7aH)-yl)-4-oxobutanoic acid.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a solvate, hydrate or physiologically functional derivative thereof can be used as active ingredients in pharmaceutical compositions, specifically as GPR119 receptor modulators. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds of the present invention can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis conducted or on whether further active compounds are administered in addition to the compounds of the present invention. Representative doses of the present invention include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt, solvate, or hydrate of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size. The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” refers to the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well known to the person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain compounds of the present invention which contain a carboxylic acid functional group may optionally exist as pharmaceutically acceptable salts containing non-toxic, pharmaceutically acceptable metal cations and cations derived from organic bases. Representative metals include, but are not limited to, aluminium, calcium, lithium, magnesium, potassium, sodium, zinc and the like. In some embodiments the pharmaceutically acceptable metal is sodium. Representative organic bases include, but are not limited to, benzathine (N¹,N²-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et al., Journal of Pharmaceutical Sciences, 66:1-19 (1977).

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

It is noted that when the GPR119 receptor modulators are utilized as active ingredients in pharmaceutical compositions, these are not intended for use in humans only, but in non-human mammals as well. Recent advances in the area of animal health-care mandate that consideration be given for the use of active agents, such as GPR119 receptor modulators, for the treatment of a GPR119 receptor-associated disease or disorder in companionship animals (e.g., cats, dogs, etc.) and in livestock animals (e.g., horses, cows, etc.) Those of ordinary skill in the art are readily credited with understanding the utility of such compounds in such settings.

### Hydrates and Solvates

It is understood that when the phrase “pharmaceutically acceptable salts, solvates, and hydrates” or the phrase “pharmaceutically acceptable salt, solvate, or hydrate” is used when referring to compounds described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used when referring to salts described herein, it embraces pharmaceutically acceptable solvates and/or hydrates of such salts.

It will be apparent to those skilled in the art that the dosage forms described herein may comprise, as the active component, either a compound described herein or a pharmaceutically acceptable salt or as a pharmaceutically acceptable solvate or hydrate thereof. Moreover, various hydrates and solvates of the compounds described herein and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present invention pertains to methods of administering hydrates and solvates of compounds described herein and/or their pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

### Combination Therapy

A compound of the invention can be administered as the sole active pharmaceutical agent (i.e., mono-therapy), or it can be used in combination with one or more pharmaceutical agents (i.e., combination-therapy), such as pharmaceutical agents, such as, known anti-diabetic agents, either administered together or separately for the treatment of the diseases, conditions, and disorders described herein. Therefore, another aspect of the present invention includes methods of treatment of a metabolic related disorder, including a weight-related disorder, such as obesity, comprising administering to an individual in need thereof a therapeutically effective amount of a compound of Formula I and pharmaceutically acceptable salts, solvates and hydrates thereof, in combination with one or more pharmaceutical agents, such as anti-diabetic agents, as described herein.

In accordance with the present invention, the combination can be used by mixing the respective active components, a compound of Formula I and a pharmaceutical agent, either together or independently optionally with a physiologically acceptable carrier, excipient, binder, diluent, etc., as described herein, and administering the mixture or mixtures either orally or non-orally as a pharmaceutical composition(s). When a compound of Formula I is administered as a combination therapy with another active compound the compound of Formula I and the pharmaceutical agent can be formulated as separate pharmaceutical compositions given at the same time or at different times; or the compound of Formula I and the pharmaceutical agent can be formulated together as a single unit dosage.

One aspect of the present invention pertains to first pharmaceutical agents selected from a compound according of the present invention, for use in combination with a second pharmaceutical agent for use in a method of treatment of the human or animal body by therapy.

One aspect of the present invention pertains to first pharmaceutical agents selected from a compound according of the present invention, for use in combination with a second pharmaceutical agent for use in a method of modulating the activity of a GPR119 receptor in an individual.

One aspect of the present invention pertains to first pharmaceutical agents selected from a compound according of the present invention, for use in combination with a second pharmaceutical agent for use in a method of agonizing a GPR119 receptor in an individual.

One aspect of the present invention pertains to first pharmaceutical agents selected from a compound according of the present invention, for use in combination with a second pharmaceutical agent for use in a method of increasing the secretion of an incretin in an individual.

One aspect of the present invention pertains to first pharmaceutical agents selected from a compound according of the present invention, for use in combination with a second pharmaceutical agent for use in a method of increasing a blood incretin level in an individual.

One aspect of the present invention pertains to first pharmaceutical agents selected from a compound according of the present invention, for use in combination with a second pharmaceutical agent for use in a method of treatment of a disorder selected from: a GPR119-receptor-related disorder; a condition ameliorated by increasing a blood incretin level; a condition characterized by low bone mass; a neurological disorder; a metabolic-related disorder; nd obesity; in an individual.

One aspect of the present invention pertains to first pharmaceutical agents for use in combination with a second pharmaceutical agent selected from a compound of the present invention, for use in a method of treatment of the human or animal body by therapy.

One aspect of the present invention pertains to first pharmaceutical agents for use in combination with a second pharmaceutical agent selected from a compound of the present invention, for use in a method of modulating the activity of a GPR119 receptor in an individual.

One aspect of the present invention pertains to first pharmaceutical agents for use in combination with a second pharmaceutical agent selected from a compound of the present invention, for use in a method of agonizing a GPR119 receptor in an individual.

One aspect of the present invention pertains to first pharmaceutical agents for use in combination with a second pharmaceutical agent selected from a compound of the present invention, for use in a method of increasing the secretion of an incretin in an individual.

One aspect of the present invention pertains to first pharmaceutical agents for use in combination with a second pharmaceutical agent selected from a compound of the present invention, for use in a method of increasing a blood incretin level in an individual.

One aspect of the present invention pertains to first pharmaceutical agents for use in combination with a second pharmaceutical agent selected from a compound of the present invention, for use in a method of treatment of a disorder selected from: a GPR119-receptor-related disorder; a condition ameliorated by increasing a blood incretin level; a condition characterized by low bone mass; a neurological disorder; a metabolic-related disorder; and obesity; in an individual.

In some embodiments, the method comprises administering the first pharmaceutical agent and the second pharmaceutical agent simultaneously, separately, or sequentially.

In some embodiments, the method comprises administering the first pharmaceutical agent and the second pharmaceutical agent simultaneously.

In some embodiments, the method comprises administering the first pharmaceutical agent and the second pharmaceutical agent separately.

In some embodiments, the method comprises administering the first pharmaceutical agent and the second pharmaceutical agent sequentially.

In some embodiments, the first pharmaceutical agent and the second pharmaceutical agent are provided in amounts which give a synergistic effect in treating the disorder.

In some embodiments, the amount of the first pharmaceutical agent alone is substantially therapeutically ineffective at treating the disorder.

In some embodiments, the amount of the second pharmaceutical agent alone is substantially therapeutically ineffective at treating the disorder.

Suitable pharmaceutical agents that can be used in combination with the compounds of the present invention include anti-obesity agents such as apolipoprotein-B secretion/microsomal triglyceride transfer protein (apo-B/MTP) inhibitors; MCR-4 agonists, cholescystokinin-A (CCK-A) agonists; serotonin and norepinephrine reuptake inhibitors (for example, sibutramine); sympathomimetic agents; β3 adrenergic receptor agonists; dopamine agonists (for example, bromocriptine); melanocyte-stimulating hormone receptor analogues; cannabinoid 1 receptor antagonists [for example, SR141716: N-(piperidin-1-yl)-5-(4-chlorophenyl)-1-(2,4-dichlorophenyl)-4-methyl-1H-pyrazole-3-carboxamide]; melanin concentrating hormone antagonists; leptin (the OB protein); leptin analogues; leptin receptor agonists; galanin antagonists; lipase inhibitors (such as tetrahydrolipstatin, i.e., Orlistat); anorectic agents (such as a bombesin agonist); neuropeptide-Y antagonists; thyromimetic agents; dehydroepiandrosterone or an analogue thereof; glucocorticoid receptor agonists or antagonists; orexin receptor antagonists; urocortin binding protein antagonists; glucagon-like peptide-1 (GLP-1) receptor agonists; ciliary neutrotrophic factors (such as Axokine™ available from Regeneron Pharmaceuticals, Inc., Tarrytown, N.Y. and Procter & Gamble Company, Cincinnati, Ohio); human agouti-related proteins (AGRP); ghrelin receptor antagonists; histamine 3 receptor (H3R) antagonists or inverse agonists; neuromedin U receptor agonists; noradrenergic anorectic agents (for example, phentermine, mazindol and the like); and appetite suppressants (for example, bupropion).

Other anti-obesity agents, including the agents set forth infra, are well known, or will be readily apparent in light of the instant disclosure, to one of ordinary skill in the art. In some embodiments, the anti-obesity agents are selected from the group consisting of orlistat, sibutramine, bromocriptine, ephedrine, leptin, and pseudoephedrine. In a further embodiment, compounds of the present invention and combination therapies are administered in conjunction with exercise and/or a calorie-controlled diet.

It is understood that the scope of combination-therapy of the compounds of the present invention with anti-obesity agents, anorectic agents, appetite suppressant and related agents is not limited to those listed above, but includes in principle any combination with any pharmaceutical agent or pharmaceutical composition useful for the treatment of overweight and obese individuals.

It is understood that the scope of combination-therapy of the compounds of the present invention with other pharmaceutical agents is not limited to those listed herein, supra or infra, but includes in principle any combination with any pharmaceutical agent or pharmaceutical composition useful for the treatment of diseases, conditions or disorders that are linked to metabolic related disorders.

Some embodiments of the present invention include methods of treatment of a disease, disorder, condition or complication thereof as described herein, comprising administering to an individual in need of such treatment a therapeutically effective amount or dose of a compound of Formula I in combination with at least one pharmaceutical agent selected from the group consisting of: sulfonylureas (for example, tolbutamide (Orinase); acetohexamide (Dymelor); tolazamide (Tolinase); chlorpropamide (Diabinese); glipizide (Glucotrol); glyburide (Diabeta, Micronase, Glynase); glimepiride (Amaryl); gliclazide (Diamicron); and sulfonylureas known in the art); meglitinides (for example, repaglinide (Prandin), nateglinide (Starlix), mitiglinide, and other meglitinides known in the art); biguanides (for example, phenformin, metformin, buformin, and biguanides known in the art); α-glucosidase inhibitors (for example, acarbose, miglitol, and α-glucosidase inhibitors known in the art); thiazolidinediones-peroxisome proliferators-activated receptor-γ (i.e., PPAR-γ) agonists (for example, rosiglitazone (Avandia), pioglitazone (Actos), troglitazone (Rezulin), rivoglitazone, ciglitazone, and thiazolidinediones known in the art); insulin and insulin analogues; anti-diabetic peptide analogues (for example, exenatide, liraglutide, taspoglutide, and anti-diabetic peptides analogues know in the art); HMG-CoA reductase inhibitors (for example, rosuvastatin, pravastatin and its sodium salt, simvastatin, lovastatin, atorvastatin, fluvastatin, cerivastatin, rosuvastatin, pitavastatin, pravastatin, and other HMG-CoA reductase inhibitors known in the art); cholesterol-lowering drugs (for example, fibrates that include: bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, theofibrate, and other fibrates known in the art; bile acid sequestrants which include: cholestyramine, colestipol and the like; and niacin); antiplatelet agents (for example, aspirin and adenosine diphosphate receptor antagonists that include: clopidogrel, ticlopidine and the like); angiotensin-converting enzyme inhibitors (for example, captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and other angiotensin converting enzyme inhibitors known in the art); angiotensin II receptor antagonists [for example, losartan (and the potassium salt form), and other angiotensin II receptor antagonists known in the art; adiponectin; squalene synthesis inhibitors {for example, (S)-α-[bis[2,2-dimethyl-1-oxopropoxy)methoxy]phosphinyl]-3-phenoxybenzenebutanesulfonic acid, mono potassium salt (BMS-188494) and other squalene synthesis inhibitors known in the art}; and the like. In some embodiments, compounds of the present invention and the pharmaceutical agents are administered separately. In further embodiments, compounds of the present invention and the pharmaceutical agents are administered simultaneously.

Suitable pharmaceutical agents that can be used in conjunction with compounds of the present invention include, but are not limited to: amylin agonists (for example, pramlintide); insulin secretagogues (for example, GLP-1 agonists, exendin-4, and insulinotropin (NN2211)); acyl CoA cholesterol acetyltransferase inhibitors (for example, ezetimibe, eflucimibe, and other acyl CoA cholesterol acetyltransferase inhibitors known in the art); cholesterol absorption inhibitors (for example, ezetimibe, pamaqueside and other cholesterol absorption inhibitors known in the art); cholesterol ester transfer protein inhibitors (for example, CP-529414, JTT-705, CETi-1, and other cholesterol ester transfer protein inhibitors known in the art); microsomal triglyceride transfer protein inhibitors (for example, implitapide, and other microsomal triglyceride transfer protein inhibitors known in the art); cholesterol modulators (for example, NO-1886, and other cholesterol modulators known in the art); bile acid modulators (for example, GT103-279 and other bile acid modulators known in the art); insulin signaling pathway modulators; inhibitors of protein tyrosine phosphatases (PTPases); non-small molecule mimetics and inhibitors of glutamine-fructose-6-phosphate amidotransferase (GFAT); compounds influencing a dysregulated hepatic glucose production; inhibitors of glucose-6-phosphatase (G6Pase); inhibitors of fructose-1,6-bisphosphatase (F-1,6-BPase); inhibitors of glycogen phosphorylase (GP); glucagon receptor antagonists; inhibitors of phosphoenolpyruvate carboxykinase (PEPCK); pyruvate dehydrogenase kinase (PDHK) inhibitors; insulin sensitivity enhancers; insulin secretion enhancers; inhibitors of gastric emptying; α₂-adrenergic antagonists; retinoid X receptor (RXR) agonists; and dipeptidyl peptidase-4 (DPP-IV) inhibitors; and the like.

### Tripartite Combinations

Some aspects of the present invention include compounds of Formula I that can be employed in any of the methods, pharmaceutical products, uses, compounds, and pharmaceutical agents, as described herein, in combination with two distinct pharmaceutical agents.

In some embodiments, the two distinct pharmaceutical agents are selected from any of the pharmaceutical agents, or classes of pharmaceutical agents described herein. In some embodiments, the two distinct pharmaceutical agents are selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue. In some embodiments, the two distinct pharmaceutical agents include every combination selected from pharmaceutical agents of the following group: a DPP-IV inhibitor, a biguanide, an alpha-glucosidase inhibitor, a sulfonylurea, and an SGLT2 inhibitor.

Some embodiments of the present invention include every combination of one or more compounds selected from compounds of the following group and pharmaceutically acceptable salts, solvates, and hydrates thereof: a DPP-IV inhibitor selected from: 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one; 1-[2-(3-hydroxyadamant-1-ylamino)acetyl]pyrrolidine-2(S)-carbonitrile; (1S,3S,5S)-2-[2(S)-amino-2-(3-hydroxyadamantan-1-yl)acetyl]-2-azabicyclo[3.1.0]hexane-3-carbonitrile; 2-[6-[3(R)-aminopiperidin-1-yl]-3-methyl-2,4-dioxo-1,2,3,4-tetrahydropyrimidin-1-ylmethyl]benzonitrile; 8-[3(R)-aminopiperidin-1-yl]-7-(2-butynyl)-3-methyl-1-(4-methylquinazolin-2-ylmethyl)xanthine; 1-[N-[3(R)-pyrrolidinyl]glycyl]pyrrolidin-2(R)-yl boronic acid; 4(S)-fluoro-1-[2-[(1R,3S)-3-(1H-1,2,4-triazol-1-ylmethyl)cyclopentylamino]acetyl]pyrrolidine-2(S)-carbonitrile; 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one; (2S,4S)-2-cyano-4-fluoro-1-[(2-hydroxy-1,1-dimethyl) ethylamino]acetylpyrrolidine; 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione; 1-((3S,4S)-4-amino-1-(4-(3,3-difluoropyrrolidin-1-yl)-1,3,5-triazin-2-yl)pyrrolidin-3-yl)-5,5-difluoropiperidin-2-one;
(R)-2-((6-(3-aminopiperidin-1-yl)-3-methyl-2,4-dioxo-3,4-dihydropyrimidin-[(2H)-yl)methyl)-4-fluorobenzonitrile; 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide; ((2S,4S)-4-(4-(3-methyl-1-phenyl-1H-pyrazol-5-yl)piperazin-1-yl)pyrrolidin-2-yl)(thiazolidin-3-yl)methanone; (2S,4S)-1-[2-[(4-ethoxycarbonylbicyclo[2.2.2]oct-1-yl)amino]acetyl]-4-fluoropyrrolidine-2-carbonitrile; 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione; 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile; (2S)-1-{[2-(5-methyl-2-phenyl-oxazol-4-yl)-ethylamino]-acetyl}-pyrrolidine-2-carbonitrile; (2S)-1-{[1,1-dimethyl-3-(4-pyridin-3-yl-imidazol-1-yl)-propylamino]-acetyl}-pyrrolidine-2-carbonitrile; (3,3-difluoropyrrolidin-1-yl)-((2S,4S)-4-(4-(pyrimidin-2-yl)piperazin-1-yl)pyrrolidin-2-yl)methanone; (2S,4S)-1-[(2S)-2-amino-3,3-bis(4-fluorophenyl)propanoyl]-4-fluoropyrrolidine-2-carbonitrile; (2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine; a biguanide selected from: phenformin ((phenylethyl)biguanide); metformin (dimethylbiguanide); buformin (butylbiguanide); and proguanil (1-(p-chlorophenyl)-5-isopropylbiguanide); an α-glucosidase inhibitor selected from: acarbose ((2R,3R,4R,5R)-4-(2R,3R,4R,5S,6R)-5-(2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal); miglitol ((2R,3R,4R,5S)-1-(2-hydroxyethyl)-2-(hydroxymethyl)piperidine-3,4,5-triol); and voglibose ((1S,2S,3R,4S,5S)-5-(1,3-dihydroxypropan-2-ylamino)-1-(hydroxymethyl)cyclohexane-1,2,3,4-tetraol); an insulin analogue selected from: NPH insulin (also known as Humulin N, Novolin N, NPH Lletin II, and insulin isophane); insulin lispro (28B-L-lysine-29B-L-proline-insulin, wherein insulin is human insulin); insulin aspart (28B-L-aspartic acid-insulin, wherein insulin is human insulin); and insulin glulisine (3B-L-lysine-29B-L-glutamic acid-insulin, wherein insulin is human insulin); a sulfonylurea selected from: tolbutamide (Orinase, N-(butylcarbamoyl)-4-methylbenzenesulfonamide); acetohexamide (Dymelor, 4-acetyl-N-(cyclohexylcarbamoyl)benzenesulfonamide); tolazamide (Tolinase, N-(azepan-1-ylcarbamoyl)-4-methylbenzenesulfonamide); chlorpropamide (Diabinese, 4-chloro-N-(propylcarbamoyl)benzenesulfonamide); glipizide (Glucotrol, N-(4-(N-(cyclohexylcarbamoyl)sulfamoyl)phenethyl)-5-methylpyrazine-2-carboxamide); glibenclamide, also known as glyburide (Diabeta, Micronase, Glynase, 5-chloro-N-(4-(N-(cyclohexylcarbamoyl)sulfamoyl)phenethyl)-2-methoxybenzamide); glimepiride (Amaryl, 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide); and gliclazide (Diamicron, N-(hexahydrocyclopenta[c]pyrrol-2(1H)-ylcarbamoyl)-4-methylbenzenesulfonamide); an SGLT2 inhibitor selected from: dapagliflozin ((2S,3R,4R,5S,6R)-2-(4-chloro-3-(4-ethoxybenzyl)phenyl)-6-(hydroxymethyl)tetrahydro-2H-pyran-3,4,5-triol); remogliflozin (ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate); ASP1941, canagliflozin ((2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol); ISIS 388626; sergliflozin (ethyl ((2R,3S,4S,5R,6S)-3,4,5-trihydroxy-6-(2-(4-methoxybenzyl)phenoxy)tetrahydro-2H-pyran-2-yl)methyl carbonate), AVE2268 ((2R,3S,4S,5R,6S)-2-(hydroxymethyl)-6-(2-(4-methoxybenzyl)thiophen-3-yloxy)tetrahydro-2H-pyran-3,4,5-triol), BI10773, CSG453; and LX4211; a meglitinide selected from: repaglinide (Prandin, (S)-2-ethoxy-4-(2-(3-methyl-1-(2-(piperidin-1-yl)phenyl)butylamino)-2-oxoethyl)benzoic acid); nateglinide (Starlix, (R)-2-((1r,4R)-4-isopropylcyclohexanecarboxamido)-3-phenylpropanoic acid); and mitiglinide ((S)-2-benzyl-4-((3aR,7aS)-1H-isoindol-2(3H,3aH,4H,5H,6H,7H,7aH)-yl)-4-oxobutanoic acid); a thiazolidinedione selected from: rosiglitazone (Avandia, 5-(4-(2-(methyl(pyridin-2-yl)amino)ethoxy)benzyl)thiazolidine-2,4-dione); pioglitazone (Actos, 5-(4-(2-(5-ethylpyridin-2-yl)ethoxy)benzyl)thiazolidine-2,4-dione); troglitazone (Rezulin, 5-(4-((6-hydroxy-2,5,7,8-tetramethylchroman-2-yl)methoxy)benzyl)thiazolidine-2,4-dione); rivoglitazone (5-(4-((6-methoxy-1-methyl-1H-benzo[d]imidazol-2-yl)methoxy)benzyl)thiazolidine-2,4-dione); and ciglitazone (5-(4-((1-methylcyclohexyl)methoxy)benzyl)thiazolidine-2,4-dione); and an anti-diabetic peptide analogue selected from: exenatide; liraglutide; and taspoglutide.

In some embodiments, the two distinct pharmaceutical agents include every combination selected from pharmaceutical agents of the following group: sitagliptin, vildagliptin, saxagliptin, alogliptin, linagliptin, phenformin, metformin, buformin, acarbose, miglitol, voglibose, tolbutamide, acetohexamide, tolazamide, chlorpropamide, glipizide, glibenclamide, glimepiride, gliclazide, dapagliflozin, remigliflozin, and sergliflozin.

### Dipeptidyl Peptidase IV Inhibitors

Dipeptidyl peptidase IV (DPP-IV, EC 3.4.14.5) exhibits catalytic activity against a broad range of peptide substrates that includes peptide hormones, neuropeptides, and chemokines. The incretins glucagon-like peptide 1 (GLP-1), and glucose-dependent insulinotropic polypeptide (GIP), which stimulate glucose-dependent insulin secretion and otherwise promote blood glucose homeostasis, are rapidly cleaved by DPP-IV at the position-2 alanine leading to inactivation of their biological activity. Peptide YY (PYY) is a gut peptide that has been implicated in modulating satiety (Chaudhri et al, Annu Rev Physiol (2008) 70:239-255). PYY is released into the circulation as PYY₁₋₃₆ and PYY₃₋₃₆ (Eberlein et al, Peptides (1989) 10:797-803). PYY₃₋₃₆ is generated from PYY₁₋₃₆ by cleavage of the N-terminal Tyr and Pro residues by DPP-IV. Both pharmacological and genetic attenuation of DPP-IV activity is associated with enhanced incretin action, increased insulin, and lower blood glucose in vivo. Genetic attenuation of DPP-IV activity has been shown to provide resistance to obesity and to improve insulin sensitivity. DPP-IV inhibitors have shown to be useful as therapeutics, for example, oral administration of vildagliptin (1-[2-(3-hydroxyadamant-1-ylamino)acetyl]pyrrolidine-2(S)-carbonitrile) or sitagliptin (3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one) to human patients suffering with type 2 diabetes has been found to reduce fasting glucose and postprandial glucose excursion in association with significantly reduced HbA_{1c} levels. For reviews on the application of DPP-IV inhibitors for the treatment of type 2 diabetes, reference is made to the following publications: (1) H.-U. Demuth, et al., “Type 2 diabetes-therapy with DPP-IV inhibitors,” Biochim. Biophys. Acta, 1751: 33-44 (2005), and (2) K. Augustyns, et al., “Inhibitors of proline-specific dipeptidyl peptidases: DPP-IV inhibitors as a novel approach for the treatment of type 2 diabetes,” Expert Opin. Ther. Patents, 15: 1387-1407 (2005).

Accordingly, suitable pharmaceutical agents include DPP-IV inhibitors that can be used in conjunction with compounds of the present invention either dosed separately or together. DPP-IV inhibitors are well-known in the art or can be readily identified and their in vitro biological activity determined using any number of methods available, for example, O'Brien, M., Daily, B., Schurria, M., “Assay for DPPIV activity using a homogeneous, luminescent method,” Cell Notes, Issue 11, 2005; see also the DPPIV-Glo™ Protease Assay Technical Bulletin #TB339.

Examples of DPP-IV inhibitors are described in Villhauer et al., J. Med. Chem. (2003) 46:2774-2789, for LAF237; Ahren et al., J. Clin. Endocrinol. Metab. (2004) 89:2078-2084; Villhauer et al., J. Med. Chem. (2002) 45:2362-2365 for NVP-DPP728; Ahren et al., Diabetes Care (2002) 25:869-875 for NVP-DPP728; Peters et al., Bioorg. Med. Chem. Lett. (2004) 14:1491-1493; Caldwell et al., Bioorg. Med. Chem. Lett. (2004) 14:1265-1268; Edmondson et al., Bioorg. Med. Chem. Lett. (2004) 14:5151-5155; and Abe et al., J. Na.t Prod. (2004) 67:999-1004.

Specific examples of DPP-IV inhibitors include, but are not limited to, dipeptide derivatives or dipeptide mimetics such as alanine-pyrrolidide, isoleucine-thiazolidide, and the pseudosubstrate N-valyl prolyl, O-benzoyl hydroxylamine, as described, for example, in U.S. Pat. No. 6,303,661.

Some embodiments of the present invention include every combination of one or more DPP-IV inhibitors selected from the DPP-IV inhibitors found in U.S. Pat. Nos. 6,869,947, 6,867,205, 6,861,440, 6,849,622, 6,812,350, 6,803,357, 6,800,650, 6,727,261, 6,716,843, 6,710,040, 6,706,742, 6,645,995, 6,617,340, 6,699,871, 6,573,287, 6,432,969, 6,395,767, 6,380,398, 6,303,661, 6,242,422, 6,166,063, 6,100,234, and 6,040,145.

Some embodiments of the present invention include every combination of one or more DPP-IV inhibitors selected from the DPP-IV inhibitors found in U.S. Pat. Nos. 2005059724, 2005059716, 2005043292, 2005038020, 2005032804, 2005004205, 2004259903, 2004259902, 2004259883, 2004254226, 2004242898, 2004229926, 2004180925, 2004176406, 2004138214, 2004116328, 2004110817, 2004106656, 2004097510, 2004087587, 2004082570, 2004077645, 2004072892, 2004063935, 2004034014, 2003232788, 2003225102, 2003216450, 2003216382, 2003199528, 2003195188,2003162820, 2003149071, 2003134802, 2003130281, 2003130199, 2003125304, 2003119750, 2003119738, 2003105077, 2003100563, 2003087950, 2003078247, 2002198205, 2002183367, 2002103384, 2002049164, and 2002006899.

Some embodiments of the present invention include every combination of one or more DPP-IV inhibitors selected from the DPP-IV inhibitors found in International Patent Application Publication Nos. WO 2005/087235, WO 2005/082348, WO 2005/082849, WO 2005/079795, WO 2005/075426, WO 2005/072530, WO 2005/063750, WO 2005/058849, WO 2005/049022, WO 2005/047297, WO 2005/044195, WO 2005/042488, WO 2005/040095, WO 2005/037828, WO 2005/037779, WO 2005/034940, WO 2005/033099, WO 2005/032590, WO 2005/030751, WO 2005/030127, WO 2005/026148, WO 2005/025554, WO 2005/023762, WO 2005/020920, WO 05/19168, WO 05/12312, WO 05/12308, WO 05/12249, WO 05/11581, WO 05/09956, WO 05/03135, WO 05/00848, WO 05/00846, WO 04/112701, WO 04/111051, WO 04/111041, WO 04/110436, WO 04/110375, WO 04/108730, WO 04/104216, WO 04/104215, WO 04/103993, WO 04/103276, WO 04/99134, WO 04/96806, WO 04/92128, WO 04/87650, WO 04/87053, WO 04/85661, WO 04/85378, WO 04/76434, WO 04/76433, WO 04/71454, WO 04/69162, WO 04/67509, WO 04/64778, WO 04/58266, WO 04/52362, WO 04/52850, WO 04/50022, WO 04/50658, WO 04/48379, WO 04/46106, WO 04/43940, WO 04/41820, WO 04/41795, WO 04/37169, WO 04/37181, WO 04/33455, WO 04/32836, WO 04/20407, WO 04/18469, WO 04/18468, WO 04/18467, WO 04/14860, WO 04/09544, WO 04/07468, WO 04/07446, WO 04/04661, WO 04/00327, WO 03/106456, WO 03/104229, WO 03/101958, WO 03/101448, WO 03/99279, WO 03/95425, WO 03/84940, WO 03/82817, WO 03/80633, WO 03/74500, WO 03/72556, WO 03/72528, WO 03/68757, WO 03/68748, WO 03/57666, WO 03/57144, WO 03/55881, WO 03/45228, WO 03/40174, WO 03/38123, WO 03/37327, WO 03/35067, WO 03/35057, WO 03/24965, WO 03/24942, WO 03/22871, WO 03/15775, WO 03/04498, WO 03/04496, WO 03/02530, WO 03/02596, WO 03/02595, WO 03/02593, WO 03/02553, WO 03/02531, WO 03/00181, WO 03/00180, WO 03/00250, WO 02/83109, WO 02/83128, WO 02/76450, WO 02/68420, WO 02/62764, WO 02/55088, WO 02/51836, WO 02/38541, WO 02/34900, WO 02/30891, WO 02/30890, WO 02/14271, WO 02/02560, WO 01/97808, WO 01/96295, WO 01/81337, WO 01/81304, WO 01/68603, WO 01/55105, WO 01/52825, WO 01/34594, WO 00/71135, WO 00/69868, WO 00/56297, WO 00/56296, WO 00/34241, WO 00/23421, WO 00/10549, WO 99/67278, WO 99/62914, WO 99/61431, WO 99/56753, WO 99/25719, WO 99/16864, WO 98/50066, WO 98/50046, WO 98/19998, WO 98/18763, WO 97/40832, WO 95/29691, WO 95/15309, WO 93/10127, WO 93/08259, and WO 91/16339.

Some embodiments of the present invention include every combination of one or more DPP-IV inhibitors selected from the DPP-IV inhibitors found in Patent Publication Nos. EP 1517907, EP 1513808, EP 1492777, EP 1490335, EP 1489088, EP 1480961, EP 1476435, EP 1476429, EP 1469873, EP 1465891, EP 1463727, EP 1461337, EP 1450794, EP 1446116, EP 1442049, EP 1441719, EP 1426366, EP 1412357, EP1406873, EP 1406872, EP 1406622, EP 1404675, EP 1399420, EP 1399471, EP 1399470, EP 1399469, EP 1399433, EP 1399154, EP 1385508, EP 1377288, EP 1355886, EP 1354882, EP 1338592, EP 1333025, EP 1304327, EP 1301187, EP 1296974, EP 1280797, EP 1282600, EP 1261586, EP 1258476, EP 1254113, EP 1248604, EP 1245568, EP 1215207, EP 1228061, EP 1137635, EP 1123272, EP 1104293, EP 1082314, EP 1050540, EP 1043328, EP 0995440, EP 0980249, EP 0975359, EP 0731789, EP 0641347, EP 0610317, EP 0528858, CA 2466870, CA 2433090, CA 2339537, CA 2289125, CA 2289124, CA 2123128, DD 296075, DE 19834591, DE 19828113, DE 19823831, DE 19616486, DE 10333935, DE 10327439, DE 10256264, DE 10251927, DE 10238477, DE 10238470, DE 10238243, DE 10143840, FR 2824825, FR 2822826, JP2005507261, JP 2005505531, JP 2005502624, JP 2005500321, JP 2005500308, JP2005023038, JP 2004536115, JP 2004535445, JP 2004535433, JP 2004534836, JP 2004534815, JP 2004532220, JP 2004530729, JP 2004525929, JP 2004525179, JP 2004522786, JP 2004521149, JP 2004503531, JP 2004315496, JP 2004244412, JP 2004043429, JP 2004035574, JP 2004026820, JP 2004026678, JP 2004002368, JP 2004002367, JP 2003535898, JP 2003535034, JP 2003531204, JP 2003531191, JP 2003531118, JP 2003524591, JP 2003520849, JP 2003327532, JP 2003300977, JP 2003238566, JP 2002531547, JP 2002527504, JP 2002517401, JP 2002516318, JP 2002363157, JP 2002356472, JP 2002356471, JP 2002265439, JP 2001510442, JP 2000511559, JP 2000327689, JP 2000191616, JP 1998182613, JP 1998081666, JP 1997509921, JP 1995501078, and JP 1993508624.

In some embodiments, the DPP-IV inhibitor has an IC₅₀ of less than about 10 μM, less than about 1 μM, less than about 100 nM, less than about 75 nM, less than about 50 nM, less than about 25 nM, less than about 20 nM, less than about 15 nM, less than about 10 nM, less than about 5 nM, less than about 4 nM, less than about 3 nM, less than about 2 nM, or less than about 1 nM, in any one of the DPP-IV inhibition assays known in the art, including in the references disclosed herein. In some embodiments, the DPP-IV inhibitor has an IC₅₀ of less than about 50 nM, less than about 25 nM, less than about 20 nM, less than about 15 nM, less than about 10 nM, less than about 5 nM, less than about 4 nM, less than about 3 nM, less than about 2 nM, or less than about 1 nM, in any one of the DPP-IV inhibition assays known in the art, including in the references disclosed herein.

In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 10-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 100-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 10-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 1000-fold.

In some embodiments, the DPP-IV inhibitor is orally active.

In some embodiments, the DPP-IV inhibitor is an inhibitor of human DPP-IV.

Some embodiments of the present invention include every combination of one or more compounds selected from compounds of the following group and pharmaceutically acceptable salts, solvates, and hydrates thereof: 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one; 1-[2-(3-hydroxyadamant-1-ylamino)acetyl]pyrrolidine-2(S)-carbonitrile; (1S,3S,5S)-2-[2(S)-amino-2-(3-hydroxyadamantan-1-yl)acetyl]-2-azabicyclo[3.1.0]hexane-3-carbonitrile; 2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine; 1-[N-[3(R)-pyrrolidinyl]glycyl]pyrrolidin-2(R)-yl boronic acid; 4(S)-fluoro-1-[2-[(1R,3S)-3-(1H-1,2,4-triazol-1-ylmethyl)cyclopentylamino]acetyl]pyrrolidine-2(S)-carbonitrile; 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one; (2S,4S)-2-cyano-4-fluoro-1-[(2-hydroxy-1,1-dimethyl)ethylamino]acetylpyrrolidine; 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione; 1-((3S,4S)-4-amino-1-(4-(3,3-difluoropyrrolidin-1-yl)-1,3,5-triazin-2-yl)pyrrolidin-3-yl)-5,5-difluoropiperidin-2-one; (R)-2-((6-(3-aminopiperidin-1-yl)-3-methyl-2,4-dioxo-3,4-dihydropyrimidin-1(2H)-yl)methyl)-4-fluorobenzonitrile; 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide; ((2S,4S)-4-(4-(3-methyl-1-phenyl-1H-pyrazol-5-yl)piperazin-1-yl)pyrrolidin-2-yl)(thiazolidin-3-yl)methanone; (2S,4S)-1-[2-[(4-ethoxycarbonylbicyclo[2.2.2]oct-1-yl)amino]acetyl]-4-fluoropyrrolidine-2-carbonitrile; 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione; 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile; (3,3-difluoropyrrolidin-1-yl)-((2S,4S)-4-(4-(pyrimidin-2-yl)piperazin-1-yl)pyrrolidin-2-yl)methanone; (2S,4S)-1-[(2S)-2-amino-3,3-bis(4-fluorophenyl)propanoyl]-4-fluoropyrrolidine-2-carbonitrile; (2S,5R)-5-ethynyl-1-{N-(4-methyl-1-(4-carboxy-pyridin-2-yl)piperidin-4-yl)glycyl}pyrrolidine-2-carbonitrile; and (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine

Sitagliptin phosphate (Januvia®, MK-0431, dihydrogenphosphate salt of 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one) is marketed by Merck & Co. for once-daily oral treatment of type 2 diabetes. Januvia was first launched in Mexico followed by commercialization in the U.S. In 2007, the product was approved by the European Medicines Evaluation Agency (EMEA) and is currently available in the U.K., Germany and Spain. In 2009, Januvia was approved and launched in Japan. In addition, Merck has also filed for approval of Januvia in the U.S. as an adjunct to diet and exercise and in combination with other therapies to improve glycemic control in the treatment of diabetes. The compound, 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one, and pharmaceutically acceptable salts thereof are disclosed in international patent publication WO2003/004498. Some embodiments of the present invention include every combination of one or more compounds selected from compounds disclosed in WO2003/004498 and pharmaceutically acceptable salts, solvates, and hydrates thereof. In some embodiments, the DPP-IV inhibitor is selected from 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one, and pharmaceutically acceptable salts, solvates, and hydrates thereof:

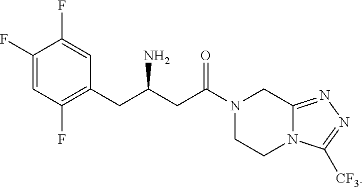 [SMILES: N[C@@H](CC(=O)N1CCn2c(nnc2C(F)(F)F)C1)Cc1cc(F)c(F)cc1F]

In some embodiments, the DPP-IV inhibitor is 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate:

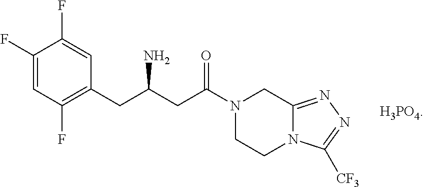

The crystalline form of 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate salt monohydrate is disclosed in international patent publication WO2005/003135. In some embodiments, the DPP-IV inhibitor is crystalline 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate monohydrate.

Vildagliptin (Galvus®, LAF-237, 1-[2-(3-hydroxyadamant-1-ylamino)acetyl]pyrrolidine-2(S)-carbonitrile) is another DPP-IV inhibitor and was first commercialized in Brazil and Mexico by Novartis for oral, once-daily treatment of type 2 diabetes. In 2008, a marketing authorization application (MAA) was approved in the E.U. for this indication and launch took place in the U.K. in March, 2008. An approvable letter has been received for the regulatory application filed in the U.S. Vildagliptin was approved in Japan in 2010. The compound, 1-[2-(3-hydroxyadamant-1-ylamino)acetyl]pyrrolidine-2(S)-carbonitrile, is disclosed in international patent publication WO2000/034241. Some embodiments of the present invention include every combination of one or more compounds selected from compounds disclosed in WO2000/034241 and pharmaceutically acceptable salts, solvates, and hydrates thereof. In some embodiments, the DPP-IV inhibitor is selected from 1-[2-(3-hydroxyadamant-1-ylamino)acetyl]pyrrolidine-2(S)-carbonitrile, and pharmaceutically acceptable salts, solvates, and hydrates thereof:

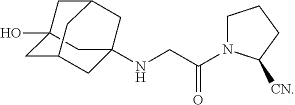 [SMILES: N#C[C@@H]1CCCN1C(=O)CNC12CC3CC(CC(O)(C3)C1)C2]

Certain salts of the compound, 1-[2-(3-hydroxyadamant-1-ylamino)acetyl]pyrrolidine-2(S)-carbonitrile, are disclosed in international patent publication WO2007/019255. In some embodiments, the DPP-IV inhibitor is 1-[2-(3-hydroxyadamant-1-ylamino)acetyl]pyrrolidine-2(S)-carbonitrile HCl:

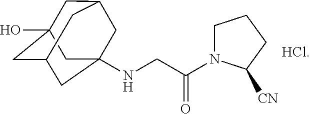 [SMILES: N#C[C@@H]1CCCN1C(=O)CNC12CC3CC(CC(O)(C3)C1)C2]

Saxagliptin (Onglyza™, BMS-477118, (1S,3S,5S)-2-[2(S)-amino-2-(3-hydroxyadamantan-1-yl)acetyl]-2-azabicyclo[3.1.0]hexane-3-carbonitrile) is another DPP-IV inhibitor, which was launched in 2009 by AstraZeneca and Bristol-Myers Squibb in the U.S. for the treatment of type 2 diabetes. In 2009, the product was approved in the E.U. for the treatment of type 2 diabetes independently or in combination with metformin Phase 3 clinical studies are ongoing in Japan for the treatment of type 2 diabetes. The compound, (1S,3S,5S)-2-[2(S)-amino-2-(3-hydroxyadamantan-1-yl)acetyl]-2-azabicyclo[3.1.0]hexane-3-carbonitrile, is disclosed in international patent publication WO2001/068603. Some embodiments of the present invention include every combination of one or more compounds selected from compounds disclosed in WO2001/068603 and pharmaceutically acceptable salts, solvates, and hydrates thereof. In some embodiments, the DPP-IV inhibitor is selected from (1S,3S,5S)-2-[2(S)-amino-2-(3-hydroxyadamantan-1-yl)acetyl]-2-azabicyclo[3.1.0]hexane-3-carbonitrile, and pharmaceutically acceptable salts, solvates, and hydrates thereof:

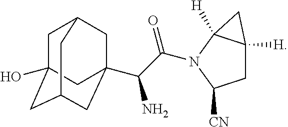

Takeda has filed for regulatory approval of the DPP-IV inhibitor, alogliptin (SYR-322, 2-[6-[3(R)-aminopiperidin-1-yl]-3-methyl-2,4-dioxo-1,2,3,4-tetrahydropyrimidin-1-ylmethyl]benzonitrile) in Japan and the U.S for the once-daily, oral treatment of type 2 diabetes. The compound, 2-[6-[3(R)-aminopiperidin-1-yl]-3-methyl-2,4-dioxo-1,2,3,4-tetrahydropyrimidin-1-ylmethyl]benzonitrile, and pharmaceutically acceptable salts thereof are disclosed in international patent publication WO 2005/095381. Some embodiments of the present invention include every combination of one or more compounds selected from compounds disclosed in WO 2005/095381 and pharmaceutically acceptable salts, solvates, and hydrates thereof. In some embodiments, the DPP-IV inhibitor is selected from 2-[6-[3(R)-aminopiperidin-1-yl]-3-methyl-2,4-dioxo-1,2,3,4-tetrahydropyrimidin-1-ylmethyl]benzonitrile, and pharmaceutically acceptable salts, solvates, and hydrates thereof:

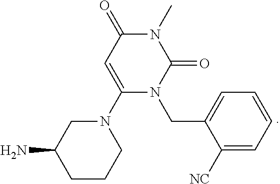 [SMILES: Cn1c(=O)cc(N2CCC[C@@H](N)C2)n(Cc2ccccc2C#N)c1=O]

The crystalline form of 2-[6-[3(R)-aminopiperidin-1-yl]-3-methyl-2,4-dioxo-1,2,3,4-tetrahydropyrimidin-1-ylmethyl]benzonitrile is disclosed in international patent publication WO2007/035372. In some embodiments, the DPP-IV inhibitor is 2-[6-[3(R)-aminopiperidin-1-yl]-3-methyl-2,4-dioxo-1,2,3,4-tetrahydropyrimidin-1-ylmethyl]benzonitrile benzoate:

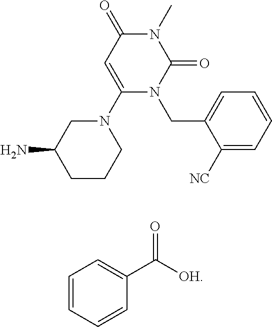 [SMILES: Cn1c(=O)cc(N2CCC[C@@H](N)C2)n(Cc2ccccc2C#N)c1=O ; O=C(O)c1ccccc1]

Linagliptin (BI-1356, Ondero®, 8-[3(R)-aminopiperidin-1-yl]-7-(2-butynyl)-3-methyl-1-(4-methylquinazolin-2-ylmethyl)xanthine) is a DPP-IV inhibitor in phase 3 clinical development at Boehringer Ingelheim to evaluate its potential as add-on therapy to metformin for the treatment of type 2 diabetes. The compound, 8-[3(R)-aminopiperidin-1-yl]-7-(2-butynyl)-3-methyl-1-(4-methylquinazolin-2-ylmethyl)xanthine, is disclosed in international patent publication WO2004/018468. Some embodiments of the present invention include every combination of one or more compounds selected from compounds disclosed in WO2004/018468 and pharmaceutically acceptable salts, solvates, and hydrates thereof. In some embodiments, the DPP-IV inhibitor is selected from 8-[3(R)-aminopiperidin-1-yl]-7-(2-butynyl)-3-methyl-1-(4-methylquinazolin-2-ylmethyl)xanthine, and pharmaceutically acceptable salts, solvates, and hydrates thereof:

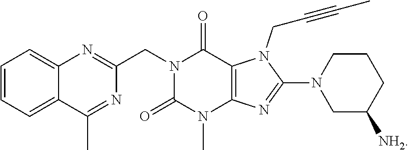 [SMILES: CC#CCn1c(N2CCC[C@@H](N)C2)nc2c1c(=O)n(Cc1nc(C)c3ccccc3n1)c(=O)n2C]

Certain polymorphs of the compound, 8-[3(R)-aminopiperidin-1-yl]-7-(2-butynyl)-3-methyl-1-(4-methylquinazolin-2-ylmethyl)xanthine, are disclosed in international patent publication WO 2007/128721. In some embodiments, the DPP-IV inhibitor is a crystalline form of 8-[3(R)-aminopiperidin-1-yl]-7-(2-butynyl)-3-methyl-1-(4-methylquinazolin-2-ylmethyl)xanthine.

Dutogliptin (PHX-1149, 1-[N-[3(R)-pyrrolidinyl]glycyl]pyrrolidin-2(R)-yl boronic acid) is a DPP-IV inhibitor in phase 3 clinical trials by Phenomix and Forest for the oral, once-daily treatment of type 2 diabetes. The compound, 1-[N-[3(R)-pyrrolidinyl]glycyl]pyrrolidin-2(R)-yl boronic acid, and pharmaceutically acceptable salts thereof are disclosed in international patent publication WO2005/047297. Some embodiments of the present invention include every combination of one or more compounds selected from compounds disclosed in WO2005/047297 and pharmaceutically acceptable salts, solvates, and hydrates thereof. In some embodiments, the DPP-IV inhibitor is selected from 1-[N-[3(R)-pyrrolidinyl]glycyl]pyrrolidin-2(R)-yl boronic acid, and pharmaceutically acceptable salts, solvates, and hydrates thereof:

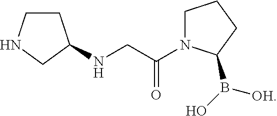 [SMILES: O=C(CN[C@@H]1CCNC1)N1CCC[C@H]1B(O)O]

The crystalline form of 1-[N-[3(R)-pyrrolidinyl]glycyl]pyrrolidin-2(R)-yl boronic acid tartrate is disclosed in international patent publication WO2008/027273. In some embodiments, the DPP-IV inhibitor is 1-[N-[3(R)-pyrrolidinyl]glycyl]pyrrolidin-2(R)-yl boronic acid tartrate:

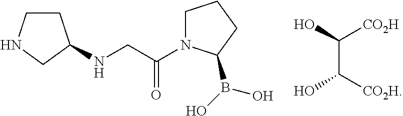 [SMILES: O=C(CN[C@@H]1CCNC1)N1CCC[C@H]1B(O)O ; O=C(O)[C@H](O)[C@@H](O)C(=O)O]

Melogliptin (GRC-8200, 4(S)-fluoro-1-[2-[(1R,3S)-3-(1H-1,2,4-triazol-1-ylmethyl)cyclopentylamino]acetyl]pyrrolidine-2(S)-carbonitrile) is a DPP-IV inhibitor currently undergoing phase 2 clinical trials by Glenmark Pharmaceuticals and Merck KGaA for the treatment of type 2 diabetes. The compound, 4(S)-fluoro-1-[2-[(1R,3S)-3-(1H-1,2,4-triazol-1-ylmethyl)cyclopentylamino]acetyl]pyrrolidine-2(S)-carbonitrile, is disclosed in international patent publication WO2006/040625. Some embodiments of the present invention include every combination of one or more compounds selected from compounds disclosed in WO2006/040625 and pharmaceutically acceptable salts, solvates, and hydrates thereof. In some embodiments, the DPP-IV inhibitor is selected from 4(S)-fluoro-1-[2-[(1R,3S)-3-(1H-1,2,4-triazol-1-ylmethyl)cyclopentylamino]acetyl]pyrrolidine-2(S)-carbonitrile, and pharmaceutically acceptable salts, solvates, and hydrates thereof:

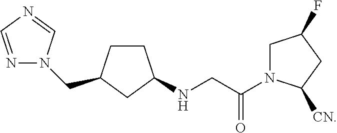 [SMILES: N#C[C@@H]1C[C@H](F)CN1C(=O)CN[C@@H]1CC[C@H](Cn2cncn2)C1]

Carmegliptin (R-1579, 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one) is a DPP-IV inhibitor. The compound, 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one, is disclosed in international patent publication WO2005/000848. Some embodiments of the present invention include every combination of one or more compounds selected from compounds disclosed in WO2005/000848 and pharmaceutically acceptable salts, solvates, and hydrates thereof. In some embodiments, the DPP-IV inhibitor is selected from 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one, and pharmaceutically acceptable salts, solvates, and hydrates thereof:

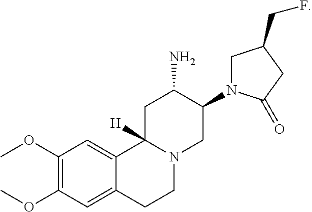 [SMILES: COc1cc2c(cc1OC)[C@@H]1C[C@H](N)[C@@H](N3C[C@@H](CF)CC3=O)CN1CC2]

Taisho disclosed (2S,4S)-2-cyano-4-fluoro-1-[(2-hydroxy-1,1-dimethyl) ethylamino]acetylpyrrolidine, a DPP-IV inhibitor in US patent publication US 2007/0112059. Some embodiments of the present invention include every combination of one or more compounds selected from compounds disclosed in US 2007/0112059 and pharmaceutically acceptable salts, solvates, and hydrates thereof. In some embodiments, the DPP-IV inhibitor is selected from (2S,4S)-2-cyano-4-fluoro-1-[(2-hydroxy-1,1-dimethyl)ethylamino]acetylpyrrolidine, and pharmaceutically acceptable salts, solvates, and hydrates thereof:

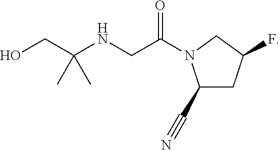 [SMILES: CC(C)(CO)NCC(=O)N1C[C@@H](F)C[C@H]1C#N]

Sanofi-Aventis disclosed a series of substituted bicyclic 8-pyrrolidineoxanthine derivatives as DPP-IV inhibitors in US publication US 2007/0167468. Some embodiments of the present invention include every combination of one or more compounds selected from compounds disclosed in US publication US 2007/0167468 and pharmaceutically acceptable salts, solvates, and hydrates thereof. In some embodiments, the DPP-IV inhibitor is selected from 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione, and pharmaceutically acceptable salts, solvates, and hydrates thereof:

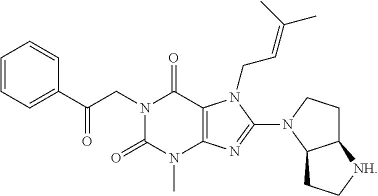 [SMILES: CC(C)=CCn1c(N2CC[C@H]3NCC[C@H]32)nc2c1c(=O)n(CC(=O)c1ccccc1)c(=O)n2C]

Pfizer disclosed a series of 3-amino-pyrrolidine-4-lactam derivatives as DPP-IV inhibitors in international patent publication WO2007/148185. Some embodiments of the present invention include every combination of one or more compounds selected from compounds disclosed in WO2007/148185 and pharmaceutically acceptable salts, solvates, and hydrates thereof. One such compound is 1-((3S,4S)-4-amino-1-(4-(3,3-difluoropyrrolidin-1-yl)-1,3,5-triazin-2-yl)pyrrolidin-3-yl)-5,5-difluoropiperidin-2-one. In some embodiments, the DPP-IV inhibitor is selected from 1-((3S,4S)-4-amino-1-(4-(3,3-difluoropyrrolidin-1-yl)-1,3,5-triazin-2-yl)pyrrolidin-3-yl)-5,5-difluoropiperidin-2-one, and pharmaceutically acceptable salts, solvates, and hydrates thereof:

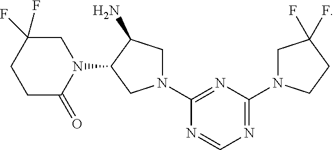 [SMILES: N[C@H]1CN(c2ncnc(N3CCC(F)(F)C3)n2)C[C@@H]1N1CC(F)(F)CCC1=O]

Syrrx disclosed a series of substituted pyrimidine-2,4(1H,3H)-dione derivatives as DPP-IV inhibitors in international patent publication WO2005/095381. Some embodiments of the present invention include every combination of one or more compounds selected from compounds disclosed in WO2005/095381 and pharmaceutically acceptable salts, solvates, and hydrates thereof. One such compound is (R)-2-(6-(3-aminopiperidin-1-yl)-3-methyl-2,4-dioxo-3,4-dihydropyrimidin-1(2H)-yl)methyl)-4-fluorobenzonitrile. In some embodiments, the DPP-IV inhibitor is selected from (R)-2-((6-(3-aminopiperidin-1-yl)-3-methyl-2,4-dioxo-3,4-dihydropyrimidin-1(2H)-yl)methyl)-4-fluorobenzonitrile, and pharmaceutically acceptable salts, solvates, and hydrates thereof:

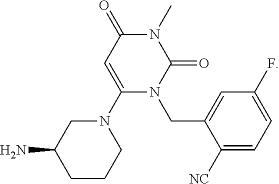 [SMILES: Cn1c(=O)cc(N2CCC[C@@H](N)C2)n(Cc2cc(F)ccc2C#N)c1=O]

Various crystalline forms of (R)-2-((6-(3-aminopiperidin-1-yl)-3-methyl-2,4-dioxo-3,4-dihydropyrimidin-1(2H)-yl)methyl)-4-fluorobenzonitrile succinic acid salt are disclosed in international patent publication WO2008/067465. One embodiment of the present invention pertains to any one or more crystalline forms of (R)-2-(6-(3-aminopiperidin-1-yl)-3-methyl-2,4-dioxo-3,4-dihydropyrimidin-1(2H)-yl)methyl)-4-fluorobenzonitrile succinic acid salt as described in international patent publication WO2008/067465. In some embodiments, the DPP-IV inhibitor is crystalline (R)-2-((6-(3-aminopiperidin-1-yl)-3-methyl-2,4-dioxo-3,4-dihydropyrimidin-1(2H)-yl)methyl)-4-fluorobenzonitrile succinic acid salt:

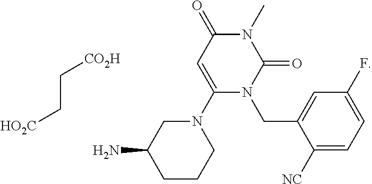 [SMILES: O=C(O)CCC(=O)O ; Cn1c(=O)cc(N2CCC[C@@H](N)C2)n(Cc2cc(F)ccc2C#N)c1=O]

Alantos disclosed a series of substituted 2-cyano-pyrrolidine derivatives as DPP-IV inhibitors in international patent publication WO2006/116157. Some embodiments of the present invention include every combination of one or more compounds selected from compounds disclosed in WO2006/116157 and pharmaceutically acceptable salts, solvates, and hydrates thereof. One such compound is 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide. In some embodiments, the DPP-IV inhibitor is selected from 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide, and pharmaceutically acceptable salts, solvates, and hydrates thereof:

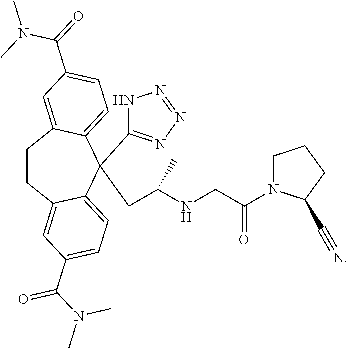 [SMILES: C[C@@H](CC1(c2nnn[nH]2)c2ccc(C(=O)N(C)C)cc2CCc2cc(C(=O)N(C)C)ccc21)NCC(=O)N1CCC[C@H]1C#N]

Mitsubishi disclosed a series of 2,4-disubstituted pyrrolidine derivatives as DPP-IV inhibitors in international patent publication WO2002/0014271. Some embodiments of the present invention include every combination of one or more compounds selected from compounds disclosed in WO2002/0014271 and pharmaceutically acceptable salts, solvates, and hydrates thereof. One such compound is ((2S,4S)-4-(4-(3-methyl-1-phenyl-1H-pyrazol-5-yl)piperazin-1-yl)pyrrolidin-2-yl)(thiazolidin-3-yl)methanone. In some embodiments, the DPP-IV inhibitor is selected from ((2S,4S)-4-(4-(3-methyl-1-phenyl-1H-pyrazol-5-yl)piperazin-1-yl)pyrrolidin-2-yl)(thiazolidin-3-yl)methanone, and pharmaceutically acceptable salts, solvates, and hydrates thereof:

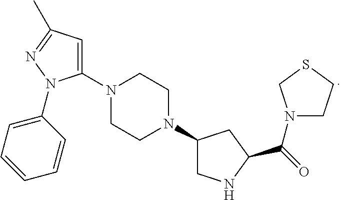 [SMILES: Cc1cc(N2CCN([C@@H]3CN[C@H](C(=O)N4CCSC4)C3)CC2)n(-c2ccccc2)n1 ; "."]

Various crystalline forms of ((2S,4S)-4-(4-(3-methyl-1-phenyl-1H-pyrazol-5-yl)piperazin-1-yl)pyrrolidin-2-yl)(thiazolidin-3-yl)methanone salts are disclosed in international patent publication WO2006/088129 and US publication 2009/0216016. One embodiment of the present invention pertains to any one or more crystalline forms of ((2S,4S)-4-(4-(3-methyl-1-phenyl-1H-pyrazol-5-yl)piperazin-1-yl)pyrrolidin-2-yl)(thiazolidin-3-yl)methanone salt as described in international patent publication WO2006/088129 and US publication 2009/0216016. In some embodiments, the DPP-IV inhibitor is crystalline ((2S,4S)-4-(4-(3-methyl-1-phenyl-1H-pyrazol-5-yl)piperazin-1-yl)pyrrolidin-2-yl)(thiazolidin-3-yl)methanone 2.5 hydrobromide salt:

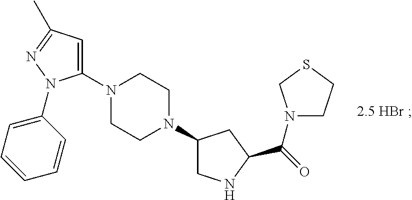

or a mono or a dihydrate thereof. In some embodiments, the DPP-IV inhibitor is crystalline ((2S,4S)-4-(4-(3-methyl-1-phenyl-1H-pyrazol-5-yl)piperazin-1-yl)pyrrolidin-2-yl)(thiazolidin-3-yl)methanone di-hydrobromide salt.

Kyorin disclosed a series of pyrrolidinecarbonitrile derivatives as DPP-IV inhibitors in international patent publication WO2008/114857 and US publication US 2008/0146818. Some embodiments of the present invention include every combination of one or more compounds selected from compounds disclosed in WO2008/114857 and US publication US 2008/0146818, and pharmaceutically acceptable salts, solvates, and hydrates thereof. One such compound is (2S,4S)-1-[2-[(4-ethoxycarbonylbicyclo[2.2.2]oct-1-yl)amino]acetyl]-4-fluoropyrrolidine-2-carbonitrile. In some embodiments, the DPP-IV inhibitor is selected from (2S,4S)-1-[2-[(4-ethoxycarbonylbicyclo[2.2.2]oct-1-yl)amino]acetyl]-4-fluoropyrrolidine-2-carbonitrile, and pharmaceutically acceptable salts, solvates, and hydrates thereof:

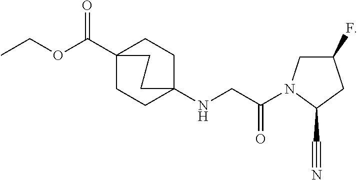 [SMILES: CCOC(=O)C12CCC(NCC(=O)N3C[C@@H](F)C[C@H]3C#N)(CC1)CC2]

Dainippon Sumitomo disclosed a series of bicyclic pyrrole derivatives as DPP-IV inhibitors in international patent publication WO2006/068163 and US publication US 2009/0192129. Some embodiments of the present invention include every combination of one or more compounds selected from compounds disclosed in WO2006/068163 and US publication US 2009/0192129 and pharmaceutically acceptable salts, solvates, and hydrates thereof. One such compound is (6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione. In some embodiments, the DPP-IV inhibitor is selected from (6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione, and pharmaceutically acceptable salts, solvates, and hydrates thereof:

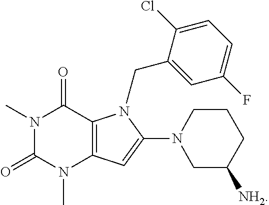 [SMILES: Cn1c(=O)c2c(cc(N3CCC[C@@H](N)C3)n2Cc2cc(F)ccc2Cl)n(C)c1=O]

Dainippon Sumitomo disclosed 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile as a DPP-IV inhibitor in international patent publication WO2009/084497. In some embodiments, the DPP-IV inhibitor is selected from 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile, and pharmaceutically acceptable salts, solvates, and hydrates thereof:

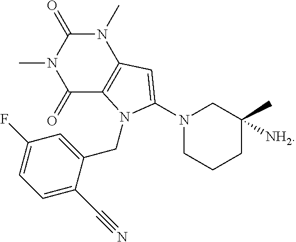 [SMILES: Cn1c(=O)c2c(cc(N3CCC[C@@](C)(N)C3)n2Cc2cc(F)ccc2C#N)n(C)c1=O]

Hoffmann-La Roche disclosed a series of N-substituted pyrrolidine derivatives as DPP-IV inhibitors in international patent publication WO 03/037327. Some embodiments of the present invention include every combination of one or more compounds selected from compounds disclosed in WO 03/037327 and pharmaceutically acceptable salts, solvates, and hydrates thereof. One such compound is (2S)-1-{[2-(5-methyl-2-phenyl-oxazol-4-yl)-ethylamino]-acetyl}-pyrrolidine-2-carbonitrile. In some embodiments, the DPP-IV inhibitor is selected from (2S)-1-{[2-(5-methyl-2-phenyl-oxazol-4-yl)-ethylamino]-acetyl}-pyrrolidine-2-carbonitrile, and pharmaceutically acceptable salts, solvates, and hydrates thereof:

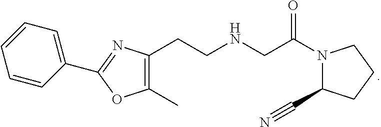 [SMILES: Cc1oc(-c2ccccc2)nc1CCNCC(=O)N1CCC[C@H]1C#N]

Various crystalline forms of (2S)-1-{[2-(5-methyl-2-phenyl-oxazol-4-yl)-ethylamino]-acetyl}-pyrrolidine-2-carbonitrile methansulfonic acid salt are disclosed in international patent publication WO2006/100181. In some embodiments, the DPP-IV inhibitor is (2S)-1-{[2-(5-methyl-2-phenyl-oxazol-4-yl)-ethylamino]-acetyl}-pyrrolidine-2-carbonitrile methansulfonic acid salt (i.e., mesylate):

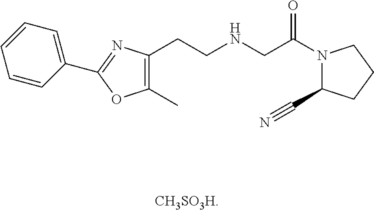 [SMILES: Cc1oc(-c2ccccc2)nc1CCNCC(=O)N1CCC[C@H]1C#N]

Other compounds disclosed by Hoffmann-La Roche in international patent publication WO 03/037327 include (2S)-1-{[1,1-dimethyl-3-(4-pyridin-3-yl-imidazol-1-yl)-propylamino]-acetyl}-pyrrolidine-2-carbonitrile, and pharmaceutically acceptable salts thereof, such as the methansulfonic acid salt. In some embodiments, the DPP-IV inhibitor is selected from (2S)-1-{[1,1-dimethyl-3-(4-pyridin-3-yl-imidazol-1-yl)-propylamino]-acetyl}-pyrrolidine-2-carbonitrile, and pharmaceutically acceptable salts, solvates, and hydrates thereof:

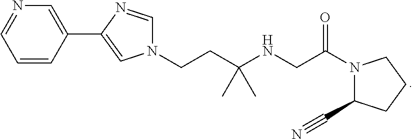 [SMILES: CC(C)(CCn1cnc(-c2cccnc2)c1)NCC(=O)N1CCC[C@H]1C#N]

In some embodiments, the DPP-IV inhibitor is (2S)-1-{[1,1-dimethyl-3-(4-pyridin-3-yl-imidazol-1-yl)-propylamino]-acetyl}-pyrrolidine-2-carbonitrile methansulfonic acid:

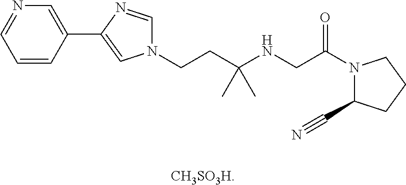 [SMILES: CC(C)(CCn1cnc(-c2cccnc2)c1)NCC(=O)N1CCC[C@H]1C#N]

Various crystalline forms of (2S)-1-{[1,1-dimethyl-3-(4-pyridin-3-yl-imidazol-1-yl)-propylamino]-acetyl}-pyrrolidine-2-carbonitrile fumaric acid salt are disclosed in international patent publication WO2007/071576. In some embodiments, the DPP-IV inhibitor is (2S)-1-{[1,1-dimethyl-3-(4-pyridin-3-yl-imidazol-1-yl)-propylamino]-acetyl}-pyrrolidine-2-carbonitrile fumaric acid salt (i.e., fumarate):

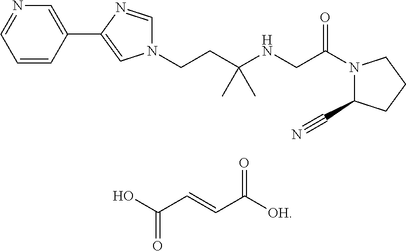 [SMILES: CC(C)(CCn1cnc(-c2cccnc2)c1)NCC(=O)N1CCC[C@H]1C#N ; O=C(O)/C=C/C(=O)O]

Pfizer disclosed a series of proline derivatives as DPP-IV inhibitors in international patent publication WO2005/116014. Some embodiments of the present invention include every combination of one or more compounds selected from compounds disclosed in WO2005/116014 and pharmaceutically acceptable salts, solvates, and hydrates thereof. One such compound is (3,3-difluoropyrrolidin-1-yl)-((2S,4S)-4-(4-(pyrimidin-2-yl)piperazin-1-yl)pyrrolidin-2-yl)methanone. In some embodiments, the DPP-IV inhibitor is selected from (3,3-difluoropyrrolidin-1-yl)-((2S,4S)-4-(4-(pyrimidin-2-yl)piperazin-1-yl)pyrrolidin-2-yl)methanone, and pharmaceutically acceptable salts, solvates, and hydrates thereof:

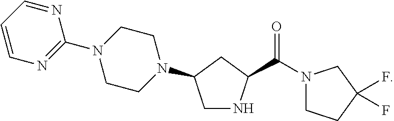 [SMILES: O=C([C@@H]1C[C@H](N2CCN(c3ncccn3)CC2)CN1)N1CCC(F)(F)C1]

GlaxoSmithKline disclosed a series of fluoropyrrolidine derivatives as DPP-IV inhibitors in international patent publication WO 03/002531. Some embodiments of the present invention include every combination of one or more compounds selected from the DPP-IV inhibitors disclosed in WO 03/037327 and pharmaceutically acceptable salts, solvates, and hydrates thereof. One such compound is (2S,4S)-1-[(2S)-2-amino-3,3-bis(4-fluorophenyl)propanoyl]-4-fluoropyrrolidine-2-carbonitrile (Denagliptin). In some embodiments, the DPP-IV inhibitor is selected from (2S,4S)-1-[(2S)-2-amino-3,3-bis(4-fluorophenyl)propanoyl]-4-fluoropyrrolidine-2-carbonitrile, and pharmaceutically acceptable salts, solvates, and hydrates thereof:

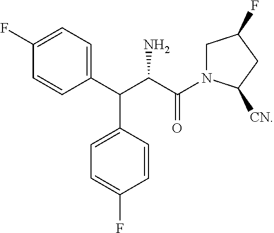 [SMILES: N#C[C@@H]1C[C@H](F)CN1C(=O)[C@@H](N)C(c1ccc(F)cc1)c1ccc(F)cc1]

Various crystalline forms of (2S,4S)-1-[(2S)-2-amino-3,3-bis(4-fluorophenyl)propanoyl]-4-fluoropyrrolidine-2-carbonitrile and salts have been disclosed in international patent publication WO 2005/009956. One salt disclosed is (2S,4S)-1-[(2S)-2-amino-3,3-bis(4-fluorophenyl)propanoyl]-4-fluoropyrrolidine-2-carbonitrile p-toluenesulfonic acid salt (also referred to as (2S,4S)-4-fluoro-1-[4-fluoro-β-(4-fluorophenyl)-L-phenylalanyl]-2-pyrrolidinecarbonitrile p-toluenesulfonic acid salt, or Denagliptin tosylate). In some embodiments, the DPP-IV inhibitor is (2S,4S)-1-[(2S)-2-amino-3,3-bis(4-fluorophenyl)propanoyl]-4-fluoropyrrolidine-2-carbonitrile p-toluenesulfonic acid salt:

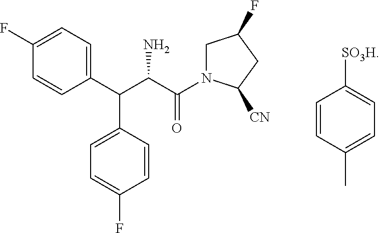 [SMILES: N#C[C@@H]1C[C@H](F)CN1C(=O)[C@@H](N)C(c1ccc(F)cc1)c1ccc(F)cc1 ; Cc1ccc(S(=O)(=O)O)cc1]

Abbott disclosed a series of substituted pyrrolidinyl derivatives as DPP-IV inhibitors in international patent publication WO 2004/026822. Some embodiments of the present invention include every combination of one or more compounds selected from the DPP-IV inhibitors disclosed in WO 2004/026822 and pharmaceutically acceptable salts, solvates, and hydrates thereof. One such compound is (2S,5R)-5-ethynyl-1-{N-(4-methyl-1-(4-carboxy-pyridin-2-yl)piperidin-4-yl)glycyl}pyrrolidine-2-carbonitrile. In some embodiments, the DPP-IV inhibitor is selected from (2S,5R)-5-ethynyl-1-{N-(4-methyl-1-(4-carboxy-pyridin-2-yl)piperidin-4-yl)glycyl}pyrrolidine-2-carbonitrile, and pharmaceutically acceptable salts, solvates, and hydrates thereof:

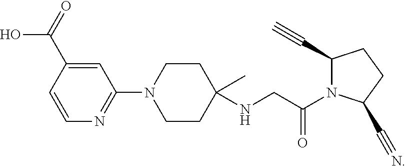 [SMILES: C#C[C@H]1CC[C@@H](C#N)N1C(=O)CNC1(C)CCN(c2cc(C(=O)O)ccn2)CC1]

Abbott has further disclosed a series of substituted cyclohexanyl/cyclohexenyl derivatives as DPP-IV inhibitors in international patent publication WO 2007/027651. Some embodiments of the present invention include every combination of one or more compounds selected from the DPP-IV inhibitors disclosed in WO 2007/027651 and pharmaceutically acceptable salts, solvates, and hydrates thereof. One such compound is (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine 1n some embodiments, the DPP-IV inhibitor is selected from (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine, and pharmaceutically acceptable salts, solvates, and hydrates thereof:

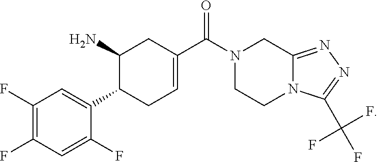 [SMILES: N[C@H]1CC(C(=O)N2CCn3c(nnc3C(F)(F)F)C2)=CC[C@@H]1c1cc(F)c(F)cc1F]

### Biguanides

The biguanides are a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin ((phenylethyl)biguanide), metformin (dimethylbiguanide), buformin (butylbiguanide), proguanil (1-(p-chlorophenyl)-5-isopropylbiguanide), and biguanides known in the art.

In some embodiments, the pharmaceutical agent or the second pharmaceutical agent is a biguanide selected from the following biguanide:

(phenylethyl)biguanide, dimethylbiguanide, butylbiguanide, 1-(p-chlorophenyl)-5-isopropylbiguanide, and pharmaceutically acceptable salts, solvates, and hydrates thereof.

In some embodiments, the pharmaceutical agent or the second pharmaceutical agent is a biguanide selected from (phenylethyl)biguanide (chemical structure shown below) and pharmaceutically acceptable salts, solvates, and hydrates thereof:

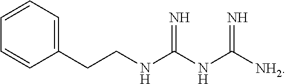 [SMILES: N=C(N)NC(=N)NCCc1ccccc1]

In some embodiments, the pharmaceutical agent or the second pharmaceutical agent is a biguanide selected from dimethylbiguanide (chemical structure shown below) and pharmaceutically acceptable salts, solvates, and hydrates thereof; the chemical structure is as follows:

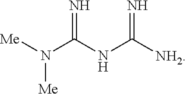

In some embodiments, the pharmaceutical agent or the second pharmaceutical agent is a biguanide selected from butylbiguanide (chemical structure shown below) and pharmaceutically acceptable salts, solvates, and hydrates thereof; the chemical structure is as follows:

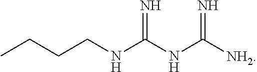

In some embodiments, the pharmaceutical agent or the second pharmaceutical agent is a biguanide selected from 1-(p-chlorophenyl)-5-isopropylbiguanide (chemical structure shown below) and pharmaceutically acceptable salts, solvates, and hydrates thereof; the chemical structure is as follows:

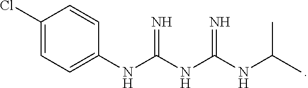 [SMILES: CC(C)NC(=N)NC(=N)Nc1ccc(Cl)cc1]

In some embodiments, the pharmaceutical agent or the second pharmaceutical agent is a biguanide selected from the following biguanides: metformin, phenformin, buformin, and proguanil. In some embodiments, the pharmaceutical agent or the second pharmaceutical agent is metformin. In some embodiments, the pharmaceutical agent or the second pharmaceutical agent is phenformin. In some embodiments, the pharmaceutical agent or the second pharmaceutical agent is buformin. In some embodiments, the pharmaceutical agent or the second pharmaceutical agent is proguanil.

### Alpha-Glucosidase Inhibitors

Alpha-Glucosidase inhibitors belong to the class of drugs which competitively inhibit digestive enzymes such as alpha-amylase, maltase, alpha-dextrinase, sucrase, etc. in the pancreas and or small intestine. The reversible inhibition by alpha-glucosidase inhibitors retard, diminish or otherwise reduce blood glucose levels by delaying the digestion of starch and sugars. Some representative examples of α-glucosidase inhibitors include acarbose ((2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal), miglitol ((2R,3R,4R,5S)-1-(2-hydroxyethyl)-2-(hydroxymethyl)piperidine-3,4,5-triol), voglibose ((1S,2S,3R,4S,5S)-5-(1,3-dihydroxypropan-2-ylamino)-1-(hydroxymethyl)cyclohexane-1,2,3,4-tetraol), and α-glucosidase inhibitors known in the art.

In some embodiments, the pharmaceutical agent or the second pharmaceutical agent is a α-glucosidase inhibitor selected from the following α-glucosidase inhibitors:

(2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal; (2R,3R,4R,5S)-1-(2-hydroxyethyl)-2-(hydroxymethyl)piperidine-3,4,5-triol; (1S,2S,3R,4S,5S)-5-(1,3-dihydroxypropan-2-ylamino)-1-(hydroxymethyl)cyclohexane-1,2,3,4-tetraol; and pharmaceutically acceptable salts, solvates, and hydrates thereof.

In some embodiments, the pharmaceutical agent or the second pharmaceutical agent is a α-glucosidase inhibitor selected from (2R,3R,4R,5R)-4-(2R,3R,4R,5S,6R)-5-(2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal (chemical structure shown below) and pharmaceutically acceptable salts, solvates, and hydrates thereof:

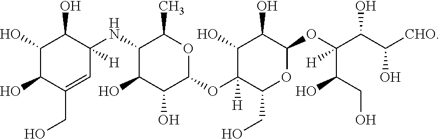 [SMILES: C[C@H]1O[C@H](O[C@H]2[C@H](O)[C@@H](O)[C@@H](O[C@@H]([C@H](O)[C@@H](O)C=O)[C@H](O)CO)O[C@@H]2CO)[C@H](O)[C@@H](O)[C@@H]1N[C@H]1C=C(CO)[C@@H](O)[C@H](O)[C@H]1O]

In some embodiments, the pharmaceutical agent or the second pharmaceutical agent is a α-glucosidase inhibitor selected from (2R,3R,4R,5S)-1-(2-hydroxyethyl)-2-(hydroxymethyl)piperidine-3,4,5-triol (chemical structure shown below) and pharmaceutically acceptable salts, solvates, and hydrates thereof:

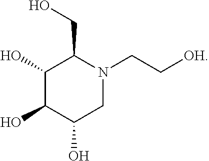 [SMILES: OCCN1C[C@H](O)[C@@H](O)[C@H](O)[C@H]1CO]

In some embodiments, the pharmaceutical agent or the second pharmaceutical agent is a α-glucosidase inhibitor selected from (1S,2S,3R,4S,5S)-5-(1,3-dihydroxypropan-2-ylamino)-1-(hydroxymethyl)cyclohexane-1,2,3,4-tetraol (chemical structure shown below) and pharmaceutically acceptable salts, solvates, and hydrates thereof:

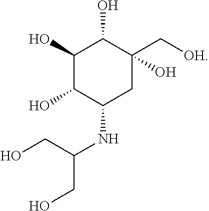 [SMILES: OCC(CO)N[C@H]1C[C@](O)(CO)[C@@H](O)[C@H](O)[C@H]1O]

In some embodiments, the pharmaceutical agent or the second pharmaceutical agent is an alpha-glucosidase inhibitor selected from: acarbose, miglitol, and voglibose. In some embodiments, the pharmaceutical agent or the second pharmaceutical agent is acarbose. In some embodiments, the pharmaceutical agent or the second pharmaceutical agent is miglitol. In some embodiments, the pharmaceutical agent or the second pharmaceutical agent is voglibose.

### Insulin and Insulin Analogues

The term “insulin analogue” refers to the naturally occurring human hormone and insulin receptor ligands (i.e., synthetic insulin analogues). Insulin receptor ligands are structurally different from the natural human hormone, but have substantially the same activity as human insulin in terms of glycemic control. Examples of an insulin analogue include, NPH insulin (also known as Humulin N, Novolin N, NPH Lletin II, and insulin isophane), insulin lispro (28B-L-lysine-29B-L-proline-insulin, wherein insulin is human insulin), insulin aspart (28B-L-aspartic acid-insulin, wherein insulin is human insulin), insulin glulisine (3B-L-lysine-29B-L-glutamic acid-insulin, wherein insulin is human insulin), and insulin analogues known in the art.

NPH insulin is marketed by Eli Lilly and Company under the name Humulin N, and is considered as an intermediate-acting insulin analogue given to help control the blood sugar level of those with diabetes. Insulin lispro is marketed by Eli Lilly and Company under the name Humalog, and is considered a rapid acting insulin analogue. Insulin aspart is marketed by Novo Nordisk and sold as NovoRapid. Insulin aspart is considered a fast acting insulin analogue. Insulin glulisine was developed by Sanofi-Aventis and is sold under the trade name Apidra. Insulin glulisine is considered a rapid acting insulin analogue but shorter duration of action compared to human insulin.

In some embodiments, the pharmaceutical agent or the second pharmaceutical agent is an insulin analogue selected from NPH insulin and pharmaceutically acceptable salts, solvates, and hydrates thereof. In some embodiments, the pharmaceutical agent or the second pharmaceutical agent is an insulin analogue selected from insulin lispro and pharmaceutically acceptable salts, solvates, and hydrates thereof. In some embodiments, the pharmaceutical agent or the second pharmaceutical agent is an insulin analogue selected from insulin aspart and pharmaceutically acceptable salts, solvates, and hydrates thereof. In some embodiments, the pharmaceutical agent or the second pharmaceutical agent is an insulin analogue selected from insulin glulisine and pharmaceutically acceptable salts, solvates, and hydrates thereof.

### Sulfonylureas

The sulfonylureas are drugs which promote secretion of insulin from pancreatic beta cells by transmitting signals of insulin secretion via receptors in the cell membranes. Examples of a sulfonylurea include tolbutamide (Orinase, N-(butylcarbamoyl)-4-methylbenzenesulfonamide); acetohexamide (Dymelor, 4-acetyl-N-(cyclohexylcarbamoyl)benzenesulfonamide); tolazamide (Tolinase, N-(azepan-1-ylcarbamoyl)-4-methylbenzenesulfonamide); chlorpropamide (Diabinese, 4-chloro-N-(propylcarbamoyl)benzenesulfonamide); glipizide (Glucotrol, N-(4-(N-(cyclohexylcarbamoyl)sulfamoyl)phenethyl)-5-methylpyrazine-2-carboxamide); glibenclamide, also known as glyburide (Diabeta, Micronase, Glynase, 5-chloro-N-(4-(N-(cyclohexylcarbamoyl)sulfamoyl)phenethyl)-2-methoxybenzamide); glimepiride (Amaryl, 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide); gliclazide (Diamicron, N-(hexahydrocyclopenta[c]pyrrol-2(1H)-ylcarbamoyl)-4-methylbenzenesulfonamide); and sulfonylureas known in the art.

In some embodiments, the pharmaceutical agent or the second pharmaceutical agent is a sulfonylurea selected from sulfonylureas:

N-(4-(N-(cyclohexylcarbamoyl)sulfamoyl)phenethyl)-5-methylpyrazine-2-carboxamide); 5-chloro-N-(4-(N-(cyclohexylcarbamoyl)sulfamoyl)phenethyl)-2-methoxybenzamide; 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide; and pharmaceutically acceptable salts, solvates, and hydrates thereof.

In some embodiments, the pharmaceutical agent or the second pharmaceutical agent is a sulfonylurea selected from N-(butylcarbamoyl)-4-methylbenzenesulfonamide (chemical structure shown below) and pharmaceutically acceptable salts, solvates, and hydrates thereof:

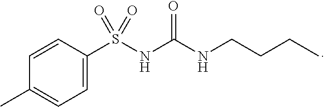 [SMILES: CCCCNC(=O)NS(=O)(=O)c1ccc(C)cc1]

In some embodiments, the pharmaceutical agent or the second pharmaceutical agent is a sulfonylurea selected from 4-acetyl-N-(cyclohexylcarbamoyl)benzenesulfonamide (chemical structure shown below) and pharmaceutically acceptable salts, solvates, and hydrates thereof:

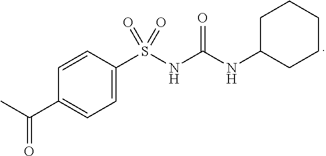 [SMILES: CC(=O)c1ccc(S(=O)(=O)NC(=O)NC2CCCCC2)cc1]

In some embodiments, the pharmaceutical agent or the second pharmaceutical agent is a sulfonylurea selected from N-(azepan-1-ylcarbamoyl)-4-methylbenzenesulfonamide (chemical structure shown below) and pharmaceutically acceptable salts. solvates. and hydrates thereof:

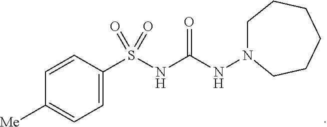 [SMILES: Cc1ccc(S(=O)(=O)NC(=O)NN2CCCCCC2)cc1]

In some embodiments, the pharmaceutical agent or the second pharmaceutical agent is a sulfonylurea selected from 4-chloro-N-(propylcarbamoyl)benzenesulfonamide (chemical structure shown below) and pharmaceutically acceptable salts, solvates, and hydrates thereof:

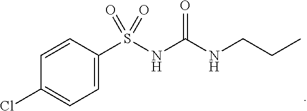 [SMILES: CCCNC(=O)NS(=O)(=O)c1ccc(Cl)cc1]

In some embodiments, the pharmaceutical agent or the second pharmaceutical agent is a sulfonylurea selected from N-(4-(N-(cyclohexylcarbamoyl)sulfamoyl)phenethyl)-5-methylpyrazine-2-carboxamide (chemical structure shown below) and pharmaceutically acceptable salts, solvates, and hydrates thereof:

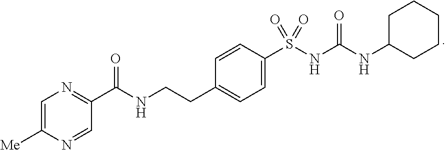 [SMILES: Cc1cnc(C(=O)NCCc2ccc(S(=O)(=O)NC(=O)NC3CCCCC3)cc2)cn1]

In some embodiments, the pharmaceutical agent or the second pharmaceutical agent is a sulfonylurea selected from 5-chloro-N-(4-(N-(cyclohexylcarbamoyl)sulfamoyl)phenethyl)-2-methoxybenzamide (chemical structure shown below) and pharmaceutically acceptable salts, solvates, and hydrates thereof:

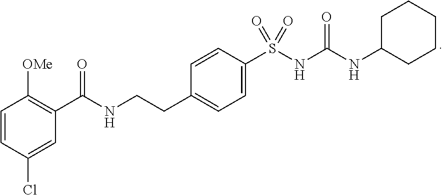 [SMILES: COc1ccc(Cl)cc1C(=O)NCCc1ccc(S(=O)(=O)NC(=O)NC2CCCCC2)cc1]

In some embodiments, the pharmaceutical agent or the second pharmaceutical agent is a sulfonylurea selected from 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide (chemical structure shown below) and pharmaceutically acceptable salts, solvates, and hydrates thereof:

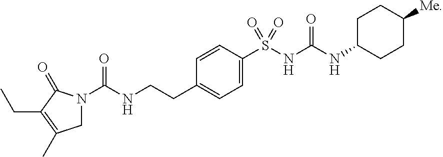 [SMILES: CCC1=C(C)CN(C(=O)NCCc2ccc(S(=O)(=O)NC(=O)N[C@H]3CC[C@H](C)CC3)cc2)C1=O]

In some embodiments, the pharmaceutical agent or the second pharmaceutical agent is a sulfonylurea selected from N-(hexahydrocyclopenta[c]pyrrol-2(1H)-ylcarbamoyl)-4-methylbenzenesulfonamide (chemical structure shown below) and pharmaceutically acceptable salts, solvates, and hydrates thereof:

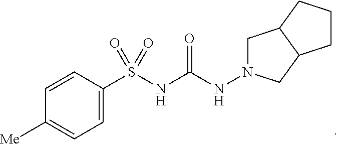 [SMILES: Cc1ccc(S(=O)(=O)NC(=O)NN2CC3CCCC3C2)cc1]

In some embodiments, the pharmaceutical agent or the second pharmaceutical agent is a sulfonylurea selected from the following sulfonylureas and pharmaceutically acceptable salts, solvates, and hydrates thereof: glipizide, glimepiride, and glibenclamide. In some embodiments, the pharmaceutical agent or the second pharmaceutical agent is tolbutamide. In some embodiments, the pharmaceutical agent or the second pharmaceutical agent is acetohexamide. In some embodiments, the pharmaceutical agent or the second pharmaceutical agent is tolazamide. In some embodiments, the pharmaceutical agent or the second pharmaceutical agent is chlorpropamide. In some embodiments, the pharmaceutical agent or the second pharmaceutical agent is glipizide. In some embodiments, the pharmaceutical agent or the second pharmaceutical agent is glyburide. In some embodiments, the pharmaceutical agent or the second pharmaceutical agent is glimepiride. In some embodiments, the pharmaceutical agent or the second pharmaceutical agent is gliclazide.

### SGLT2 Inhibitors

Sodium-glucose transporter-2 (SGLT2) inhibitors belong to the class of drugs which inhibit the protein SGLT2 and the reabsorption of glucose in the kidney. The inhibition by SGLT2 inhibitors retard, diminish, or otherwise reduce the amount of glucose that is reabsorbed and therefore is eliminated in the urine. Some representative examples of SGLT2 inhibitors include dapagliflozin ((2S,3R,4R,5S,6R)-2-(4-chloro-3-(4-ethoxybenzyl)phenyl)-6-(hydroxymethyl)tetrahydro-2H-pyran-3,4,5-triol, Bristol-Myers Squibb and AstraZeneca), remogliflozin (ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate, GlaxoSmithKline), ASP1941 (Kotobuki/Astellas), canagliflozin ((2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol, Johnson & Johnson/Mitsubishi/Tanabe), ISIS 388626 (an antisense oligonucleotide, Isis Pharmaceuticals), sergliflozin (ethyl ((2R,3S,4S,5R,6S)-3,4,5-trihydroxy-6-(2-(4-methoxybenzyl)phenoxy)tetrahydro-2H-pyran-2-yl)methyl carbonate, GlaxoSmithKline), AVE2268 ((2R,3S,4S,5R,6S)-2-(hydroxymethyl)-6-(2-(4-methoxybenzyl)thiophen-3-yloxy)tetrahydro-2H-pyran-3,4,5-triol, Sanofi-Aventis), BI10773 (Boehringer Ingelheim), CSG453 (Chugai/Roche), LX4211 (Lexicon), and SGLT2 inhibitors known in the art.

In some embodiments, the pharmaceutical agent or the second pharmaceutical agent is an SGLT2 inhibitor selected from the following SGLT2 inhibitors:

(2S,3R,4R,5S,6R)-2-(4-chloro-3-(4-ethoxybenzyl)phenyl)-6-(hydroxymethyl)tetrahydro-2H-pyran-3,4,5-triol; ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; ethyl ((2R,3S,4S,5R,6S)-3,4,5-trihydroxy-6-(2-(4-methoxybenzyl)phenoxy)tetrahydro-2H-pyran-2-yl)methyl carbonate; (2R,3S,4S,5R,6S)-2-(hydroxymethyl)-6-(2-(4-methoxybenzyl)thiophen-3-yloxy)tetrahydro-2H-pyran-3,4,5-triol; (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol; and pharmaceutically acceptable salts, solvates, and hydrates thereof.

In some embodiments, the pharmaceutical agent or the second pharmaceutical agent is a sulfonylurea selected from (2S,3R,4R,5S,6R)-2-(4-chloro-3-(4-ethoxybenzyl)phenyl)-6-(hydroxymethyl)tetrahydro-2H-pyran-3,4,5-triol (chemical structure shown below) and pharmaceutically acceptable salts, solvates, and hydrates thereof:

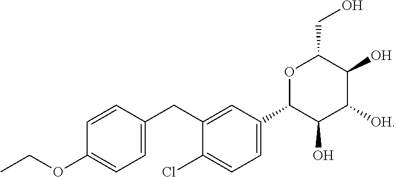 [SMILES: CCOc1ccc(Cc2cc([C@@H]3O[C@H](CO)[C@@H](O)[C@H](O)[C@H]3O)ccc2Cl)cc1]

In some embodiments, the pharmaceutical agent or the second pharmaceutical agent is a sulfonylurea selected from ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate (chemical structure shown below) and pharmaceutically acceptable salts, solvates, and hydrates thereof:

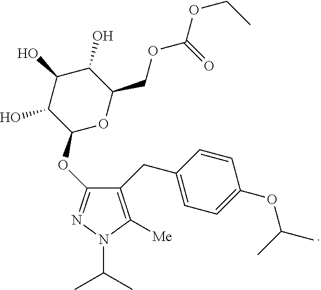 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

In some embodiments, the pharmaceutical agent or the second pharmaceutical agent is a sulfonylurea selected from ethyl ((2R,3S,4S,5R,6S)-3,4,5-trihydroxy-6-(2-(4-methoxybenzyl)phenoxy)tetrahydro-2H-pyran-2-yl)methyl carbonate (chemical structure shown below) and pharmaceutically acceptable salts, solvates, and hydrates thereof:

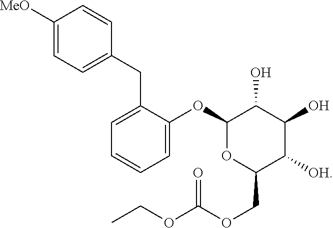 [SMILES: CCOC(=O)OC[C@H]1O[C@@H](Oc2ccccc2Cc2ccc(OC)cc2)[C@H](O)[C@@H](O)[C@@H]1O]

In some embodiments, the pharmaceutical agent or the second pharmaceutical agent is an SGLT2 inhibitor selected from: dapagliflozin, remigliflozin, and sergliflozin. In some embodiments, the pharmaceutical agent or the second pharmaceutical agent is dapagliflozin. In some embodiments, the pharmaceutical agent or the second pharmaceutical agent is remigliflozin. In some embodiments, the pharmaceutical agent or the second pharmaceutical agent is sergliflozin.

Astellas and Kotobuki disclosed a series of SGLT2 inhibitors in international patent publication WO2004/080990. Some embodiments of the present invention include every combination of one or more compounds selected from compounds disclosed in WO2004/080990 and pharmaceutically acceptable salts, solvates, and hydrates thereof.

Aventis disclosed a series of SGLT2 inhibitors in international patent publication WO2004/007517. Some embodiments of the present invention include every combination of one or more compounds selected from compounds disclosed in WO2004/007517 and pharmaceutically acceptable salts, solvates, and hydrates thereof. One such compound is (2R,3S,4S,5R,6S)-2-(hydroxymethyl)-6-(2-(4-methoxybenzyl)thiophen-3-yloxy)tetrahydro-2H-pyran-3,4,5-triol. In some embodiments, the SGLT2 inhibitor is selected from (2R,3S,4S,5R,6S)-2-(hydroxymethyl)-6-(2-(4-methoxybenzyl)thiophen-3-yloxy)tetrahydro-2H-pyran-3,4,5-triol, and pharmaceutically acceptable salts, solvates, and hydrates thereof:

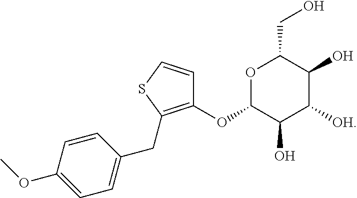 [SMILES: COc1ccc(Cc2sccc2O[C@@H]2O[C@H](CO)[C@@H](O)[C@H](O)[C@H]2O)cc1]

Tanabe disclosed a series of SGLT2 inhibitors in international patent publication WO2005/012326. Some embodiments of the present invention include every combination of one or more compounds selected from compounds disclosed in WO2005/012326 and pharmaceutically acceptable salts, solvates, and hydrates thereof. One such compound is (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol. In some embodiments, the SGLT2 inhibitor is selected from (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol, and pharmaceutically acceptable salts, solvates, and hydrates thereof:

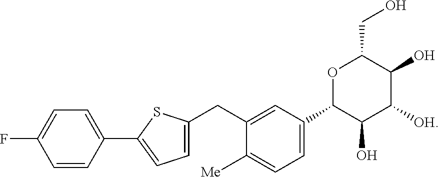 [SMILES: Cc1ccc([C@@H]2O[C@H](CO)[C@@H](O)[C@H](O)[C@H]2O)cc1Cc1ccc(-c2ccc(F)cc2)s1]

Boehringer Ingelheim disclosed a series of SGLT2 inhibitors in international patent publication WO2005/092877. Some embodiments of the present invention include every combination of one or more compounds selected from compounds disclosed in WO2005/092877 and pharmaceutically acceptable salts, solvates, and hydrates thereof.

Chugai disclosed a series of SGLT2 inhibitors in international patent publication WO2006/080421. Some embodiments of the present invention include every combination of one or more compounds selected from compounds disclosed in WO2006/080421 and pharmaceutically acceptable salts, solvates, and hydrates thereof.

Lexicon disclosed a series of SGLT2 inhibitors in international patent publication WO2008/109591. Some embodiments of the present invention include every combination of one or more compounds selected from compounds disclosed in WO2008/109591 and pharmaceutically acceptable salts, solvates, and hydrates thereof.

### Meglitinides

The meglitinides promote secretion of insulin by binding to the pancreatic beta cells in a similar manner as sulfonylureas but at an alternative binding site. Examples of meglitinides include Novo Nordisk's repaglinide (Prandin, (S)-2-ethoxy-4-(2-(3-methyl-1-(2-(piperidin-1-yl)phenyl)butylamino)-2-oxoethyl)benzoic acid), nateglinide (Starlix, (R)-2-((1r,4R)-4-isopropylcyclohexanecarboxamido)-3-phenylpropanoic acid), mitiglinide ((S)-2-benzyl-4-((3aR,7aS)-1H-isoindol-2(3H,3aH,4H,5H,6H,7H,7aH)-yl)-4-oxobutanoic acid), and the like.

In some embodiments, the pharmaceutical agent or the second pharmaceutical agent is a meglitinide selected from the following meglitinides: (S)-2-ethoxy-4-(2-(3-methyl-1-(2-(piperidin-1-yl)phenyl)butylamino)-2-oxoethyl)benzoic acid; (R)-2-((1r,4R)-4-isopropylcyclohexanecarboxamido)-3-phenylpropanoic acid; (S)-2-benzyl-4-((3aR,7aS)-1H-isoindol-2(3H,3aH,4H,5H,6H,7H,7aH)-yl)-4-oxobutanoic acid; and pharmaceutically acceptable salts, solvates, and hydrates thereof.

In some embodiments, the pharmaceutical agent or the second pharmaceutical agent is
(S)-2-ethoxy-4-(2-(3-methyl-1-(2-(piperidin-1-yl)phenyl)butylamino)-2-oxoethyl)benzoic acid chemical structure shown below) and pharmaceutically acceptable salts, solvates, and hydrates thereof:

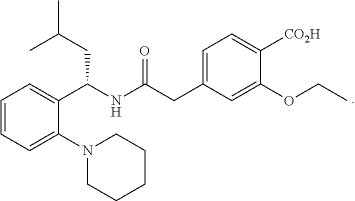 [SMILES: CCOc1cc(CC(=O)N[C@@H](CC(C)C)c2ccccc2N2CCCCC2)ccc1C(=O)O]

In some embodiments, the pharmaceutical agent or the second pharmaceutical agent is a sulfonylurea selected from (R)-2-((1r,4R)-4-isopropylcyclohexanecarboxamido)-3-phenylpropanoic acid (chemical structure shown below) and pharmaceutically acceptable salts, solvates, and hydrates thereof:

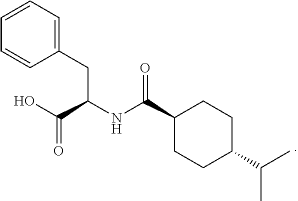 [SMILES: CC(C)[C@H]1CC[C@H](C(=O)N[C@H](Cc2ccccc2)C(=O)O)CC1]

In some embodiments, the pharmaceutical agent or the second pharmaceutical agent is a sulfonylurea selected from (S)-2-benzyl-4-((3aR,7aS)-1H-isoindol-2(3H,3aH,4H,5H,6H,7H,7aH)-yl)-4-oxobutanoic acid (chemical structure shown below) and pharmaceutically acceptable salts, solvates, and hydrates thereof:

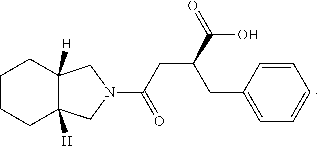 [SMILES: O=C(O)[C@H](CC(=O)N1C[C@H]2CCCC[C@H]2C1)Cc1ccccc1]

In some embodiments, the pharmaceutical agent or the second pharmaceutical agent is a meglitinide selected from the following meglitinides: repaglinide, nateglinide, mitiglinide, and pharmaceutically acceptable salts, solvates, and hydrates thereof. In some embodiments, the pharmaceutical agent or the second pharmaceutical agent is a meglitinide selected from repaglinide and pharmaceutically acceptable salts, solvates, and hydrates thereof. In some embodiments, the pharmaceutical agent or the second pharmaceutical agent is a meglitinide selected from nateglinide and pharmaceutically acceptable salts, solvates, and hydrates thereof. In some embodiments, the pharmaceutical agent or the second pharmaceutical agent is a meglitinide selected from mitiglinide and pharmaceutically acceptable salts, solvates, and hydrates thereof.

### Thiazolidinediones

Thiazolidinediones belong to the class of drugs more commonly known as TZDs. These drugs act by binding to the nuclear receptor peroxisome proliferator-activated receptor gamma (PPARγ) activate transcription of a number of specific genes leading to a decrease in insulin resistance. Examples of thiazolidinediones include rosiglitazone (Avandia, 5-(4-(2-(methyl(pyridin-2-yl)amino)ethoxy)benzyl)thiazolidine-2,4-dione), pioglitazone (Actos, 5-(4-(2-(5-ethylpyridin-2-yl)ethoxy)benzyl)thiazolidine-2,4-dione), troglitazone (Rezulin, 5-(4-((6-hydroxy-2,5,7,8-tetramethylchroman-2-yl)methoxy)benzyl)thiazolidine-2,4-dione), rivoglitazone (5-(4-((6-methoxy-1-methyl-1H-benzo[d]imidazol-2-yl)methoxy)benzyl)thiazolidine-2,4-dione), ciglitazone(5-(4-((1-methylcyclohexyl)methoxy)benzyl)thiazolidine-2,4-dione), and thiazolidinediones known in the art.

In some embodiments, the pharmaceutical agent or the second pharmaceutical agent is a meglitinide selected from: 5-(4-(2-(methyl(pyridin-2-yl)amino)ethoxy)benzyl)thiazolidine-2,4-dione; 5-(4-(2-(5-ethylpyridin-2-yl)ethoxy)benzyl)thiazolidine-2,4-dione; 5-(4-((6-methoxy-1H-benzo[d]imidazol-2-yl)methoxy)benzyl)thiazolidine-2,4-dione; 5-(4-((1-methylcyclohexyl)methoxy)benzyl)thiazolidine-2,4-dione; and pharmaceutically acceptable salts, solvates, and hydrates thereof.

In some embodiments, the pharmaceutical agent or the second pharmaceutical agent is 5-(4-(2-(methyl(pyridin-2-yl)amino)ethoxy)benzyl)thiazolidine-2,4-dione (chemical structure shown below) and pharmaceutically acceptable salts, solvates, and hydrates thereof:

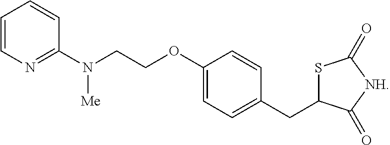 [SMILES: CN(CCOc1ccc(CC2SC(=O)NC2=O)cc1)c1ccccn1]

In some embodiments, the pharmaceutical agent or the second pharmaceutical agent is 5-(4-(2-(5-ethylpyridin-2-yl)ethoxy)benzyl)thiazolidine-2,4-dione (chemical structure shown below) and pharmaceutically acceptable salts, solvates, and hydrates thereof:

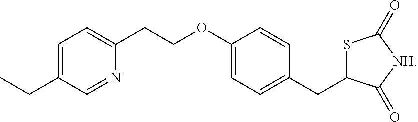 [SMILES: CCc1ccc(CCOc2ccc(CC3SC(=O)NC3=O)cc2)nc1]

In some embodiments, the pharmaceutical agent or the second pharmaceutical agent is 5-(4-((6-hydroxy-2,5,7,8-tetramethylchroman-2-yl)methoxy)benzyl)thiazolidine-2,4-dione (chemical structure shown below) and pharmaceutically acceptable salts, solvates, and hydrates thereof:

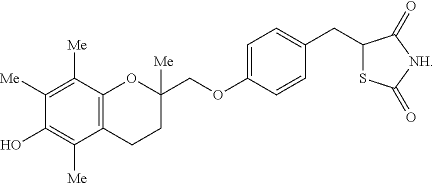 [SMILES: Cc1c(C)c2c(c(C)c1O)CCC(C)(COc1ccc(CC3SC(=O)NC3=O)cc1)O2]

In some embodiments, the pharmaceutical agent or the second pharmaceutical agent is 5-(4-((6-methoxy-1-methyl-1H-benzo[d]imidazol-2-yl)methoxy)benzyl)thiazolidine-2,4-dione (chemical structure shown below) and pharmaceutically acceptable salts, solvates, and hydrates thereof:

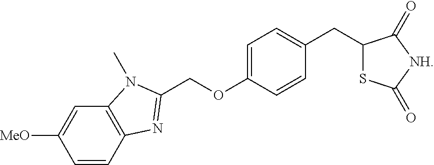 [SMILES: COc1ccc2nc(COc3ccc(CC4SC(=O)NC4=O)cc3)n(C)c2c1]

In some embodiments, the pharmaceutical agent or the second pharmaceutical agent is 5-(4-((1-methylcyclohexyl)methoxy)benzyl)thiazolidine-2,4-dione (chemical structure shown below) and pharmaceutically acceptable salts, solvates, and hydrates thereof:

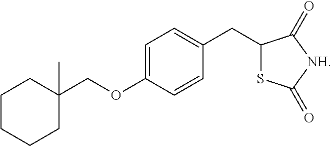 [SMILES: CC1(COc2ccc(CC3SC(=O)NC3=O)cc2)CCCCC1]

In some embodiments, the pharmaceutical agent or the second pharmaceutical agent is a thiazolidinedione selected from rosiglitazone and pharmaceutically acceptable salts, solvates, and hydrates thereof. In some embodiments, the pharmaceutical agent or the second pharmaceutical agent is a thiazolidinedione selected from pioglitazone and pharmaceutically acceptable salts, solvates, and hydrates thereof. In some embodiments, the pharmaceutical agent or the second pharmaceutical agent is a thiazolidinedione selected from troglitazone and pharmaceutically acceptable salts, solvates, and hydrates thereof. In some embodiments, the pharmaceutical agent or the second pharmaceutical agent is a thiazolidinedione selected from rivoglitazone and pharmaceutically acceptable salts, solvates, and hydrates thereof. In some embodiments, the pharmaceutical agent or the second pharmaceutical agent is a thiazolidinedione selected from ciglitazone and pharmaceutically acceptable salts, solvates, and hydrates thereof.

### Anti-Diabetic Peptide Analogues

Anti-diabetic peptide analogues are peptides that promote secretion of insulin by acting as an incretin mimetic, such as, GLP-1 and GIP. Examples of an anti-diabetic peptide analog include, exenatide, liraglutide, taspoglutide, and anti-diabetic peptides analogues know in the art.

In some embodiments, the pharmaceutical agent or the second pharmaceutical agent is an anti-diabetic peptide analogue selected from: exenatide; liraglutide; and taspoglutide. In some embodiments, the pharmaceutical agent or the second pharmaceutical agent is exenatide. In some embodiments, the pharmaceutical agent or the second pharmaceutical agent is liraglutide. In some embodiments, the pharmaceutical agent or the second pharmaceutical agent is taspoglutide.

In some embodiments, the pharmaceutical agent or the second pharmaceutical agent is L-histidylglycyl-L-α-glutamylglycyl-L-threonyl-L-phenylalanyl-L-threonyl-L-seryl-L-α-aspartyl-L-leucyl-L-seryl-L-lysyl-L-glutaminyl-L-methionyl-L-α-glutamyl-L-α-glutamyl-L-α-glutamyl-L-alanyl-L-valyl-L-arginyl-L-leucyl-L-phenylalanyl-L-isoleucyl-L-α-glutamyl-L-tryptophyl-L-leucyl-L-lysyl-L-asparaginylglycylglycyl-L-prolyl-L-seryl-L-serylglycyl-L-alanyl-L-prolyl-L-prolyl-L-prolyl-L-serinamide (i.e., exenatide) and pharmaceutically acceptable salts, solvates, and hydrates thereof.

In some embodiments, the pharmaceutical agent or the second pharmaceutical agent is L-histidyl-L-alanyl-L-α-glutamylglycyl-L-threonyl-L-phenylalanyl-L-threonyl-L-seryl-L-α-aspartyl-L-valyl-L-seryl-L-seryl-L-tyrosyl-L-leucyl-L-α-glutamylglycyl-L-glutaminyl-L-alanyl-L-alanyl-N6-[N-(1-oxohexadecyl)-L-α-glutamyl]-L-lysyl-L-α-glutamyl-L-phenylalanyl-L-isoleucyl-L-alanyl-L-tryptophyl-L-leucyl-L-valyl-L-arginylglycyl-L-arginyl-glycine (liraglutide) and pharmaceutically acceptable salts, solvates, and hydrates thereof.

In some embodiments, the pharmaceutical agent or the second pharmaceutical agent is H₂N-His-2-methyl-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-2-methyl-Ala-Arg-CONH₂ (taspoglutide) and pharmaceutically acceptable salts, solvates, and hydrates thereof.

### Other Utilities

Another object of the present invention relates to radio-labeled compounds of the present invention that would be useful not only in radio-imaging but also in assays, both in vitro and in vivo, for localizing and quantitating GPR119 receptors in tissue samples, including human and for identifying GPR119 receptor ligands by inhibition binding of a radio-labeled compound. It is a further object of this invention to develop novel GPR119 receptor assays of which comprise such radio-labeled compounds.

The present disclosure includes all isotopes of atoms occurring in the present compounds, intermediates, salts and crystalline forms thereof. Isotopes include those atoms having the same atomic number but different mass numbers. One aspect of the present invention includes every combination of one or more atoms in the present compounds, intermediates, salts, and crystalline forms thereof that is replaced with an atom having the same atomic number but a different mass number. One such example is the replacement of an atom that is the most naturally abundant isotope, such as ¹H or ¹²C, found in one the present compounds, intermediates, salts, and crystalline forms thereof, with a different atom that is not the most naturally abundant isotope, such as ²H or ³H (replacing ¹H), or ¹¹C, ¹³C, or ¹⁴C (replacing ¹²C). A compound wherein such a replacement has taken place is commonly referred to as being an isotopically-labeled compound. Isotopic-labeling of the present compounds, intermediates, salts, and crystalline forms thereof can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present compounds, intermediates, salts, and crystalline forms thereof, wherein the naturally occurring distribution of the isotopes in the composition is perturbed. Another aspect of the present invention includes compositions and pharmaceutical compositions comprising compounds as described herein wherein the compound is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Drawings and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or a scarcer radio-isotope or nonradioactive isotope.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas: This procedure normally yields high specific activity products and requires halogenated or unsaturated precursors.

B. Reduction with Sodium Borohydride [³H]: This procedure is rather inexpensive and requires precursors containing reducible functional groups such as aldehydes, ketones, lactones, esters and the like.

C. Reduction with Lithium Aluminum Hydride [³H]: This procedure offers products at almost theoretical specific activities. It also requires precursors containing reducible functional groups such as aldehydes, ketones, lactones, esters and the like.

D. Tritium Gas Exposure Labeling: This procedure involves exposing precursors containing exchangeable protons to tritium gas in the presence of a suitable catalyst.

E. N-Methylation using Methyl Iodide [³H]: This procedure is usually employed to prepare O-methyl or N-methyl (3H) products by treating appropriate precursors with high specific activity methyl iodide (3H). This method in general allows for higher specific activity, such as for example, about 70-90 Ci/mmol

Synthetic methods for incorporating activity levels of ¹²⁵I into target molecules include:

A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A represented procedure was reported by Zhu, G-D. and co-workers in J. Org. Chem., 2002, 67, 943-948.

B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.

C. Aryl and heteroaryl bromide exchange with ¹²⁵I: This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm. 2001, 44, S280-S282.

A radiolabeled GPR119 receptor compound of Formula I can be used in a screening assay to identify/evaluate compounds. In general terms, a newly synthesized or identified compound (i.e., test compound) can be evaluated for its ability to reduce binding of a radio-labeled compound of Formula I to a GPR119 receptor. The ability of a test compound to compete with a radio-labeled compound of Formula I for the binding to a GPR119 receptor directly correlates to its binding affinity.

Certain labeled compounds of the present invention bind to certain GPR119 receptors. In one embodiment the labeled compound has an IC₅₀ less than about 500 μM. In one embodiment the labeled compound has an IC₅₀ less than about 100 μM. In one embodiment the labeled compound has an IC₅₀ less than about 10 μM. In one embodiment the labeled compound has an IC₅₀ less than about 1 μM. In one embodiment the labeled compound has an IC₅₀ less than about 0.1 μM. In one embodiment the labeled compound has an IC₅₀ less than about 0.01 μM. In one embodiment the labeled compound has an IC₅₀ less than about 0.005 μM.

Other uses of the disclosed receptors and methods will become apparent to those skilled in the art based upon, inter alia, a review of this disclosure.

As will be recognized, the steps of the methods of the present invention need not be performed any particular number of times or in any particular sequence. Additional objects, advantages and novel features of this invention will become apparent to those skilled in the art upon examination of the following examples thereof, which are intended to be illustrative and not intended to be limiting.

### EXAMPLES

### Example 1

### Syntheses of Compounds of the Present Invention

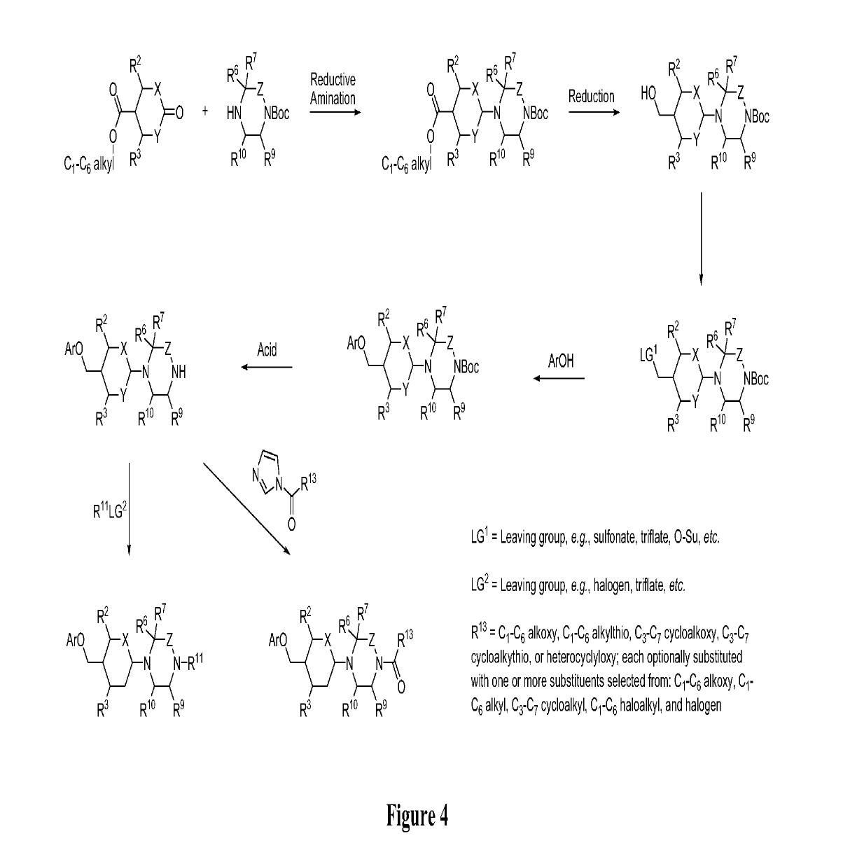

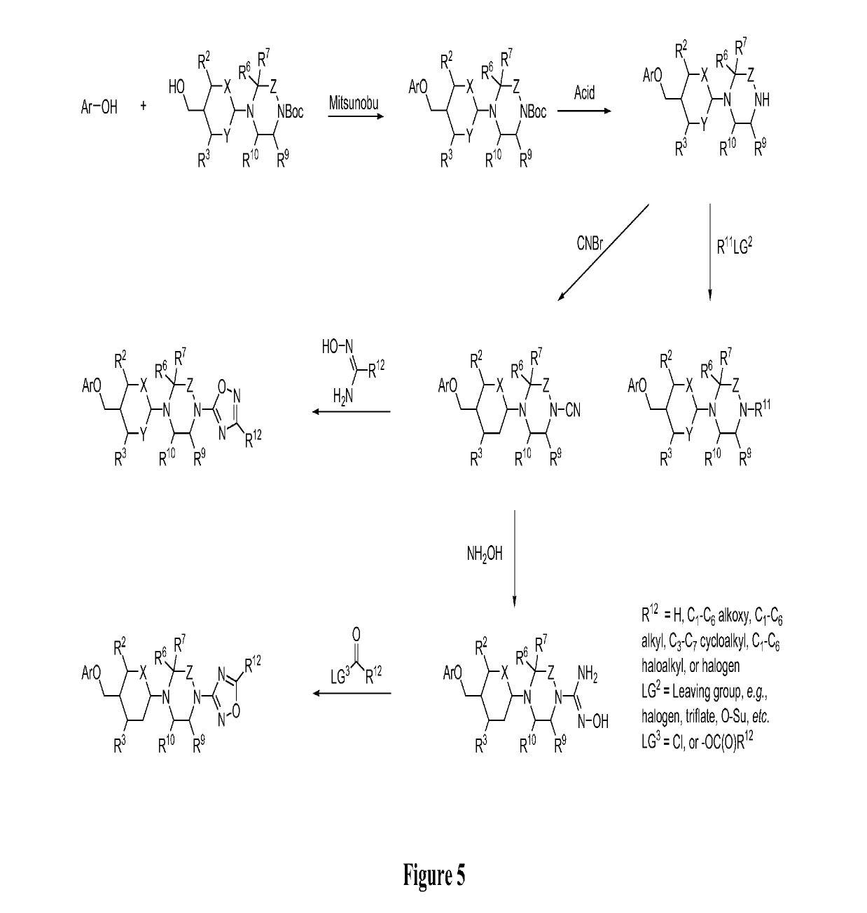

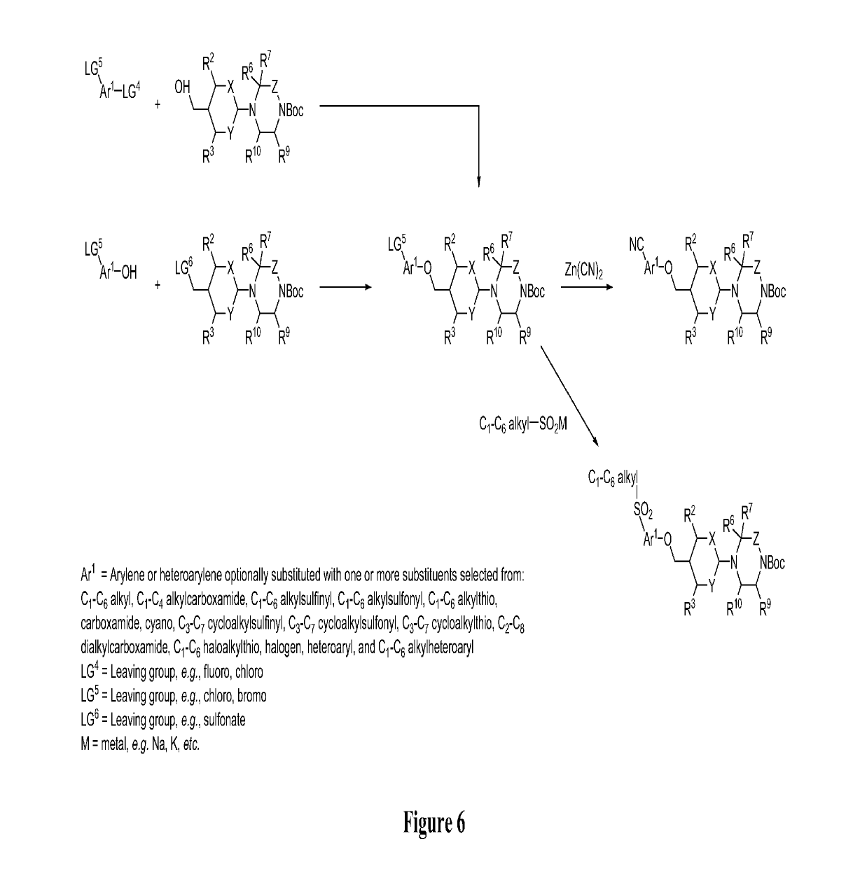

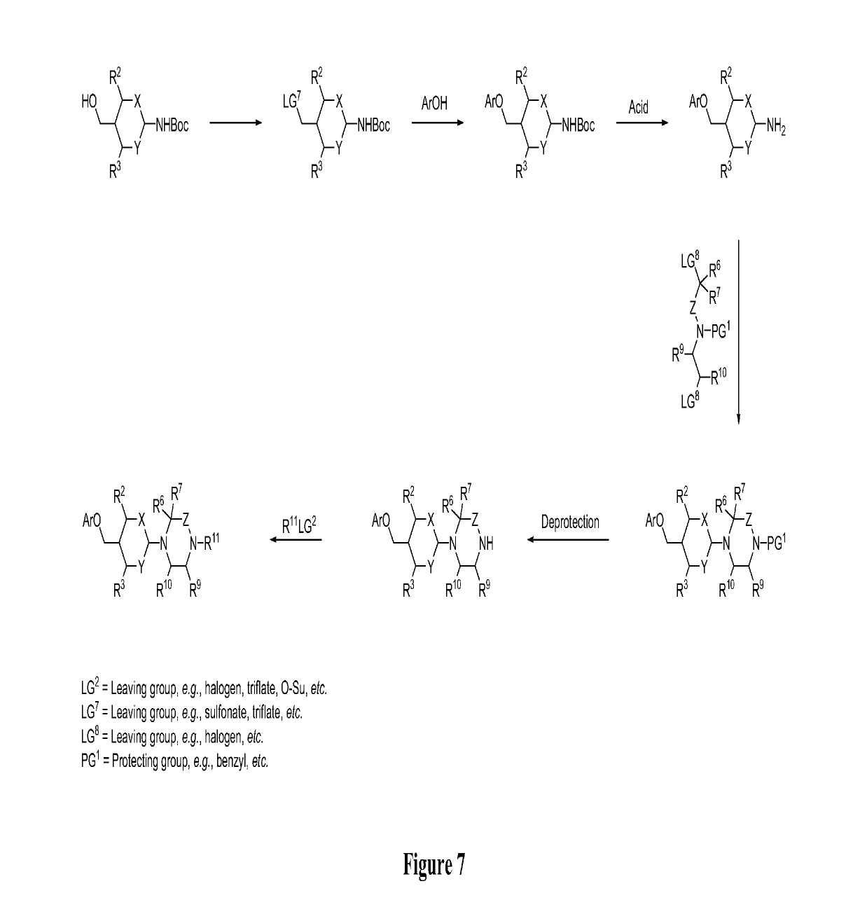

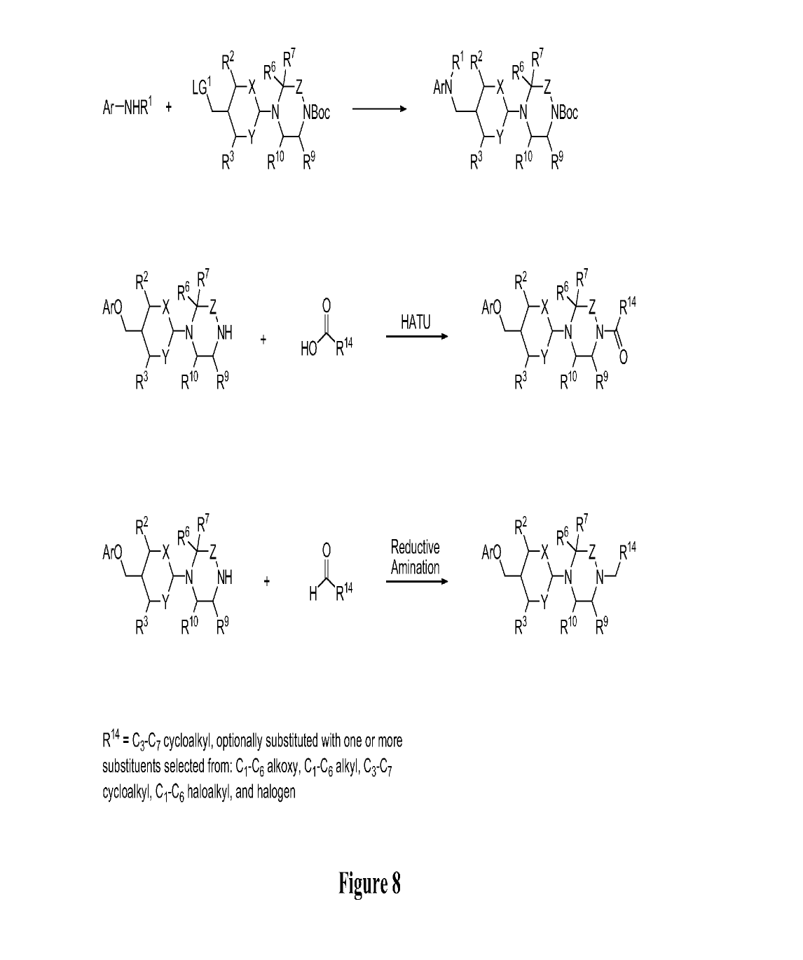

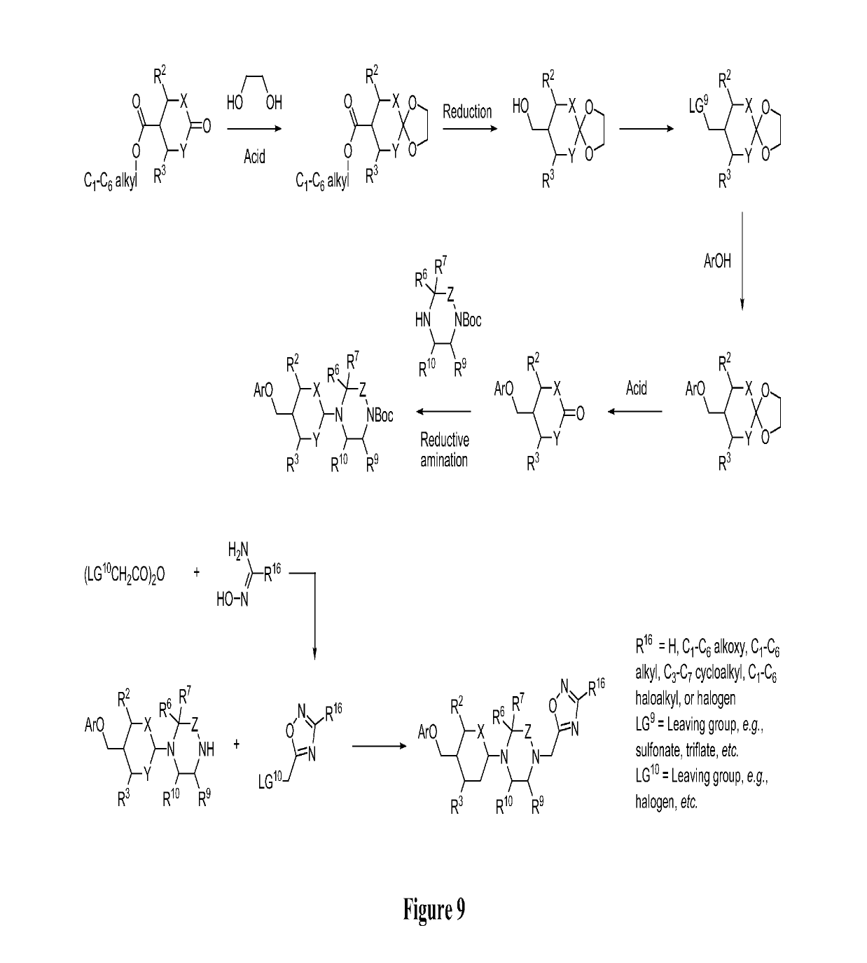

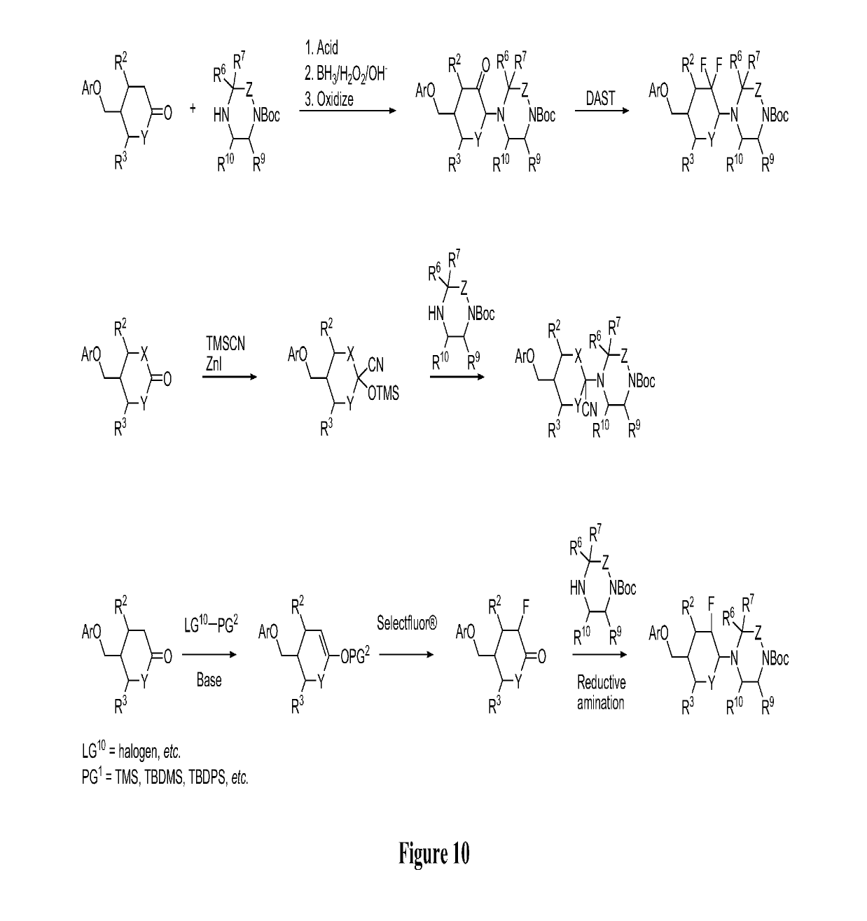

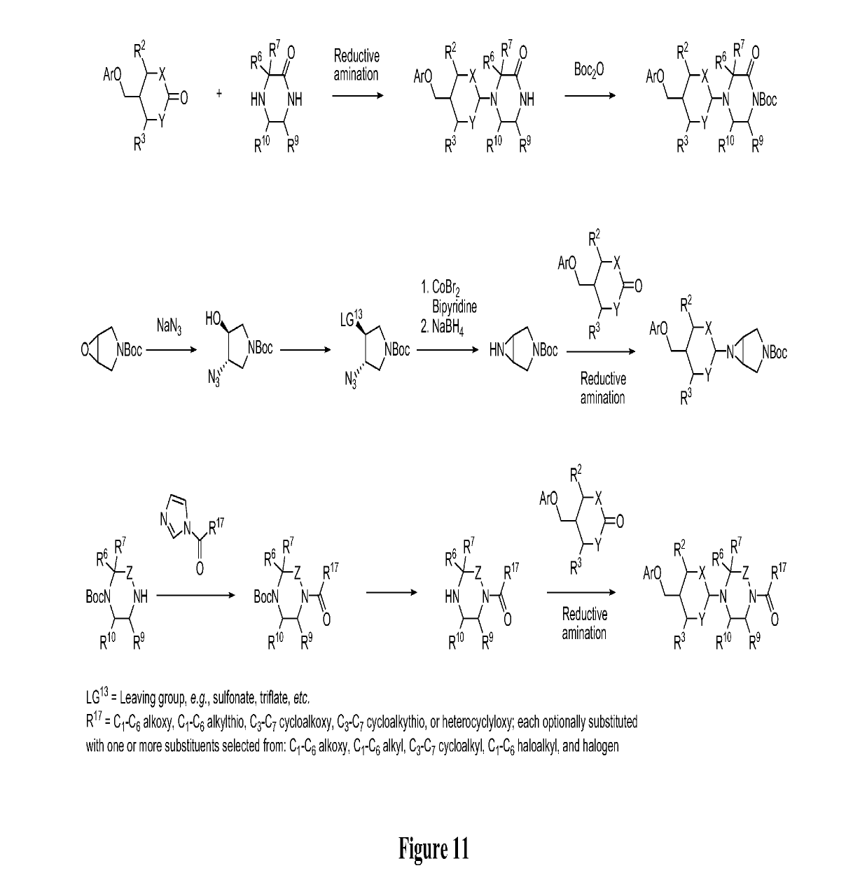

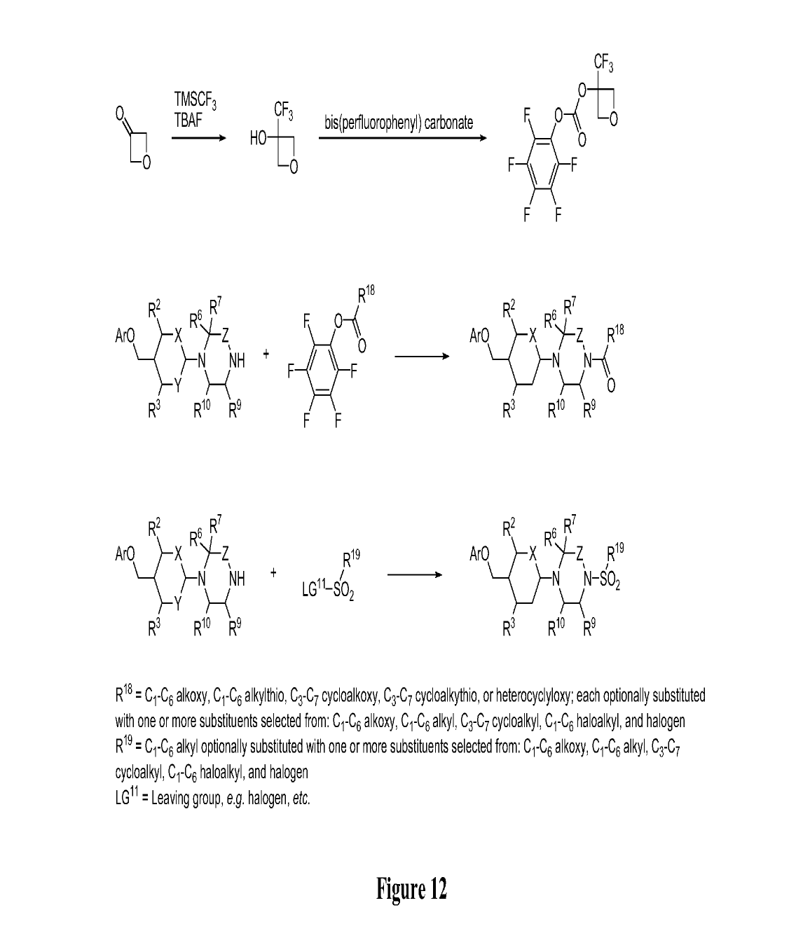

Illustrated syntheses for compounds of the present invention are shown in FIGS. 4-12 wherein the variables Ar, W, X, Y, Z, and R¹⁻¹⁹ have the same definitions as used throughout this disclosure.

The compounds of the invention and their syntheses are further illustrated by the following examples. The following examples are provided to further define the invention without, however, limiting the invention to the particulars of these examples. The compounds described herein, supra and infra, are named according to AutoNom version 2.2, AutoNom 2000, CS ChemDraw Ultra Version 7.0.1, or CS ChemDraw Ultra Version 9.0.7. In certain instances common names are used and it is understood that these common names would be recognized by those skilled in the art.

Proton nuclear magnetic resonance (¹H NMR) spectra were recorded on a Bruker Avance-400 equipped with a QNP (Quad Nucleus Probe) or a BBI (Broad Band Inverse) and z-gradient. Chemical shifts are given in parts per million (ppm) with the residual solvent signal used as reference. NMR abbreviations are used as follows: s=singlet, d=doublet, dd=doublet of doublets, ddd=doublet of doublet of doublets, dt=doublet of triplets, t=triplet, td=triplet of doublets, tt=triplet of triplets, q=quartet, m=multiplet, bs=broad singlet, bt=broad triplet. Microwave irradiations were carried out using a Smith Synthesizer™ or an Emrys Optimizer™ (Biotage). Thin-layer chromatography (TLC) was performed on silica gel 60 F₂₅₄ (Merck), preparatory thin-layer chromatography (prep TLC) was preformed on PK6F silica gel 60 A 1 mm plates (Whatman) and column chromatography was carried out on a silica gel column using Kieselgel 60, 0.063-0.200 mm (Merck). Evaporation was done under reduced pressure on a Büchi rotary evaporator.

LCMS spec: HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: API 150EX with Turbo Ion Spray source, AB/MDS Sciex; Software: Analyst 1.2.

### Example 1.1

### Preparation of tert-Butyl 4-((1r,4r)-4-((2-Fluoro-4-(methylsulfonyl)phenoxy)methyl)cyclohexyl)piperazine-1-carboxylate (Compound 1)

### Step A: Preparation of tert-Butyl 4-(4-(Ethoxycarbonyl)cyclohexyl)piperazine-1-carboxylate

To a mixture of ethyl 4-oxocyclohexanecarboxylate (10 g, 58.8 mmol) and tert-butyl piperazine-1-carboxylate (13.13 g, 70.5 mmol) in dichloroethane (100 mL) was added acetic acid (6.72 mL, 118 mmol). The reaction mixture was degassed and charged with nitrogen. Sodium triacetoxyborohydride (31.1 g, 147 mmol) was added. The reaction mixture was stirred overnight at room temperature, then diluted with water, adjusted pH to 8 with 1 N NaOH aqueous solution and extracted with DCM. The organic phases were combined, dried over anhydrous Na₂SO₄, filtered, and then concentrated. The residue was purified by column chromatography, eluting with 100% ethyl acetate to give the title compound as a mixture of cis/trans isomers (18 g, 90%). Exact mass calculated for C₁₈H₃₂N₂O₄: 340.2. found: LCMS m/z=341.2 [M+H]⁺; ¹H NMR (400 MHz, CDCl₃) δ ppm 1.22-1.28 (m, 3H), 1.45 (s, 9H), 1.48-1.72 (m, 5H), 1.94-2.35 (m, 5H), 2.48-2.58 (m, 4H), 3.35-3.50 (m, 4H), 4.06-4.18 (m, 2H).

### Step B: Preparation of Cis and Trans Isomers of tert-Butyl 4-(4-(Hydroxymethyl)cyclohexyl)piperazine-1-carboxylate

To a stirred solution of tert-butyl 4-(4-(ethoxycarbonyl)cyclohexyl)piperazine-1-carboxylate (5 g, 14.69 mmol) in THF (30 mL) was added a solution of 2 M lithium borohydride in THF (9.55 mL, 19.09 mmol) under nitrogen. The reaction was stirred at reflux overnight. The mixture was acidified to pH 3-4 with 3 M HCl aqueous solution, then basified to pH 8 with aqueous 1 N NaOH solution and extracted with IPA/DCM (1:4). The organic phases were combined, dried over anhydrous Na₂SO₄, filtered then concentrated. The residue was purified by column chromatography (100% ethyl acetate, then 5% MeOH/ethyl acetate) to give first the cis isomer of the title compound (870 mg, 19.8%) and then the trans isomer (400 mg, 9.1%). Cis: exact mass calculated for C₁₆H₃₀N₂O₃: 298.2. found: LCMS m/z=299.4 [M+H]⁺; ¹H NMR (400 MHz, CDCl₃) δ ppm 1.45 (s, 9H), 1.45-1.75 (m, 10H), 2.24-2.32 (m, 1H), 2.44-2.54 (m, 4H), 3.41-3.48 (m, 4H), 3.57 (dd, J=6.8 and 5.6 Hz, 2H). Trans: exact mass calculated for C₁₆H₃₀N₂O₃: 298.2. found: LCMS m/z=299.4 [M+H]⁺; ¹H NMR (400 MHz, CDCl₃) δ ppm 0.94-1.05 (m, 2H), 1.24-1.40 (m, 4H), 1.45 (s, 9H), 1.85-2.00 (m, 4H), 2.28-2.36 (m, 1H), 2.52-2.64 (m, 4H), 3.43-3.50 (m, 6H).

### Step C: Preparation of tert-Butyl 4-((1r,4r)-4-((Methylsulfonyloxy)methyl)cyclohexyl)piperazine-1-carboxylate

To a stirred solution of tert-butyl 4-((1r,4r)-4-(hydroxymethyl)cyclohexyl)piperazine-1-carboxylate (100 mg, 0.335 mmol), prepared in Step B above, and triethylamine (93 μL, 0.670 mmol) in DCM (2 mL) was added methanesulfonyl chloride (46.1 mg, 0.402 mmol) dropwise, with cooling in an ice-water bath. The reaction mixture was slowly warmed to room temperature and stirred for 2 h. The mixture was diluted with DCM and washed with water. The organic layer was dried over anhydrous Na₂SO₄ and concentrated to give the title compound (125 mg, 99%). Exact mass calculated for C₁₇H₃₂N₂O₅S: 376.2. found: LCMS m/z=377.3 [M+H]⁺.

### Step D: Preparation of tert-Butyl 4-((1r,4r)-4-((2-Fluoro-4-(methylsulfonyl)phenoxy)methyl)cyclohexyl)piperazine-1-carboxylate (Compound 1)

tert-Butyl 4-((1r,4r)-4-((methylsulfonyloxy)methyl)cyclohexyl)piperazine-1-carboxylate (125 mg, 0.332 mmol), prepared above in Step C, was dissolved in DMF (2 mL). 2-Fluoro-4-(methylsulfonyl)phenol (76 mg, 0.398 mmol) and cesium carbonate (216 mg, 0.664 mmol) were added. The reaction was stirred at room temperature overnight and heated at 100° C. for 2 h under microwave irradiation. The solid was filtered off, washed with ethyl acetate. The filtrate was concentrated, the residue was purified by column chromatography (99:1 EtOAc:Et₃N) to give the title compound as a white solid (115 mg, 73.6%). Exact mass calculated for C₂₃H₃₅FN₂O₅S: 470.2. found: LCMS m/z=471.2 [M+H]⁺; ¹H NMR (400 MHz, CDCl₃) δ ppm 1.10-1.20 (m, 2H), 1.25-1.45 (m, 2H), 1.46 (s, 9H), 1.78-1.90 (m, 1H), 1.96-2.08 (m, 4H), 2.30-2.60 (m, 5H), 3.03 (s, 3H), 3.38-3.60 (m, 4H), 3.90 (d, J=6.3 Hz, 2H), 7.05 (t, J=8.0 Hz, 1H), 7.62-7.69 (m, 2H).

### Example 1.2

### Preparation of 5-Ethyl-2-(4-((1r,4r)-4-((2-Fluoro-4-(methylsulfonyl)phenoxy)methyl)cyclohexyl)piperazin-1-yl)pyrimidine (Compound 2)

### Step A: Preparation of 1-((1r,4r)-4-((2-Fluoro-4-(methylsulfonyl)phenoxy)methyl)cyclohexyl)piperazine Dihydrochloride

To a solution of tert-butyl 4-((1r,4r)-4-((2-fluoro-4-(methylsulfonyl)phenoxy)methyl)cyclohexyl)piperazine-1-carboxylate (prepared in Example 1.1) (110 mg, 0.234 mmol) in dioxane (1.5 mL) was added a solution of 4 M HCl in dioxane (1.75 mL, 7.01 mmol). The reaction was stirred at room temperature overnight. The solid was filtered, and washed with dioxane and hexanes to give the title compound (104 mg, 0.214 mmol, 92%). Exact mass calculated for C₁₈H₂₇FN₂O₃S: 370.2. found: LCMS m/z=371.0 [M+H]⁺; ¹H NMR (400 MHz, DMSO-d₆) δ ppm 1.14-1.24 (m, 2H), 1.48-1.60 (m, 2H), 1.75-1.85 (m, 1H), 1.95-2.02 (m, 2H), 2.10-2.20 (m, 2H), 3.20 (s, 3H), 3.20-3.65 (m, 9H), 4.03 (d, J=6.3 Hz, 2H), 7.42 (t, J=8.4 Hz, 1H), 7.69-7.73 (m, 1H), 7.78 (dd, J=10.6 and 2.2 Hz, 1H), 9.40 (bs, 2H), 11.6 (bs, 1H).

### Step B: Preparation of 5-Ethyl-2-(4-((1r,4r)-4-((2-fluoro-4-(methylsulfonyl)phenoxy)methyl)cyclohexyl)piperazin-1-yl)pyrimidine (Compound 2)

A mixture of 1-((1r,4r)-4-((2-fluoro-4-(methylsulfonyl)phenoxy)methyl)cyclohexyl)piperazine dihydrochloride (prepared in Step A above) (65 mg, 0.147 mmol), 2-chloro-5-ethylpyrimidine (27 mg, 0.191 mmol) and triethylamine (78 μL, 0.586 mmol) in IPA (1.5 mL) was heated at 130° C. for 80 min under microwave irradiation. The mixture was cooled down. The solid precipitate was collected, washed with IPA and dried to give the title compound (40 mg, 57.1%). Exact mass calculated for C₂₄H₃₃FN₄O₃S: 476.2. found: LCMS m/z=477.2 [M+H]⁺; ¹H NMR (400 MHz, CDCl₃) δ ppm 1.19 (t, J=7.6 Hz, 3H), 1.10-1.20 (m, 2H), 1.35-1.45 (m, 2H), 1.78-1.90 (m, 1H), 1.98-2.10 (m, 4H), 2.45 (q, J=7.6 Hz, 2H), 2.40-2.50 (m, 1H), 2.65-2.75 (m, 4H), 3.03 (s, 3H), 3.80-3.90 (m, 4H), 3.90 (d, J=6.3 Hz, 2H), 7.05 (t, J=8.0 Hz, 1H), 7.62-7.69 (m, 2H), 8.18 (s, 2H).

### Example 1.3

### Preparation of 2-(4-((1r,4r)-4-((2-Fluoro-4-(methylsulfonyl)phenoxy)methyl)cyclohexyl)piperazin-1-yl)-5-(trifluoromethyl)pyrimidine (Compound 3)

A mixture of 1-((1r,4r)-4-((2-fluoro-4-(methylsulfonyl)phenoxy)methyl)cyclohexyl)piperazine dihydrochloride (29 mg, 0.065 mmol), 2-chloro-5-(trifluoromethyl)pyrimidine (16 mg, 0.085 mmol) and triethylamine (34 μL, 0.262 mmol) in IPA (1.5 mL) was heated at 130° C. for 40 min under microwave irradiation. The mixture was cooled down. The solid precipitate was collected, washed with IPA and dried to give the title compound (30 mg, 89.2%). Exact mass calculated for C₂₃H₂₈F₄N₄O₃S: 516.2. found: LCMS m/z=517.2 [M+H]⁺; ¹H NMR (400 MHz, CDCl₃) δ ppm 1.10-1.20 (m, 2H), 1.35-1.45 (m, 2H), 1.78-1.90 (m, 1H), 1.98-2.10 (m, 4H), 2.40-2.50 (m, 1H), 2.65-2.75 (m, 4H), 3.03 (s, 3H), 3.85-4.05 (m, 4H), 3.90 (d, J=6.2 Hz, 2H), 7.05 (t, J=8.0 Hz, 1H), 7.62-7.69 (m, 2H), 8.49 (s, 2H).

### Example 1.4

### Preparation of Isopropyl 4-((1r,4r)-4-((4-(Methylsulfonyl)phenoxy)methyl)cyclohexyl)piperazine-1-carboxylate (Compound 4)

### Step A: Preparation of 1-(4-(((1r,4r)-4-(Methylsulfonyl)phenoxy)methyl)cyclohexyl)piperazine Dihydrochloride

To a solution of tert-butyl 4-((1r,4r)-4-(hydroxymethyl)cyclohexyl)piperazine-1-carboxylate (1.2 g, 4.02 mmol) in THF (12 mL) was added 4-(methylsulfonyl)phenol (0.762 g, 4.42 mmol), triphenylphosphine (1.16 g, 4.42 mmol), followed by diisopropyl diazene-1,2-dicarboxylate (0.86 mL, 4.42 mmol) under argon at room temperature. The reaction was stirred at room temperature for 5 h. The resulting solution was evaporated and purified by column chromatography to give tert-butyl 4-((1r,4r)-4-((4-(methylsulfonyl)phenoxy)methyl)cyclohexyl)piperazine-1-carboxylate, which was then dissolved in dioxane (10 mL), added a solution of 4 N HCl in dioxane (10 mL), and stirred at room temperature for 5 h. The precipitate was collected, washed with dioxane, and dried to give the title compound (0.6 g, 35.1%). Exact mass calculated for C₁₈H₂₈N₂O₃S: 352.2. found: LCMS m/z=353.4 [M+H]⁺; ¹H NMR (400 MHz, DMSO-d₆) δ ppm 1.10-1.24 (m, 2H), 1.48-1.60 (m, 2H), 1.70-1.82 (m, 1H), 1.95-2.02 (m, 2H), 2.10-2.20 (m, 2H), 3.15 (s, 3H), 3.20-3.65 (m, 9H), 3.92 (d, J=6.3 Hz, 2H), 7.13-7.17 (m, 2H), 7.81-7.85 (m, 2H), 9.50 (bs, 2H), 11.7 (bs, 1H).

### Step B: Preparation of Isopropyl 4-(1r,4r)-4-((4-(Methylsulfonyl)phenoxy)methyl)cyclohexyl)piperazine-1-carboxylate Hydrochloride (Compound 4)

To a solution of 1-((1r,4r)-4-((4-(methylsulfonyl)phenoxy)methyl)cyclohexyl)piperazine dihydrochloride (140 mg, 0.329 mmol) in DCM (6 mL) was added a solution of 1 M isopropyl chloroformate in toluene (0.49 mL, 0.494 mmol) and triethylamine (0.18 mL, 1.316 mmol) under N₂ at room temperature. The reaction was stirred at room temperature for 2 h and then quenched with water. The organic layer was washed with saturated NaHCO₃ solution and saline, dried, and concentrated to give isopropyl 4-((1r,4r)-4-((4-(methylsulfonyl)phenoxy)methyl)cyclohexyl)piperazine-1-carboxylate. The material obtained above was dissolved in dioxane (3 mL), and 4 N HCl in dioxane (0.33 mL, 1.316 mmol) was added. After stirring for 30 min, the solvent was removed by evaporation. The solid residue was washed with ether and dried to give the title compound (70 mg, 44.8%). Exact mass calculated for C₂₂H₃₄N₂O₅S: 438.2. found: LCMS m/z=439.4 [M+H]⁺; ¹H NMR (400 MHz, DMSO-d₆) δ ppm 1.21 (d, J=6.2 Hz, 6H), 1.16-1.22 (m, 2H), 1.45-1.56 (m, 2H), 1.70-1.82 (m, 1H), 1.94-2.01 (m, 2H), 2.14-2.20 (m, 2H), 2.95-3.10 (m, 3H), 3.14 (s, 3H), 3.15-3.50 (m, 4H), 3.92 (d, J=6.3 Hz, 2H), 4.01-4.08 (m, 2H), 4.80 (q, J=6.2 Hz, 1H), 7.13-7.17 (m, 2H), 7.81-7.85 (m, 2H), 10.69 (bs, 1H).

### Example 1.5

### Preparation of 5-Ethyl-2-(4-((1r,4r)-4-((4-(methylsulfonyl)phenoxy)methyl)cyclohexyl)piperazin-1-yl)pyrimidine (Compound 5)

A solution of 1-((1r,4r)-4-((4-(methylsulfonyl)phenoxy)methyl)cyclohexyl)piperazine dihydrochloride (130 mg, 0.306 mmol), 2-chloro-5-ethylpyrimidine (65 mg, 0.458 mmol) and triethylamine (0.163 mL, 1.222 mmol) in IPA (2 mL) was heated at 150° C. for 1 h under microwave irradiation. The mixture was cooled down. The solid was filtered, washed with IPA, and dried to give the title compound (70 mg, 49.9%). Exact mass calculated for C₂₄H₃₄N₄O₃S: 458.2. found: LCMS m/z=459.3 [M+H]⁺; ¹H NMR (400 MHz, CDCl₃) δ ppm 1.10-1.20 (m, 2H), 1.20 (t, J=7.6 Hz, 3H), 1.30-1.42 (m, 2H), 1.74-1.84 (m, 1H), 1.96-2.07 (m, 4H), 2.30-2.40 (m, 1H), 2.45 (q, J=7.6 Hz, 2H), 2.60-2.70 (m, 4H), 3.02 (s, 3H), 3.77-3.84 (m, 4H), 3.84 (d, J=6.3 Hz, 2H), 6.98-7.02 (m, 2H), 7.83-7.87 (m, 2H), 8.16 (s, 2H).

### Example 1.6

### Preparation of 3-Isopropyl-5-(4-((1r,4r)-4-((4-(methylsulfonyl)phenoxy)methyl)cyclohexyl)piperazin-1-yl)-1,2,4-oxadiazole (Compound 6)

To a solution of 1-((1r,4r)-4-((4-(methylsulfonyl)phenoxy)methyl)cyclohexyl)piperazine dihydrochloride (100 mg, 0.235 mmol) in DCM (1 mL) was added DIEA (82 μL, 0.47 mmol), followed by CNBr (26 mg, 0.247 mmol) in DCM (1 mL) at room temperature. The reaction mixture was stirred for 1 h at the same temperature, then washed with water twice, dried, and concentrated. The residue was triturated with IPA to give 4-((1r,4r)-4-((4-(methylsulfonyl)phenoxy)methyl)cyclohexyl)piperazine-1-carbonitrile (0.09 g, 0.238 mmol). The material was dissolved in THF (1 mL), and N-hydroxyisobutyrimidamide (41 mg, 0.4 mmol), was added followed by a solution of 0.5 M ZnCl₂ in THF (0.8 mL, 0.4 mmol). After stirring for 3 h at room temperature, a solution of 4 M HCl in dioxane (0.59 mL, 2.35 mmol) was added. The reaction was stirred overnight at 65° C., diluted with ethyl acetate, washed with water and 1 N HCl aqueous solution, dried with MgSO₄, and concentrated. The residue was purified by preparative HPLC to give the title compound (45 mg, 41.4%). Exact mass calculated for C₂₃H₃₄N₄O₄S: 462.2. found: LCMS m/z=463.4 [M+H]⁺; ¹H NMR (400 MHz, CDCl₃) δ ppm 1.10-1.35 (m, 4H), 1.20 (d, J=6.9 Hz, 6H), 1.70-1.80 (m, 1H), 1.86-2.05 (m, 4H), 2.25-2.65 (m, 4H), 2.82-2.95 (m, 2H), 2.95 (s, 3H), 3.50-3.60 (m, 3H), 3.78 (d, J=6.0 Hz, 2H), 4.10-4.20 (m, 1H), 6.90-6.95 (m, 2H), 7.77-7.81 (m, 2H).

### Example 1.7

### Preparation of 5-Ethyl-2-(4-((1r,4r)-4-((5-(methylsulfonyl)pyrazin-2-yloxy)methyl)cyclohexyl)piperazin-1-yl)pyrimidine (Compound 7)

### Step A: Preparation of tert-Butyl 4-((1r,4r)-4-((5-(Methylsulfonyl)pyrazin-2-yloxy)methyl)cyclohexyl)piperazine-1-carboxylate

To a solution of tert-butyl 4-((1r,4r)-4-(hydroxymethyl)cyclohexyl)piperazine-1-carboxylate (400 mg, 1.34 mmol) in THF (8 mL) was added 5-(methylsulfonyl)pyrazin-2-ol (0.257 g, 1.474 mmol), triphenylphosphine (0.387 g, 1.474 mmol), and followed by diisopropyl diazene-1,2-dicarboxylate (0.298 g, 1.474 mmol) under argon at room temperature. The reaction was stirred for 5 h at the same temperature. The mixture was concentrated and the residue was purified by silica gel column chromatography to give the title compound (110 mg, 36.7%). Exact mass calculated for C₂₁H₃₄N₄O₅S: 454.2. found: LCMS m/z=455.4 [M+H]⁺; ¹H NMR (400 MHz, CDCl₃) δ ppm 1.05-1.15 (m, 2H), 1.25-1.40 (m, 2H), 1.39 (s, 9H), 1.70-1.80 (m, 1H), 1.85-2.10 (m, 4H), 2.40-2.70 (m, 5H), 3.12 (s, 3H), 3.35-3.65 (m, 4H), 4.19 (d, J=6.4 Hz, 2H), 8.17 (d, J=1.2 Hz, 1H), 8.72 (d, J=1.2 Hz, 1H).

### Step B: Preparation of 5-Ethyl-2-(4-((1r,4r)-4-((5-(methylsulfonyl)pyrazin-2-yloxy)methyl)cyclohexyl)piperazin-1-yl)pyrimidine (Compound 7)

To a solution of tert-butyl 4-((1r,4r)-4-((5-(methylsulfonyl)pyrazin-2-yloxy)methyl)cyclohexyl)piperazine-1-carboxylate (60 mg, 0.132 mmol) in DCM (1 mL) was added TFA (0.1 mL, 1.32 mmol). The reaction was stirred at room temperature for 1 h. The mixture was concentrated to give 2-(methylsulfonyl)-5-(((1r,4r)-4-(piperazin-1-yl)cyclohexyl)methoxy)pyrazine. The above material was dissolved in IPA (1 mL), and 2-chloro-5-ethylpyrimidine (28 mg, 0.198 mmol), and triethylamine (55 μL, 0.396 mmol) were added. The reaction was heated at 130° C. under microwave irradiation. After removal of the solvent, the residue was purified by preparative HPLC to give the title compound (20 mg, 32.9%). Exact mass calculated for C₂₂H₃₂N₆O₃S: 460.2. found: LCMS m/z=461.4 [M+H]⁺; ¹H NMR (400 MHz, CDCl₃) δ ppm 1.12 (t, J=7.6 Hz, 3H), 1.05-1.15 (m, 2H), 1.25-1.40 (m, 2H), 1.70-1.80 (m, 1H), 1.85-2.10 (m, 4H), 2.41 (q, J=7.6 Hz, 2H), 2.30-2.45 (m, 1H), 2.60-2.80 (m, 4H), 3.12 (s, 3H), 3.70-3.90 (m, 4H), 4.19 (d, J=6.4 Hz, 2H), 8.11 (s, 2H), 8.17 (d, J=1.2 Hz, 1H), 8.72 (d, J=1.2 Hz, 1H).

### Example 1.8

### Preparation of tert-Butyl 4-((1s,4s)-4-((2-Methyl-6-(methylsulfonyl)pyridin-3-yloxy)methyl)cyclohexyl)piperazine-1-carboxylate (Compound 8)

To a solution of tert-butyl 4-((1s,4s)-4-(hydroxymethyl)cyclohexyl)piperazine-1-carboxylate (140 mg, 0.469 mmol) in THF (3 mL) was added a 1 M solution of potassium tert-butoxide in THF (0.61 mL, 0.61 mmol). After stirring for 30 min at room temperature, 6-bromo-3-fluoro-2-methylpyridine (98 mg, 0.516 mmol) in THF (0.5 mL) was added. The reaction was stirred for 4 h at room temperature, quenched with water, and extracted with DCM. The organic extracts were dried with MgSO₄, concentrated, and purified by column chromatography to give tert-butyl 4-((1s,4s)-4-((6-bromo-2-methylpyridin-3-yloxy)methyl)cyclohexyl)piperazine-1-carboxylate (40 mg, 18.1%). A solution of the above material, NaSO₂Me (20 mg, 0.19 mmol), copper(I) trifluoromethanesulfonate benzene complex (5 mg, 0.01 mmol), N¹,N²-dimethylethane-1,2-diamine (2 mg, 0.02 mmol) in DMSO (0.7 mL) was heated at 130° C. for 1 h under microwave irradiation. The mixture was purified by preparative HPLC to give the title compound (4 mg, 10%). Exact mass calculated for C₂₃H₃₇N₃O₅S: 467.2. found: LCMS m/z=468.4 [M+H]⁺; ¹H NMR (400 MHz, CDCl₃) δ ppm 1.39 (s, 9H), 1.40-1.70 (m, 4H), 1.70-2.00 (m, 5H), 2.10-2.20 (m, 1H), 2.44 (s, 3H), 2.35-2.65 (m, 4H), 3.10 (s, 3H), 3.30-3.70 (m, 4H), 3.94-4.02 (m, 2H), 7.14-7.20 (m, 1H), 7.84 (d, J=8.4 Hz, 1H).

### Example 1.9

### Preparation of tert-Butyl 4-(1r,4r)-4-((2-Methyl-6-(methylsulfonyl)pyridin-3-yloxy)methyl)cyclohexyl)piperazine-1-carboxylate (Compound 9)

The title compound was prepared in a manner similar to that described in Example 1.8 using tert-butyl 4-((1r,4r)-4-(hydroxymethyl)cyclohexyl)piperazine-1-carboxylate. Exact mass calculated for C₂₃H₃₇N₃O₅S: 467.2. found: LCMS m/z=468.4 [M+H]⁺; ¹H NMR (400 MHz, CDCl₃) δ ppm 1.10-1.20 (m, 2H), 1.30-1.45 (m, 2H), 1.39 (s, 9H), 1.70-1.85 (m, 1H), 1.90-2.08 (m, 4H), 2.45 (s, 3H), 2.40-2.70 (m, 5H), 3.10 (s, 3H), 3.35-3.60 (m, 4H), 3.78 (d, J=6.3 Hz, 2H), 7.07 (d, J=8.5 Hz, 1H), 7.83 (d, J=8.5 Hz, 1H).

### Example 1.10

### Preparation of 5-Ethyl-2-(4-((1r,4r)-4-((2-methyl-6-(methylsulfonyl)pyridin-3-yloxy)methyl)cyclohexyl)piperazin-1-yl)pyrimidine (Compound 10)

### Step A: Preparation of tert-Butyl 4-((1r,4r)-4-((2-Methyl-6-(methylsulfonyl)pyridin-3-yloxy)methyl)cyclohexyl)piperazine-1-carboxylate

To a solution of tert-butyl 4-((1r,4r)-4-(hydroxymethyl)cyclohexyl)piperazine-1-carboxylate (50 mg, 0.168 mmol) in THF (2 mL) was added 2-methyl-6-(methylsulfonyl)pyridin-3-ol (35 mg, 0.184 mmol), triphenylphosphine (48 mg, 0.184 mmol), and diisopropyl diazene-1,2-dicarboxylate (37 mg, 0.184 mmol) under argon at room temperature. The reaction mixture was stirred overnight at room temperature. The solvent was removed by evaporation, and the residue was purified by column chromatography on silica gel to give the title compound (45 mg, 57.4%). Exact mass calculated for C₂₃H₃₇N₃O₅S: 467.2. found: LCMS m/z=468.4 [M+H]⁺.

### Step B: Preparation of 1-(1r,4r)-4-((2-Methyl-6-(methylsulfonyl)pyridin-3-yloxy)methyl)cyclohexyl)piperazine

To a solution of tert-butyl 4-((1r,4r)-4-((2-methyl-6-(methylsulfonyl)pyridin-3-yloxy)methyl)cyclohexyl)piperazine-1-carboxylate (40 mg, 0.086 mmol) in DCM (1.5 mL) was added TFA (0.3 mL, 0.855 mmol). The reaction was stirred for 2 h at room temperature. The mixture was concentrated to give 1-((1r,4r)-4-((2-methyl-6-(methylsulfonyl)pyridin-3-yloxy)methyl)cyclohexyl)piperazine, which was used in Step C without further purification. Exact mass calculated for C₁₈H₂₉N₃O₃S: 367.2. found: LCMS m/z=368.2 [M+H]⁺.

### Step C: Preparation of 5-Ethyl-2-(4-((1r,4r)-4-((2-methyl-6-(methylsulfonyl)pyridin-3-yloxy)methyl)cyclohexyl)piperazin-1-yl)pyrimidine (Compound 10)

A mixture of 1-((1r,4r)-4-((2-methyl-6-(methylsulfonyl)pyridin-3-yloxy)methyl)cyclohexyl)piperazine (prepared in Step B above), 2-chloro-5-ethylpyrimidine (15 mg, 0.103 mmol), triethylamine (68 μL, 0.513 mmol) in IPA (1 mL) was heated at 140° C. for 1 h under microwave irradiation. The mixture was concentrated and the residue was purified by preparative TLC to give the title compound (6 mg, 14.8%). Exact mass calculated for C₂₄H₃₅N₅O₃S: 473.2. found: LCMS m/z=474.2 [M+H]⁺; ¹H NMR (400 MHz, CDCl₃) δ ppm 1.10-1.20 (m, 2H), 1.12 (t, J=7.6 Hz, 3H), 1.30-1.45 (m, 2H), 1.75-1.85 (m, 1H), 1.90-2.08 (m, 4H), 2.40 (q, J=7.6 Hz, 2H), 2.45 (s, 3H), 2.40-2.70 (m, 5H), 3.10 (s, 3H), 3.70-3.90 (m, 4H), 3.78 (d, J=6.3 Hz, 2H), 7.08 (d, J=8.6 Hz, 1H), 7.83 (d, J=8.6 Hz, 1H), 8.10 (s, 2H).

### Example 1.11

### Preparation of 3-(2-Fluoropropan-2-yl)-5-(4-((1r,4r)-4-((4-(methylsulfonyl)phenoxy)methyl)cyclohexyl)piperazin-1-yl)-1,2,4-oxadiazole (Compound 11)

To a solution of 4-((1r,4r)-4-((4-(methylsulfonyl)phenoxy)methyl)cyclohexyl)piperazine-1-carbonitrile (150 mg, 0.397 mmol), as prepared in Example 1.6, and 2-fluoro-N′-hydroxy-2-methylpropanimidamide (81 mg, 0.675 mmol) in THF (4 mL) was added a solution of 0.5 M ZnCl₂ in THF (1.35 mL, 0.675 mmol). The reaction mixture was stirred for 2 h at room temperature. Then, 4 M HCl in dioxane (0.695 mL, 2.78 mmol) was added. The mixture was stirred overnight at 65° C. The solid intermediate was filtered, washed with dioxane, and dried. A solution of the intermediate (˜190 mg) in DMF (2 mL) was heated at 160° C. under microwave irradiation for 1 h. After removal of the solvent, the residue was purified by column chromatography to give the title compound (95 mg, 49.7%). Exact mass calculated for C₂₃H₃₃FN₄O₄S: 480.2. found: LCMS m/z=481.0 [M+H]⁺; ¹H NMR (400 MHz, CDCl₃) δ ppm 1.10-1.35 (m, 4H), 1.78 (d, J=22.0 Hz, 6H), 1.70-1.80 (m, 1H), 1.95-2.10 (m, 4H), 2.35-2.80 (m, 5H), 3.07 (s, 3H), 3.65-3.75 (m, 3H), 3.87 (d, J=6.1 Hz, 2H), 4.20-4.30 (m, 1H), 7.02-7.06 (m, 2H), 7.88-7.92 (m, 2H).

### Example 1.12

### Preparation of 2-(4-((1r,4r)-4-((2-Methyl-6-(methylsulfonyl)pyridin-3-yloxy)methyl)cyclohexyl)piperazin-1-yl)-5-(trifluoromethyl)pyrimidine (Compound 12)

### Step A: Preparation of ((1r,4r)-4-(tert-Butoxycarbonylamino)cyclohexyl)methyl methanesulfonate

To a solution of tert-butyl (1r,4r)-4-(hydroxymethyl)cyclohexylcarbamate (1.4 g, 6.11 mmol) and triethylamine (1.63, 12.3 mmol) in DCM (10 mL) in an ice bath was added dropwise methanesulfonyl chloride (0.84 g, 7.33 mmol). The reaction was slowly warmed to room temperature over 3 h with stirring. The mixture was diluted with DCM, washed with H₂O and saturated NaHCO₃ aqueous solution, dried, and concentrated to give the title compound (1.6 g, 85%) as a white powder. Exact mass calculated for C₁₃H₂₅NO₅S: 307.4. found LCMS m/z=308.2 [M+H]⁺.

### Step B: Preparation of tert-Butyl (1r,4r)-4-((2-Methyl-6-(methylsulfonyl)pyridin-3-yloxy)methyl)cyclohexylcarbamate

A mixture of ((1r,4r)-4-(tert-butoxycarbonylamino)cyclohexyl)methyl methanesulfonate (0.2 g, 0.65 mmol), 2-methyl-6-(methylsulfonyl)pyridin-3-ol (0.146 g, 0.781 mmol), and K₂CO₃ (0.181 g, 1.30 mmol) in DMF (8 mL) was heated at 130° C. under microwave irradiation for 1 h. The reaction was diluted with DCM, and washed with H₂O and saturated NaHCO₃ aqueous solution. The organic layer was dried and concentrated. The solid was triturated with IPA to give the title compound (0.19 g, 73%). Exact mass calculated for C₁₉H₃₀N₂O₅S: 398.2. found LCMS m/z=399.2 [M+H]⁺; ¹H NMR (400 MHz, CDCl₃) δ ppm 1.10-1.30 (m, 4H), 1.47 (s, 9H), 1.78-1.80 (m, 1H), 1.80-1.90 (m, 2H), 2.08-2.12 (m, 2H), 2.53 (s, 3H), 3.18 (s, 3H), 3.38-3.50 (m, 1H), 3.86 (d, J=6.2 Hz, 2H), 4.35-4.45 (m, 1H), 7.14 (d, J=8.5 Hz, 1H), 7.90 (d, J=8.5 Hz, 1H).

### Step C: Preparation of (1r,4r)-4-((2-Methyl-6-(methylsulfonyl)pyridin-3-yloxy)methyl)cyclohexanamine Hydrochloride

To a solution of tert-butyl (1r,4r)-4-((2-methyl-6-(methylsulfonyl)pyridin-3-yloxy)methyl)cyclohexylcarbamate (0.19 g, 0.48 mmol) in dioxane (2 mL) was added a solution of 4 M HCl in dioxane (1.2 mL, 4.8 mmol). The reaction was stirred overnight at room temperature. The solid was filtered, washed with dioxane and hexane, and dried to give the title compound (0.13 g, 0.43 mmol, 81%) as a white powder. Exact mass calculated for C₁₄H₂₂N₂O₃S: 298.2. found LCMS m/z=299.2 [M+H]⁺; ¹H NMR (400 MHz, DMSO-d₆) δ ppm 1.12-1.25 (m, 2H), 1.33-1.44 (m, 2H), 1.70-1.80 (m, 1H), 1.87-1.95 (m, 2H), 1.98-2.06 (m, 2H), 2.45 (s, 3H), 2.93-3.00 (m, 1H), 3.18 (s, 3H), 3.98 (d, J=6.2 Hz, 2H), 7.56 (d, J=8.5 Hz, 1H), 7.85 (d, J=8.5 Hz, 1H), 8.06 (bs, 3H).

### Step D: Preparation of 1-Benzyl-4-(1r,4r)-4-((2-methyl-6-(methylsulfonyl)pyridin-3-yloxy)methyl)cyclohexyl)piperazine

A mixture of N-benzyl-2-chloro-N-(2-chloroethyl)ethanamine hydrochloride (0.115 g, 0.427 mmol), potassium carbonate (0.135 g, 0.971 mmol), sodium iodide (0.151 g, 1.01 mmol), and (1r,4r)-4-((2-methyl-6-(methylsulfonyl)pyridin-3-yloxy)methyl)cyclohexanamine hydrochloride (0.13 g, 0.388 mmol) in DMF (3 mL) was stirred at 60° C. for 3 h. The reaction was diluted with DCM, washed with saturated NaHCO₃ aqueous solution, dried with anhydrous MgSO₄, and concentrated. The residue was purified by column chromatography to give the title compound (65 mg, 37%). Exact mass calculated for C₂₅H₃₅N₃O₃S: 457.2. found LCMS m/z=458.2 [M+H]⁺.

### Step E: Preparation of 1-(1r,4r)-4-((2-Methyl-6-(methylsulfonyl)pyridin-3-yloxy)methyl)cyclohexyl)piperazine

To a solution of 1-benzyl-4-((1r,4r)-4-((2-methyl-6-(methylsulfonyl)pyridin-3-yloxy)methyl)cyclohexyl)piperazine (60 mg, 0.131 mmol) and DIEA (51 mg, 0.393 mmol) in DCM (2.0 mL) was added 1-chloroethyl chloroformate (37 mg, 0.262 mmol) at room temperature. After stirring for 1 h, the reaction was diluted with DCM, washed with saturated NaHCO₃ aqueous solution, dried and concentrated to give 1-chloroethyl 4-((1r,4r)-4-((2-methyl-6-(methylsulfonyl)pyridin-3-yloxy)methyl)cyclohexyl)piperazine-1-carboxylate. The above material was dissolved in MeOH (2 mL), and then stirred for 2 h at 70° C. The mixture was concentrated to give the title compound (35 mg, 72%). Exact mass calculated for C₁₈H₂₉N₃O₃S: 367.2. found LCMS m/z=368.0 [M+H]⁺.

### Step F: Preparation of 2-(4-((1r,4r)-4-((2-Methyl-6-(methylsulfonyl)pyridin-3-yloxy)methyl)cyclohexyl)piperazin-1-yl)-5-(trifluoromethyl)pyrimidine (Compound 12)

A mixture of 1-((1r,4r)-4-((2-methyl-6-(methylsulfonyl)pyridin-3-yloxy)methyl)cyclohexyl)piperazine (35 mg, 0.095 mmol), 2-chloro-5-(trifluoromethyl)pyrimidine (21 mg, 0.114 mmol), and triethylamine (38 μL, 0.29 mmol) in IPA (1.5 mL) was heated at 130° C. under microwave irradiation for 50 min. The mixture was cooled down to room temperature. The solid was filtered and triturated with IPA to give the title compound (20 mg, 40.9%). Exact mass calculated for C₂₃H₃₀F₃N₅O₃S: 513.2. found LCMS m/z=514.2 [M+H]⁺; ¹H NMR (400 MHz, CDCl₃) δ ppm 1.10-1.20 (m, 2H), 1.30-1.45 (m, 2H), 1.75-2.08 (m, 5H), 2.46 (s, 3H), 2.20-2.40 (m, 1H), 2.55-2.80 (m, 4H), 3.11 (s, 3H), 3.40-3.50 (m, 1H), 3.80 (d, J=6.0 Hz, 2H), 3.80-4.00 (m, 3H), 7.08 (d, J=8.6 Hz, 1H), 7.83 (d, J=8.6 Hz, 1H), 8.43 (s, 2H).

### Example 1.13

### Preparation of 1-Methylcyclopropyl 4-((1r,4r)-4-((2-Fluoro-4-(methylsulfonyl)phenoxy)methyl)cyclohexyl)piperazine-1-carboxylate (Compound 13)

To a solution of 1-((1r,4r)-4-((2-fluoro-4-(methylsulfonyl)phenoxy)methyl)cyclohexyl)piperazine (25 mg, 0.067 mmol) (prepared in a similar manner to the one described in Example 1.12, Steps A to E) and triethylamine (20 mg, 0.202 mmol) in DCM (1.5 mL) was added 2,5-dioxopyrrolidin-1-yl 1-methylcyclopropyl carbonate (17 mg, 0.081 mmol) at room temperature. The reaction was stirred at 35° C. for 1 h and diluted with DCM (3 mL). The organic layer was washed with saturated NaHCO₃ aqueous solution, dried over anhydrous MgSO₄, and concentrated. The residue was purified by column chromatography to give the title compound (23 mg, 72.7%). Exact mass calculated for C₂₃H₃₃FN₂O₅S: 468.2. found LCMS m/z=469.2 [M+H]⁺; ¹H NMR (400 MHz, CDCl₃) δ ppm 0.54-0.59 (m, 2H), 0.76-0.82 (m, 2H), 1.03-1.21 (m, 2H), 1.21-1.41 (m, 2H), 1.48 (s, 3H), 1.72-1.85 (m, 1H), 1.89-2.04 (m, 4H), 2.23-2.79 (m, 5H), 2.97 (s, 3H), 3.14-3.72 (m, 4H), 3.83 (d, J=6.1 Hz, 2H), 6.98 (t, J=7.8 Hz, 1H), 7.55-7.64 (m, 2H).

### Example 1.14

### Preparation of tert-Butyl 4-(1r,4r)-4-((4-(1H-1,2,4-Triazol-1-yl)phenoxy)methyl)cyclohexyl)piperazine-1-carboxylate (Compound 14)

A mixture of tert-butyl 4-((1r,4r)-4-((methylsulfonyloxy)methyl)cyclohexyl)piperazine-1-carboxylate (504 mg, 1.339 mmol), Cs₂CO₃ (602 mg, 1.848 mmol), and 4-(1H-1,2,4-triazol-1-yl)phenol (220 mg, 1.365 mmol) in 10 mL DMF was heated under microwave irradiation at 100° C. for 2 h. The mixture was partitioned between 1 M NaOH and DCM. Organic phases were concentrated and the residue was purified by HPLC (5-95% CH₃CN/H₂O with 0.1% TFA). Fractions containing the title compound were combined, basified with 1 M NaOH aqueous solution, and partially concentrated, whereupon a solid precipitated. The solid was removed by filtration, washed with water, and dried under high vacuum to give the title compound (152 mg, 25.7%) as a white solid. Exact mass calculated for C₂₄H₃₅N₅O₃: 441.2. found LCMS m/z=442.2 [M+H]⁺; ¹H NMR (400 MHz, CDCl₃) δ ppm 1.08-1.18 (m, 2H), 1.25-1.35 (m, 2H), 1.46 (s, 9H), 1.72-1.82 (m, 1H), 1.93-2.03 (m, 4H), 2.27-2.35 (m, 1H), 2.50-2.54 (m, 4H), 3.41-3.45 (m, 4H), 3.80 (d, J=6.3 Hz, 2H), 6.96-7.00 (m, 2H), 7.53-7.57 (m, 2H), 8.07 (s, 1H), 8.43 (s, 1H).

### Example 1.15

### Preparation of 2-(4-((1r,4r)-4-((4-(1H-1,2,4-Triazol-1-yl)phenoxy)methyl)cyclohexyl)piperazin-1-yl)-5-(trifluoromethyl)pyrimidine (Compound 15)

### Step A: Preparation of 1-(1r,4r)-4-((4-(1H-1,2,4-Triazol-1-yl)phenoxy)methyl)cyclohexyl)piperazine Dihydrochloride

To a solution of tert-butyl 4-((1r,4r)-4-((4-(1H-1,2,4-triazol-1-yl)phenoxy)methyl)cyclohexyl)piperazine-1-carboxylate (148 mg, 0.335 mmol) in DCM (1 mL), a solution of 4 M hydrogen chloride in dioxane (2 mL, 8.00 mmol) was added. After stirring at room temperature for 2 h, the mixture was concentrated and dried under high vacuum to give the title compound (139 mg, 100%) as a white solid. Exact mass calculated for C₁₉H₂₇N₅O: 341.2. found LCMS m/z=342.4 [M+H]⁺.

### Step B: Preparation of 2-(4-((1r,4r)-4-((4-(1H-1,2,4-Triazol-1-yl)phenoxy)methyl)cyclohexyl)piperazin-1-yl)-5-(trifluoromethyl)pyrimidine (Compound 15)

A mixture of 1-((1r,4r)-4-((4-(1H-1,2,4-triazol-1-yl)phenoxy)methyl)cyclohexyl)piperazine dihydrochloride (34.0 mg, 0.082 mmol), 2-chloro-5-(trifluoromethyl)pyrimidine (21.3 mg, 0.117 mmol), and triethylamine (57 μL, 0.409 mmol) in IPA (1.5 mL) was heated under microwave irradiation at 150° C. for 1 h. The mixture was purified by HPLC (5-95% CH₃CN/H₂O with 0.1% TFA). Fractions containing the desired product were partially concentrated and the residue was partitioned between 1 M NaOH and DCM. The organic phases were combined, dried over MgSO₄, filtered, and concentrated to give the title compound as a white solid (26.7 mg, 66.7%). Exact mass calculated for C₂₄H₂₈F₃N₇O: 487.2. found LCMS m/z=488.4 [M+H]⁺; ¹H NMR (400 MHz, CDCl₃) δ ppm 1.10-1.20 (m, 2H), 1.28-1.38 (m, 2H), 1.72-1.82 (m, 1H), 1.93-2.03 (m, 4H), 2.32-2.42 (m, 1H), 2.62-2.68 (m, 4H), 3.81 (d, J=6.3 Hz, 2H), 3.89-3.93 (m, 4H), 6.99-7.01 (m, 2H), 7.52-7.57 (m, 2H), 8.07 (s, 1H), 8.43 (s, 1H), 8.47 (s, 2H).

### Example 1.16

### Preparation of 1,1,1,3,3,3-Hexafluoropropan-2-yl 4-(1r,4r)-4-((4-(1H-1,2,4-Triazol-1-yl)phenoxy)methyl)cyclohexyl)piperazine-1-carboxylate (Compound 16)

A mixture of 1,1,1,3,3,3-hexafluoropropan-2-ol (75.2 μL, 0.724 mmol) and CDI (105 mg, 0.648 mmol) in 1.5 mL THF was stirred at room temperature. After 40 min, 1-((1r,4r)-4-((4-(1H-1,2,4-triazol-1-yl)phenoxy)methyl)cyclohexyl)piperazine dihydrochloride (32 mg, 0.077 mmol) and triethylamine (53.8 μL, 0.386 mmol) were added. The reaction mixture was stirred under microwave irradiation at 150° C. for 2 h, then concentrated and the residue was purified by HPLC (5-95% CH₃CN/H₂O with 0.1% TFA). The fractions containing the title compound were partially concentrated and the residue was partitioned between 1 M NaOH aqueous solution and DCM. The organic phases were combined, dried over MgSO₄, filtered, and concentrated to give the title compound (20.2 mg, 48.8%) as a white solid. Exact mass calculated for C₂₃H₂₇F₆N₅O₃: 535.2. found LCMS m/z=536.4 [M+H]⁺; ¹H NMR (400 MHz, CDCl₃) δ ppm 1.08-1.20 (m, 2H), 1.25-1.38 (m, 2H), 1.72-1.80 (m, 1H), 1.93-2.03 (m, 4H), 2.32-2.40 (m, 1H), 2.57-2.65 (m, 4H), 3.52-3.57 (m, 4H), 3.81 (d, J=6.3 Hz, 2H), 5.72-5.78 (m, 1H), 6.97-7.01 (m, 2H), 7.54-7.57 (m, 2H), 8.07 (s, 1H), 8.43 (s, 1H).

### Example 1.17

### Preparation of 5-Chloro-2-(4-((1r,4r)-4-((2-fluoro-4-(methylsulfonyl)phenoxy)methyl)cyclohexyl)piperazin-1-yl)pyrimidine (Compound 17)

A solution of 1-((1r,4r)-4-((2-fluoro-4-(methylsulfonyl)phenoxy)methyl)cyclohexyl)piperazine dihydrochloride (50 mg, 0.114 mmol), 5-chloro-2-iodopyrimidine (44 mg, 0.184 mmol), and triethylamine (82 μL, 0.614 mmol) in IPA (1.6 mL) was heated under microwave irradiation for 30 min at 160° C. The reaction was cooled to room temperature, and the product was precipitated. The solid was filtered, washed with water, 1 N HCl aqueous solution, and IPA, and then dried to afford the title compound (42 mg, 70.8% yield). Exact mass calculated for C₂₄H₂₈ClF₃N₄O₃S: 482.2. found LCMS m/z=483.2 [M+H]⁺; ¹H NMR (400 MHz, CDCl₃) δ ppm 1.10-1.20 (m, 2H), 1.28-1.38 (m, 2H), 1.78-1.88 (m, 1H), 1.95-2.05 (m, 4H), 2.30-2.38 (m, 1H), 2.62-2.66 (m, 4H), 3.03 (s, 3H), 3.78-3.82 (m, 4H), 3.90 (d, J=6.3 Hz, 2H), 7.06 (t, J=8.1 Hz, 1H), 7.62-7.70 (m, 2H), 8.22 (s, 2H).

### Example 1.18

### Preparation of 5-(4-((1r,4r)-4-((2-Fluoro-4-(methylsulfonyl)phenoxy)methyl)cyclohexyl)piperazin-1-yl)-3-(2-fluoropropan-2-yl)-1,2,4-oxadiazole (Compound 18)

### Step A: Preparation of 4-((1r,4r)-4-((2-Fluoro-4-(methylsulfonyl)phenoxy)methyl)cyclohexyl)piperazine-1-carbonitrile

To a mixture of 1-((1r,4r)-4-((2-fluoro-4-(methylsulfonyl)phenoxy)methyl)cyclohexyl)piperazine dihydrochloride and DIEA (186 μL, 1.062 mmol) in DCM (4 mL) was added CNBr (55 mg, 0.531 mmol). The reaction was stirred at room temperature for 1 h, washed with saturated NaHCO₃, dried, and concentrated. The residue was washed with IPA to give the title compound (180 mg, 0.455 mmol, 86%). Exact mass calculated for C₁₉H₂₆FN₃O₃S: 395.2. found: LCMS m/z=396.0 [M+H]⁺.

### Step B: Preparation of 5-(4-((1r,4r)-4-((2-Fluoro-4-(methylsulfonyl)phenoxy)methyl)cyclohexyl)piperazin-1-yl)-3-(2-fluoropropan-2-yl)-1,2,4-oxadiazole (Compound 18)

The title compound (12 mg, 24%) was prepared in a manner similar to that described in Example 1.11 using 4-((1r,4r)-4-((2-fluoro-4-(methylsulfonyl)phenoxy)methyl)cyclohexyl)piperazine-1-carbonitrile. Exact mass calculated for C₂₃H₃₂F₂N₄O₄S: 498.2. found: LCMS m/z=499.2 [M+H]⁺; ¹H NMR (400 MHz, CDCl₃) δ ppm 1.10-1.50 (m, 4H), 1.73 (d, J=20.7 Hz, 6H), 1.75-2.10 (m, 5H), 2.35-2.48 (m, 1H), 2.60-2.78 (m, 4H), 3.03 (s, 3H), 3.55-3.80 (m, 4H), 3.91 (d, J=6.2 Hz, 2H), 7.05 (t, J=8.0 Hz, 1H), 7.62-7.69 (m, 2H).

### Example 1.19

### Preparation of 3-(4-((1r,4r)-4-((2-Fluoro-4-(methylsulfonyl)phenoxy)methyl)cyclohexyl)piperazin-1-yl)-5-(2-fluoropropan-2-yl)-1,2,4-oxadiazole (Compound 19)

A solution of 4-((1r,4r)-4-((2-fluoro-4-(methylsulfonyl)phenoxy)methyl)cyclohexyl)piperazine-1-carbonitrile (110 mg, 0.278 mmol), prepared in Example 1.18, Step A, and hydroxylamine (˜50% in H₂O, 0.464 mL, 6.95 mmol) in EtOH (1.5 mL) was stirred for 1 h at 60° C. The solvent was removed by evaporation to leave 4-((1r,4r)-4-((2-fluoro-4-(methylsulfonyl)phenoxy)methyl)cyclohexyl)-N′-hydroxypiperazine-1-carboximidamide as a white solid. To a solution of 4-((1r,4r)-4-((2-fluoro-4-(methylsulfonyl)phenoxy)methyl)cyclohexyl)-Ar-hydroxypiperazine-1-carboximidamide in THF (3 mL) was added 2-fluoro-2-methylpropanoyl chloride (42 mg, 0.334 mmol) (prepared by reacting 2-fluoro-2-methylpropanoic acid (35 mg, 0.334 mmol) with oxalyl chloride (1 M in DCM, 0.5 mL)). triethylamine (0.111 mL, 0.834 mmol) was added and the reaction was stirred overnight at 40° C. The reaction was then quenched with water and diluted with DCM. The unreacted carboximidamide was extracted with aqueous 1 N HCl, and the organic layer was dried and concentrated. The residue was purified by preparative TLC to give the title compound (20 mg, 14.4%). Exact mass calculated for C₂₃H₃₂F₂N₄O₄S: 498.2. found: LCMS m/z=499.2 [M+H]⁺; ¹H NMR (400 MHz, CDCl₃) δ ppm 1.10-1.22 (m, 2H), 1.30-1.42 (m, 2H), 1.78 (d, J=20.7 Hz, 6H), 1.80-2.08 (m, 5H), 2.35-2.42 (m, 1H), 2.63-2.74 (m, 4H), 3.03 (s, 3H), 3.45-3.55 (m, 4H), 3.91 (d, J=6.2 Hz, 2H), 7.05 (t, J=8.1 Hz, 1H), 7.62-7.69 (m, 2H).

### Example 1.20

### Preparation of tert-Butyl 4-(1r,4r)-4-((4-(1H-Tetrazol-1-yl)phenoxy)methyl)cyclohexyl)piperazine-1-carboxylate (Compound 20)

A mixture of tert-butyl 4-((1r,4r)-4-((methylsulfonyloxy)methyl)cyclohexyl)piperazine-1-carboxylate (175 mg, 0.465 mmol), 4-(1H-tetrazol-1-yl)phenol (79 mg, 0.487 mmol), and cesium carbonate (204 mg, 0.626 mmol) in DMF (3 mL) was stirred at 80° C. (oil bath) overnight. The reaction mixture was purified by HPLC (5-95% CH₃CN/H₂O with 0.1% TFA). The fractions containing the title compound were combined, basified by the addition of aqueous 1 M NaOH solution, and partially concentrated, whereupon a solid precipitated. The solid was filtered off, washed with additional water, and dried under high vacuum to give the title compound (23.4 mg, 0.053 mmol, 11.4%) as a white solid. Exact mass calculated for C₂₃H₃₄N₆O₃: 442.2. found LCMS m/z=443.4 [M+H]⁺; ¹H NMR (400 MHz, CDCl₃) δ ppm 1.08-1.18 (m, 2H), 1.25-1.35 (m, 2H), 1.46 (s, 9H), 1.74-1.82 (m, 1H), 1.95-2.03 (m, 4H), 2.27-2.35 (m, 1H), 2.48-2.54 (m, 4H), 3.41-3.45 (m, 4H), 3.82 (d, J=6.3 Hz, 2H), 6.99-7.06 (m, 2H), 7.52-7.59 (m, 2H), 8.87 (s, 1H).

### Example 1.21

### Preparation of 2-(4-((1r,4r)-4-((4-(1H-Tetrazol-1-yl)phenoxy)methyl)cyclohexyl)piperazin-1-yl)-5-(trifluoromethyl)pyrimidine (Compound 21)

### Step A: Preparation of 1-(1r,4r)-4-((4-(1H-Tetrazol-1-yl)phenoxy)methyl)cyclohexyl)piperazine Dihydrochloride

To a solution of tert-butyl 4-((1r,4r)-4-((4-(1H-tetrazol-1-yl)phenoxy)methyl)cyclohexyl)piperazine-1-carboxylate (20 mg, 0.045 mmol) in 1 mL DCM, was added a solution of 4 M hydrogen chloride in dioxane (2 mL, 8.00 mmol). After stirring at room temperature for 3 h, the mixture was concentrated. The residue was dried under high vacuum to give the title compound (18.8 mg, 100%) as a white solid. Exact mass calculated for C₁₈H₂₆N₆O: 342.2. found LCMS m/z=343.4 [M+H]⁺.

### Step B: Preparation of 2-(4-((1r,4r)-4-((4-(1H-Tetrazol-1-yl)phenoxy)methyl)cyclohexyl)piperazin-1-yl)-5-(trifluoromethyl)pyrimidine (Compound 21

A mixture of 1-((1r,4r)-4-((4-(1H-tetrazol-1-yl)phenoxy)methyl)cyclohexyl)piperazine dihydrochloride (18.8 mg, 0.045 mmol), 2-chloro-5-(trifluoromethyl)pyrimidine (12 mg, 0.066 mmol), and triethylamine (30 μL, 0.215 mmol) in IPA (1 mL) was heated under microwave irradiation at 150° C. for 1 h. The mixture was purified by HPLC (5-95% CH₃CN/H₂O with 0.1% TFA over 40 min). The factions containing the title compound were combined and partially concentrated. The residue was partitioned between 1 M NaOH and DCM. The organic phases were combined, dried over MgSO₄, filtered, and concentrated. The residue was purified by silica gel flash column chromatography to give the title compound (4.1 mg, 18.5%) as a white solid. Exact mass calculated for C₂₃H₂₇F₃N₈O: 488.2. found LCMS m/z=489.4 [M+H]⁺; ¹H NMR (400 MHz, CDCl₃) δ ppm 1.08-1.20 (m, 2H), 1.25-1.40 (m, 2H), 1.74-1.84 (m, 1H), 1.97-2.05 (m, 4H), 2.34-2.44 (m, 1H), 2.65-2.68 (m, 4H), 3.83 (d, J=6.3 Hz, 2H), 3.88-3.94 (m, 4H), 7.02-7.07 (m, 2H), 7.55-7.60 (m, 2H), 8.47 (s, 2H), 8.88 (s, 1H).

### Example 1.22

### Preparation of tert-Butyl 4-((1r,4r)-4-((4-Cyanophenoxy)methyl)cyclohexyl)piperazine-1-carboxylate (Compound 22)

A mixture of tert-butyl 4-((1r,4r)-4-((methylsulfonyloxy)methyl)cyclohexyl)piperazine-1-carboxylate (191 mg, 0.507 mmol), 4-hydroxybenzonitrile (76 mg, 0.638 mmol), and cesium carbonate (200 mg, 0.614 mmol) in DMF (3 mL) was heated under microwave irradiation at 100° C. for 1 h. The reaction mixture was purified by HPLC (5-95% CH₃CN/H₂O with 0.1% TFA). The factions containing the title compound were combined, basified by the addition of 1 M NaOH aqueous solution, partially concentrated, and extracted with DCM. The organic phases were combined, dried over MgSO₄, filtered, and concentrated to give the title compound (57.32 mg, 28.3%) as a white solid. Exact mass calculated for C₂₃H₃₃N₃O₃: 399.2. found LCMS m/z=400.0 [ M+H]⁺; ¹H NMR (400 MHz, CDCl₃) δ ppm 1.08-1.18 (m, 2H), 1.25-1.35 (m, 2H), 1.46 (s, 9H), 1.72-1.80 (m, 1H), 1.95-2.01 (m, 4H), 2.27-2.35 (m, 1H), 2.49-2.54 (m, 4H), 3.41-3.45 (m, 4H), 3.80 (d, J=6.3 Hz, 2H), 6.90-6.94 (m, 2H), 7.55-7.58 (m, 2H).

### Example 1.23

### Preparation of 4(((1r,4r)-4-(4-(5-(Trifluoromethyl)pyrimidin-2-yl)piperazin-1-yl)cyclohexyl)methoxy)benzonitrile (Compound 23)

### Step A: Preparation of 4-(((1r,4r)-4-(piperazin-1-yl)cyclohexyl)methoxy)benzonitrile Dihydrochloride

To a solution of tert-butyl 4-((1r,4r)-4-((4-cyanophenoxy)methyl)cyclohexyl)piperazine-1-carboxylate (53 mg, 0.133 mmol) in DCM (1 mL), was added a solution of 4 M hydrogen chloride in dioxane (2 mL, 8.00 mmol). After stirring at room temperature for 3 h, the reaction mixture was concentrated and dried under high vacuum to give the title compound (49.4 mg, 100%) as a white solid. Exact mass calculated for C₁₈H₂₅N₃O: 299.2. found LCMS m/z=300.2 [M+H]⁺.

### Step B: Preparation of 4-((1r,4r)-4-(4-(5-(Trifluoromethyl)pyrimidin-2-yl)piperazin-1-yl)cyclohexyl)methoxy)benzonitrile (Compound 23)

A mixture of 4-(((1r,4r)-4-(piperazin-1-yl)cyclohexyl)methoxy)benzonitrile dihydrochloride (26 mg, 0.070 mmol), 2-chloro-5-(trifluoromethyl)pyrimidine (15.1 mg, 0.083 mmol), and triethylamine (45 μL, 0.323 mmol) in IPA (1 mL) was heated under microwave irradiation at 150° C. for 1 h. The reaction mixture was purified by HPLC (5-95% CH₃CN/H₂O with 0.1% TFA over 40 min). The factions containing the title compound were combined and partially concentrated. The residue was partitioned between 1 M NaOH and DCM. The organic phases were combined, dried over MgSO₄, filtered, and concentrated to give the title compound (22.9 mg, 73.4%) as a white solid. Exact mass calculated for C₂₃H₂₆F₃N₅O: 445.2. found LCMS m/z=446.6 [M+H]⁺; ¹H NMR (400 MHz, CDCl₃) δ ppm 1.08-1.18 (m, 2H), 1.25-1.39 (m, 2H), 1.72-1.82 (m, 1H), 1.95-2.02 (m, 4H), 2.31-2.38 (m, 1H), 2.62-2.67 (m, 4H), 3.80 (d, J=6.3 Hz, 2H), 3.88-3.92 (m, 4H), 6.90-6.94 (m, 2H), 7.55-7.59 (m, 2H), 8.47 (s, 2H).

### Example 1.24

### Preparation of 2-((S)-4-((1r,4S)-4-((2-Fluoro-4-(methylsulfonyl)phenoxy)methyl)cyclohexyl)-3-methylpiperazin-1-yl)-5-(trifluoromethyl)pyrimidine (Compound 24)

### Step A: Preparation of (S)-tert-Butyl 4-(4-(Ethoxycarbonyl)cyclohexyl)-3-methylpiperazine-1-carboxylate

To a mixture of ethyl 4-oxocyclohexanecarboxylate (10 g, 58.8 mmol) and (S)-tert-butyl 3-methylpiperazine-1-carboxylate (14.12 g, 70.5 mmol) in DCM (200 mL) was added acetic acid (6.72 mL, 118 mmol). Sodium triacetoxyborohydride (31.1 g, 147 mmol) was added under a nitrogen atmosphere and the reaction was stirred overnight at room temperature. The mixture was diluted with water, adjusted pH to 8 with 1 N NaOH, and extracted with DCM. The organic phase were combined, dried over anhydrous Na₂SO₄, filtered, and then concentrated. The residue was purified by column chromatography (EtOAc) to give a mixture of cis and trans isomers of the title compound as an oil (16 g, 77%). Exact mass calculated for C₁₉H₃₄N₂O₄: 354.2. found LCMS m/z=355.2 [M+H]⁺.

### Step B: Preparation of (S)-tert-Butyl 4-(4-(hydroxymethyl)cyclohexyl)-3-methylpiperazine-1-carboxylate

To a stirred solution of (S)-tert-butyl 4-(4-(ethoxycarbonyl)cyclohexyl)-3-methylpiperazine-1-carboxylate (5 g, 14.10 mmol) in THF (30 mL) was added a 2 M solution of lithium borohydride in THF (9.17 mL, 18.34 mmol) under N₂. The reaction mixture was stirred at reflux overnight. The mixture was acidified to pH 3-4 with 3M HCl aqueous solution, then basified to pH 8 with 1 N NaOH aqueous solution, extracted with IPA/DCM (1:4). The combined organics were dried over anhydrous Na₂SO₄, filtered then concentrated. The residue was purified by column chromatography with 100% ethyl acetate, then 5% MeOH/ethyl acetate to give the title compound (2.62 g, 8.39 mmol, 59.4%) as a mixture of cis and trans isomers. Exact mass calculated for C₁₇H₃₂N₂O₃: 312.2. found LCMS m/z=313.2 [M+H]⁺.

### Step C: Preparation of (S)-tert-Butyl 3-Methyl-4-(4-((methylsulfonyloxy)methyl)cyclohexyl)piperazine-1-carboxylate

To a stirred solution of (S)-tert-butyl 4-(4-(hydroxymethyl)cyclohexyl)-3-methylpiperazine-1-carboxylate (100 mg, 0.320 mmol) and triethylamine (89 μL, 0.640 mmol) in DCM (2 mL) was added dropwise methanesulfonyl chloride (44.0 mg, 0.384 mmol) in an ice-water bath. The reaction was slowly warmed to room temperature and stirred for 2 h. The mixture was diluted with DCM and washed with water. The organic phases were combined, dried over anhydrous Na₂SO₄. and concentrated to give the title compound (100 mg, 80%). Exact mass calculated for C₁₈H₃₄N₂O₅S: 390.2. found LCMS m/z=391.2 [M+H]⁺.

### Step D: Preparation of (S)-tert-Butyl 4-(1r,4S)-4-((2-Fluoro-4-(methylsulfonyl)phenoxy)methypcyclohexyl)-3-methylpiperazine-1-carboxylate

(S)-tert-Butyl 3-methyl-4-(4-((methylsulfonyloxy)methyl)cyclohexyl)piperazine-1-carboxylate (0.90 g, 2.305 mmol) was dissolved in DMF (20 mL), 2-fluoro-4-(methylsulfonyl)phenol (0.526 g, 2.77 mmol) and K₂CO₃ (0.637 g, 4.61 mmol) were added. The reaction was heated at 100° C. for 2 h under microwave irradiation. The solid was filtered off, washed with ethyl acetate. The filtrate was concentrated, the residue was purified by column chromatography (99:1 EtOAc:Et₃N) to give the title compound (400 mg, 35.8%) as white solid. Exact mass calculated for C₂₄H₃₇FN₂O₅S: 484.2. found LCMS m/z=485.2 [M+H]⁺.

### Step E: Preparation of (S)-1-((1r,4S)-4-((2-Fluoro-4-(methylsulfonyl)phenoxy)methyl)cyclohexyl)-2-methylpiperazine

To a solution of (S)-tert-butyl 4-((1r,4S)-4-(2-fluoro-4-(methylsulfonyl)phenoxy)methyl)cyclohexyl)-3-methylpiperazine-1-carboxylate (100 mg, 0.206 mmol) in DCM (5 mL) was added TFA (1 mL). The reaction was stirred at room temperature for 1 h. The mixture was concentrated to give the title compound (79 mg, 0.205 mmol, 100%) without further purification. Exact mass calculated for C₁₉H₂₉FN₂O₃S: 384.2. found LCMS m/z=385.0 [M+H]⁺.

### Step F: Preparation of 2-((S)-4-(1r,4S)-4-((2-Fluoro-4-(methylsulfonyl)phenoxy)methyl)cyclohexyl)-3-methylpiperazin-1-yl)-5-(trifluoromethyl)pyrimidine (Compound 24)

A mixture of (S)-1-((1r,4S)-4-(2-fluoro-4-(methylsulfonyl)phenoxy)methyl)cyclohexyl)-2-methylpiperazine (0.040 g, 0.104 mmol), 2-chloro-5-(trifluoromethyl)pyrimidine (0.023 g, 0.125 mmol), and triethylamine (0.042 mL, 0.312 mmol) in 2-propanol (1.5 mL) was heated at 130° C. under microwave irradiation for 50 min. The reaction was cooled down to room temperature. The mixture was filtered, and the cake was washed with IPA, dried. The residue was purified by silica gel flash column chromatography to give the title compound (0.02 g, 36.2%) as a white solid. Exact mass calculated for C₂₄H₃₀F₄N₄O₃S: 530.2. found LCMS m/z=531.2 [M+H]⁺; ¹H NMR (400 MHz, CDCl₃) δ ppm 1.13-1.33 (m, 6H), 1.54-1.67 (m, 2H), 1.82-1.94 (m, 3H), 2.05-2.10 (m, 2H), 2.46-2.56 (m, 1H), 2.78-3.00 (m, 2H), 3.06 (s, 3H), 3.06-3.20 (m, 1H), 3.32-3.45 (m, 1H), 3.93 (d, J=6.1 Hz, 2H), 4.37-4.52 (m, 2H), 7.08 (t, J=8.1 Hz, 1H), 7.65-7.72 (m, 2H), 8.50 (s, 2H).

### Example 1.25

### Preparation of 5-((S)-4-(1r,4S)-4-((2-Fluoro-4-(methylsulfonyl)phenoxy)methyl)cyclohexyl)-3-methylpiperazin-1-yl)-3-(2-fluoropropan-2-yl)-1,2,4-oxadiazole (Compound 25)

### Step A: Preparation of (S)-4-(1r,4S)-4-((2-Fluoro-4-(methylsulfonyl)phenoxy)methyl)cyclohexyl)-3-methylpiperazine-1-carbonitrile

To a solution of (S)-1-((1r,4S)-4-(2-fluoro-4-(methylsulfonyl)phenoxy)methyl)cyclohexyl)-2-methylpiperazine (70 mg, 0.182 mmol) and DIEA (71 mg, 0.546 mmol) in DCM (3 mL) was added CNBr (21 mg, 0.20 mmol) at room temperature under a nitrogen atmosphere. The reaction was stirred for 1 h. The mixture was diluted with DCM, washed with saturated NaHCO₃, dried with MgSO₄, and concentrated to give the title compound as a white solid. Exact mass calculated for C₂₀H₂₈FN₃O₃S: 409.2. found LCMS m/z=410.2 [M+11]⁺.

### Step B: Preparation of 5-((S)-4-(1r,4S)-4-((2-Fluoro-4-(methylsulfonyl)phenoxy)methyl)cyclohexyl)-3-methylpiperazin-1-yl)-3-(2-fluoropropan-2-yl)-1,2,4-oxadiazole (Compound 25)

To a solution of (S)-4-((1r,4S)-4-((2-fluoro-4-(methylsulfonyl)phenoxy)methyl)cyclohexyl)-3-methylpiperazine-1-carbonitrile in THF (4 mL) was added 2-fluoro-Ar-hydroxy-2-methylpropanimidamide (37 mg, 0.309 mmol) and a solution of 0.5 M ZnCl₂ in THF (0.619 mL, 0.309 mmol). The reaction was stirred at room temperature for 2 h. After adding a solution of 4 M HCl in dioxane (0.182 mL, 0.728 mmol), the reaction was heated at 150° C. under microwave irradiation for 50 min. The mixture was then diluted with DCM, washed with saturated NaHCO₃, dried, and concentrated. The residue was purified by preparative HPLC to give the title compound (30 mg, 32.1%). Exact mass calculated for C₂₄H₃₄F₂N₄O₄S: 512.2. found LCMS m/z=513.4 [M+H]⁺; ¹H NMR (400 MHz, CDCl₃) δ ppm 1.05-1.30 (m, 6H), 1.50-1.60 (m, 2H), 1.73 (d, J=21.7 Hz, 6H), 1.76-1.90 (m, 3H), 1.95-2.08 (m, 2H), 2.45-2.53 (m, 1H), 2.75-2.92 (m, 2H), 2.98-3.08 (m, 1H), 3.03 (s, 3H), 3.26-3.36 (m, 1H), 3.83-3.95 (m, 2H), 3.90 (d, J=6.1 Hz, 2H), 7.05 (t, J=8.1 Hz, 1H), 7.62-7.70 (m, 2H).

### Example 1.26

### Preparation of 2-((S)-3-Methyl-4-(1r,4S)-4-((2-methyl-6-(methylsulfonyl)pyridin-3-yloxy)methyl)cyclohexyl)piperazin-1-yl)-5-(trifluoromethyl)pyrimidine (Compound 26)

### Step A: Preparation of (S)-2-Methyl-1-(1r,4S)-4-((2-methyl-6-(methylsulfonyl)pyridin-3-yloxy)methyl)cyclohexyl)piperazine dihydrochloride

A solution of (S)-tert-butyl 3-methyl-4-(4-((methylsulfonyloxy)methyl)cyclohexyl)piperazine-1-carboxylate (400 mg, 1.024 mmol), 2-methyl-6-(methylsulfonyl)pyridin-3-ol (268 mg, 1.434 mmol), and K₂CO₃ (283 mg, 2.048 mmol) in DMF (12 mL) was stirred overnight at 80° C. The reaction was diluted with DCM, washed with saturated aqueous NaHCO₃ solution twice, dried, and concentrated. The residue was purified by column chromatography to give (S)-tert-butyl 3-methyl-4-((1r,4S)-4-(2-methyl-6-(methylsulfonyl)pyridin-3-yloxy)methyl)cyclohexyl)piperazine-1-carboxylate. The boc-deprotection was performed under standard conditions using 4 N HCl in dioxane (2.06 mL, 10.24 mmol) to give the title compound (150 mg, 0.33 mmol, 32.2%). Exact mass calculated for C₁₉H₃₁N₃O₃S: 381.2. found LCMS m/z=382.2 [M+H]⁺.

### Step B: Preparation of 2-((S)-3-Methyl-4-(1r,4S)-4-((2-methyl-6-(methylsulfonyl)pyridin-3-yloxy)methyl)cyclohexyl)piperazin-1-yl)-5-(trifluoromethyl)pyrimidine (Compound 26)

A solution of (S)-2-methyl-1-((1r,4S)-4-(2-methyl-6-(methylsulfonyl)pyridin-3-yloxy)methyl)cyclohexyl)piperazine dihydrochloride (40 mg, 0.088 mmol), 2-chloro-5-(trifluoromethyl)pyrimidine (18 mg, 0.097 mmol), triethylamine (47 μL, 0.352 mmol) in IPA (1 mL) was heated at 130° C. under microwave irradiation for 50 min. After removal of the solvent, the residue was purified by preparative HPLC to give the title compound (20 mg, 43.1%). Exact mass calculated for C₂₄H₃₂F₃N₅O₃S: 527.2. found LCMS m/z=528.2 [M+H]⁺; ¹H NMR (400 MHz, CDCl₃) δ ppm 1.10-1.35 (m, 6H), 1.50-1.65 (m, 2H), 1.80-2.10 (m, 5H), 2.40-2.50 (m, 1H), 2.52 (s, 3H), 2.75-3.00 (m, 2H), 3.00-3.15 (m, 1H), 3.17 (s, 3H), 3.25-3.40 (m, 1H), 3.85 (d, J=6.2 Hz, 2H), 4.30-4.50 (m, 2H), 7.14 (d, J=8.5 Hz, 1H), 7.89 (d, J=8.5 Hz, 1H), 8.47 (s, 2H).

### Example 1.27

### Preparation of 2-((R)-4-((1r,4R)-4-((2-Fluoro-4-(methylsulfonyl)phenoxy)methyl)cyclohexyl)-3-methylpiperazin-1-yl)-5-(trifluoromethyl)pyrimidine (Compound 27)

### Step A: Preparation of (R)-tert-Butyl 4-(4-(Ethoxycarbonyl)cyclohexyl)-3-methylpiperazine-1-carboxylate

The title compound was prepared in a manner similar to that described in Example 1.24, Step A using (R)-tert-butyl 3-methylpiperazine-1-carboxylate. Exact mass calculated for C₁₉H₃₄N₂O₄: 354.2. found LCMS m/z=355.2 [M+H]⁺.

### Step B: Preparation of (R)-tert-Butyl 4-(4-(Hydroxymethyl)cyclohexyl)-3-methylpiperazine-1-carboxylate

The title compound was prepared in a manner similar to that described in Example 1.24, Step B using (R)-tert-butyl 4-(4-(ethoxycarbonyl)cyclohexyl)-3-methylpiperazine-1-carboxylate. Exact mass calculated for C₁₇H₃₂N₂O₃: 312.2. found LCMS m/z=313.2 [M+H]⁺.

### Step C: Preparation of (R)-tert-Butyl 3-Methyl-4-(4-((methylsulfonyloxy)methyl)cyclohexyl)piperazine-1-carboxylate

The title compound was prepared in a manner similar to that described in Example 1.24, Step C using (R)-tert-butyl 4-(4-(hydroxymethyl)cyclohexyl)-3-methylpiperazine-1-carboxylate. Exact mass calculated for C₁₈H₃₄N₂O₅S: 390.2. found LCMS m/z=391.2 [M+H]⁺.

### Step D: Preparation of (R)-tert-Butyl 4-(1r,4r)-4-((2-Fluoro-4-(methylsulfonyl)phenoxy)methyl)cyclohexyl)-3-methylpiperazine-1-carboxylate

The title compound was prepared in a manner similar to that described in Example 1.24, Step D using (R)-tert-butyl 3-methyl-4-(4-((methylsulfonyloxy)methyl)cyclohexyl)piperazine-1-carboxylate. Exact mass calculated for C₂₄H₃₇FN₂O₅S: 484.2. found LCMS m/z=485.2 [M+H]⁺.

### Step E: Preparation of (R)-1-(1r,4r)-4-((2-Fluoro-4-(methylsulfonyl)phenoxy)methyl)cyclohexyl)-2-methylpiperazine

The title compound was prepared in a manner similar to that described in Example 1.24, Step E using (R)-tert-butyl 4-((1r,4r)-4-((2-fluoro-4-(methylsulfonyl)phenoxy)methyl)cyclohexyl)-3-methylpiperazine-1-carboxylate. Exact mass calculated for C₁₉H₂₉FN₂O₃S: 384.2. found LCMS m/z=385.0 [M+H]⁺.

### Step F: Preparation of 2-((R)-4-((1r,4r)-4-((2-Fluoro-4-(methylsulfonyl)phenoxy)methyl)cyclohexyl)-3-methylpiperazin-1-yl)-5-(trifluoromethyl)pyrimidine (Compound 27)

The title compound was prepared in a manner similar to that described in Example 1.24, Step F using (R)-1-((1r,4r)-4-((2-fluoro-4-(methylsulfonyl)phenoxy)methyl)cyclohexyl)-2-methylpiperazine. Exact mass calculated for C₂₄H₃₀F₄N₄O₃S: 530.2. found LCMS m/z=531.4 [M+H]⁺; ¹H NMR (400 MHz, CDCl₃) δ ppm 1.13-1.33 (m, 6H), 1.54-1.67 (m, 2H), 1.82-1.94 (m, 3H), 2.05-2.10 (m, 2H), 2.46-2.56 (m, 1H), 2.78-3.00 (m, 2H), 3.00-3.15 (m, 1H), 3.06 (s, 3H), 3.25-3.45 (m, 1H), 3.91 (d, J=6.1 Hz, 2H), 4.37-4.52 (m, 2H), 7.04 (t, J=8.1 Hz, 1H), 7.63-7.70 (m, 2H), 8.48 (s, 2H).

### Example 1.28

### Preparation of (R)-1-Fluoropropan-2-yl 4-((1r,4R)-4-((2-Fluoro-4-(methylsulfonyl)phenoxy)methyl)cyclohexyl)piperazine-1-carboxylate (Compound 28) and

(S)-1-Fluoropropan-2-yl 4-((1r,4S)-4-((2-Fluoro-4-(methylsulfonyl)phenoxy)methyl)cyclohexyl)piperazine-1-carboxylate (Compound 29)

A mixture of CDI (0.055 g, 0.338 mmol) and 1-fluoropropan-2-ol (0.032 g, 0.406 mmol) were stirred in THF (1.5 mL) at room temperature for 24 h. 1-((1r,4r)-4-((2-Fluoro-4-(methylsulfonyl)phenoxy)methyl)cyclohexyl)piperazine dihydrochloride (0.15 g, 0.338 mmol), triethylamine (0.375 mL, 2.71 mmol), and THF (1.5 mL) were added and the reaction mixture was warmed to 150° C. After stirring for 1 h, the reaction was diluted with water and extracted two times with EtOAc. The combined organic extracts were dried over sodium sulfate, filtered, and concentrated. The residue was purified by column chromatography (silica gel, 0-5% MeOH in DCM) to give the title compounds (121 mg, 75%).

### Resolution via Chiral HPLC.

Column: normal phase preparative Chiralpak® IA 250×20 mm ID, 5 μM particle size

Eluent: 10% DCM in MeCN with 0.1% diethylamine

Gradient: isocratic

Flow: 8 mL/min

Detector: 254 nm

Retention Times: 1^{st} enantiomer: 19.6 min; 2^{nd} enantiomer: 25.2 min

1^{st} enantiomer: Exact mass calculated for C₂₂H₃₂F₂N₂O₅S: 474.2. found: LCMS m/z=475.4 [M+H]⁺; ¹H NMR (400 MHz, CDCl₃) δ ppm 1.07-1.39 (m, 7H), 1.77-2.06 (m, 5H), 2.27-2.39 (m, 1H), 2.47-2.62 (m, 4H), 3.03 (s, 3H), 3.44-3.54 (m, 4H), 3.90 (d, J=6.1 Hz, 2H), 4.29-4.54 (m, 2H), 4.96-5.10 (m, 1H), 7.02-7.08 (m, 1H), 7.62-7.70 (m, 2H).
2^{nd} enantiomer: Exact mass calculated for C₂₂H₃₂F₂N₂O₅S: 474.2. found: LCMS m/z=475.4 [M+H]⁺; ¹H NMR (400 MHz, CDCl₃) δ ppm 1.07-1.39 (m, 7H), 1.76-2.06 (m, 5H), 2.27-2.38 (m, 1H), 2.48-2.62 (m, 4H), 3.03 (s, 3H), 3.44-3.54 (m, 4H), 3.90 (d, J=6.1 Hz, 2H), 4.30-4.54 (m, 2H), 4.96-5.10 (m, 1H), 7.02-7.09 (m, 1H), 7.61-7.71 (m, 2H).

### Example 1.29

### Preparation of 1-Fluoro-2-methylpropan-2-yl 4-(1r,4r)-4-((2-fluoro-4-(methylsulfonyl)phenoxy)methyl)cyclohexyl)piperazine-1-carboxylate (Compound 30)

A mixture of CDI (0.05 g, 0.308 mmol) and 1-fluoro-2-methylpropan-2-ol (0.12 g, 1.303 mmol) were stirred in THF (1.0 mL) at 60° C. for 24 h. 1-((1r,4r)-4-((2-fluoro-4-(methylsulfonyl)phenoxy)methyl)cyclohexyl)piperazine dihydrochloride (0.1 g, 0.226 mmol) and THF (2.0 mL) were added and the reaction was stirred at 100° C. for 12 h. The reaction was diluted with water and extracted three times with EtOAc. The combined extracts were dried (sodium sulfate), filtered, and concentrated. The residue was purified by column chromatography (silica gel, 5% MeOH in DCM) and reverse-phase HPLC to give the title compound as a TFA salt. The TFA salt was taken up in DCM and washed with saturated aqueous sodium carbonate solution and then water. The DCM layer was dried (sodium sulfate), filtered, and concentrated to give the title compound as a white solid (40.1 mg, 36%). Exact mass calculated for C₂₃H₃₄F₂N₂O₅S: 488.22. found: LCMS m/z=489.2 [M+H]⁺; ¹H NMR (400 MHz, CDCl₃) δ ppm 1.07-1.39 (m, 4H), 1.48 (s, 6H), 1.76-1.89 (m, 1H), 1.91-2.05 (m, 4H), 2.27-2.37 (m, 1H), 2.48-2.59 (m, 4H), 3.03 (s, 3H), 3.40-3.48 (m, 4H), 3.90 (d, J=6.4 Hz, 2H), 4.48 (d, J=47.8 Hz, 2H), 7.02-7.08 (m, 1H), 7.62-7.70 (m, 2H).

### Example 1.30

### Preparation of 5-Chloro-2-((R)-4-((1r,4R)-4-((2-fluoro-4-(methylsulfonyl)phenoxy)methyl)cyclohexyl)-3-methylpiperazin-1-yl)pyrimidine (Compound 31)

A mixture of (R)-1-((1r,4R)-4-(2-fluoro-4-(methylsulfonyl)phenoxy)methyl)cyclohexyl)-2-methylpiperazine (100 mg, 0.260 mmol), 2,5-dichloropyrimidine (46.5 mg, 0.312 mmol), and triethylamine (0.104 mL, 0.780 mmol) in IPA (1.5 mL) was heated under microwave irradiation for 50 min at 130° C. The reaction mixture was cooled to room temperature and a solid precipitated. The solid was filtered off, washed with IPA, dried, and purified by column chromatography to give the title compound (0.065 g, 50.3%) as a white solid. Exact mass calculated for C₂₄H₃₀ClFN₄O₃S: 496.2. found LCMS m/z=497.2 [M+H]⁺; ¹H NMR (400 MHz, CDCl₃) δ ppm 1.13-1.33 (m, 6H), 1.54-1.67 (m, 2H), 1.82-2.00 (m, 3H), 2.00-2.10 (m, 2H), 2.40-2.56 (m, 1H), 2.78-3.00 (m, 2H), 3.00-3.15 (m, 1H), 3.06 (s, 3H), 3.15-3.30 (m, 1H), 3.94 (d, J=6.1 Hz, 2H), 4.20-4.45 (m, 2H), 7.08 (t, J=8.1 Hz, 1H), 7.65-7.71 (m, 2H), 8.24 (s, 2H).

### Example 1.31

### Preparation of (R)—((R)-1,1,1-Trifluoropropan-2-yl) 4-(1r,4R)-4-((2-Fluoro-4-(methylsulfonyl)phenoxy)methyl)cyclohexyl)-3-methylpiperazine-1-carboxylate (Compound 32)

A solution of (R)-1,1,1-trifluoropropan-2-ol in ether (50%) (148 mg, 0.974 mmol) and CDI (154 mg, 0.950 mmol) in anhydrous THF (4 mL) was stirred at room temperature for 2.5 h. Next, (R)-1-((1r,4R)-4-(2-fluoro-4-(methylsulfonyl)phenoxy)methyl)cyclohexyl)-2-methylpiperazine hydrochloride (100 mg, 0.238 mmol) was added, followed by triethylamine (4.0 mL, 28.5 mmol). The heterogeneous mixture was stirred at 70° C. for 20 h. The mixture was diluted with water and extracted with EtOAc. The organic phase was dried over MgSO₄ and concentrated. The residue was purified by column chromatography to give the title compound as a white solid (40 mg, 32.1%). Exact mass calculated for C₂₃H₃₂F₄N₂O₅S: 524.2. found LCMS m/z=525.0 [M+H]⁺; ¹H NMR (400 MHz, CDCl₃) δ ppm 0.80-1.08 (m, 6H), 1.19 (d, J=6.6 Hz, 3H), 1.20-1.35 (m, 1H), 1.53-1.70 (m, 2H), 1.75-1.86 (m, 2H), 2.10-2.28 (m, 1H), 2.45-2.65 (m, 4H), 2.73-3.10 (m, 2H), 2.82 (s, 3H), 3.40-3.70 (m, 2H), 3.70 (d, J=6.2 Hz, 2H), 5.00-5.07 (m, 1H), 6.85 (t, J=8.1 Hz, 1H), 7.41-7.47 (m, 2H).

### Example 1.32

### Preparation of tert-Butyl 4-((1r,4r)-4-((2-Fluoro-4-(methylsulfonyl)phenylamino)methyl)cyclohexyl)piperazine-1-carboxylate (Compound 33) and tert-Butyl 4-((1s,4s)-4-((2-Fluoro-4-(methylsulfonyl)phenylamino)methyl)cyclohexyl)piperazine-1-carboxylate (Compound 35)

To a solution of tert-butyl 4-(4-((methylsulfonyloxy)methyl)cyclohexyl)piperazine-1-carboxylate (111 mg, 0.295 mmol) and 2-fluoro-4-(methylsulfonyl)aniline (63.9 mg, 0.338 mmol) in DMA (3 mL) was added 60% sodium hydride (11.79 mg, 0.295 mmol). After stirring at room temperature for 10 min, the reaction was heated under microwave irradiation at 100° C. for 5 min. The mixture was purified by reverse-phase HPLC (5-95% CH₃CN/H₂O+0.01% TFA). Fractions containing mainly the trans isomer (more polar) were partially concentrated and the residue was partitioned between 1 M NaOH and DCM. The organic phases were combined, dried over MgSO₄, filtered, and concentrated to give tert-butyl 4-((1r,4r)-4-((2-fluoro-4-(methylsulfonyl)phenylamino)methyl)cyclohexyl)piperazine-1-carboxylate (35.2 mg, 0.075 mmol, 25.4%) as a white solid. Fractions containing mainly the cis isomer (less polar) were partially concentrated and the residue was partitioned between 1 M NaOH and DCM. The organic phases were combined, dried over MgSO4, filtered, and concentrated to give tert-butyl 4-((1s,4s)-4-(2-fluoro-4-(methylsulfonyl)phenylamino)methyl)cyclohexyl)piperazine-1-carboxylate (6.4 mg, 0.014 mmol, 5%). Trans isomer: Exact mass calculated for C₂₃H₃₆FN₃O₄S: 469.24. found: LCMS m/z=470.2 [M+H]⁺; ¹H NMR (400 MHz, CDCl₃) δ ppm 1.01-1.10 (m, 2H), 1.23-1.32 (m, 2H), 1.46 (s, 9H), 1.51-1.61 (m, 1H), 1.93-1.95 (m, 4H), 2.27-2.33 (m, 1H), 2.49-2.52 (m, 4H), 3.00 (s, 3H), 3.06-3.09 (m, 2H), 3.41-3.42 (m, 4H), 4.49-4.52 (m, 1H), 6.67 (m, 1H), 7.48-7.51 (m, 1H), 7.56-7.59 (m, 1H). Cis isomer: Exact mass calculated for C₂₃H₃₆FN₃O₄S: 469.24. found: LCMS m/z=470.2 [M+H]⁺; ¹H NMR (400 MHz, CDCl₃) δ ppm 1.24-1.30 (m, 1H), 1.54 (s, 9H), 1.57-1.72 (m, 7H), 1.87-1.90 (m, 1H), 2.26-2.30 (m, 1H), 2.47-2.50 (m, 4H), 3.01 (s, 3H), 3.15-3.19 (m, 2H), 3.42-3.45 (m, 4H), 4.46-4.49 (m, 1H), 6.69-7.26 (m, 1H), 7.48-7.51 (m, 1H), 7.57-7.59 (m, 1H).

### Example 1.33

### Preparation of tert-Butyl 4-(1r,4r)-4-((4-(Trifluoromethylthio)phenoxy)methyl)cyclohexyl)piperazine-1-carboxylate (Compound 34)

A mixture of tert-butyl 4-(4-((methylsulfonyloxy)methyl)cyclohexyl)piperazine-1-carboxylate (120 mg, 0.319 mmol), 4-(trifluoromethylthio)phenol (74 mg, 0.382 mmol) and K₂CO₃ (88 mg, 0.637 mmol) in DMF (2 mL) was stirred overnight at 90° C. The reaction was diluted with EtOAc (10 mL), washed with saturated NaHCO₃ (2×10 mL), dried, and concentrated. The solid residue was triturated with IPA to give the title compound (120 mg, 79%). Exact mass calculated for C₂₃H₃₃F₃N₂O₃S: 474.2. found LCMS m/z=475.4 [M+H]⁺; ¹H NMR (400 MHz, CDCl₃) δ ppm 1.05-1.17 (m, 2H), 1.23-1.36 (m, 2H), 1.46 (s, 9H), 1.67-1.80 (m, 1H), 1.91-2.01 (m, 4H), 2.30 (t, J=11.8 Hz, 1H), 2.48-2.55 (m, 4H), 3.39-3.46 (m, 4H), 3.77 (d, J=6.4 Hz, 2H), 6.89 (d, J=8.4 Hz, 2H), 7.55 (d, J=8.4 Hz, 2H).

### Example 1.34

### Preparation of tert-Butyl 4-(1r,4r)-4-((4-(Dimethylcarbamoyl)-2-fluorophenoxy)methyl)cyclohexyl)piperazine-1-carboxylate (Compound 38)

The title compound was prepared in a manner similar to that described in Example 1.33 using tert-butyl 4-(4-((methylsulfonyloxy)methyl)cyclohexyl)piperazine-1-carboxylate and 3-fluoro-4-hydroxy-N,N-dimethylbenzamide obtained from the HATU coupling of 3-fluoro-4-hydroxybenzoic acid with dimethylamine Exact mass calculated for C₂₅H₃₈FN₃O₄: 463.2. found LCMS m/z=464.4 [M+H]⁺.

### Example 1.35

### Preparation of (4-((1r,4r)-4-((2-Fluoro-4-(methylsulfonyl)phenoxy)methyl)cyclohexyl)piperazin-1-yl)(1-(trifluoromethyl)cyclopropyl)methanone (Compound 36)

A mixture of 1-(trifluoromethyl)cyclopropanecarboxylic acid (40 mg, 0.26 mmol), HATU (104 mg, 0.27 mmol) and triethylamine (41 μL, 0.31 mmol) in acetonitrile (1 mL) was stirred for 10 min at room temperature. 1-((1r,4r)-4-((2-Fluoro-4-(methylsulfonyl)phenoxy)methyl)cyclohexyl)piperazine dihydrochloride (115 mg, 0.26 mmol) was added to the reaction, and followed by triethylamine (85 μL, 0.65 mmol). The reaction was stirred for 1 h at room temperature, diluted with EtOAc (5 mL), washed with saturated NaHCO₃ (2×5 mL) and brine, dried with anhydrous MgSO₄ and concentrated. The residue was purified by preparative TLC to give the title compound (65 mg, 49%). Exact mass calculated for C₂₃H₃₀F₄N₂O₄S: 506.2. found LCMS m/z=507.4 [M+H]⁺; ¹H NMR (400 MHz, CDCl₃) δ ppm 1.08-1.21 (m, 4H), 1.26-1.37 (m, 4H), 1.76-1.88 (m, 1H), 1.92-2.04 (m, 4H), 2.33 (tt, J=3.2 and 11.4 Hz, 1H), 2.55-2.60 (m, 4H), 3.03 (s, 3H), 3.63-3.70 (m, 4H), 3.90 (d, J=6.0 Hz, 2H), 7.05 (t, J=8.6 Hz, 1H), 7.61-7.70 (m, 2H).

### Example 1.36

### Preparation of 1-((1r,4r)-4-((2-Fluoro-4-(methylsulfonyl)phenoxy)methyl)cyclohexyl)-4-((1-(trifluoromethyl)cyclopropyl)methyl)piperazine (Compound 39)

### Step A: Preparation of N-Methoxy-N-methyl-1-(trifluoromethyl)cyclopropane carboxamide

A mixture of 1-(trifluoromethyl)cyclopropanecarboxylic acid (2.2 g, 14.3 mmol), HATU (5.7 g, 15 mmol), and triethylamine (1.45 g, 14.3 mmol) in acetonitrile (10 mL) was stirred for 10 min at room temperature. N,O-Dimethylhydroxylamine hydrochloride (1.53 g, 15.7 mmol) was added into the reaction, and followed by triethylamine (1.74 g, 16.8 mmol). The reaction was stirred for 3 h at room temperature, diluted with EtOAc, washed with 1 N HCl (twice) and brine, dried, and concentrated. The residue was purified by column chromatography to give the title compound (2.2 g, 78%). Exact mass calculated for C₇H₁₀F₃NO₂: 197.1. found LCMS m/z=198.2 [M+H]⁺.

### Step B: Preparation of 1-(Trifluoromethyl)cyclopropanecarbaldehyde

Powdered LiAlH₄ (385 mg, 10.1 mmol) was added to anhydrous Et₂O (10 mL) and cooled to 0° C. under an inert atmosphere. N-Methoxy-N-methyl-1-(trifluoromethyl)cyclopropanecarboxamide (2.0 g, 10.1 mmol) in Et₂O (4 mL) was added dropwise to the cloudy LiAlH₄ solution over 3 min with vigorous stirring. The reaction was stirred for 1 h at the same temperature, quenched carefully with H₂O (0.45 mL), added NaOH (15 wt % in water, 0.45 mL) dropwise, and followed by H₂O again (0.45 mL). The reaction slurry was filtered though a pad of Celite®, and washed with Et₂O (2×10 mL). About two thirds of the volatile solvent was carefully removed under ˜0.5 atm without using a heating bath. The resulting solution of 1-(trifluoromethyl)cyclopropanecarbaldehyde in Et₂O was used in the next step without further purification.

### Step C: Preparation of 1-(1r,4r)-4-((2-Fluoro-4-(methylsulfonyl)phenoxy)methyl)cyclohexyl)-4-((1-(trifluoromethyl)cyclopropyl)methyl)piperazine (Compound 39)

To a suspension of 1-((1r,4r)-4-((2-fluoro-4-(methylsulfonyl)phenoxy)methyl)cyclohexyl)piperazine dihydrochloride (70 mg, 0.16 mmol) in DCM (2 mL) was added triethylamine (42 μL, 0.32 mmol), followed by 1-(trifluoromethyl)cyclopropanecarbaldehyde (27 mg, 0.19 mmol) and AcOH (19 mg, 0.32 mmol). After stirring for 10 min at room temperature, NaBH(OAc)₃ (86 mg, 0.405 mmol) was added into the reaction mixture. The reaction was stirred overnight at 30° C., cooled to room temperature and quenched with saturated NaHCO₃. The reaction was diluted with H₂O, and extracted with DCM (twice). The organic phases were combined, washed with saturated NaHCO₃ and brine, dried, and concentrated. The residue was purified by preparative TLC to give the title compound (25 mg, 31%). Exact mass calculated for C₂₃H₃₂F₄N₂O₃S: 492.2. found LCMS m/z=493.4 [M+H]⁺; ¹H NMR (400 MHz, CDCl₃) δ ppm 0.60-0.66 (m, 2H), 0.96-1.00 (m, 2H), 1.07-1.19 (m, 2H), 1.25-1.37 (m, 2H), 1.76-1.87 (m, 1H), 1.95-2.05 (m, 4H), 2.27 (t, J=11.2 Hz, 1H), 2.45-2.55 (m, 6H), 2.59 (bs, 4H), 3.03 (s, 3H), 3.89 (d, J=6.4 Hz, 2H), 7.05 (t, J=7.8 Hz, 1H), 7.61-7.69 (m, 2H).

### Example 1.37

### Preparation of tert-Butyl 4-(1r,4r)-4-((4-(Cyclopropylsulfonyl)-2-fluorophenoxy)methyl)cyclohexyl)piperazine-1-carboxylate (Compound 61)

To a solution of tert-butyl 4-((1r,4r)-4-((methylsulfonyloxy)methyl)cyclohexyl)piperazine-1-carboxylate (0.697 g, 1.85 mmol) and 4-(cyclopropylsulfonyl)-2-fluorophenol (0.560 g, 2.59 mmol) in DMF (10 mL) was added potassium carbonate (0.511 g, 3.70 mmol). The mixture was heated at 80° C. for 20 h. The mixture was filtered and the solid cake was washed with EtOAc. The filtrate was concentrated under reduced pressure to remove EtOAc. The DMF solution was poured into water (50 mL). The mixture was concentrated under reduced pressure to dryness and the residue was purified by preparative HPLC and column chromatography (basic column) (2^{nd} purification) to give the title compound (0.0955 g, 10.39%) as a white solid. Exact mass calculated for C₂₅H₃₇FN₂O₅S: 496.2. found: LCMS m/z=497.6 [M+H]⁺; ¹H NMR (400 MHz, CDCl₃) δ ppm 1.00-1.05 (m, 2H), 1.09-1.18 (m, 2H), 1.24-1.36 (m, 4H), 1.46 (s, 9H), 1.78-1.86 (m, 1H), 1.95-2.04 (m, 4H), 2.28-2.34 (m, 1H), 2.41-2.45 (m, 1H), 2.51-2.53 (m, 4H), 3.41-3.44 (m, 4H), 3.90 (d, J=4.0 Hz, 2H), 7.04 (t, J=8.0 Hz, 1H), 7.57-7.64 (m, 2H).

### Example 1.38

### Preparation of 3-Cyclopropyl-5-(4-((1r,4r)-4-((2-fluoro-4-(methylsulfonyl)phenoxy)methyl)cyclohexyl)piperazin-1-yl)-1,2,4-oxadiazole (Compound 40)

### Step A: Preparation of Hydroxycyclopropanecarboximidamide

A solution of cyclopropanecarbonitrile (5 g, 74.6 mmol) and hydroxylamine (50 wt. % in water, 5.91 g, 89.5 mmol) in EtOH (15 mL) was stirred overnight at 80° C. The mixture was cooled to room temperature and the volatile solvent was removed under reduced pressure to give the title compound (7.0 g, 94%). The compound was used in the next step without further purification. Exact mass calculated for C₄H₈N₂O: 100.1. found LCMS m/z=101.2 [M+H]⁺. ¹H NMR (400 MHz, DMSO-d₆) δ ppm 0.54-0.60 (m, 2H), 0.61-0.66 (m, 2H), 1.28-1.36 (m, 1H), 5.18 (bs, 2H), 8.68 (bs, 1H).

### Step B: Preparation of 3-Cyclopropyl-5-(4-((1r,4r)-4-((2-fluoro-4-(methylsulfonyl)phenoxy)methyl)cyclohexyl)piperazin-1-yl)-1,2,4-oxadiazole (Compound 40)

To a solution of 4-((1r,4r)-4-((2-fluoro-4-(methylsulfonyl)phenoxy)methyl)cyclohexyl)piperazine-1-carbonitrile (150 mg, 0.38 mmol) as described in Example 1.18, Step A, and hydroxycyclopropanecarboximidamide (65 mg, 0.65 mmol) in THF (5 mL) was added 0.5 M ZnCl₂ in THF (1.35 mL, 0.67 mmol) at room temperature. After stirring for 3 h at room temperature, 4 M HCl in dioxane (0.68 mL, 2.71 mmol) was added into the reaction. The reaction was stirred for 4 h at 80° C. and cooled to room temperature. The reaction was diluted with DCM (15 mL), washed with saturated NaHCO₃ (2×10 mL), dried with anhydrous MgSO₄ and concentrated. The solid residue was triturated with IPA to give the title compound (105 mg, 58%). Exact mass calculated for C₂₃H₃₁FN₄O₄S: 478.2. found LCMS m/z=479.4 [M+H]⁺; ¹H NMR (400 MHz, CDCl₃) δ ppm 0.91-0.97 (m, 4H), 1.08-1.21 (m, 2H), 1.26-1.38 (m, 2H), 1.77-1.90 (m, 2H), 1.91-2.05 (m, 4H), 2.36 (t, J=11.4 Hz, 1H), 2.60-2.66 (m, 4H), 3.03 (s, 3H), 3.54-3.60 (m, 4H), 3.90 (d, J=6.4 Hz, 2H), 7.05 (t, J=7.8 Hz, 1H), 7.62-7.69 (m, 2H).

### Example 1.39

### Preparation of tert-Butyl 4-(1r,4r)-4-((2-Fluoro-4-(1H-tetrazol-1-yl)phenoxy)methyl)cyclohexyl)piperazine-1-carboxylate (Compound 49)

### Step A: Preparation of 4-Amino-2-fluorophenol

A mixture of 2-fluoro-4-nitrophenol (5.78 g, 36.8 mmol) and palladium on carbon (10%, 50% water, Degussa type, 0.525 g, 0.247 mmol) in 100 mL EtOH was shaken in a Parr shaker at 40 psi for 30 min. The reaction mixture was filtered though Celite®, washing with additional EtOH. The filtrate and washings were combined, concentrated, and dried under high vacuum to give the title compound (4.47 g, 96%) as a brown solid. Exact mass calculated for C₆H₆FNO: 127.04. found: LCMS m/z=128.0 [M+H]⁺; ¹H NMR (400 MHz, DMSO-d₆) δ ppm 4.69 (s, 2H), 6.19-6.22 (m, 1H), 6.33-6.37 (m, 1H), 6.60-6.65 (m, 1H), 8.54 (s, 1H).

### Step B: Preparation of 2-Fluoro-4-(1H-tetrazol-1-yl)phenol

A mixture of 4-amino-2-fluorophenol (1.01 g, 7.95 mmol), sodium azide (0.73 g, 11.23 mmol), and triethoxymethane (1.7 mL, 10.29 mmol) in AcOH (10 mL) was stirred at 100° C. (oil bath). After 20 min, the mixture was partitioned between water and EtOAc. The organic phases were combined, dried over MgSO₄, filtered, and concentrated. The residue was purified by column chromatography (SiO₂, hexane/EtOAc gradient). Fractions containing the desired product were combined and concentrated. The residue was triturated with MTBE to give the title compound (0.403 g, 26.7%) as a tan solid. Exact mass calculated for C₇H₅FN₄O: 180.04. found: LCMS m/z=181.2 [M+H]⁺; ¹H NMR (400 MHz, DMSO) δ ppm 7.15-7.19 (m, 1H), 7.54-7.56 (m, 1H), 7.79-7.82 (m, 1H), 9.94 (s, 1H), 10.6 (s, 1H).

### Step C: Preparation of tert-Butyl 4-(1r,4r)-4-((2-Fluoro-4-(1H-tetrazol-1-yl)phenoxy)methyl)cyclohexyl)piperazine-1-carboxylate (Compound 49)

A mixture of tert-butyl 4-(4-((methylsulfonyloxy)methyl)cyclohexyl)piperazine-1-carboxylate (527 mg, 1.260 mmol), 2-fluoro-4-(1H-tetrazol-1-yl)phenol (201 mg, 1.060 mmol), and cesium carbonate (460 mg, 1.412 mmol) in DMF (20 mL) was stirred at 80° C. (oil bath) for 7 h. The mixture was poured into ice. After the ice melted, the resulting suspension was filtered, and the cake was washed with additional water and dried under reduced pressure. The solid was purified by column chromatography (Biotage® KP-NH™, hexane/EtOAc gradient) to give the title compound (208 mg, 42.6%) as a white solid. Exact mass calculated for C₂₃H₃₃FN₆O₃: 460.26. found: LCMS m/z=461.6 [M+H]⁺; ¹H NMR (400 MHz, CDCl₃) δ ppm 1.11-1.20 (m, 2H), 1.28-1.37 (m, 2H), 1.46 (s, 9H), 1.80-1.85 (m, 1H), 1.96-2.04 (m, 4H), 2.29-2.35 (m, 1H), 2.51-2.54 (m, 4H), 3.42-3.44 (m, 4H), 3.90 (d, J=6.3 Hz, 2H), 7.07-7.12 (m, 1H), 7.38-7.41 (m, 1H), 7.46-7.49 (m, 1H), 8.89 (s, 1H).

### Example 1.40

### Preparation of tert-Butyl 4-((1r,4r)-4-((2-Fluoro-4-(4H-1,2,4-triazol-4-yl)phenoxy)methyl)cyclohexyl)piperazine-1-carboxylate (Compound 41)

### Step A: Preparation of 2-Fluoro-4-(4H-1,2,4-triazol-4-yl)phenol

A mixture of 4-amino-2-fluorophenol (509 mg, 4.00 mmol), N′-formylformohydrazide (426 mg, 4.84 mmol), and p-toluenesulfonic acid (826 mg, 4.34 mmol) in toluene (15 mL) and DMF (1.5 mL) was heated under microwave irradiation at 120° C. for 3 h. The mixture was concentrated and the residue was partitioned between DCM and water. The organic phases were combined, dried over MgSO₄, filtered, and concentrated. The residue was purified by column chromatography (SiO₂, hexane/EtOAc gradient and then EtOAc/MeOH 10:1) to give the title compound as a tan solid (262 mg, 36.5%). Exact mass calculated for C₈H₆FN₃O: 179.05. found: LCMS m/z=180.2 [M+H]⁺; ¹H NMR (400 MHz, DMSO-d₆) δ ppm 7.06-7.10 (m, 1H), 7.32-7.35 (m, 1H), 7.62-7.66 (m, 1H), 8.98 (s, 2H). 10.2 (s, 1H).

### Step B: Preparation of tert-Butyl 4-((1r,4r)-4-((2-Fluoro-4-(4H-1,2,4-triazol-4-yl)phenoxy)methyl)cyclohexyl)piperazine-1-carboxylate (Compound 41)

A mixture of tert-butyl 4-(4-((methylsulfonyloxy)methyl)cyclohexyl)piperazine-1-carboxylate (627 mg, 1.499 mmol), 2-fluoro-4-(4H-1,2,4-triazol-4-yl)phenol (204 mg, 1.139 mmol), and cesium carbonate (580 mg, 1.780 mmol) in DMF (20 mL) was heated under microwave irradiation at 100° C. for 1 h. The mixture was poured into ice. After the ice melted, the resulting suspension was filtered, and the cake was washed with additional water and dried under reduced pressure to give the title compound as a white solid (343 mg, 65.5%). Exact mass calculated for C₂₄H₃₄FN₅O₃: 459.26. found: LCMS m/z=460.2 [M+H]⁺; ¹H NMR (400 MHz, CDCl₃) δ ppm 1.10-1.19 (m, 2H), 1.28-1.36 (m, 2H), 1.46 (s, 9H), 1.80-1.85 (m, 1H), 1.95-2.03 (m, 4H), 2.29-2.35 (m, 1H), 2.51-2.54 (m, 4H), 3.42-3.44 (m, 4H), 3.90 (d, J=6.2 Hz, 2H), 7.03-7.16 (m, 3H), 8.38 (s, 2H).

### Example 1.41

### Preparation of tert-Butyl 4-((1r,4r)-4-((2-Fluoro-4-(1H-1,2,3-triazol-1-yl)phenoxy)methyl)cyclohexyl)piperazine-1-carboxylate (Compound 56)

### Step A: Preparation of 2-Fluoro-4-(1H-1,2,3-triazol-1-yl)phenol and 2-fluoro-4-(2H-1,2,3-triazol-2-yl)phenol

A mixture of 4-bromo-2-fluorophenol (1.05 g, 5.50 mmol), 2H-1,2,3-triazole (1.14 mL, 19.68 mmol), copper(I) iodide (97 mg, 0.509 mmol), potassium phosphate (2.25 g, 10.60 mmol), and N¹,N²-dimethylethane-1,2-diamine (0.110 mL, 1.022 mmol) in DMF (15 mL) was heated under microwave irradiation at 150° C. for 16 h. The mixture was poured into water, neutralized by the addition of 6 M HCl, and extracted with EtOAc. The organic phases were combined, dried over MgSO₄, filtered, and concentrated. The residue was purified by column chromatography (SiO₂, hexane/EtOAc gradient) to give 2-fluoro-4-(1H-1,2,3-triazol-1-yl)phenol (113 mg, 11%). Exact mass calculated for C₈H₆FN₃O: 179.05. found: LCMS m/z=180.2 [M+H]⁺; ¹H NMR (400 MHz, DMSO) δ ppm 7.11-7.15 (m, 1H), 7.55-7.57 (m, 1H), 7.74-7.78 (m, 1H), 7.92 (s, 1H), 8.70 (s, 1H). 10.3 (s, 1H). Also isolated was 2-fluoro-4-(2H-1,2,3-triazol-2-yl)phenol (152 mg, 13%). Exact mass calculated for C₈H₆FN₃O: 179.05. found: LCMS m/z=180.0 [M+H]⁺; ¹H NMR (400 MHz, DMSO) δ ppm 7.09-7.14 (m, 1H), 7.66-7.68 (m, 1H), 7.74-7.77 (m, 1H), 8.06 (s, 2H). 10.2 (s, 1H).

### Step B: Preparation of tert-Butyl 4-(1r,4r)-4-((2-Fluoro-4-(1H-1,2,3-triazol-1-yl)phenoxy)methyl)cyclohexyl)piperazine-1-carboxylate (Compound 56)

A mixture of tert-butyl 4-(4-((methylsulfonyloxy)methyl)cyclohexyl)piperazine-1-carboxylate (348 mg, 0.832 mmol), 2-fluoro-4-(1H-1,2,3-triazol-1-yl)phenol (100 mg, 0.558 mmol), and cesium carbonate (267 mg, 0.819 mmol) in 10 mL DMF was heated under microwave irradiation at 100° C. for 1 h. The mixture was poured into ice. After the ice melted, the resulting suspension was filtered, and the cake was washed with additional water and dried under reduced pressure to give the title compound as a white solid (194 mg, 50.7%). Exact mass calculated for C₂₄H₃₄FN₅O₃: 459.26. found: LCMS m/z=460.4 [M+H]⁺; ¹H NMR (400 MHz, CDCl₃) δ ppm 1.10-1.19 (m, 2H), 1.26-1.36 (m, 2H), 1.46 (s, 9H), 1.80-1.85 (m, 1H), 1.95-2.04 (m, 4H), 2.29-2.35 (m, 1H), 2.51-2.54 (m, 4H), 3.42-3.44 (m, 4H), 3.88 (d, J=6.2 Hz, 2H), 7.03-7.08 (m, 1H), 7.42-7.44 (m, 1H), 7.50-7.53 (m, 1H), 7.83 (s, 1H), 7.90 (s, 1H).

### Example 1.42

### Preparation of tert-Butyl 4-((1r,4r)-4-((2-Fluoro-4-(2H-1,2,3-triazol-2-yl)phenoxy)methyl)cyclohexyl)piperazine-1-carboxylate (Compound 43)

A mixture of tert-butyl 4-(4-((methylsulfonyloxy)methyl)cyclohexyl)piperazine-1-carboxylate (370 mg, 0.884 mmol), cesium carbonate (340 mg, 1.044 mmol), and 2-fluoro-4-(2H-1,2,3-triazol-2-yl)phenol (140 mg, 0.664 mmol) in DMF (10 mL) was heated under microwave irradiation at 100° C. for 1 h. The mixture was poured into ice. After the ice melted, the resulting suspension was filtered, and the cake was washed with additional water, and purified by reverse-phase HPLC (5-95% CH₃CN/H₂O+0.1% TFA). Fractions containing the desired product were partially concentrated and the residue was partitioned between 1 M NaOH and DCM. The organic phases were combined, dried over MgSO₄, filtered, and concentrated to give the title compound as a white solid (165 mg, 54.1%). Exact mass calculated for C₂₄H₃₄FN₅O₃: 459.26. found: LCMS m/z=460.4 [M+H]⁺; ¹H NMR (400 MHz, CDCl₃) δ ppm 1.10-1.19 (m, 2H), 1.26-1.36 (m, 2H), 1.46 (s, 9H), 1.80-1.85 (m, 1H), 1.94-2.04 (m, 4H), 2.29-2.35 (m, 1H), 2.51-2.53 (m, 4H), 3.41-3.44 (m, 4H), 3.87 (d, J=6.3 Hz, 2H), 7.00-7.04 (m, 1H), 7.76-7.85 (m, 4H).

### Example 1.43

### Preparation of 5-Chloro-2-(4-((1r,4r)-4-((2-fluoro-4-(1H-tetrazol-1-yl)phenoxy)methyl)cyclohexyl)piperazin-1-yl)pyrimidine (Compound 45)

### Step A: Preparation of 1-(1r,4r)-4-((2-Fluoro-4-(1H-tetrazol-1-yl)phenoxy)methyl)cyclohexyl)piperazine Dihydrochloride

To a solution of tert-butyl 4-((1r,4r)-4-((2-fluoro-4-(1H-tetrazol-1-yl)phenoxy)methyl)cyclohexyl)piperazine-1-carboxylate (162 mg, 0.352 mmol) in DCM (2 mL), was added 4 M hydrogen chloride in dioxane (2 mL, 8.00 mmol). After stirring at room temperature for 2 h, the mixture was concentrated and dried under high vacuum to give the title compound as a white solid (152 mg, 100%). Exact mass calculated for C₁₈H₂₆N₆O: 360.21. found: LCMS m/z=361.4 [M+H]⁺.

### Step B: Preparation of 5-Chloro-2-(4-((1r,4r)-4-((2-fluoro-4-(1H-tetrazol-1-yl)phenoxy)methyl)cyclohexyl)piperazin-1-yl)pyrimidine (Compound 45)

A mixture of 1-((1r,4r)-4-((2-fluoro-4-(1H-tetrazol-1-yl)phenoxy)methyl)cyclohexyl)piperazine dihydrochloride (50 mg, 0.115 mmol), 2,5-dichloropyrimidine (53 mg, 0.356 mmol), and triethylamine (80 μL, 0.574 mmol) in IPA (2 mL) was stirred at 80° C. (oil bath) for 3 h. The mixture was partitioned between DCM and 1 M HCl. The organic phases were combined, dried over MgSO₄, filtered, and concentrated. The residue was triturated with MTBE and hexane. The solid was filtered off, washed with additional MTBE/hexane, and dried under high vacuum to give the title compound as a white solid (53.1 mg, 97%). Exact mass calculated for C₂₂H₂₆ClFN₈O: 472.19. found: LCMS m/z=473.2 [M+H]⁺; ¹H NMR (400 MHz, CDCl₃) δ ppm 1.12-1.22 (m, 2H), 1.31-1.39 (m, 2H), 1.82-1.87 (m, 1H), 2.00-2.05 (m, 4H), 2.33-2.39 (m, 1H), 2.62-2.65 (m, 4H), 3.78-3.81 (m, 4H), 3.91 (d, J=6.3 Hz, 2H), 7.08-7.12 (m, 1H), 7.38-7.40 (m, 1H), 7.46-7.49 (m, 1H), 8.22 (s, 2H), 8.88 (s, 1H).

### Example 1.44

### Preparation of (R)-1,1,1-Trifluoropropan-2-yl 4-((1r,4R)-4-((2-Fluoro-4-(1H-tetrazol-1-yl)phenoxy)methyl)cyclohexyl)piperazine-1-carboxylate (Compound 44)

A solution of (R)-1,1,1-trifluoropropan-2-ol (100 μL, 0.331 mmol) and CDI (60 mg, 0.370 mmol) in THF (2 mL) was stirred at room temperature. After 45 min, 1-((1r,4r)-4-((2-fluoro-4-(1H-tetrazol-1-yl)phenoxy)methyl)cyclohexyl)piperazine dihydrochloride (39.2 mg, 0.090 mmol) and triethylamine (63 μL, 0.452 mmol) were added. The mixture was stirred at 80° C. (oil bath) for 18 h and then purified by reverse-phase HPLC (5-95% CH₃CN/H₂O+0.1% TFA). Fractions containing desired product were partially concentrated and the residue was partitioned between 1 M NaOH and DCM. The organic phases were combined, dried over MgSO₄, filtered, and concentrated to give the title compound (22.8 mg, 0.046 mmol, 50.4%) as a white solid. Exact mass calculated for C₂₂H₂₈F₄N₆O₃: 500.22. found: LCMS m/z=501.6 [M+H]⁺; ¹H NMR (400 MHz, CDCl₃) δ ppm 1.11-1.37 (m, 4H), 1.40 (d, J=6.6 Hz, 3H), 1.80-1.85 (m, 1H), 1.95-2.04 (m, 4H), 2.31-2.37 (m, 1H), 2.54-2.58 (m, 4H), 3.48-3.53 (m, 4H), 3.90 (d, J=6.3 Hz, 2H), 5.21-5.28 (m, 1H), 7.07-7.11 (m, 1H), 7.38-7.40 (m, 1H), 7.46-7.49 (m, 1H), 8.89 (s, 1H).

### Example 1.45

### Preparation of 2,2,2-Trifluoroethyl 4-(1r,4r)-4-((2-Fluoro-4-(methylsulfonyl)phenoxy)methyl)cyclohexyl)piperazine-1-carboxylate (Compound 46)

A solution of 2,2,2-trifluoroethanol (50.8 mg, 0.507 mmol) and CDI (73.1 mg, 0.451 mmol) in THF (2 mL) was heated at 60° C. for 1 h. The mixture was allowed to cool to room temperature and 1-((1r,4r)-4-((2-fluoro-4-(methylsulfonyl)phenoxy)methyl)cyclohexyl)piperazine dihydrochloride (50 mg, 0.113 mmol) and triethylamine (0.126 mL, 0.902 mmol) were added. The mixture was heated at 90° C. for 15 h. The reaction mixture was diluted with EtOAc, washed with brine, dried over anhydrous Na₂SO₄, and concentrated under reduced pressure. The residue was triturated with IPA/hexanes (5:1, 1.2 mL) to give the title compound as a white solid (42.5 mg, 76%). Exact mass calculated for C₂₁H₂₈F₄N₂O₅S: 496.2. found: LCMS m/z=497.2 [M+H]⁺; ¹H NMR (400 MHz, CDCl₃) δ ppm 1.13-1.20 (m, 2H), 1.27-1.34 (m, 2H), 1.82 (m, 1H), 1.94-2.03 (m, 4H), 2.31-2.35 (m, 1H), 2.57 (bs, 4H), 3.03 (s, 3H), 3.50-3.53 (m, 4H), 3.90 (d, J=6.0 Hz, 2H), 4.48 (q, J=8.0 Hz, 2H), 7.05 (t, J=8.0 Hz, 1H), 7.63-7.69 (m, 2H).

### Example 1.46

### Preparation of 2,2,3,3,3-Pentafluoropropyl 4-(1r,4r)-4-((2-Fluoro-4-(methylsulfonyl)phenoxy)methyl)cyclohexyl)piperazine-1-carboxylate (Compound 48)

A solution of 2,2,3,3,3-pentafluoropropan-1-ol (76 mg, 0.507 mmol) and CDI (73.1 mg, 0.451 mmol) in THF (2 mL) was heated at 60° C. for 1 h. The reaction mixture was allowed to cool to room temperature and 1-((1r,4r)-4-((2-fluoro-4-(methylsulfonyl)phenoxy)methyl)cyclohexyl)piperazine dihydrochloride (50 mg, 0.113 mmol) and triethylamine (0.126 mL, 0.902 mmol) were added. The mixture was heated at 90° C. for 15 h. The reaction mixture was diluted with EtOAc, washed with brine, dried over anhydrous Na₂SO₄, and concentrated under reduced pressure. The residue was triturated with IPA/hexanes (5:1, 1.2 mL) to give the title compound as a white solid (20.5 mg, 33.3%).

The filtrate was concentrated under reduced pressure and purified by preparative HPLC. The fractions containing the desired product were combined, neutralized with saturated aqueous NaHCO₃ solution, and concentrated under reduced pressure to remove acetonitrile. The resulting precipitate was collected to give a second batch of the title compound as a white solid (22.6 mg, 36.7%). Exact mass calculated for C₂₂H₂₈F₆N₂O₅S: 546.2. found: LCMS m/z=547.2 [M+H]⁺; ¹H NMR (400 MHz, CDCl₃) δ ppm 1.10-1.20 (m, 2H), 1.26-1.36 (m, 2H), 1.80-1.84 (m, 1H), 1.94-2.02 (m, 4H), 2.31-2.37 (m, 1H), 2.56 (bs, 4H), 3.03 (s, 3H), 3.50 (bs, 4H), 3.90 (d, J=6.0 Hz, 2H), 4.56 (t, J=12.0 Hz, 2H), 7.05 (t, J=8.0 Hz, 1H), 7.63-7.69 (m, 2H).

### Example 1.47

### Preparation of 1,1,1,3,3,3-Hexafluoropropan-2-yl 4-((1r,4r)-4-((2-Fluoro-4-(methylsulfonyl)phenoxy)methyl)cyclohexyl)piperazine-1-carboxylate (Compound 42)

A solution of 1,1,1,3,3,3-hexafluoropropan-2-ol (34 μL, 0.327 mmol) and CDI (53 mg, 0.327 mmol) in THF (1 mL) was stirred at room temperature. After 50 min, 1-((1r,4r)-4-((2-fluoro-4-(methylsulfonyl)phenoxy)methyl)cyclohexyl)piperazine dihydrochloride (54 mg, 0.122 mmol) and triethylamine (85 μL, 0.610 mmol) were added. The reaction was stirred at 80° C. (oil bath) for 18 h. The mixture was purified by reverse-phase HPLC (5-95% CH₃CN/H₂O+0.1% TFA). Fractions containing desired product were partially concentrated and the residue was partitioned between 1 M NaOH and DCM. The organic phases were combined, dried over MgSO₄, filtered, and concentrated to give the title compound as a white solid (35.3 mg, 51.3%). Exact mass calculated for C₂₂H₂₇F₇N₂O₅S: 564.15. found: LCMS m/z=565.4 [M+H]⁺; ¹H NMR (400 MHz, CDCl₃) δ ppm 1.10-1.17 (m, 2H), 1.27-1.36 (m, 2H), 1.80-1.85 (m, 1H), 1.93-2.03 (m, 4H), 2.33-2.38 (m, 1H), 2.57-2.60 (m, 4H), 3.03 (s, 3H), 3.54-3.56 (m, 4H), 3.90 (d, J=6.2 Hz, 2H), 5.72-5.78 (m, 1H), 7.03-7.07 (m, 1H), 7.63-7.69 (m, 2H).

### Example 1.48

### Preparation of 1-Methylcyclobutyl 4-((1r,4r)-4-((2-Fluoro-4-(methylsulfonyl)phenoxy)methyl)cyclohexyl)piperazine-1-carboxylate (Compound 37)

In a dry 40 mL vial, cyclobutanone (0.856 mL, 11.41 mmol) was dissolved in dry ether (5.01 mL, 11.41 mmol). Methylmagnesium bromide (3 M in ether,5.71 mL, 17.12 mmol) was added dropwise at room temperature under a nitrogen atmosphere with stirring. An exotherm was observed and after 1 h, a grey suspension had formed. The reaction mixture was cooled to 0° C. then diluted with saturated aqueous NH₄Cl solution (4 mL) to give a gum which was dissolved in ether (100 mL) and saturated aqueous NH₄Cl solution (30 mL). The mixture was shaken, the organic layer was removed and dried with MgSO₄, and the solvent was evaporated to give an oil. This oil was dissolved in DCM (30 mL) and bis(2,5-dioxopyrrolidin-1-yl) carbonate (5.85 g, 22.83 mmol) was added at room temperature to give a white suspension. Next, triethylamine (4.77 mL, 34.2 mmol) was added, and the mixture was stirred for 3 days. The mixture was then concentrated and the residue was partitioned between EtOAc and 2 M aqueous NaHCO₃ solution. The organic phases were combined, dried over MgSO4, filtered, and concentrated to give crude 2,5-dioxopyrrolidin-1-yl 1-methylcyclobutyl carbonate as a brownish viscous oil that turned solid upon storage (2.67 g).

Crude 2,5-dioxopyrrolidin-1-yl 1-methylcyclobutyl carbonate (500 mg, 2.1 mmol) was dissolved in THF (3 mL) and 1-((1r,4r)-4-((2-fluoro-4-(methylsulfonyl)phenoxy)methyl)cyclohexyl)piperazine dihydrochloride (49.5 mg, 0.112 mmol) and triethylamine (77 μL, 0.552 mmol) were added. After stirring at room temperature for 1 h, the mixture was purified by reverse-phase HPLC (5-95% CH₃CN/H₂O+0.1% TFA). Fractions containing the desired product were basified by the addition of 1 M NaOH and partially concentrated, whereupon a solid precipitated. The solid was filtered off, washed with additional water, and dried under high vacuum to give the title compound as a white solid (20.5 mg, 38.0%). Exact mass calculated for C₂₄H₃₅FN₂O₅S: 482.23. found: LCMS m/z=483.4 [M+H]⁺; ¹H NMR (400 MHz, CDCl₃) δ ppm 1.10-1.18 (m, 2H), 1.27-1.36 (m, 2H), 1.45-1.87 (m, 3H), 1.52 (s, 3H), 1.94-2.02 (m, 4H), 2.09-2.14 (m, 2H), 2.28-2.35 (m, 3H), 2.52-2.54 (m, 4H), 3.03 (s, 3H), 3.42-3.45 (m, 4H), 3.90 (d, J=6.2 Hz, 2H), 7.03-7.07 (m, 1H), 7.62-7.68 (m, 2H).

### Example 1.49

### Preparation of tert-Butyl 4-((1r,4r)-4-((6-Cyano-2-methylpyrimidin-4-yloxy)methyl)cyclohexyl)piperazine-1-carboxylate (Compound 47)

### Step A: Preparation of tert-Butyl 4-(4-((6-Chloro-2-methylpyrimidin-4-yloxy)methyl)cyclohexyl)piperazine-1-carboxylate

To an ice-cooled solution of tert-butyl 4-(4-(hydroxymethyl)cyclohexyl)piperazine-1-carboxylate (446 mg, 1.495 mmol) and 4,6-dichloro-2-methylpyrimidine (363 mg, 2.227 mmol) in THF (40 mL), 1 M potassium tert-butoxide solution (2 mL, 2.000 mmol) was added slowly. After stirring at 0° C. for 30 min, the mixture was partially concentrated and the residue was partitioned between water and DCM. The organic phases were combined, dried over MgSO₄, filtered, and concentrated. The residue was purified by column chromatography (SiO₂, hexane/EtOAc+7 M NaOH in MeOH gradient) to give the title compound as a white solid containing approximately 22% of the corresponding cis isomer (470 mg, 74.0%). Exact mass calculated for C₂₁H₃₃ClN₄O₃: 424.22. found: LCMS m/z=425.4 [M+H]⁺; ¹H NMR (400 MHz, CDCl₃) δ ppm 1.04-1.13 (m, 2H), 1.24-1.32 (m, 2H), 1.46 (s, 9H), 1.68-1.74 (m, 1H), 1.92-2.04 (m, 4H), 2.44-2.47 (m, 1H), 2.48-2.52 (m, 4H), 2.58 (s, 3H), 3.41-4.43 (m, 4H), 4.16 (d, J=6.2 Hz, 2H), 6.55 (s, 1H).

### Step B: Preparation of tert-Butyl 4-((1r,4r)-4-((6-Cyano-2-methylpyrimidin-4-yloxy)methyl)cyclohexyl)piperazine-1-carboxylate (Compound 47)

A mixture of tert-butyl 4-(4-((6-chloro-2-methylpyrimidin-4-yloxy)methyl)cyclohexyl)piperazine-1-carboxylate (210 mg, 0.494 mmol), dicyanozinc (116 mg, 0.988 mmol), diacetoxypalladium (10 mg, 0.045 mmol), and 1,1′-bis(di-tert-butylphosphino)ferrocene (42 mg, 0.089 mmol) in DMA (10 mL) was heated under microwave irradiation at 120° C. for 4 h. The mixture was purified by column chromatography (SiO₂, hexane/EtOAc/MeOH+7 M NH₃ gradient) to give the title compound (48 mg, 23.4%). Exact mass calculated for C₂₂H₃₃N₂O₃: 415.26. found: LCMS m/z=416.3 [M+H]⁺; ¹H NMR (400 MHz, CDCl₃) δ ppm 1.04-1.14 (m, 2H), 1.24-1.34 (m, 2H), 1.46 (s, 9H), 1.68-1.78 (m, 1H), 1.90-1.97 (m, 4H), 2.26-2.32 (m, 1H), 2.50-2.52 (m, 4H), 2.63 (s, 3H), 3.41-3.43 (m, 4H), 4.20 (d, J=6.2 Hz, 2H), 6.86 (s, 1H).

### Example 1.50

### Preparation of (R)-1,1,1-Trifluoropropan-2-yl 4-(1r,4R)-4-((2-Fluoro-4-(methylsulfonyl)phenoxy)methyl)cyclohexyl)piperazine-1-carboxylate (Compound 50)

A mixture of (R)-1,1,1,trifluoropropan-2-ol (60 mg, 0.526 mmol) and CDI (85 mg, 0.526 mmol) in THF (2 mL) was stirred for 3 h at room temperature. 1-((1r,4r)-4-((2-Fluoro-4-(methylsulfonyl)phenoxy)methyl)cyclohexyl)piperazine dihydrochloride (65 mg, 0.175 mmol) and triethylamine (117 μL, 0.877 mmol) were added into the reaction. The reaction was stirred overnight at 70° C., and then cooled to room temperature. The reaction mixture was concentrated and the residue was purified by preparative TLC to give the title compound (35 mg, 39%). Exact mass calculated for C₂₂H₃₀F₄N₂O₅S: 510.1. found LCMS m/z=511.2 [M+H]⁺; ¹H NMR (400 MHz, CDCl₃) δ ppm 0.95-1.09 (m, 2H), 1.10-1.24 (m, 2H), 1.27 (d, J=6.6 Hz, 3H), 1.64-1.75 (m, 1H), 1.76-1.94 (m, 4H), 2.14-2.30 (m, 1H), 2.44 (bs, 4H), 2.89 (s, 3H), 3.38 (bs, 4H), 3.76 (d, J=6.1 Hz, 2H), 5.06-5.15 (m, 1H), 6.91 (t, J=8.4 Hz, 1H), 7.48-7.57 (m, 2H).

### Example 1.51

### Preparation of (S)-1,1,1-Trifluoropropan-2-yl 4-(1r,4S)-4-((2-Fluoro-4-(methylsulfonyl)phenoxy)methyl)cyclohexyl)piperazine-1-carboxylate (Compound 51)

The title compound was prepared in a manner similar to that described in Example 1.50 by using (S)-1,1,1,trifluoropropan-2-ol and CDI. Exact mass calculated for C₂₂H₃₀F₄N₂O₅S: 510.1. found LCMS m/z=511.2 [M+H]⁺; ¹H NMR (400 MHz, CDCl₃) δ ppm 0.88-1.03 (m, 2H), 1.05-1.18 (m, 2H), 1.22 (d, J=6.6 Hz, 3H), 1.60-1.71 (m, 1H), 1.72-1.89 (m, 4H), 2.10-2.24 (m, 1H), 2.39 (bs, 4H), 2.85 (s, 3H), 3.32 (bs, 4H), 3.72 (d, J=6.1 Hz, 2H), 5.02-5.10 (m, 1H), 6.87 (t, J=8.6 Hz, 1H), 7.43-7.52 (m, 2H).

### Example 1.52

### Preparation of 1,1,1-Trifluoro-2-methylpropan-2-yl 4-(1r,4r)-4-((2-Fluoro-4-(methylsulfonyl)phenoxy)methyl)cyclohexyl)piperazine-1-carboxylate (Compound 53)

A solution of 1,1,1-trifluoro-2-methylpropan-2-ol (116 mg, 0.902 mmol) and CDI (91 mg, 0.564 mmol) in THF (2 mL) was stirred overnight at 50° C. 1-((1r,4r)-4-((2-Fluoro-4-(methylsulfonyl)phenoxy)methyl)cyclohexyl)piperazine (100 mg, 0.226 mmol) and triethylamine (180 μL, 1.35 mmol) were added to the reaction, and then the reaction was heated under microwave irradiation for 1 h at 130° C. The reaction was concentrated and the residue was purified by preparative TLC to give the title compound (25 mg, 21%). Exact mass calculated for C₂₃H₃₂F₄N₂O₅S: 524.2. found LCMS m/z=525.4 [M+H]⁺; ¹H NMR (400 MHz, CDCl₃) δ ppm 1.04-1.21 (m, 2H), 1.22-1.43 (m, 2H), 1.62 (s, 6H), 1.73-1.85 (m, 1H), 1.92-2.05 (m, 4H), 2.40-2.75 (m, 5H), 2.96 (s, 3H), 3.28-3.74 (m, 4H), 3.84 (d, J=6.1 Hz, 2H), 6.98 (t, J=8.1 Hz, 1H), 7.55-7.64 (m, 2H).

### Example 1.53

### Preparation of S-Cyclopropyl 4-(1r,4r)-4-((2-fluoro-4-(methylsulfonyl)phenoxy)methyl)cyclohexyl)piperazine-1-carbothioate (Compound 54)

To a suspension of 1-((1r,4r)-4-((2-fluoro-4-(methylsulfonyl)phenoxy)methyl)cyclohexyl)piperazine dihydrochloride (150 mg, 0.338 mmol) and S-cyclopropyl 1H-imidazole-1-carbothioate (114 mg, 0.677 mmol) in THF (10 mL) in a sealed tube was added triethylamine (0.472 mL, 3.38 mmol). The mixture was heated at 100° C. for 18 h. The mixture was then concentrated under reduced pressure and purified by column chromatography to give the title compound as a beige solid (82.5 mg, 51.8%). Exact mass calculated for C₂₂H₃₁FN₂O₄S₂: 470.2. found LCMS m/z=471.6 [M+H]⁺; ¹H NMR (400 MHz, CDCl₃) δ ppm 0.60-0.63 (m, 2H), 1.01-1.06 (m, 2H), 1.10-1.18 (m, 2H), 1.26-1.35 (m, 2H), 1.79-1.83 (m, 1H), 1.93-2.02 (m, 4H), 2.11-2.17 (m, 1H), 2.31-2.36 (m, 1H), 2.55-2.58 (m, 4H), 3.03 (s, 3H), 3.44-3.50 (m, 4H), 3.90 (d, J=6.4 Hz, 2H), 7.05 (t, J=8.0 Hz, 1H), 7.62-7.69 (m, 2H).

### Example 1.54

### Preparation of S-Cyclobutyl 4-((1r,4r)-4-((2-Fluoro-4-(methylsulfonyl)phenoxy)methyl)cyclohexyl)piperazine-1-carbothioate (Compound 55)

A suspension of 1-((1r,4r)-4-((2-fluoro-4-(methylsulfonyl)phenoxy)methyl)cyclohexyl)piperazine dihydrochloride (150 mg, 0.338 mmol), S-cyclobutyl 1H-imidazole-1-carbothioate (123 mg, 0.677 mmol)) in THF (10 mL) and triethylamine (0.472 mL, 3.38 mmol) was heated in a sealed tube at 100° C. for 18 h. The mixture was concentrated under reduced pressure and purified by column chromatography to give the title compound as a light brown solid (93.3 mg, 56.9%). Exact mass calculated for C₂₃H₃₃FN₂O₄S₂: 484.2. found LCMS m/z=485.4 [M+H]⁺; ¹H NMR (400 MHz, CDCl₃) δ ppm 1.09-1.18 (m, 2H), 1.26-1.35 (m, 2H), 1.79-1.84 (m, 1H), 1.93-2.01 (m, 6H), 2.04-2.12 (m, 2H), 2.30-2.36 (m, 1H), 2.41-2.47 (m, 2H), 2.54-2.55 (m, 4H), 3.03 (s, 3H), 3.47-3.55 (m, 4H), 3.90 (d, J=6.4 Hz, 2H), 4.06-4.10 (m, 1H), 7.05 (t, J=8.0 Hz, 1H), 7.63-7.69 (m, 2H).

### Example 1.55

### Preparation of tert-Butyl 4-(1r,4r)-4-((2-Fluoro-4-(1H-1,2,4-triazol-1-yl)phenoxy)methyl)cyclohexyl)piperazine-1-carboxylate (Compound 57)

### Step A: Preparation of 2-Fluoro-4-(1H-1,2,4-triazol-1-yl)phenol

A mixture of 4-bromo-2-fluorophenol (1.7 g, 8.90 mmol), 1H-1,2,4-triazole (0.922 g, 13.35 mmol), copper(I) iodide (0.085 g, 0.445 mmol), potassium phosphate (3.78 g, 17.80 mmol), and N¹,N²-dimethylethane-1,2-diamine (0.157 g, 1.780 mmol) in DMF (30 mL) was heated under microwave irradiation at 110° C. for 17 h. The mixture was poured into water, neutralized with 6 M HCl, and extracted with EtOAc. The organic phases were combined, dried over MgSO₄, filtered, and concentrated. The residue was purified by column chromatography (0-100% EtOAc/hexanes) to give the title compound (62.8 mg, 3.94%). Exact mass calculated for C₈H₆FN₃O: 179.0. found LCMS m/z=180.1 [M+H]⁺.

### Step B: Preparation of tert-Butyl 4-(1r,4r)-4-((2-Fluoro-4-(1H-1,2,4-triazol-1-yl)phenoxy)methyl)cyclohexyl)piperazine-1-carboxylate (Compound 57)

A mixture of tert-butyl 4-(4-((methylsulfonyloxy)methyl)cyclohexyl)piperazine-1-carboxylate (151 mg, 0.401 mmol), cesium carbonate (163 mg, 0.501 mmol), and 2-fluoro-4-(1H-1,2,4-triazol-1-yl)phenol (59.9 mg, 0.334 mmol) in DMF (5 mL) was heated under microwave irradiation at 100° C. for 1 h. The mixture was poured into ice. After the ice melted, the resulting suspension was filtered, and the cake was washed with additional water and dried under reduced pressure. The residue was purified by column chromatography (0-100% EtOAc/hexanes) to give the title compound as a white solid (35.8 mg, 23.31%). Exact mass calculated for C₂₄H₃₄FN₅O₃: 459.3. found LCMS m/z=460.5 [M+H]⁺; ¹H NMR (400 MHz, CDCl₃) δ ppm 1.19-1.34 (m, 2H), 1.47 (s, 9H), 1.56-1.65 (m, 2H), 1.88 (bs, 1H), 2.12-2.24 (m, 4H), 2.81 (bs, 2H), 3.16-3.21 (m, 1H), 3.48 (bs, 4H), 3.89 (d, J=6.1 Hz, 2H), 4.19 (bs, 2H), 7.00-7.05 (m, 1H), 7.35-7.37 (m, 1H), 7.45-7.48 (m, 1H), 8.08 (s, 1H), 8.46 (s, 1H).

### Example 1.56

### Preparation of tert-Butyl 4-(1r,4r)-4-((2-Fluoro-4-(1H-pyrrol-1-yl)phenoxy)methyl)cyclohexyl)piperazine-1-carboxylate (Compound 58)

### Step A: Preparation of 2-Fluoro-4-(1H-pyrrol-1-yl)phenol

A mixture of 4-bromo-2-fluorophenol (2 g, 10.47 mmol), 1H-pyrrole (1.054 g, 15.71 mmol), copper(I) iodide (0.100 g, 0.524 mmol), potassium phosphate (4.45 g, 20.94 mmol), and N¹,N²-dimethylethane-1,2-diamine (0.185 g, 2.094 mmol) in DMF (30 mL) was heated under microwave irradiation at 110° C. for 17 h. The mixture was poured into water, neutralized by the addition of 6 M HCl, and extracted with EtOAc. The organic phases were combined, dried over MgSO₄, filtered, and concentrated. The residue was purified by column chromatography (0-100% EtOAc/hexanes) to give the title compound (91.3 mg, 0.515 mmol, 4.92%). Exact mass calculated for C₁₀H₈FNO: 177.0. found LCMS m/z=175.8 [M−H]⁺.

### Step B: Preparation of tert-Butyl 4-(1r,4r)-4-((2-Fluoro-4-(1H-pyrrol-1-yl)phenoxy)methyl)cyclohexyl)piperazine-1-carboxylate (Compound 58)

A mixture of tert-butyl 4-(4-((methylsulfonyloxy)methyl)cyclohexyl)piperazine-1-carboxylate (409 mg, 0.760 mmol), cesium carbonate (248 mg, 0.760 mmol), and 2-fluoro-4-(1H-pyrrol-1-yl)phenol (89.8 mg, 0.507 mmol) in DMF (5 mL) was heated under microwave irradiation at 100° C. for 1 h. The mixture was poured into ice. After the ice melted, the resulting suspension was filtered, and the cake was washed with additional water and dried under reduced pressure. The residue was purified by column chromatography (0-100% EtOAc/hexanes) to give the title compound as a white solid (54.8 mg, 23.63%). Exact mass calculated for C₂₆H₃₆FN₃O₃: 457.3. found LCMS m/z=458.4 [M+H]⁺; ¹H NMR (400 MHz, CDCl₃) δ ppm 1.09-1.19 (m, 2H), 1.26-1.36 (m, 2H), 1.46 (s, 9H), 1.78-1.80 (m, 1H), 1.94-2.03 (m, 4H), 2.28-2.34 (m, 1H), 2.51-2.53 (m, 4H), 3.42-3.44 (m, 4H), 3.84 (d, J=6.1 Hz, 2H), 6.32 (s, 2H), 6.95-6.98 (m, 3H), 7.05-7.08 (m, 1H), 7.12-7.15 (m, 1H).

### Example 1.57

### Preparation of tert-Butyl 4-((1r,4r)-4-((5-(Methylsulfonyl)pyridin-2-yloxy)methyl)cyclohexyl)piperazine-1-carboxylate (Compound 60)

### Step A: Preparation of tert-Butyl 4-((1r,4r)-4-((5-Bromopyridin-2-yloxy)methyl)cyclohexyl)piperazine-1-carboxylate

A mixture of tert-butyl 4-(4-((methylsulfonyloxy)methyl)cyclohexyl)piperazine-1-carboxylate (0.4 g, 0.744 mmol), cesium carbonate (0.242 g, 0.744 mmol), and 5-bromopyridin-2-ol (0.086 g, 0.496 mmol) in DMF (5 mL) was heated under microwave irradiation at 100° C. for 1 h. The mixture was poured into ice. After the ice melted, the resulting suspension was filtered, and the cake was washed with additional water and dried under reduced pressure. The residue was purified by column chromatography (0-100% EtOAc/hexanes) to give the title compound (62.8 mg, 27.9%). Exact mass calculated for C₂₁H₃₂BrN₃O₃: 453.2. found LCMS m/z=454.3 [M+H]⁺.

### Step B: Preparation of tert-Butyl 4-((1r,4r)-4-((5-(Methylsulfonyl)pyridin-2-yloxy)methyl)cyclohexyl)piperazine-1-carboxylate (Compound 60)

A mixture of tert-butyl 4-((1r,4r)-4-((5-bromopyridin-2-yloxy)methyl)cyclohexyl)piperazine-1-carboxylate (62.8 mg, 0.138 mmol), sodium methanesulfinate (35.3 mg, 0.346 mmol), copper(I) trifluoromethanesulfonate benzene complex (10.43 mg, 0.021 mmol), and N¹,N²-dimethylethane-1,2-diamine (4.46 μL, 0.041 mmol) in DMSO (3 mL) was heated under microwave irradiation at 120° C. for 2 h. The reaction was purified by preparative HPLC and then by column chromatography (10% MeOH in DCM). The fractions containing the desired product were concentrated, and the residue was sonicated in MTBE and filtered to give the title compound as a solid (21.7 mg, 34.6%). Exact mass calculated for C₂₂H₃₅N₃O₅S: 453.2. found LCMS m/z=454.4 [M+H]⁺; ¹H NMR (400 MHz, CDCl₃) δ ppm 1.09-1.15 (m, 2H), 1.25-1.33 (m, 2H), 1.46 (s, 9H), 1.75 (bs, 1H), 1.94-1.96 (m, 4H), 2.27-2.33 (m, 1H), 2.51 (s, 4H), 3.06 (s, 3H), 3.42 (s, 4H), 4.2 (d, J=6.1 Hz, 2H), 6.84 (d, J=8.9 Hz, 1H), 8.02 (d, J=8.9 Hz, 1H), 8.70 (s, 1H).

### Example 1.58

### Preparation of tert-Butyl 4-(1r,4r)-4-((2-Fluoro-4-(4-methyl-1H-pyrazol-1-yl)phenoxy)methyl)cyclohexyl)piperazine-1-carboxylate (Compound 59)

### Step A: Preparation of 2-Fluoro-4-(4-methyl-1H-pyrazol-1-yl)phenol

A mixture of 4-bromo-2-fluorophenol (3 g, 15.71 mmol), 4-methyl-1H-pyrazole (1.934 g, 23.56 mmol), copper(I) iodide (0.150 g, 0.785 mmol), potassium phosphate (6.67 g, 31.4 mmol), and N¹,N²-dimethylethane-1,2-diamine (0.277 g, 3.14 mmol) in DMF (45 mL) was heated under microwave irradiation at 150° C. for 17 h. The mixture was poured into water, neutralized with 6 M HCl, and extracted with EtOAc. The organic phases were combined, dried over MgSO₄, filtered, and concentrated. The residue was purified by column chromatography (hexane/EtOAc gradient) to give the title compound (100 mg, 3.31% yield). Exact mass calculated for C₁₀H₉FN₂O: 192.1. found LCMS m/z=193.2 [M+H]⁺.

### Step B: Preparation of tert-Butyl 4-(1r,4r)-4-((2-Fluoro-4-(4-methyl-1H-pyrazol-1-yl)phenoxy)methyl)cyclohexyl)piperazine-1-carboxylate (Compound 59)

A mixture of tert-butyl 4-(4-((methylsulfonyloxy)methyl)cyclohexyl)piperazine-1-carboxylate (294 mg, 0.780 mmol), cesium carbonate (254 mg, 0.780 mmol), and 2-fluoro-4-(4-methyl-1H-pyrazol-1-yl)phenol (100 mg, 0.520 mmol) in DMF (5 mL) was heated under microwave irradiation at 100° C. for 1 h. The mixture was poured into ice. After the ice melted, the resulting solid was filtered off, washed with additional water, and dried under high vacuum. The residue was purified by column chromatography (Biotage® SNAP KP-NH™, 0-100% EtOAc/Hexanes) to give the title compound (54.8 mg, 22.29% yield). Exact mass calculated for C₂₆H₃₇FN₄O₃: 472.3. found LCMS m/z=473.2 [M+H]⁺; ¹H NMR (400 MHz, CDCl₃) δ ppm 1.08-1.18 (m, 2H), 1.26-1.35 (m, 2H), 1.46 (s, 9H), 1.76-1.81 (m, 1H), 1.94-2.03 (m, 4H), 2.14 (s, 3H), 2.28-2.34 (m, 1H), 2.51-2.53 (m, 4H), 3.41-3.44 (m, 4H), 3.84 (d, J=6.1 Hz, 2H), 6.95-6.99 (m, 1H), 7.29 (d, J=2.78 Hz, 1H), 7.42 (d, J=2.78 Hz, 1H), 7.49 (s, 1H), 7.59 (s, 1H).

### Example 1.59

### Preparation of tert-Butyl 4-((1r,4r)-4-((2-Chloro-4-(methylsulfonyl)phenoxy)methyl)cyclohexyl)piperazine-1-carboxylate (Compound 62)

### Step A: Preparation of tert-Butyl 4-((1r,4r)-4-((4-Bromo-2-chlorophenoxy)methyl)cyclohexyl)piperazine-1-carboxylate

A mixture of tert-butyl 4-(4-((methylsulfonyloxy)methyl)cyclohexyl)piperazine-1-carboxylate (1.0662 g, 2.55 mmol), cesium carbonate (0.996 g, 3.06 mmol), and 4-bromo-2-chlorophenol (0.634 g, 3.06 mmol) in DMF (15 mL) was heated under microwave irradiation at 100° C. for 1 h. The mixture was poured into ice. After the ice melted, the resulting suspension was filtered, and the cake was washed with additional water and dried under reduced pressure to give the title compound (1.0184 g, 76%). Exact mass calculated for C₂₂H₃₂BrClN₂O₃: 486.1. found LCMS m/z=487.4 [M+H]⁺.

### Step B: Preparation of tert-Butyl 4-((1r,4r)-4-((2-Chloro-4-(methylsulfonyl)phenoxy)methyl)cyclohexyl)piperazine-1-carboxylate (Compound 62)

A mixture of tert-butyl 4-((1r,4r)-4-((4-bromo-2-chlorophenoxy)methyl)cyclohexyl)piperazine-1-carboxylate (1.0184 g, 2.087 mmol), sodium methanesulfinate (0.533 g, 5.22 mmol), copper(I) trifluoromethanesulfonate benzene complex (0.158 g, 0.313 mmol), and N¹,N²-dimethylethane-1,2-diamine (0.067 mL, 0.626 mmol) in DMSO (15 mL) was heated under microwave irradiation at 120° C. for 2 h. The reaction was purified by reverse-phase HPLC (5-95% CH₃CN/H₂O with 0.1% TFA). Fractions containing the desired product were concentrated, and the residue was sonicated in MTBE and filtered to give the title compound as solid (523.9 mg, 51.5%). Exact mass calculated for C₂₃H₃₅ClN₂O₅S: 486.2. found LCMS m/z=487.3 [M+H]⁺; ¹H NMR (400 MHz, CDCl₃) δ ppm 1.15-1.19 (m, 2H), 1.31-1.34 (m, 2H), 1.46 (s, 9H), 1.78-1.89 (m, 1H), 1.95-2.03 (m, 4H), 2.29-2.35 (m, 1H), 2.51-2.54 (m, 4H), 3.03 (s, 3H), 3.42-3.44 (m, 4H), 3.90 (d, J=6.3 Hz, 2H), 7.00 (d, J=8.59 Hz, 1H), 7.79 (dd, J=8.59 Hz, 1H), 7.93-7.94 (m, 1H).

### Example 1.60

### Preparation of tert-Butyl 4-((1r,4r)-4-((3-Chloro-4-(methylsulfonyl)phenoxy)methyl)cyclohexyl)piperazine-1-carboxylate (Compound 63)

### Step A: Preparation of 3-Chloro-4-(methylsulfonyl)phenol

A mixture of 4-bromo-3-chlorophenol (589 mg, 2.84 mmol), sodium methanesulfinate (725 mg, 7.10 mmol), copper(I) trifluoromethanesulfonate benzene complex (214 mg, 0.426 mmol), and N¹,N²-dimethylethane-1,2-diamine (0.092 mL, 0.852 mmol) in DMSO (15 mL) was heated under microwave irradiation at 120° C. for 2 h. The reaction mixture was purified by preparative, reverse-phase HPLC (5-95% CH₃CN/H₂O) to give the title compound (89.2 mg, 15.20%). Exact mass calculated for C₇H₇ClO₃S: 206.0. found LCMS m/z=206.6 [M+H]⁺.

### Step B: Preparation of tert-Butyl 4-((1r,4r)-4-((3-Chloro-4-(methylsulfonyl)phenoxy)methyl)cyclohexyl)piperazine-1-carboxylate (Compound 63)

A mixture of tert-butyl 4-(4-((methylsulfonyloxy)methyl)cyclohexyl)piperazine-1-carboxylate (181 mg, 0.432 mmol), potassium carbonate (149 mg, 1.079 mmol), and 3-chloro-4-(methylsulfonyl)phenol (89.2 mg, 0.432 mmol) in DMF (10 mL) was heated under microwave irradiation at 100° C. for 1 h. The mixture was purified by reverse-phase HPLC (5-95% CH₃CN/H₂O with 0.1% TFA) to give the title compound (96 mg, 43.4%). Exact mass calculated for C₂₃H₃₅ClN₂O₅S: 486.2. found LCMS m/z=487.4 [M+H]⁺; ¹H NMR (400 MHz, CDCl₃) δ ppm 1.19 (s, 1H), 1.21-1.31 (m, 2H), 1.47 (s, 9H), 1.55-1.65 (m, 2H), 1.81-1.88 (m, 1H), 2.07-2.11 (m, 2H), 2.21-2.24 (m, 2H), 2.80 (bs, 2H), 3.23 (s, 3H), 3.39-3.48 (m, 4H), 3.84 (d, J=6.1 Hz, 2H), 4.19 (bs, 2H), 6.89 (d, J=2.27 Hz, 1H), 7.01 (d, J=2.27 Hz, 1H), 8.04-8.05 (m, 1H).

### Example 1.61

### Preparation of 3-(4-((1r,4r)-4-((2-Fluoro-4-(methylsulfonyl)phenoxy)methyl)cyclohexyl)piperazin-1-yl)-5-(trifluoromethyl)-1,2,4-oxadiazole (Compound 65)

### Step A: Preparation of 4-((1r,4r)-4-((2-Fluoro-4-(methylsulfonyl)phenoxy)methyl)cyclohexyl)-N′-hydroxypiperazine-1-carboximidamide

To a solution of 4-((1r,4r)-4-((2-fluoro-4-(methylsulfonyl)phenoxy)methyl)cyclohexyl)piperazine-1-carbonitrile (150 mg, 0.379 mmol) described in Example 1.18, Step A, in EtOH (2 mL) was added hydroxylamine (50 wt % in H₂O, 32 mg, 0.493 mmol). The reaction was stirred at 75° C. for 4 h, during which time the hydroxylamine adduct precipitated. The reaction was cooled to room temperature, and then the solid was filtered, washed with IPA and hexane, and dried to give the title compound (120 mg, 75%). Exact mass calculated for C₁₉H₂₉FN₄O₄S: 428.1. found LCMS m/z=429.2 [M+H]⁺.

### Step B: Preparation of 3-(4-((1r,4r)-4-((2-Fluoro-4-(methylsulfonyl)phenoxy)methyl)cyclohexyl)piperazin-1-yl)-5-(trifluoromethyl)-1,2,4-oxadiazole (Compound 65)

4-((1r,4r)-4-((2-Fluoro-4-(methylsulfonyl)phenoxy)methyl)cyclohexyl)-N′-hydroxypiperazine-1-carboximidamide (120 mg, 0.28 mmol) was dissolved in pyridine (2 mL). 2,2,2-Trifluoroacetic anhydride (180 mg, 0.86 mmol) was added and the reaction was stirred for 2 h at 45° C. and then overnight at 25° C. The reaction was cooled to room temperature, and diluted with water to precipitate the product. The solid was collected by filtration, washed with H₂O and hexane, and dried to give the title compound (110 mg, 78.5%). Exact mass calculated for C₂₁H₂₆P₄N₄O₄S: 506.1. found LCMS m/z=507.2 [M+H]⁺; ¹H NMR (400 MHz, CDCl₃) δ ppm 1.09-1.22 (m, 2H), 1.28-1.40 (m, 2H), 1.77-1.89 (m, 1H), 1.94-2.06 (m, 4H), 2.32-2.42 (m, 1H), 2.63-2.72 (m, 4H), 3.03 (s, 3H), 3.49-3.54 (m, 4H), 3.90 (d, J=6.0 Hz, 2H), 7.05 (t, J=8.3 Hz, 1H), 7.62-7.70 (m, 2H).

### Example 1.62

### Preparation of 5-(4-((1r,4r)-4-((2-Fluoro-4-(methylsulfonyl)phenoxy)methyl)cyclohexyl)piperazin-1-yl)-3-(trifluoromethyl)-1,2,4-oxadiazole (Compound 64)

### Step A: Preparation of 4-((1r,4r)-4-((2-Fluoro-4-(methylsulfonyl)phenoxy)methyl)cyclohexyl)piperazine-1-carbonitrile

To a solution of 1-((1r,4r)-4-((2-fluoro-4-(methylsulfonyl)phenoxy)methyl)cyclohexyl)piperazine dihydrochloride (200 mg, 0.451 mmol) and diisopropylethylamine (0.393 mL, 2.255 mmol) in DCM (5 mL) at 0° C. was added cyanogen bromide (52.6 mg, 0.496 mmol). The mixture was stirred at room temperature for 30 min. The reaction was quenched with water and the organic layer was separated, dried over anhydrous Na₂SO₄, filtered, and concentrated under reduced pressure. The solid residue was triturated with IPA and the solid was collected to give the title compound (159.8 mg, 90%) as an off-white solid. Exact mass calculated for C₁₉H₂₆FN₃O₃S: 395.2. found: LCMS m/z=396.4 [M+H]⁺; ¹H NMR (400 MHz, CDCl₃) δ ppm 1.12-1.19 (m, 2H), 1.25-1.32 (m, 2H), 1.81 (m, 1H). 1.91-2.02 (m, 4H), 2.31-2.34 (m, 1H), 2.63-2.66 (m, 4H), 3.03 (s, 3H), 3.23-3.25 (m, 4H), 3.90 (d, J=8.0 Hz, 2H), 7.05 (t, J=8.0 Hz, 1H), 7.63-7.69 (m, 2H).

### Step B: Preparation of 5-(4-((1r,4r)-4-((2-Fluoro-4-(methylsulfonyl)phenoxy)methyl)cyclohexyl)piperazin-1-yl)-3-(trifluoromethyl)-1,2,4-oxadiazole (Compound 64)

To a solution of 4-((1r,4r)-4-((2-fluoro-4-(methylsulfonyl)phenoxy)methyl)cyclohexyl)piperazine-1-carbonitrile (152 mg, 0.384 mmol) and 2,2,2-trifluoro-N′-hydroxyacetimidamide (73.8 mg, 0.576 mmol) in THF (5 mL) was added 0.5 M zinc(II) chloride solution in THF (1.153 mL, 0.576 mmol) at room temperature. The mixture was stirred at room temperature for 3 h. To the solution was added 4 N hydrogen chloride solution in dioxane (0.576 mL, 2.306 mmol) and the mixture was heated at 80° C. for 20 h (16% conversion to product). To this mixture was added more 4 N hydrogen chloride solution in dioxane (0.576 mL, 2.306 mmol) and the mixture was continued to heat at 120° C. for 5 h under microwave irradiation. The mixture was filtered to remove the solid and the filtrate was diluted with DCM, washed with saturated aqueous NaHCO₃ solution, and concentrated to dryness under reduced pressure. The residue was purified by semi-prep HPLC and neutralized with saturated aqueous NaHCO₃ solution to give the title compound as a white solid (43.4 mg, 22.29%). Exact mass calculated for C₂₁H₂₆F₄N₄O₄S: 506.2. found: LCMS m/z=507.4 [M+H]⁺; ¹H NMR (400 MHz, CDCl₃) δ ppm 1.12-1.21 (m, 2H), 1.26-1.38 (m, 2H), 1.80-1.85 (m, 1H). 1.94-2.04 (m, 4H), 2.37-2.43 (m, 1H), 2.68-2.70 (m, 4H), 3.03 (s, 3H), 3.67-3.69 (m, 4H), 3.91 (d, J=8.0 Hz, 2H), 7.06 (t, J=8.0 Hz, 1H), 7.63-7.70 (m, 2H).

### Example 1.63

### Preparation of tert-Butyl 4-(1r,4r)-4-((2-Fluoro-4-(1H-imidazol-1-yl)phenoxy)methyl)cyclohexyl)piperazine-1-carboxylate (Compound 52)

### Step A: Preparation of 2-Fluoro-4-(1H-imidazol-1-yl)phenol

A mixture of 4-bromo-2-fluorophenol (3 g, 15.71 mmol), 1H-imidazole (1.069 g, 15.71 mmol), copper(I) iodide (0.150 g, 0.785 mmol), potassium phosphate (6.67 g, 31.4 mmol), and N¹,N²-dimethylethane-1,2-diamine (0.277 g, 3.14 mmol) in DMF (45 mL) was heated under microwave irradiation at 150° C. for 16 h. The mixture was poured into water, neutralized with 6 M HCl, and extracted with EtOAc. The organic phases were combined, dried over MgSO₄, filtered, and concentrated. The residue was purified by column chromatography (SiO₂, hexane/EtOAc gradient) to give the title compound (224 mg, 8.00% yield). Exact mass calculated for C₉H₇FN₂O: 178.1. found LCMS m/z=179.0 μM+H1⁺.

### Step B: Preparation of tert-Butyl 4-(1r,4r)-4-((2-Fluoro-4-(1H-imidazol-1-yl)phenoxy)methyl)cyclohexyl)piperazine-1-carboxylate (Compound 52)

A mixture of tert-butyl 4-(4-((methylsulfonyloxy)methyl)cyclohexyl)piperazine-1-carboxylate (251 mg, 0.667 mmol), 2-fluoro-4-(1H-imidazol-1-yl)phenol (178 mg, 1.000 mmol), and cesium carbonate (326 mg, 1.000 mmol) in DMF (5 mL) was heated under microwave irradiation at 100° C. for 1 h. The mixture was poured into ice. After the ice melted, the resulting suspension was filtered, and the cake was washed with additional water and dried under reduced pressure. The residue was purified by column chromatography (0-100% EtOAc/hexanes) to give the title compound (121.2 mg, 39.6% yield). Exact mass calculated for C₂₅H₃₅FN₄O₃: 458.3. found LCMS m/z=459.4 [M+H]⁺; ¹H NMR (400 MHz, CDCl₃) δ ppm 1.09-1.19 (m, 2H), 1.26-1.36 (m, 2H), 1.46 (s, 9H), 1.79-1.82 (m, 1H), 1.95-2.04 (m, 4H), 2.29-2.34 (m, 1H), 2.51-2.54 (m, 4H), 3.42-3.44 (m, 4H), 3.86 (d, J=6.1 Hz, 2H), 6.99-7.03 (m, 1H), 7.07-7.10 (m, 1H), 7.12-7.19 (m, 1H), 7.18-7.19 (m, 2H), 7.75 (s, 1H).

### Example 1.64

### Preparation of 1-(Trifluoromethyl)cyclobutyl 4-(1r,4r)-4-((2-Fluoro-4-(methylsulfonyl)phenoxy)methyl)cyclohexyl)piperazine-1-carboxylate (Compound 66)

### Step A: Preparation of 1-(Trifluoromethyl)cyclobutanol

To a solution of cyclobutanone (2.5 mL, 33.4 mmol) and trimethyl(trifluoromethyl)silane (6.25 mL, 40.0 mmol) in THF (10 mL) at 0° C. was slowly added 1 M tetrabutylammonium fluoride solution in THF (0.334 mL, 0.334 mmol). The mixture was stirred at 0° C. for 2 h. The mixture was diluted with water and extracted with ether. The organic layer was washed with brine, dried over anhydrous Na₂SO₄, filtered, and concentrated under reduced pressure (150 mmHg) at room temperature to afford the title compound. ¹H NMR (400 MHz, CDCl₃) δ ppm 1.71-1.88 (m, 2H), 2.23-2.31 (m, 2H), 2.44-2.51 (m, 2H).

### Step B: Preparation of 1-(Trifluoromethyl)cyclobutyl 1H-Imidazole-1-carboxylate

To one half of the crude 1-(trifluoromethyl)cyclobutanol from Step A was added THF (15 mL) and CDI (4.04 g, 24.95 mmol). The resulting suspension was heated at 70° C. overnight. The reaction was carefully quenched with water and extracted twice with MTBE. The organic phases were combined, concentrated under reduced pressure, and purified by column chromatography to afford 1-(trifluoromethyl)cyclobutyl 1H-imidazole-1-carboxylate (0.2328 g, 5.98%) (over 2 steps) as an orange solid. Exact mass calculated for C₉H₉F₃N₂O₂: 234.1. found: LCMS m/z=235.0 [M+H]⁺; ¹H NMR (400 MHz, CDCl₃) δ ppm 1.94-2.04 (m, 1H), 2.06-2.13 (m, 1H), 2.68-2.75 (m, 2H), 2.85-2.95 (m, 2H), 7.09 (m, 1H), 7.40 (m, 1H), 8.11 (s, 1H).

### Step C: Preparation of 1-(Trifluoromethyl)cyclobutyl 4-(1r,4r)-4-((2-Fluoro-4-(methylsulfonyl)phenoxy)methyl)cyclohexyl)piperazine-1-carboxylate (Compound 66)

To a suspension of 1-((1r,4r)-4-((2-fluoro-4-(methylsulfonyl)phenoxy)methyl)cyclohexyl)piperazine dihydrochloride (100 mg, 0.226 mmol) and 1-(trifluoromethyl)cyclobutyl 1H-imidazole-1-carboxylate (58.1 mg, 0.248 mmol) in THF (3 mL) was added triethylamine (0.157 mL, 1.128 mmol). The mixture was heated at 90° C. overnight. The reaction mixture was diluted with EtOAc and washed with brine. The organic layer was removed and the aqueous layer was extracted with EtOAc. The combined organic phases were dried over anhydrous Na₂SO₄, filtered, and concentrated under reduced pressure to dryness. The residue was triturated with IPA to afford the title compound as an off-white solid (72.8 mg, 60.2%). The filtrate was concentrated under reduced pressure and purified by semi-prep HPLC. The fractions containing the desired product were neutralized with saturated aqueous NaHCO₃ solution to afford a second batch of the title compound as a white solid (20 mg, 16.53%). Exact mass calculated for C₂₄H₃₂F₄N₂O₅S: 536.2. found: LCMS m/z=537.4 [M+H]⁺; ¹H NMR (400 MHz, CDCl₃) δ ppm 1.12-1.19 (m, 2H), 1.26-1.33 (m, 2H), 1.81-1.89 (m, 2H), 1.94-2.04 (m, 5H), 2.29-2.37 (m, 1H), 2.50-2.57 (m, 6H), 2.82-2.85 (m, 2H), 3.03 (s, 3H), 3.45-3.48 (m, 4H), 3.90 (d, J=8.0 Hz, 2H), 7.05 (t, J=8.0 Hz, 1H), 7.63-7.69 (m, 2H).

### Example 1.65

### Preparation of tert-Butyl 4-((1r,4r)-4-((3-Cyanopyridin-4-yloxy)methyl)cyclohexyl)piperazine-1-carboxylate (Compound 67)

tert-Butyl 4-((1r,4r)-4-((methylsulfonyloxy)methyl)cyclohexyl)piperazine-1-carboxylate (784 mg, 2.081 mmol) was dissolved in DMF (10 mL), and 4-hydroxynicotinonitrile (300 mg, 2.498 mmol) and cesium carbonate (1356 mg, 4.16 mmol) were added. The reaction mixture was heated at 100° C. for 3 h under microwave irradiation, then poured into water, and extracted with DCM. The organic phase was dried over anhydrous Na₂SO₄, and concentrated. The residue was purified by preparative HPLC (5-50% CH₃CN/H₂O with 0.1% TFA over 30 min). Fractions containing the title compound were combined, neutralized with saturated NaHCO₃, and extracted with DCM. The organic phase was dried over anhydrous Na₂SO₄ and concentrated to give the title compound as a white solid (95 mg, 0.237 mmol, 11.40%). Another 165 mg of the title compound was obtained in 80% purity. Exact mass calculated for C₂₂H₃₂N₄O₃: 400.2. found: LCMS m/z=401.2 [M+H]⁺; ¹H NMR (400 MHz, CDCl₃) δ ppm 1.10-1.20 (m, 2H), 1.25-1.40 (m, 2H), 1.46 (s, 9H), 1.80-1.90 (m, 1H), 1.92-2.05 (m, 4H), 2.28-2.35 (m, 1H), 2.48-2.54 (m, 4H), 3.40-3.45 (m, 4H), 3.94 (d, J=6.3 Hz, 2H), 6.86 (d, J=6.0 Hz, 1H), 8.60 (d, J=6.0 Hz, 1H), 8.67 (s, 1H).

### Example 1.66

### Preparation of tert-Butyl 4-((1r,4r)-4-((Pyridin-4-yloxy)methyl)cyclohexyl)piperazine-1-carboxylate (Compound 68)

tert-Butyl 4-((1r,4r)-4-((methylsulfonyloxy)methyl)cyclohexyl)piperazine-1-carboxylate (1320 mg, 3.51 mmol) was dissolved in DMF (10 mL), and pyridin-4-ol (400 mg, 4.21 mmol) and cesium carbonate (2284 mg, 7.01 mmol) were added. The reaction mixture was heated at 100° C. for 3 h under microwave irradiation, at which time the reaction was complete. The solid was filtered off, and washed with ethyl acetate. The combined filtrate and washings were concentrated, and the residue was purified by preparative HPLC (5-50% CH₃CN/H₂O with 0.1% TFA over 30 min) to give a mixture of tert-butyl 4-((1r,4r)-4-((pyridin-4-yloxy)methyl)cyclohexyl)piperazine-1-carboxylate and tert-butyl 4-((1r,4r)-4-((4-oxopyridin-1(4H)-yl)methyl)cyclohexyl)piperazine-1-carboxylate. Approximately 90 mg of the mixture was separated by HPLC to give the title compound. Exact mass calculated for C₂H₃₃N₃O₃: 374.2. found: LCMS m/z=375.4 [M+H]⁺; ¹H NMR (400 MHz, CDCl₃) δ ppm 1.10-1.20 (m, 2H), 1.20-1.35 (m, 2H), 1.46 (s, 9H), 1.70-1.80 (m, 1H), 1.90-2.00 (m, 4H), 2.25-2.35 (m, 1H), 2.48-2.54 (m, 4H), 3.40-3.45 (m, 4H), 3.78 (d, J=6.3 Hz, 2H), 6.76 (dd, J=4.5 and 1.8 Hz, 2H), 8.38 (dd, J=4.8 and 1.5 Hz, 2H).

### HPLC Conditions.

Column: normal phase preparative Chiralpak® IA 250×20 mm ID, 5 μM particle size
Eluent: 60% IPA/Hexanes with 0.1% DEA
Gradient: isocratic
Flow: 6 mL/min

### Detector: 254 nm

### Retention Time: 15.2 min

### Example 1.67

### Preparation of (S)-tert-Butyl 4-(1r,4S)-4-((2-Fluoro-4-(methylsulfonyl)phenoxy)methyl)cyclohexyl)-2-methylpiperazine-1-carboxylate (Compound 69)

(S)-tert-Butyl 2-methyl-4-(4-((methylsulfonyloxy)methyl)cyclohexyl)piperazine-1-carboxylate (0.39 g, 0.260 mmol, corrected for 74:26 cis:trans) was dissolved in anhydrous DMF (2 mL). 2-Fluoro-4-(methylsulfonyl)phenol (94.97 mg, 0.499 mmol) was added, followed by cesium carbonate (0.651 g, 1.997 mmol). The reaction mixture was heated at 100° C. for 2 h. The solid was filtered off and washed with ethyl acetate. The filtrate was concentrated and then purified by silica gel column (75% ethyl acetate/hexanes with 1% Et₃N) to give the title compound (72 mg, 14.9%) Exact mass calculated for C₂₄H₃₇FN₂O₅S: 484.2. found: LCMS m/z=485.4 [M+H]⁺; ¹H NMR (400 MHz, CDCl₃) δ ppm 1.05-1.18 (m, 2H), 1.21 (d, J=6.7 Hz, 3H), 1.20-1.38 (m, 2H), 1.46 (s, 9H), 1.75-2.03 (m, 5H), 2.22-2.33 (m, 2H), 2.35-2.42 (m, 1H), 2.58-2.62 (m, 1H), 2.70-2.75 (m, 1H), 3.03 (s, 3H), 2.98-3.08 (m, 1H), 3.75-3.82 (m, 1H), 3.90 (d, J=6.3 Hz, 2H), 4.15-4.22 (m, 1H), 7.05 (t, J=8.0 Hz, 1H), 7.62-7.70 (m, 2H). The less polar (S)-tert-butyl 4-((1s,4R)-4-(2-fluoro-4-(methylsulfonyl)phenoxy)methyl)cyclohexyl)-2-methylpiperazine-1-carboxylate was also isolated (1.5 mg, 0.3%).

### Example 1.68

### Preparation of (R)-tert-Butyl 4-((1r,4r)-4-((2-Fluoro-4-(methylsulfonyl)phenoxy)methyl)cyclohexyl)-2-methylpiperazine-1-carboxylate (Compound 70)

(R)-tert-Butyl 2-methyl-4-(4-((methylsulfonyloxy)methyl)cyclohexyl)piperazine-1-carboxylate (0.25 g, 0.467 mmol, corrected for 73:27 trans:cis) was dissolved in anhydrous DMF (2 mL). 2-Fluoro-4-(methylsulfonyl)phenol (0.107 g, 0.561 mmol) was added, followed by cesium carbonate (0.305 g, 0.935 mmol). The reaction mixture was heated at 100° C. for 2 h. The reaction mixture was poured into water, the solid was collected to give the product in 94% purity, which was then purified by silica gel column (75% ethyl acetate/hexanes with 1% Et₃N) to give the title compound as white solid (208 mg, 91.8%). Exact mass calculated for C₂₄H₃₇FN₂O₅S: 484.2. found: LCMS m/z=485.4 [M+H]⁺; ¹H NMR (400 MHz, CDCl₃) δ ppm 1.05-1.18 (m, 2H), 1.21 (d, J=6.7 Hz, 3H), 1.20-1.35 (m, 2H), 1.46 (s, 9H), 1.75-2.03 (m, 5H), 2.22-2.33 (m, 2H), 2.35-2.42 (m, 1H), 2.58-2.62 (m, 1H), 2.70-2.75 (m, 1H), 3.03 (s, 3H), 2.98-3.08 (m, 1H), 3.75-3.82 (m, 1H), 3.90 (d, J=6.3 Hz, 2H), 4.15-4.22 (m, 1H), 7.05 (t, J=8.0 Hz, 1H), 7.62-7.70 (m, 2H).

### Example 1.69

### Preparation of 4-(((1r,4r)-4-(4-(5-(Trifluoromethyl)pyrimidin-2-yl)piperazin-1-yl)cyclohexyl)methoxy)nicotinonitrile (Compound 72)

### Step A: Preparation of 4-(((1r,4r)-4-(piperazin-1-yl)cyclohexyl)methoxy)nicotinonitrile Dihydrochloride

4 M Hydrogen chloride in dioxane (2.247 mL, 8.988 mmol) was added to tert-butyl 4-((1r,4r)-4-((3-cyanopyridin-4-yloxy)methyl)cyclohexyl)piperazine-1-carboxylate (72 mg, 0.180 mmol) in dioxane (1 mL). The reaction mixture was stirred for 4 h at room temperature. The precipitate was collected, washed with dioxane and dried to give the title compound as an off-white solid (66 mg, 98.6%). Exact mass calculated for C₁₇H₂₄N₄O: 300.2. found: LCMS m/z=301.2 [M+11]⁺.

### Step B: Preparation of 4-4(1r,4r)-4-(4-(5-(Trifluoromethyl)pyrimidin-2-yl)piperazin-1-yl)cyclohexyl)methoxy)nicotinonitrile (Compound 72)

A mixture of 4-(((1r,4r)-4-(piperazin-1-yl)cyclohexyl)methoxy)nicotinonitrile dihydrochloride (20 mg, 53.71 μmol), 2-chloro-5-(trifluoromethyl)pyrimidine (12.74 mg, 69.82 μmol) and triethylamine (29.94 μL, 0.215 mmol) in IPA (1.5 mL) was heated at 120° C. for 40 min. The precipitate was collected, washed with IPA and dried to give the title compound as an off-white solid (18 mg, 75.1%). Exact mass calculated for C₂₂H₂₅F₃N₆O: 446.2. found: LCMS m/z=447.6 [M+H]⁺; ¹H NMR (400 MHz, CDCl₃) δ ppm 1.13-1.25 (m, 2H), 1.30-1.40 (m, 2H), 1.80-1.90 (m, 1H), 1.95-2.05 (m, 4H), 2.30-2.40 (m, 1H), 2.62-2.68 (m, 4H), 3.88-3.93 (m, 4H), 3.94 (d, J=6.2 Hz, 2H), 6.86 (d, J=6.0 Hz, 1H), 8.46 (s, 2H), 8.60 (d, J=6.0 Hz, 1H), 8.67 (s, 1H).

### Example 1.70

### Preparation of (S)-3-(Trifluoromethyl)oxetan-3-yl 4-(1r,4S)-4-((2-Fluoro-4-(methylsulfonyl)phenoxy)methyl)cyclohexyl)-2-methylpiperazine-1-carboxylate (Compound 75)

### Step A: Preparation of (S)-1-(1r,4S)-4-((2-Fluoro-4-(methylsulfonyl)phenoxy)methyl)cyclohexyl)-3-methylpiperazine Dihydrochloride

(S)-tert-Butyl 4-((1r,4S)-4-(2-fluoro-4-(methylsulfonyl)phenoxy)methyl)cyclohexyl)-2-methylpiperazine-1-carboxylate (30 mg, 61.90 μmol) in 4 N HCl in dioxane (1.548 mL, 6.190 mmol) was stirred for 1 h at room temperature, and then concentrated to give the title compound (28 mg, 98.9%). Exact mass calculated for C₁₉H₂₉FN₂O₃S: 384.2. found: LCMS m/z=385.4 [M+H]⁺.

### Step B: Preparation of (S)-3-(Trifluoromethyl)oxetan-3-yl 4-(1r,4S)-4-((2-Fluoro-4-(methylsulfonyl)phenoxy)methyl)cyclohexyl)-2-methylpiperazine-1-carboxylate (Compound 75)

To a suspension of (S)-1-((1r,4S)-4-((2-fluoro-4-(methylsulfonyl)phenoxy)methyl)cyclohexyl)-3-methylpiperazine dihydrochloride (28 mg, 61.22 μmol) and perfluorophenyl 3-(trifluoromethyl)oxetan-3-yl carbonate (25.87 mg, 73.46 μmol) in DCM under nitrogen was added triethylamine (42.66 μL, 0.306 mmol). The mixture was stirred at room temperature for 1 h. The mixture was washed with saturated aqueous sodium bicarbonate, dried over anhydrous sodium sulfate, filtered, and concentrated. The residue was purified by silica gel column chromatography (75% ethyl acetate/hexanes with 1% Et₃N) to give the title compound (26 mg, 6.9%). Exact mass calculated for C₂₄H₃₂F₄N₂O₆S: 552.2. found: LCMS m/z=553.6 [M+H]⁺; ¹H NMR (400 MHz, CDCl₃) δ ppm 1.07-1.20 (m, 2H), 1.24 (d, J=6.7 Hz, 3H), 1.20-1.35 (m, 2H), 1.75-2.03 (m, 5H), 2.22-2.35 (m, 2H), 2.42-2.48 (m, 1H), 2.62-2.67 (m, 1H), 2.72-2.80 (m, 1H), 3.03 (s, 3H), 3.10-3.20 (m, 1H), 3.75-3.82 (m, 1H), 3.88 (d, J=6.3 Hz, 2H), 4.15-4.22 (m, 1H), 4.83 (t, J=7.3 Hz, 2H), 4.95-5.02 (m, 2H), 7.05 (t, J=8.0 Hz, 1H), 7.62-7.70 (m, 2H).

### Example 1.71

### Preparation of 5-((S)-4-((1r,4S)-4-((2-Fluoro-4-(methylsulfonyl)phenoxy)methyl)cyclohexyl)-2-methylpiperazin-1-yl)-3-(trifluoromethyl)-1,2,4-oxadiazole (Compound 78)

### Step A: Preparation of (S)-4-(1r,4S)-4-((2-Fluoro-4-(methylsulfonyl)phenoxy)methyl)cyclohexyl)-2-methylpiperazine-1-carbonitrile

To a suspension of (S)-1-((1r,4S)-4-((2-fluoro-4-(methylsulfonyl)phenoxy)methyl)cyclohexyl)-3-methylpiperazine hydrochloride (0.1 g, 0.238 mmol) in anhydrous DCM (1 mL) in an ice-water bath was added DIEA (0.166 mL, 0.950 mmol), followed by cyanic bromide (0.119 mL, 0.356 mmol). The reaction mixture was stirred at room temperature for 2 h, quenched with water, extracted with DCM. The combined organic phases were concentrated, the residue was purified by column chromatography (10% methanol/DCM) to give the title compound (85 mg, 0.208 mmol, 87.4%). Exact mass calculated for C₂₀H₂₈FN₃O₃S: 409.2. found: LCMS m/z=410.2 [M+H]⁺.

### Step B: Preparation of 5-((S)-4-((1r,4S)-4-((2-Fluoro-4-(methylsulfonyl)phenoxy)methyl)cyclohexyl)-2-methylpiperazin-1-yl)-3-(trifluoromethyl)-1,2,4-oxadiazole (Compound 78)

To a mixture of (S)-4-((1r,4S)-4-((2-fluoro-4-(methylsulfonyl)phenoxy)methyl)cyclohexyl)-2-methylpiperazine-1-carbonitrile (85 mg, 0.208 mmol) and 2,2,2-trifluoro-Ar-hydroxyacetimidamide (42.5 mg, 0.332 mmol) in THF (1 mL) was added 0.5 M zinc(II) chloride in THF (1.245 mL, 0.623 mmol) at room temperature under nitrogen. The reaction mixture was stirred overnight at room temperature. 4 M HCl in dioxane (0.311 mL, 1.245 mmol) was added. The reaction mixture was heated at 120° C. under microwave irradiation for 2 h, then purified by silica gel column chromatography (70% ethyl acetate/hexanes) and semi preparative HPLC (35-90% CH₃CN/H₂O with 0.1% TFA over 30 min). The combined fractions were neutralized with saturated aqueous NaHCO₃ and partially concentrated. A white solid formed, which was collected and dried to give the title compound (8.8 mg, 8.1%). Exact mass calculated for C₂₂H₂₈F₄N₄O₄S: 520.2. found: LCMS m/z=521.4 [M+H]⁺; ¹H NMR (400 MHz, CDCl₃) δ ppm 1.10-1.20 (m, 2H), 1.20-1.35 (m, 2H), 1.38 (d, J=6.7 Hz, 3H), 1.75-2.03 (m, 5H), 2.32-2.42 (m, 1H), 2.45-2.52 (m, 1H), 2.55-2.63 (m, 1H), 2.68-2.75 (m, 1H), 2.84-2.90 (m, 1H), 3.03 (s, 3H), 3.40-3.50 (m, 1H), 3.85-3.90 (m, 1H), 3.90 (d, J=6.3 Hz, 2H), 4.25-4.32 (m, 1H), 7.05 (t, J=8.0 Hz, 1H), 7.62-7.70 (m, 2H).

### Example 1.72

### Preparation of tert-Butyl 4-((1R,2S,4R)-2-Fluoro-4-((2-fluoro-4-(methylsulfonyl)phenoxy)methyl)cyclohexyl)piperazine-1-carboxylate (Compound 81) and tert-Butyl 4-((1R,2R,4R)-2-Fluoro-4-((2-fluoro-4-(methylsulfonyl)phenoxy)methyl)cyclohexyl)piperazine-1-carboxylate (Compound 82)

### Step A: Preparation of tert-Butyl(4-((2-Fluoro-4-(methylsulfonyl)phenoxy)methyl)cyclohex-1-enyloxy)dimethylsilane

To a stirred solution of 4-((2-fluoro-4-(methylsulfonyl)phenoxy)methyl)cyclohexanone (250 mg, 0.832 mmol) and DIEA (0.290 mL, 1.665 mmol) in DCM (2 mL) in an ice-water bath was added tert-butyldimethylsilyl trifluoromethanesulfonate (0.264 g, 0.999 mmol) under nitrogen. The reaction mixture was stirred for 2 h during which time it slowly warmed to room temperature. Water was added and the mixture was extracted with DCM. The combined organic phases were concentrated. The residue was purified by silica gel column chromatography (40% ethyl acetate/hexanes) to the title compound as a white solid (338 mg, 97.9%). Exact mass calculated for C₂₀H₃₁FO₄SSi: 414.2. found: LCMS m/z=415.2 [M+H]⁺; ¹H NMR (400 MHz, CDCl₃) δ ppm 0.13 (s, 6H), 0.92 (s, 9H), 1.48-1.60 (m, 1H), 1.85-2.30 (m, 6H), 3.03 (s, 3H), 3.95-4.02 (m, 2H), 4.84-4.87 (m, 1H), 7.07 (t, J=8.2 Hz, 1H), 7.62-7.70 (m, 2H).

### Step B: Preparation of 2-Fluoro-4-1((2-fluoro-4-(methylsulfonyl)phenoxy)methyl)cyclohexanone

To a solution of tert-butyl(4-((2-fluoro-4-(methylsulfonyl)phenoxy)methyl)cyclohex-1-enyloxy)dimethylsilane (255 mg, 0.615 mmol) in DMF (2 mL) was added 1-(chloromethyl)-4-fluoro-1,4-diazoniabicyclo[2.2.2]octane tetrafluoroborate (Selectfluor®) (0.240 g, 0.677 mmol) in DMF (1 mL) under nitrogen in an ice-water bath. The reaction mixture was stirred for 2 h during which time it slowly warmed to room temperature. The reaction mixture was then poured into water and extracted with DCM. The combined organic phases were concentrated. The residue was passed through a silica gel column (80% ethyl acetate/hexanes) to give the title compound (170 mg, 86.8%), with some impurity. Exact mass calculated for C₁₄H₁₆F₂N₄S: 318.2. found: LCMS m/z=319.0 [M+H]⁺.

### Step C: Preparation of tert-Butyl 4-((1R,2S,4R)-2-Fluoro-4-((2-fluoro-4-(methylsulfonyl)phenoxy)methyl)cyclohexyl)piperazine-1-carboxylate (Compound 81) and tert-Butyl 4-((1R,2R,4R)-2-Fluoro-4-((2-fluoro-4-(methylsulfonyl)phenoxy)methyl)cyclohexyl)piperazine-1-carboxylate (Compound 82)

To a mixture of 2-fluoro-4-((2-fluoro-4-(methylsulfonyl)phenoxy)methyl)cyclohexanone (170 mg, 0.534 mmol), tert-butyl piperazine-1-carboxylate (0.119 g, 0.641 mmol) and acetic acid (61.08 μL, 1.068 mmol) in DCM (3 mL) was added sodium triacetoxyborohydride (0.283 g, 1.335 mmol) under nitrogen in an ice-water bath. The reaction mixture was stirred at room temperature overnight, saturated aqueous NaHCO₃ was added, extracted with DCM. The combined organic phases were concentrated. The residue was purified by silica gel column chromatography (80% ethyl acetate/hexanes) and HPLC to give the two title compounds as follows.

Compound 81:

Exact mass calculated for C₂₃H₃₄F₂N₂O₅S: 488.2. found: LCMS m/z=489.2 [M+H]⁺; ¹H NMR (400 MHz, CDCl₃) δ ppm 1.46 (s, 9H), 1.58-1.68 (m, 2H), 1.80-1.90 (m, 2H), 1.95-2.05 (m, 1H), 2.15-2.25 (m, 1H), 2.25-2.40 (m, 2H), 2.56-2.62 (m, 4H), 3.03 (s, 3H), 3.40-3.45 (m, 4H), 4.05-4.10 (m, 1H), 4.17-4.22 (m, 1H), 5.03 (dt, J=50.0 Hz for doublet, 1H), 7.05 (t, J=8.0 Hz, 1H), 7.62-7.69 (m, 2H).

Compound 82:

Exact mass calculated for C₂₃H₃₄F₂N₂O₅S: 488.2. found: LCMS m/z=489.2 [M+H]⁺; ¹H NMR (400 MHz, CDCl₃) δ ppm 1.46 (s, 9H), 1.58-1.68 (m, 2H), 1.80-1.90 (m, 2H), 2.00-2.05 (m, 1H), 2.18-2.40 (m, 3H), 2.56-2.62 (m, 4H), 3.03 (s, 3H), 3.40-3.45 (m, 4H), 3.92-3.98 (m, 2H), 5.12 (dt, J=50.0 Hz for doublet, 1H), 7.05 (t, J=8.0 Hz, 1H), 7.62-7.69 (m, 2H).

### HPLC Conditions.

Column: normal phase preparative Chiralpak® IA 250×20 mm ID, 5 μM particle size
Eluent: 30% DCM/hexanes with 0.05% TEA
Gradient: isocratic
Flow: 10 mL/min

### Detector: 254 nm

### Example 1.73

### Preparation of tert-Butyl 4-((1r,4r)-4-((2-Fluoro-4-(methylsulfonyl)phenoxy)methyl)-2-oxocyclohexyl)piperazine-1-carboxylate (Compound 85)

### Step A: Preparation of tert-Butyl 4-(4-((2-Fluoro-4-(methylsulfonyl)phenoxy)methyl)-2-hydroxycyclohexyl)piperazine-1-carboxylate

A mixture of tert-butyl piperazine-1-carboxylate (0.651 g, 3.496 mmol) and 4-((2-fluoro-4-(methylsulfonyl)phenoxy)methyl)cyclohexanone (1 g, 3.329 mmol) in the presence of 4-methylbenzenesulfonic acid (3.440 mg, 19.98 μmol) in toluene (16 mL) was refluxed at 150° C. for 4 days with mechanical removal of water. ¹H NMR showed ˜60% conversion to the alkene. The solvent was removed by evaporation, the residue was dissolved in THF (10 mL) and the solution was cooled in an ice-water bath. Borane-methyl sulfide complex (0.208 mL, 2.197 mmol) was slowly added with stirring. The reaction mixture was warmed to room temperature and stirred for 3 h. Methanol (0.405 mL, 9.988 mmol) was added and the mixture was stirred overnight at room temperature and then concentrated. The residue was dissolved in THF (6 mL) and methanol (1 mL). To this solution was added solid sodium hydroxide (0.112 g, 2.797 mmol), followed by 30% hydrogen peroxide (0.317 g, 2.797 mmol) slowly. The reaction mixture was stirred at room temperature for 1 h, filtered, and the solids were washed with THF. The combined filtrate and washings were concentrated and the residue was dissolved in methanol (5 mL). The solution was acidified with concentrated HCl (0.2 mL) and stirred at room temperature for 15 min. The solution was then concentrated at room temperature, and the residue was made basic with 10% NaOH (0.959 mL, 2.397 mmol) and extracted with ethyl acetate. The combined organic phases were dried and the residue was purified by silica gel column chromatography (0.5% Et₃N in ethyl acetate) to give the title compound as white solid (560 mg, 46.1%, 83% purity). Exact mass calculated for C₂₃H₃₅FN₂O₆S: 486.2. found: LCMS m/z=487.4 [M+H]⁺; ¹H NMR (400 MHz, CDCl₃) δ ppm 1.46 (s, 9H), 1.55-1.65 (m, 4H), 1.65-1.75 (m, 1H), 1.95-2.02 (m, 1H), 2.22-2.33 (m, 2H), 2.33-2.45 (m, 3H), 2.65-2.72 (m, 2H), 3.03 (s, 3H), 3.36-3.50 (m, 4H), 3.60-3.68 (m, 1H), 4.08 (d, J=6.9 Hz, 2H), 7.05 (t, J=8.1 Hz, 1H), 7.62-7.70 (m, 2H).

### Step B: Preparation of tert-Butyl 4-((1r,4r)-4-((2-Fluoro-4-(methylsulfonyl)phenoxy)methyl)-2-oxocyclohexyl)piperazine-1-carboxylate (Compound 85)

tert-Butyl 4-(4-((2-fluoro-4-(methylsulfonyl)phenoxy)methyl)-2-hydroxycyclohexyl)piperazine-1-carboxylate (165 mg, 0.339 mmol) in DCM (1 mL) was added to a stirred suspension of pyridinium chlorochromate (PCC) (0.439 g, 2.035 mmol) in DCM (2 mL). The reaction mixture was stirred at room temperature overnight at which time some starting material remained by LCMS. A further 2 equivalents of PCC was added and the reaction was allowed to stir over the weekend. The solid was filtered off and washed with DCM. The combined filtrate and washings were concentrated and the residue was purified by semi-preparative HPLC to give a mixture of diastereomers (˜30 mg). The mixture was separated further by HPLC. Exact mass calculated for C₂₃H₃₃FN₂O₆S: 484.2. found: LCMS m/z=485.4 [M+H]⁺; ¹H NMR (400 MHz, CDCl₃) δ ppm ¹H NMR (400 MHz, CDCl₃) δ ppm 1.46 (s, 9H), 1.65-1.80 (m, 2H), 2.12-2.20 (m, 1H), 2.20-2.30 (m, 1H), 2.30-2.40 (m, 2H), 2.55-2.65 (m, 3H), 2.65-2.72 (m, 2H), 3.03 (s, 3H), 3.20-3.28 (m, 1H), 3.42-3.50 (m, 4H), 4.00 (m, 2H), 7.05 (t, J=8.1 Hz, 1H), 7.62-7.70 (m, 2H).

### HPLC Conditions.

Column: normal phase preparative Chiralpak® IA 250×20 mm ID, 5 μM particle size
Eluent: 70:28.5:1.5 hexanes/DCM/methanol with 0.05% TEA
Gradient: isocratic
Flow: 10 mL/min

### Detector: 254 nm

Retention time 24.4 min

### Example 1.74

### Preparation of tert-Butyl 4-((1R,4R)-2,2-Difluoro-4-((2-fluoro-4-(methylsulfonyl)phenoxy)methyl)cyclohexyl)piperazine-1-carboxylate (Compound 88)

To a stirred solution of tert-butyl 4-(4-((2-fluoro-4-(methylsulfonyl)phenoxy)methyl)-2-oxocyclohexyl)piperazine-1-carboxylate (30 mg, 61.91 μmol) in dichloroethane (1 mL) in an ice-water bath was added DAST (48.68 μL, 0.371 mmol). The reaction mixture was slowly warmed to room temperature and stirred overnight. The reaction was not complete. Saturated aqueous NaHCO₃ was added and the mixture was extracted with DCM. The combined organic phases were concentrated and the residue was purified by silica gel column chromatography (80% ethyl acetate/hexanes) and HPLC to give the title compound (1.3 mg, 4.1%, retention time=27.0 min). Exact mass calculated for C₂₃H₃₃F₃N₂O₅S: 506.2. found: LCMS m/z=507.2 [M+H]⁺; ¹H NMR (400 MHz, CDCl₃) δ ppm ¹H NMR (400 MHz, CDCl₃) δ ppm 1.46 (s, 9H), 1.60-1.80 (m, 3H), 1.85-2.08 (m, 2H), 2.15-2.18 (m, 1H), 2.35-2.45 (m, 1H), 2.60-2.75 (m, 5H), 3.03 (s, 3H), 3.34-3.43 (m, 4H), 4.00-4.05 (m, 2H), 7.05 (t, J=8.1 Hz, 1H), 7.62-7.70 (m, 2H).

Also isolated was tert-butyl 4-((1R,4S)-2,2-difluoro-4-((2-fluoro-4-(methylsulfonyl)phenoxy)methyl)cyclohexyl)piperazine-1-carboxylate (1.6 mg, 5.1%, retention time=23.2 min)

### HPLC Conditions.

Column: normal phase preparative Chiralpak® IA 250×20 mm ID, 5 μM particle size
Eluent: 80:19:1 hexanes/DCM/methanol with 0.05% TEA
Gradient: isocratic
Flow: 12 mL/min

### Detector: 254 nm

### Example 1.75

### Preparation of 1-(1r,4r)-4-((2-Fluoro-4-(methylsulfonyl)phenoxy)methyl)cyclohexyl)-4-(2,2,2-trifluoroethylsulfonyl)piperazine (Compound 71)

To a solution of 1-((1r,4r)-4-((2-fluoro-4-(methylsulfonyl)phenoxy)methyl)cyclohexyl)piperazine (0.07 g, 0.159 mmol) and 2,2,2-trifluoroethanesulfonyl chloride (31.91 mg, 0.175 mmol) in DCM (3 mL) was added triethylamine (52.90 μL, 0.397 mmol) at room temperature under nitrogen. The reaction was stirred for 30 min, diluted with DCM (10 mL), washed with saturated aqueous NaHCO₃ solution and brine, dried, and concentrated. The residue was purified by preparative TLC to afford the title compound (10 mg, 19.36 μmol, 12.2%). Exact mass calculated for C₂₀H₂₈F₄N₂O₅S₂: 516.1. found LCMS m/z=517.4 [M+H]⁺; ¹H NMR (400 MHz, CDCl₃) δ ppm 1.08-1.22 (m, 2H), 1.24-1.37 (m, 2H), 1.76-1.88 (m, 1H), 1.90-2.05 (m, 4H), 2.32-2.41 (m, 1H), 2.63-2.70 (m, 4H), 3.03 (s, 3H), 3.34-3.39 (m, 4H), 3.68 (q, J=9.3 Hz, 2H), 3.90 (d, J=6.3 Hz, 2H), 7.05 (t, J=8.1 Hz, 1H), 7.62-7.70 (m, 2H).

### Example 1.76

### Preparation of tert-Butyl 4-(1r,4r)-1-Cyano-4-((2-fluoro-4-(methylsulfonyl)phenoxy)methyl)cyclohexyl)piperazine-1-carboxylate (Compound 76)

To a solution of 4-((2-fluoro-4-(methylsulfonyl)phenoxy)methyl)cyclohexanone (1.0 g, 3.329 mmol) in Et₂O (4 mL) was added trimethylsilanecarbonitrile (0.330 g, 3.329 mmol) followed by zinc(I) iodide (3.201 mg, 16.65 μmol). After stirring for 15 min at room temperature, tert-butyl piperazine-1-carboxylate (0.620 g, 3.329 mmol) in MeOH (4.000 mL) was added to the reaction. The solution was refluxed for 3 h and then stirred overnight at room temperature. A precipitate formed which was removed by filtration and washed with IPA and hexane to afford the title compound as a white powder (1.2 g, 72.7%). Exact mass calculated for C₂₄H₃₄FN₃O₅S: 495.2. found LCMS m/z=496.6 [M+H]⁺; ¹H NMR (400 MHz, CDCl₃) δ ppm 1.41-1.52 (m, 13H), 1.86-1.98 (bs, 1H), 2.02-2.10 (m, 2H), 2.34-2.42 (m, 2H), 2.60-2.68 (m, 4H), 3.03 (s, 3H), 3.45-3.50 (m, 4H), 3.95 (d, J=6.6 Hz, 2H), 7.07 (t, J=8.2 Hz, 1H), 7.63-7.71 (m, 2H).

### Example 1.77

### Preparation of 3-(Trifluoromethl)oxetan-3-yl 4-((1r,4r)-4-((2-Fluoro-4-(methylsulfonyl)phenoxy)methyl)cyclohexyl)piperazine-1-carboxylate (Compound 73)

### Step A: Preparation of 3-(Trifluoromethyl)oxetan-3-ol

To a solution of oxetan-3-one (0.98 g, 13.6 mmol) and trimethyl(trifluoromethyl)silane (3.50 mL, 22.4 mmol) in THF (10 mL) under nitrogen was slowly added 1 M tetrabutylammonium fluoride in THF (1.40 mL, 1.40 mmol). The mixture was stirred at room temperature for 2 h and then cooled to 0° C. The mixture was quenched with 6 N HCl (aq.) (29 mL) and then stirred at room temperature for 2 h. The mixture was extracted with ether (2×50 mL) and the combined organic layers were washed with brine and dried over anhydrous Na₂SO₄. The salt was filtered off and the filtrate was concentrated under reduced pressure (350 mm Hg) at room temperature to afford the title compound as a brown solution in THF. ¹H NMR (400 MHz, CDCl₃) δ ppm 4.62 (d, J=8.34 Hz, 2H), 4.80 (d, J=7.33 Hz, 2H).

### Step B: Preparation of Perfluorophenyl 3-(Trifluoromethyl)oxetan-3-yl Carbonate

To a solution of 3-(trifluoromethyl)oxetan-3-ol in THF under nitrogen was added bis(perfluorophenyl) carbonate (6.43 g, 16.3 mmol) and MeCN (6 mL). To this at 0° C. was added triethylamine (6.10 mL, 43.5 mmol) dropwise. The mixture was stirred at room temperature for 23 h. The mixture was concentrated under reduced pressure to dryness and the residue was purified by Biotage™ column chromatography to afford the title compound as a yellow oil (2.35 g, 49.1%). Exact mass calculated for C₁₁H₄F₈O₄: 352.0. found: LCMS m/z=353.2 [M+H]⁺; ¹H NMR (400 MHz, CDCl₃) δ ppm 4.91 (d, J=9.85 Hz, 2H), 5.04 (d, J=8.59 Hz, 2H).

### Step C: Preparation of 3-(Trifluoromethyl)oxetan-3-yl 4-((1r,4r)-4-((2-Fluoro-4-(methylsulfonyl)phenoxy)methyl)cyclohexyl)piperazine-1-carboxylate (Compound 73)

To a suspension of 1-((1r,4r)-4-((2-fluoro-4-(methylsulfonyl)phenoxy)methyl)cyclohexyl)piperazine dihydrochloride (100 mg, 0.226 mmol) and perfluorophenyl 3-(trifluoromethyl)oxetan-3-yl carbonate (95.3 mg, 0.271 mmol) in DCM (5 mL) under nitrogen was added triethylamine (0.160 mL, 1.10 mmol). The mixture was stirred at room temperature for 30 min. The mixture was washed with saturated NaHCO₃ (aq.) and dried over anhydrous Na₂SO₄. The mixture was filtered and the filtrate was concentrated under reduced pressure to dryness. The residue was triturated with IPA (1 mL) to form a solid. The solid was collected by vacuum filtration and washed with IPA (3×1 mL) and hexanes (3×2 mL) to afford the title compound as an off-white solid (97.8 mg, 80.5%). Exact mass calculated for C₂₃H₃₀F₄N₂O₆S: 538.2. found: LCMS m/z=539.6 [M+H]⁺; ¹H NMR (500 MHz, CDCl₃) δ ppm 1.08-1.20 (m, 2H), 1.25-1.38 (m, 2H), 1.83 (bs, 1H), 1.95 (d, J=11.98 Hz, 2H), 2.01 (d, J=12.61 Hz, 2H), 2.34 (t, J=11.66 Hz, 1H), 2.57 (bs, 4H), 3.03 (s, 3H), 3.49 (bs, 4H), 3.90 (d, J=6.31 Hz, 2H), 4.84 (d, J=8.20 Hz, 2H), 4.99 (d, J=8.83 Hz, 2H), 7.05 (t, J=8.20 Hz, 1H), 7.66 (dd, J=18.29, 9.46 Hz, 2H).

### Example 1.78

### Preparation of 5-((4-(1r,4r)-4-((2-Fluoro-4-(methylsulfonyl)phenoxy)methyl)cyclohexyl)piperazin-1-yl)methyl)-3-(trifluoromethyl)-1,2,4-oxadiazole (Compound 74)

### Step A: Preparation of 5-(Chloromethyl)-3-(trifluoromethyl)-1,2,4-oxadiazole

A mixture of 2,2,2-trifluoro-N′-hydroxyacetimidamide (0.50 g, 3.9 mmol) and 2-chloroacetic anhydride (0.67 g, 3.9 mmol) under nitrogen was heated at 100° C. overnight in a sealed tube. The mixture was diluted with DCM. The mixture was cooled to 0° C. and neutralized with saturated NaHCO₃ (aq.) to remove 2-chloroacetic acid. The organic layer was removed, dried over anhydrous Na₂SO₄ and filtered. The filtrate was concentrated under reduced pressure to dryness to afford the crude title compound as a colorless oil (0.27 g, 33.8%, 90% purity). ¹H NMR (400 MHz, CDCl₃) δ ppm 4.77 (s, 2H). ¹⁹F NMR (376 MHz, CDCl₃) δ ppm-66.02 (s, 3F).

### Step B: Preparation of 5-((4-(1r,4r)-4-((2-Fluoro-4-(methylsulfonyl)phenoxy)methyl)cyclohexyl)piperazin-1-yl)methyl)-3-(trifluoromethyl)-1,2,4-oxadiazole (Compound 74)

To a solution of 1-((1r,4r)-4-((2-fluoro-4-(methylsulfonyl)phenoxy)methyl)cyclohexyl)piperazine dihydrochloride (50.0 mg, 0.113 mmol) and 5-(chloromethyl)-3-(trifluoromethyl)-1,2,4-oxadiazole (31.6 mg, 0.152 mmol) in IPA (1.5 mL) was added sodium iodide (16.9 mg, 0.113 mmol) and N-ethyl-N-isopropylpropan-2-amine (98.0 μL, 0.564 mmol). The mixture was heated at 120° C. for 30 min under microwave irradiation. Starting material remained so more 5-(chloromethyl)-3-(trifluoromethyl)-1,2,4-oxadiazole (16.4 mg, 0.0789 mmol) was added and the mixture was heat at 120° C. for a further 10 min under microwave irradiation. The reaction mixture was filtered and the solid was washed with DCM. The combined filtrate and washings were concentrated under reduced pressure and purified by semi-preparative HPLC. Pure fractions were combined, neutralized with saturated NaHCO₃ (aq.), and the volatile solvents was removed by evaporation. The resulting suspension was filtered to afford the title compound as a white solid (20.5 mg, 34.9%). Exact mass calculated for C₂₂H₂₈F₄N₄O₄S: 520.2. found: LCMS m/z=521.6 [M+H]⁺; ¹H NMR (500 MHz, CDCl₃) δ ppm 1.05-1.19 (m, 2H), 1.24-1.38 (m, 2H), 1.82 (bs, 1H), 1.99 (t, J=11.35 Hz, 4H), 2.30 (t, J=11.35 Hz, 1H), 2.67 (bs, 8H), 3.03 (s, 3H), 3.89 (d, J=6.31 Hz, 2H), 3.96 (s, 2H), 7.05 (t, J=7.88 Hz, 1H), 7.66 (dd, J=17.65, 9.46 Hz, 2H).

### Example 1.79

### Preparation of tert-Butyl 3-(1r,4r)-4-((2-Fluoro-4-(methylsulfonyl)phenoxy)methyl)cyclohexyl)-3,8-diazabicyclo[3.2.1]octane-8-carboxylate (Compound 77)

### Step A: Preparation of Ethyl 1,4-Dioxaspiro[4.5]decane-8-carboxylate

To a solution of ethyl 4-oxocyclohexanecarboxylate (5.00 g, 29.4 mmol) and ethane-1,2-diol (1.80 mL, 32.3 mmol) in toluene (50 mL) under nitrogen was added 4-methylbenzenesulfonic acid hydrate (55.9 mg, 0.294 mmol). The mixture was heated to reflux with mechanical removal of water for 18 h. The mixture was allowed to cool to room temperature and washed with saturated NaHCO₃ (aq.). The organic layer was dried over anhydrous Na₂SO₄ and filtered. The filtrate was concentrated under reduced pressure to dryness to afford the crude title compound as light yellow oil (6.26 g, 99.5%). Exact mass calculated for C₁₁H₁₈O₄: 214.1. found: LCMS m/z=215.2 [M+H]⁺.

### Step B: Preparation of 1,4-Dioxaspiro[4.5]decan-8-ylmethanol

To a solution of ethyl 1,4-dioxaspiro[4.5]decane-8-carboxylate (6.26 g, 29.2 mmol) in THF (100 mL) under nitrogen was added 2 M lithium borohydride in THF (29.2 mL, 58.4 mmol) portionwise. The mixture was heated at reflux for 20 h. The mixture was cooled to 0° C. and quenched with 1 N HCl (aq.) (30 mL). The mixture was diluted with DCM and stirred at room temperature for 30 min. The organic layer was separated, concentrated under reduced pressure to dryness, and purified by Biotage™ column chromatography to afford the title compound as colorless oil (3.71 g, 73.7%). Exact mass calculated for C₉H₁₆O₃: 172.1. found: LCMS m/z=173.2 [M+H]⁺; ¹H NMR (400 MHz, CDCl₃) δ ppm 1.18-1.38 (m, 2H), 1.46-1.63 (m, 3H), 1.71-1.85 (m, 4H), 3.49 (d, J=6.32 Hz, 2H), 3.90-3.98 (m, 4H).

### Step C: Preparation of 1,4-Dioxaspiro[4.5]decan-8-ylmethyl Methanesulfonate

To a solution of 1,4-dioxaspiro[4.5]decan-8-ylmethanol (3.71 g, 21.5 mmol) and triethylamine (6.00 mL, 43.1 mmol) in DCM (40 mL) at 0° C. under nitrogen was slowly added methanesulfonyl chloride (1.80 mL, 23.7 mmol) over 30 min. The mixture was stirred at room temperature for 3 h. The reaction was not complete. To the mixture at 0° C. was added more triethylamine (3.00 mL, 21.5 mmol) and methanesulfonyl chloride (0.670 mL, 8.62 mmol). The mixture was stirred at room temperature for a further 1.5 h. The mixture was quenched with ice and stirred at room temperature for 30 min. The organic layer was separated and dried over anhydrous Na₂SO₄. The mixture was filetered and the filtrate was concentrated under reduced pressure to afford the title compound as reddish brown oil (5.35 g, 99.2%) which was used in the next step without further purification. ¹H NMR (400 MHz, CDCl₃) δ ppm 1.27-1.43 (m, 2 H), 1.49-1.63 (m, 3H), 1.72-1.89 (m, 5H), 3.00 (s, 3H), 3.89-3.98 (m, 4H), 4.06 (d, J=6.32 Hz, 2H).

### Step D: Preparation of 8-((2-Fluoro-4-(methylsulfonyl)phenoxy)methyl)-1,4-dioxaspiro[4.5]decane

To a solution of 1,4-dioxaspiro[4.5]decan-8-ylmethyl methanesulfonate (5.39 g, 21.5 mmol) and 2-fluoro-4-(methylsulfonyl)phenol (4.10 g, 21.5 mmol) in DMF (20 mL) under nitrogen was added cesium carbonate (10.53 g, 32.31 mmol). The mixture was heated at 100° C. for 4 h. The mixture was diluted with EtOAc and filtered. The filtrate was concentrated under reduced pressure and purified by Biotage™ column chromatography to afford the title compound as a white solid (6.53 g, 88.0%). Exact mass calculated for C₁₆H₂₁FO₅S: 344.1. found: LCMS m/z=345.2 [M+H]⁺; ¹H NMR (400 MHz, CDCl₃) δ ppm 1.33-1.47 (m, 2H), 1.54-1.66 (m, 2H), 1.77-1.85 (m, 2H), 1.86-1.97 (m, 3H), 3.03 (s, 3H), 3.88-3.98 (m, 6H), 7.06 (t, J=8.21 Hz, 1H), 7.60-7.65 (m, 1H), 7.65-7.71 (m, 1H).

### Step E: Preparation of 4-((2-Fluoro-4-(methylsulfonyl)phenoxy)methyl)cyclohexanone

To a solution of 8-((2-fluoro-4-(methylsulfonyl)phenoxy)methyl)-1,4-dioxaspiro[4.5]decane (6.53 g, 19.0 mmol) in a mixed solvent of THF (20 mL) and water (20 mL) was added 6 N HCl (aq.) (6.32 mL, 37.9 mmol). The mixture was heated at 50° C. for 19 h. 15% of the starting material remained. The mixture was heated at 50° C. for a further 23 h after the addition of more 6 N HCl (aq.) (3.4 mL) and THF (10 mL). 9% of the starting material remained but the reaction was stopped. The resulting suspension was filtered and the filter cake was washed with THF to afford the title compound as a white solid (4.35 g, 76.4%). The filtrate was concentrated under reduced pressure to remove the organic solvent and the aqueous residue was again filtered to afford more title compound (0.95 g, 3.2 mmol, 16.7%). The combined yield was 93.1%. Exact mass calculated for C₁₄H₁₇FO₄S: 300.1. found: LCMS m/z=301.2 [M+H]⁺; ¹H NMR (400 MHz, CDCl₃) δ ppm 1.57-1.68 (m, 2H), 2.20-2.30 (m, 2H), 2.30-2.53 (m, 5H), 3.04 (s, 3H), 4.02 (d, J=6.32 Hz, 2H), 7.08 (t, J=8.21 Hz, 1H), 7.63-7.68 (m, 1H), 7.68-7.74 (m, 1H).

### Step F: Preparation of tert-Butyl 3-((1r,4r)-4-((2-Fluoro-4-(methylsulfonyl)phenoxy)methyl)cyclohexyl)-3,8-diazabicyclo[3.2.1]octane-8-carboxylate (Compound 77)

To a solution of 4-((2-fluoro-4-(methylsulfonyl)phenoxy)methyl)cyclohexanone (54.0 mg, 0.180 mmol) and tert-butyl 3,8-diazabicyclo[3.2.1]octane-8-carboxylate hydrochloride (53. 7 mg, 0.216 mmol) in DCE (3 mL) under nitrogen was added N-ethyl-N-isopropylpropan-2-amine (38.0 μL, 0.216 mmol). After stirring at room temperature for 5 min, acetic acid (21.0 μL, 0.360 mmol) and sodium triacetoxyborohydride (95.3 mg, 0.449 mmol) were added. The mixture was heated at 40° C. overnight. The reaction was quenched with saturated NaHCO₃ (aq.) and the organic layer was separated and the aqueous layer was extracted once with DCM. The combined organic phases were concentrated under reduced pressure and purified by Biotage™ column chromatography to afford the more polar title compound as a white solid (24.1 mg, 27.0%). Exact mass calculated for C₂₅H₃₇FN₂O₅S: 496.2. found: LCMS m/z=497.4 [M+H]⁺; ¹H NMR (400 MHz, CDCl₃) δ ppm 1.04-1.17 (m, 2H), 1.20-1.34 (m, 2H), 1.46 (s, 9H), 1.72-1.84 (m, 5H), 1.84-1.92 (m, 2H), 1.92-2.01 (m, 2H), 2.17-2.29 (m, 1H), 2.48 (bs, 2H), 2.55-2.60 (m, 2H), 3.03 (s, 3H), 3.89 (d, J=6.32 Hz, 2H), 4.18 (bs, 2H), 7.05 (t, J=8.08 Hz, 1H), 7.60-7.65 (m, 1H), 7.66-7.71 (m, 1H). The less polar cis analog was also obtained as a white solid (45.3 mg, 50.7%).

### Example 1.80

### Preparation of 1-(Trifluoromethyl)cyclobutyl 8-(1r,4r)-4-((2-fluoro-4-(methylsulfonyl)phenoxy)methyl)cyclohexyl)-3,8-diazabicyclo[3.2.1]octane-3-carboxylate (Compound 79)

### Step A: Preparation of 8-tert-Butyl 3-(1-(Trifluoromethyl)cyclobutyl) 3,8-diazabicyclo[3.2.1]octane-3,8-dicarboxylate

To a solution of tert-butyl 3,8-diazabicyclo[3.2.1]octane-8-carboxylate (67.1 mg, 0.316 mmol) and 1-(trifluoromethyl)cyclobutyl 1H-imidazole-1-carboxylate (88.8 mg, 0.379 mmol) in THF (2 mL) under nitrogen in a sealed tube was added triethylamine (88.0 μL, 0.632 mmol). The mixture was heated at 90° C. for 20 h. The reaction was not complete and thus more 1-(trifluoromethyl)cyclobutyl 1H-imidazole-1-carboxylate (88.8 mg, 0.379 mmol) and triethylamine (88.0 μL, 0.632 mmol) were added and the reaction was continued to heat at 90° C. for an additional day. The mixture was concentrated under reduced pressure and purified by Biotage™ column chromatography to afford the title compound as colorless oil (114.4 mg, 95.7%). Exact mass calculated for C₁₇H₂₅F₃N₂O₄: 378.2. found: LCMS m/z=379.2 [M+H]⁺; ¹H NMR (400 MHz, CDCl₃) δ ppm 1.47 (s, 9H), 1.61-1.77 (m, 2H), 1.79-2.05 (m, 4H), 2.45-2.64 (m, 2H), 2.67-2.80 (m, 1H), 2.80-2.93 (m, 1H), 3.03 (bs, 1H), 3.16 (bs, 1H), 3.69 (d, 1H), 3.83 (d, J=12.88 Hz, 1H), 4.21 (bs, 2H).

### Step B: Preparation of 1-(Trifluoromethyl)cyclobutyl 3,8-diazabicyclo[3.2.1]octane-3-carboxylate

To a solution of 8-tert-butyl 3-(1-(trifluoromethyl)cyclobutyl) 3,8-diazabicyclo[3.2.1]octane-3,8-dicarboxylate (114.4 mg, 0.302 mmol) in DCM (0.5 mL) was added 4 N hydrogen chloride in dioxane (1.00 mL, 4.01 mmol). The mixture was stirred at room temperature overnight. The reaction mixture was concentrated under reduced pressure and partitioned between DCM and saturated NaHCO₃ (aq.). The organic layer was separated and the aqueous layer was extracted once with DCM. The combined organic phases were dried over anhydrous Na₂SO₄, filtered and concentrated under reduced pressure to afford the crude title compound as a colorless gum (76.9 mg, 91.4%). Exact mass calculated for C₁₂H₁₇F₃N₂O₂: 278.1. found: LCMS m/z=279.4 [M+H]⁺; ¹H NMR (400 MHz, CDCl₃) δ ppm 1.67-1.79 (m, 4H), 1.79-1.92 (m, 1H), 1.92-2.05 (m, 1H), 2.43-2.65 (m, 2H), 2.68-2.92 (m, 2H), 2.98 (d, J=12.63 Hz, 1H), 3.08 (d, J=12.13 Hz, 1H), 3.47 (d, 2H), 3.68 (d, J=11.62 Hz, 1H), 3.79 (d, J=12.88 Hz, 1H).

### Step C: Preparation of 1-(Trifluoromethyl)cyclobutyl 8-(1r,4r)-4-((2-Fluoro-4-(methylsulfonyl)phenoxy)methyl)cyclohexyl)-3,8-diazabicyclo[3.2.1]octane-3-carboxylate (Compound 79)

To a solution of 4-((2-fluoro-4-(methylsulfonyl)phenoxy)methyl)cyclohexanone (70.0 mg, 0.233 mmol) and 1-(trifluoromethyl)cyclobutyl 3,8-diazabicyclo[3.2.1]octane-3-carboxylate (71.3 mg, 0.256 mmol) in DCE (3 mL) under nitrogen was added acetic acid (27.0 μL, 0.466 mmol) and sodium triacetoxyborohydride (123.0 mg, 0.583 mmol). The mixture was heated at 45° C. overnight. The reaction was quenched with saturated NaHCO₃ (aq.), the organic layer was separated, and the aqueous layer was extracted once with DCM. The combined organic phases were concentrated under reduced pressure and purified by Biotage™ column chromatography to afford the more polar title compound as a white solid (22.3 mg, 17.0%). Exact mass calculated for C₂₆H₃₄F₄N₂O₅S: 562.2. found: LCMS m/z=563.4 [M+H]⁺; ¹H NMR (400 MHz, CDCl₃) δ ppm 1.07-1.30 (m, 4H), 1.58-1.71 (m, 2H), 1.78-1.92 (m, 4H), 1.93-2.01 (m, 3H), 2.01-2.10 (m, 2H), 2.22-2.33 (m, 1H), 2.44-2.61 (m, 2H), 2.71-2.93 (m, 2H), 3.03 (s, 3H), 3.11 (d, J=1.52 Hz, 1H), 3.20 (d, 1H), 3.46 (bs, 1H), 3.49-3.57 (m, 2H), 3.62 (d, 1H), 3.90 (d, J=6.06 Hz, 2H), 7.06 (t, J=8.08 Hz, 1H), 7.62-7.66 (m, 1H), 7.66-7.71 (m, 1H). The less polar cis analog was also obtained as a white solid (40.8 mg, 31.1%).

### Example 1.81

### Preparation of tert-Butyl 4-((1r,4r)-4-((2-Fluoro-4-(methylsulfonyl)phenoxy)methyl)cyclohexyl)-2-oxopiperazine-1-carboxylate (Compound 80)

To a solution of 4-((2-fluoro-4-(methylsulfonyl)phenoxy)methyl)cyclohexanone (0.50 g, 1.7 mmol) and piperazin-2-one (0.18 g, 1.8 mmol) in DCE (10 mL) under nitrogen was added acetic acid (0.19 mL, 3.3 mmol) and sodium triacetoxyborohydride (0.88 g, 4.2 mmol). The mixture was heated at 45° C. for 2 h. The reaction was quenched with saturated NaHCO₃ (aq.), the organic layer was separated, and the aqueous layer was extracted once with DCM. The combined organic phases were dried over anhydrous Na₂SO₄, filtered, and concentrated under reduced pressure. The residue was dissolved in DCM (20 mL). To this DCM solution under nitrogen was added triethylamine (0.23 mL, 1.7 mmol), N,N-dimethylpyridin-4-amine (0.20 g, 1.7 mmol), and di-tert-butyl dicarbonate (0.55 g, 2.5 mmol). The mixture was stirred at room temperature for 16 h. The reaction was incomplete and so more triethylamine (0.23 mL, 1.7 mmol), N,N-dimethylpyridin-4-amine (0.20 g, 1.7 mmol), and di-tert-butyl dicarbonate (0.55 g, 2.5 mmol) were added. The mixture was stirred at room temperature for an additional day. The reaction mixture was washed with saturated NaHCO₃ (aq.) and the organic layer was concentrated under reduced pressure. The residue was purified by Biotage™ column chromatography to afford the more polar title compound as a light yellow solid (0.32 g, 39.7%). Exact mass calculated for C₂₃H₃₃FN₂O₆S: 484.2. found: LCMS m/z=485.2 [M+H]⁺; ¹H NMR (400 MHz, CDCl₃) δ ppm 1.09-1.22 (m, 2H), 1.22-1.39 (m, 2H), 1.53 (s, 9H), 1.76-1.90 (m, 1H), 1.91-2.07 (m, 4H), 2.27-2.41 (m, 1H), 2.79 (t, J=5.05 Hz, 2H), 3.03 (s, 3H), 3.39 (s, 2H), 3.67 (t, J=5.18 Hz, 2H), 3.90 (d, J=6.32 Hz, 2H), 7.05 (t, J=8.08 Hz, 1H), 7.62-7.66 (m, 1H), 7.66-7.71 (m, 1H). The less polar cis analog was also obtained as a light yellow solid (0.29 g, 36.3%).

### Example 1.82

### Preparation of tert-Butyl 4-(1r,4S)-2-Fluoro-4-((2-fluoro-4-(methylsulfonyl)phenoxy)methyl)cyclohexyl)piperazine-1-carboxylate (Compound 83)

### Step A: Preparation of tert-Butyl 4-(2-Fluoro-4-(hydroxymethyl)cyclohexyl)piperazine-1-carboxylate

To a solution of tert-butyl 4-(4-(ethoxycarbonyl)-2-fluorocyclohexyl)piperazine-1-carboxylate (0.30 g, 0.85 mmol) in THF (5 mL) under nitrogen at 0° C. was added 2 M lithium borohydride in THF (1.10 mL, 2.20 mmol). The mixture was heated at 75° C. overnight. The reaction was quenched with 1 N HCl (aq.) to pH 3-4 at 0° C. and basified with saturated NaHCO₃ (aq.). The mixture was extracted three times with DCM. The combined organic phases were dried over anhydrous Na₂SO₄, filtered and concentrated under reduced pressure to afford the title compound as a colorless gum (0.27 g, 99.9%) which was used in the next step without further purification. Exact mass calculated for C₁₆H₂₉FN₂O₃: 316.2. found: LCMS m/z=317.0 [M+H]⁺.

### Step B: Preparation of tert-Butyl 4-(2-Fluoro-4-((methylsulfonyloxy)methyl)cyclohexyl)piperazine-1-carboxylate

To a solution of tert-butyl 4-(2-fluoro-4-(hydroxymethyl)cyclohexyl)piperazine-1-carboxylate (0.27 g, 0.85 mmol) and triethylamine (0.24 mL, 1.7 mmol) in DCM (10 mL) at 0° C. under nitrogen was slowly added methanesulfonyl chloride (72.0 μL, 0.930 mmol). The mixture was stirred at room temperature for 1 h. The reaction was not complete. To the mixture at 0° C. was added more methanesulfonyl chloride (0.03 mL, 0.39 mmol) and triethylamine (0.12 mL, 0.85 mmol). The mixture was stirred at room temperature for a further 1 h. The mixture was quenched with water and stirred at room temperature for 30 min. The organic layer was separated, dried over anhydrous Na₂SO₄, filtered, and concentrated under reduced pressure to afford the crude title compound (contaminated by 5 mol % of tert-butyl 4-(2-fluoro-4-vinylcyclohexyl)piperazine-1-carboxylate) as a reddish brown oil (0.33 g, 100.0%). Exact mass calculated for C₁₇H₃₁FN₂O₅S: 394.2. found: LCMS m/z=395.2 [M+H]⁺.

### Step C: Preparation of tert-Butyl 4-(1r,4S)-2-Fluoro-4-((2-fluoro-4-(methylsulfonyl)phenoxy)methyl)cyclohexyl)piperazine-1-carboxylate (Compound 83)

To a solution of tert-butyl 4-(2-fluoro-4-((methylsulfonyloxy)methyl)cyclohexyl)piperazine-1-carboxylate (0.33 g, 0.85 mmol) and 2-fluoro-4-(methylsulfonyl)phenol (0.16 g, 0.85 mmol) in DMF (2 mL) under nitrogen was added cesium carbonate (0.41 g, 1.3 mmol). The mixture was heated at 100° C. for 2.5 h. The mixture was diluted with EtOAc and filtered. The filtrate was concentrated under reduced pressure and the residue was purified by Biotage™ column chromatography to afford the less polar title compound as a white solid (6.6 mg, 13.5 μmol, 1.6%). Exact mass calculated for C₂₃H₃₄F₂N₂O₅S: 488.2. found: LCMS m/z=489.2 [M+H]⁺; ¹H NMR (400 MHz, CDCl₃) δ ppm 1.46 (s, 9H), 1.57-1.77 (m, 3H), 1.77-1.91 (m, 2H), 1.91-2.10 (m, 1H), 2.30 (bs, 1H), 2.45-2.66 (m, 5H), 3.04 (s, 3H), 3.41 (bs, 4H), 3.89-4.04 (m, 2H), 4.68-4.88 (m, 1H), 7.07 (t, J=8.08 Hz, 2H), 7.65 (dd, J=9.98, 2.15 Hz, 1H), 7.69 (d, J=9.09 Hz, 1H). The more polar cis analog was also obtained as a white solid (38.4 mg, 9.3%).

### Example 1.83

### Preparation of tert-Butyl 5-(1r,4r)-4-((2-Fluoro-4-(methylsulfonyl)phenoxy)methyl)cyclohexyl)-2,5-diazabicyclo[4.1.0]heptane-2-carboxylate (Compound 84)

To a solution of tert-butyl 2,5-diazabicyclo[4.1.0]heptane-2-carboxylate (0.11 g, 0.56 mmol) and 4-((2-fluoro-4-(methylsulfonyl)phenoxy)methyl)cyclohexanone (0.18 g, 0.61 mmol) in DCE (5 mL) at room temperature under nitrogen was added acetic acid (63.0 μL, 1.10 mmol) and sodium triacetoxyborohydride (0.29 g, 1.4 mmol). The mixture was heated at 45° C. for 2 h. The reaction was quenched with saturated NaHCO₃ (aq.). The organic layer was separated and the aqueous layer was extracted once with DCM. The combined organic phases were concentrated under reduced pressure and purified by Biotage™ column chromatography to afford the more polar title compound as an off-white solid (101.0 mg, 37.7%). Exact mass calculated for C₂₄H₃₅FN₂O₅S: 482.2. found: LCMS m/z=483.2 [M+H]⁺; ¹H NMR (400 MHz, CDCl₃) δ ppm 0.44-0.57 (m, 1H), 0.58-0.82 (m, 1H), 1.10-1.24 (m, 2H), 1.29-1.44 (m, 2H), 1.48 (s, 9H), 1.78-1.95 (m, 1H), 2.01 (d, J=13.64 Hz, 2H), 2.07-2.22 (m, 2H), 2.35-2.52 (m, 3H), 2.66-2.87 (m, 2H), 2.96-3.16 (m, 1H), 3.03 (s, 3H), 3.44-3.73 (m, 1H), 3.92 (d, J=6.32 Hz, 2H), 7.06 (t, J=8.21 Hz, 1H), 7.61-7.66 (m, 1H), 7.68 (d, J=9.35 Hz, 1H). The less polar cis analog was also obtained (121.2 mg, 45.3%).

### Example 1.84

### Preparation of (1S,6R)-tert-Butyl 5-(1r,4R)-4-((2-Fluoro-4-(methylsulfonyl)phenoxy)methyl)cyclohexyl)-2,5-diazabicyclo[4.1.0]heptane-2-carboxylate (Compound 86) and (1R,6S)-tert-butyl 5-(1r,4S)-4-((2-fluoro-4-(methylsulfonyl)phenoxy)methyl)cyclohexyl)-2,5-diazabicyclo[4.1.0]heptane-2-carboxylate (Compound 87)

A 1:1 mixture of the title compounds was resolved using normal phase preparative chiral HPLC to afford Compound 86 as an off-white solid (34.6 mg, 34.3%, retention time: 67.516 min) and Compound 87 as an off-white solid (37.7 mg, 37.4%, retention time: 79.917 min)

### HPLC Conditions.

Sample: 10 mg crude injected in 0.5 mL of 20% DCM in EtOH
Column: normal phase preparative Chiralpak® IA 250×20 mm ID, 5 μM particle size
Eluent: hexanes/EtOH/TEA 90:10:0.05
Gradient: isocratic
Flow: 10 mL/min

### Detector: 254 nm

### Example 1.85

### Preparation of tert-Butyl 4-(1r,4r)-4-((2-Fluoro-4-(methylsulfonyl)phenoxy)methyl)cyclohexyl)-4,7-diazaspiro[2.5]octane-7-carboxylate (Compound 89)

To a solution of tert-butyl 4,7-diazaspiro[2.5]octane-7-carboxylate (0.16 g, 0.73 mmol) and 4-((2-fluoro-4-(methylsulfonyl)phenoxy)methyl)cyclohexanone (0.20 g, 0.67 mmol) in DCE (5 mL) at room temperature under nitrogen was added acetic acid (76.0 μL, 1.30 mmol) and sodium triacetoxyborohydride (0.35 g, 1.7 mmol). The mixture was heated at 45° C. for 3 days. The reaction formed a major side product (alcohol) from ketone starting material. The mixture was quenched with saturated NaHCO₃ (aq.). The organic layer was separated and the aqueous layer was extracted once with DCM. The combined organic phases were concentrated under reduced pressure and purified by preparative HPLC. The pure fractions were combined, neutralized with saturated NaHCO₃ (aq.), and concentrated under reduced pressure. The aqueous residue was extracted twice with DCM and the combined organic layers were dried over anhydrous Na₂SO₄, filtered and concentrated under reduced pressure to afford the more polar title compound as an off-white gum (7.7 mg, 2.3%). The less polar cis analog was also obtained as an off-white gum (1.2 mg, 0.4%). Exact mass calculated for C₂₅H₃₇FN₂O₅S: 496.2. found: LCMS m/z=497.4 [M+H]⁺; ¹H NMR (400 MHz, CDCl₃) δ ppm 0.55-0.64 (m, 2H), 0.66-0.72 (m, 2H), 1.08-1.36 (m, 4H), 1.45 (s, 9H), 1.75-1.89 (m, 1H), 1.93-2.02 (m, 2H), 2.09-2.18 (m, 2H), 2.87-2.97 (m, 1H), 3.03 (s, 3H), 3.08-3.13 (m, 2H), 3.17 (s, 2H), 3.40 (bs, 2H), 3.90 (d, J=6.32 Hz, 2H), 7.06 (t, J=8.21 Hz, 1H), 7.62-7.66 (m, 1H), 7.66-7.70 (m, 1H).

### Example 1.86

### Preparation of 3-((1S,6R)-5-(1r,4R)-4-((2-Fluoro-4-(methylsulfonyl)phenoxy)methyl)cyclohexyl)-2,5-diazabicyclo[4.1.0]heptan-2-yl)-5-(trifluoromethyl)-1,2,4-oxadiazole (Compound 90)

### Step A: Preparation of (1S,6R)-5-(1r,4R)-4-((2-Fluoro-4-(methylsulfonyl)phenoxy)methyl)cyclohexyl)-2,5-diazabicyclo[4.1.0]heptane-2-carbonitrile

To a solution of (1S,6R)-tert-butyl 5-((1r,4R)-4-((2-fluoro-4-(methylsulfonyl)phenoxy)methyl)cyclohexyl)-2,5-diazabicyclo[4.1.0]heptane-2-carboxylate (28.2 mg, 58.4 μmol) in DCM (0.5 mL) at room temperature under nitrogen was added 4 N hydrogen chloride in dioxane (0.47 mL, 1.9 mmol). The mixture was stirred at room temperature for 1 h. The mixture was concentrated under reduced pressure. The residue was dissolved in DCM (2 mL) and the solution was cooled to 0° C. under nitrogen. N-Ethyl-N-isopropylpropan-2-amine (51.0 μL, 0.290 mmol) and cyanogen bromide (7.4 mg, 70 μmol) were added and the mixture was stirred at room temperature for 1 h. The reaction was quenched with water and the organic layer was separated. The aqueous layer was extracted once with DCM. The combined organic phases were dried over anhydrous Na₂SO₄, filtered and concentrated under reduced pressure to afford the title compound as a light brown solid (23.8 mg, 58.4 μmol, 100.0%) which was used in the next step without further purification. Exact mass calculated for C₂₀H₂₆FN₃O₃S: 407.2. found: LCMS m/z=408.2 [M+H]⁺.

### Step B: Preparation of 3-((1S,6R)-5-((1r,4R)-4-((2-Fluoro-4-(methylsulfonyl)phenoxy)methyl)cyclohexyl)-2,5-diazabicyclo[4.1.0]heptan-2-yl)-5-(trifluoromethyl)-1,2,4-oxadiazole (Compound 90)

To a solution of (1S,6R)-5-((1r,4R)-4-(2-fluoro-4-(methylsulfonyl)phenoxy)methyl)cyclohexyl)-2,5-diazabicyclo[4.1.0]heptane-2-carbonitrile (23.8 mg, 58.4 μmol) and hydroxylamine hydrochloride (8.1 mg, 0.12 mmol) in DMF (1 mL) at room temperature under nitrogen was added potassium carbonate (8.1 mg, 58 μmol). The mixture was heated at 80° C. for 15 min. The mixture was cooled to 0° C. and then toluene (1.5 mL) and pyridine (24.0 μL, 0.290 mmol) were added, followed by slow addition of 2,2,2-trifluoroacetic anhydride (41.0 μL, 0.290 mmol). The mixture was again heated at 45° C. overnight. The mixture was quenched with water and extracted twice with EtOAc. The combined organic phases were concentrated under reduced pressure and purified by semi-preparative HPLC. The pure fractions were combined, neutralized with saturated NaHCO₃ (aq.), and concentrated. The resulting suspension was filtered to afford the title compound as a white solid (5.6 mg, 18.5%). Exact mass calculated for C₂₂H₂₆F₄N₄O₄S: 518.2. found: LCMS m/z=519.4 [M+H]⁺; ¹H NMR (400 MHz, CDCl₃) δ ppm 0.59 (q, J=5.22 Hz, 1H), 0.85 (q, J=6.48 Hz, 1H), 1.11-1.29 (m, 2H), 1.35-1.50 (m, 2H), 1.80-1.96 (m, 1H), 1.98-2.08 (m, 2H), 2.08-2.22 (m, 2H), 2.44-2.67 (m, 3H), 2.76-2.90 (m, 2H), 3.04 (s, 3H), 3.30-3.39 (m, 1H), 3.58-3.68 (m, 1H), 3.92 (d, J=6.06 Hz, 2H), 7.07 (t, J=8.08 Hz, 1H), 7.62-7.67 (m, 1H), 7.68 (d, J=8.84 Hz, 1H).

### Example 1.87

### Preparation of tert-Butyl 6-((1r,4r)-4-((2-Fluoro-4-(methylsulfonyl)phenoxy)methyl)cyclohexyl)-3,6-diazabicyclo[3.1.0]hexane-3-carboxylate (Compound 91)

### Step A: Preparation of (3S,4S)-tert-Butyl 3-Azido-4-hydroxypyrrolidine-1-carboxylate

To a solution of tert-butyl 6-oxa-3-azabicyclo[3.1.0]hexane-3-carboxylate (3.00 g, 16.2 mmol) in a mixed solvent of MeOH (40 mL) and water (10 mL) was added sodium azide (2.11 g, 32.4 mmol) and ammonium chloride (1.30 g, 24.3 mmol). The mixture was heated at 70° C. for 23 h. The mixture was diluted with water (30 mL) and extracted twice with DCM. The combined organic phases were concentrated under reduced pressure and redissovled in DCM. The DCM solution was dried over anhydrous Na₂SO₄, filtered and concentrated under reduced pressure to afford the title compound as light brown oil, which was used in the next step without further purification. Exact mass calculated for C₉H₁₆N₄O₃: 228.1. found: LCMS m/z=229.4 [M+H]⁺.

### Step B: Preparation of (3S,4S)-tert-Butyl 3-Azido-4-(methylsulfonyloxy)pyrrolidine-1-carboxylate

To a solution of the (3S,4S)-tert-butyl 3-azido-4-hydroxypyrrolidine-1-carboxylate from the previous step in pyridine (9.96 mL, 123.1 mmol) at 0° C. under nitrogen was slowly added methanesulfonyl chloride (1.38 mL, 17.8 mmol). The mixture was stirred at room temperature for 21 h. The mixture was poured into ice-cold water and extracted twice with DCM. The combined organic layers were washed with 1 N HCl (aq.) (2×50 mL) and brine, dried over anhydrous Na₂SO₄, filtered, and concentrated under reduced pressure to afford the title compound as red oil.

### Step C: Preparation of tert-Butyl 3,6-Diazabicyclo[3.1.0]hexane-3-carboxylate

To a solution of cobalt(II) bromide (0.66 g, 3.01 mmol) and 2,2′-bipyridine (1.57 g, 10.0 mmol) in EtOH (200 mL) at 0° C. was added a solution of the (3S,4S)-tert-butyl 3-azido-4-(methylsulfonyloxy)pyrrolidine-1-carboxylate from the previous step in EtOH (45 mL). To this at 0° C. was slowly added sodium borohydride (2.27 g, 59.9 mmol) over 1 h. The mixture was allowed to warm to room temperature and stirred for an additional 2 h. The mixture was then slowly poured into water (1000 mL) and stirred for 1 h. The mixture was extracted with DCM (2×200 mL) and the combined organic layers were dried over anhydrous Na₂SO₄. The salt was filtered off and the filtrate was concentrated under reduced pressure and purified by Biotage™ column chromatography to afford the title compound (1.13 g, 6.13 mmol, 37.9% over three steps) as an off-white solid. Exact mass calculated for C₉H₁₆N₂O₂: 184.1. found: LCMS m/z=185.2 [ M+H]⁺; ¹H NMR (400 MHz, CDCl₃) δ ppm 1.44 (s, 9H), 1.59 (d, J=6.57 Hz, 1H), 2.55 (bs, 2H), 3.35 (bs, 2H), 3.65 (bs, 2H).

### Step D: Preparation of tert-Butyl 6-(1r,4r)-4-((2-Fluoro-4-(methylsulfonyl)phenoxy)methyl)cyclohexyl)-3,6-diazabicyclo[3.1.0]hexane-3-carboxylate (Compound 91)

To a solution of 4-((2-fluoro-4-(methylsulfonyl)phenoxy)methyl)cyclohexanone (0.30 g, 1.0 mmol) and tert-butyl 3,6-diazabicyclo[3.1.0]hexane-3-carboxylate (0.20 g, 1.1 mmol) in DCE (5 mL) at room temperature under nitrogen was added acetic acid (0.11 mL, 2.0 mmol) and sodium triacetoxyborohydride (0.53 g, 2.5 mmol). The mixture was heated at 45° C. overnight. The reaction formed a major side product (alcohol) from the ketone starting material. The mixture was quenched with saturated NaHCO₃ (aq.). The organic layer was separated and the aqueous layer was extracted once with DCM. The combined organic phases were concentrated under reduced pressure and purified by preparative HPLC. Pure fractions were combined, neutralized with saturated NaHCO₃ (aq.), and concentrated. The resulting suspension was filtered to afford the title compound as a white solid (0.026 g, 5.5%). Exact mass calculated for C₂₃H₃₃FN₂O₅S: 468.2. found: LCMS m/z=469.4 [M+H]⁺; ¹H NMR (400 MHz, CDCl₃) δ ppm 1.00-1.14 (m, 2H), 1.20-1.32 (m, 1H), 1.41 (s, 9H), 1.45-1.61 (m, 2H), 1.82-2.03 (m, 5H), 2.26 (s, 2H), 3.03 (s, 3H), 3.32 (dd, J=17.56, 11.49 Hz, 2H), 3.55 (t, J=10.99 Hz, 2H), 3.89 (dd, J=5.94, 1.89 Hz, 2H), 7.05 (t, J=8.08 Hz, 1H), 7.61-7.66 (m, 1H), 7.67 (d, J=9.35 Hz, 1H). The cis analog was also obtained as a white solid (0.052 g, 11.1%).

### Example 2

### Effects of Compound 17 on Glucose Homeostasis in Male 129SVE Mice (Oral Glucose Tolerance Test (oGTT))

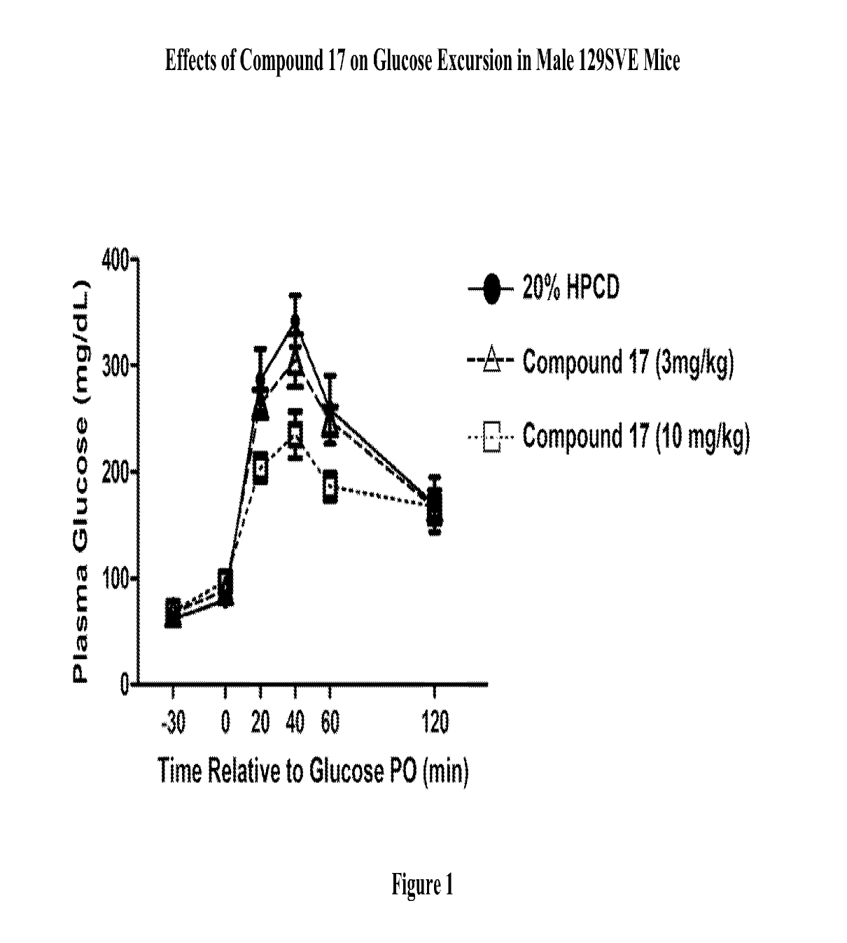

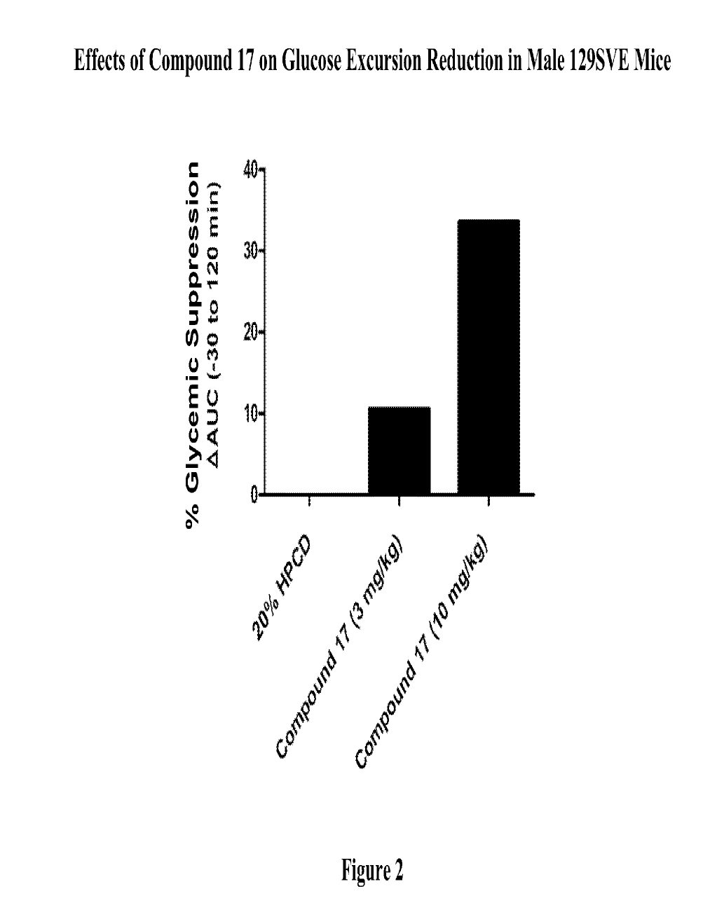

Male 129SVE mice (approximately 8 weeks old) were fasted for 18 h and randomly grouped (n=6) to receive a GPR119 agonist (Compound 17) at 3 or 10 mg/kg (mg/kg body weight). The compound was delivered orally via a gavage needle (p.o., 4 mL/kg) 30 minutes prior to glucose bolus (3 g/kg) (time=−30 min in FIG. 1), with a separate group receiving vehicle (20% hydroxypropyl-beta-cyclodextrin (HPCD)) as control. At time 0 min the glucose bolus was administered. Levels of blood glucose were assessed using a glucometer (One-Touch Ultra™, LifeScan) at time −30 min (prior to compound administration), at 0 min (at time when glucose bolus was given), and at 20, 40, 60, 120 min post glucose bolus. Plasma glucose levels (Table B) and the glucose excursion curve (FIG. 1) are shown. The reduction in the glucose excursion area under the curve (AUC) from −30 to 120 min in GPR119 agonist-treated animals relative to vehicle is shown in FIG. 2. These results demonstrated that the GPR119 agonist, Compound 17, lowered blood glucose in male 129SVE mice after challenge with glucose.

TABLE B

| | Plasma Glucose (mg/dL) | | | | | | | | |
|---|---|---|---|---|---|---|---|---|---|
| Time Relative | | | | Compound 17 | | | | | |
| to Glucose | 20% HPCD | | | (3 mg/kg) | | | (10 mg/kg) | | |
| Bolus (min) | Mean | SEM | n | Mean | SEM | n | Mean | SEM | n |
| −30 | 61.4 | 2.181742 | 5 | 67.33334 | 1.926425 | 6 | 68.5 | 2.362908 | 6 |
| 0 | 79.0 | 4.929503 | 5 | 88.0 | 2.352304 | 6 | 96.33334 | 7.666667 | 6 |
| 20 | 286.0 | 29.85465 | 5 | 263.5 | 13.23316 | 6 | 203.8333 | 12.95998 | 6 |
| 40 | 341.8 | 23.8776 | 5 | 304.8333 | 24.92311 | 6 | 234.8333 | 21.63857 | 6 |
| 60 | 258.4 | 31.86942 | 5 | 247.6667 | 13.29327 | 6 | 186.1667 | 13.10322 | 6 |
| 120 | 169.2 | 25.80581 | 5 | 166.3333 | 5.601587 | 6 | 167.3333 | 15.54706 | 6 |

### Example 3

### Effects of Compound 17 on GIP Release in Male 129SVE Mice

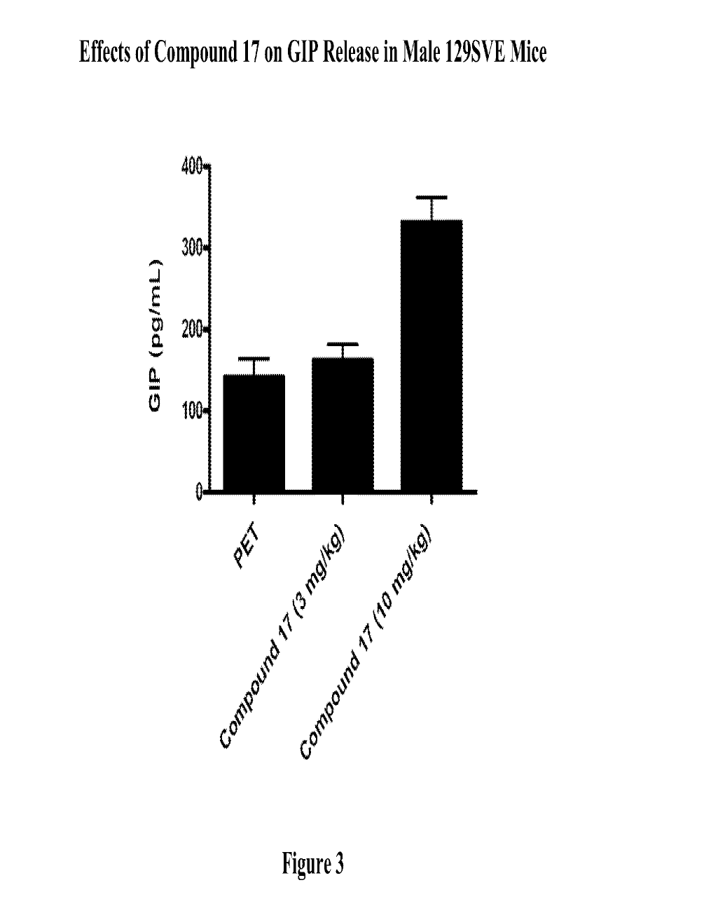

Male 129SVE mice (approximately 8 weeks old) were fasted for 18 h and randomly grouped (n=6) to receive a GPR119 agonist (Compound 17) at 3 or 30 mg/kg. Compound 17 was delivered orally via a gavage needle (p.o., 4 mL/kg), and after 45 min a blood sample was collected to determine plasma total GIP levels. A separate control group received vehicle (PET (80% PEG: 10% Ethanol: 10% Tween80™)). Plasma GIP levels were determined using a GIP (total) ELISA kit from Millipore. The results are given in FIG. 3. The results demonstrated that the GPR119 agonist, Compound 17, stimulated the release of GIP in male 129SVE mice.

### Example 4

### Homogeneous Time-Resolved Fluorescence (HTRF®) Assay For Direct cAMP Measurement

GPR119 agonists were evaluated in an HTRF® cAMP detection assay (Cisbio, cAMP Dynamic 2 Assay Kit; #62AM4PEJ) according to the manufacturer's instructions using CHO-K1 cells stably expressing the GPR119 receptor. CHO-K1 cells were transduced with a lentiviral vector encoding the nucleotide sequence of GPR119 (NCBI mRNA and protein reference sequences: NM_{—}178471.2 & NP_{—}848566). The N-terminus of the GPR119 nucleotide sequence was modified to replace the first, methionine-coding, codon with a nucleotide sequence coding for a standard, nine amino acid, hemagglutinin tag. Following transduction, cells expressing the GPR119 receptor were isolated and a single clone was isolated following standard dilution-cloning procedures. On the day of the assay, cultured CHO-GPR119 cells were harvested, suspended in assay buffer and plated into 384-well assay plates (PerkinElmer® Proxiplate® #6008280) at a density of 2,000 cells per well. A cAMP standard curve was added to each plate. Test compounds were dissolved in DMSO, serially diluted in DMSO and then diluted in assay buffer before addition to the cells. Test compounds were evaluated in triplicate, using 10-point, 5-fold serial dilutions starting at 10 μM. The final DMSO concentration in the assay was 0.5%. Compounds and cells were incubated for 1 h at room temperature and then detection reagents were added to each well (cAMP-D2 in cell lysis buffer, followed by europium cryptate-labeled anti-cAMP antibody). Plates were then incubated at room temperature for 1 h prior to reading. Time-resolved fluorescence measurements were collected on PerkinElmer Envision™ or BMG Pherastar™ microplate readers. The compound N-(2-fluoro-4-(methylsulfonyl) phenyl)-6-(4-(3-isopropyl-1,2,4-oxadiazol-5-yl)piperidin-1-yl)-5-nitropyrimidin-4-amine was used as a positive control in each runset while assay buffer containing 0.5% DMSO was used as the negative control. The HTRF® assay was used to determine EC₅₀ values for GPR119 agonists.

Certain compounds of the present invention and their corresponding EC₅₀ values are shown in Table C.

TABLE C

| Compound No. | EC₅₀ hGPR119 (nM) |
|---|---|
| 5 | 106.41 |
| 17 | 51.84 |
| 23 | 1380 |
| 26 | 599.65 |
| 31 | 70.10 |
| 38 | 219.44 |
| 44 | 22.23 |
| 64 | 13.68 |

Each of the Compounds disclosed in Table A had an hGPR119 EC₅₀ value in the range from about 9 nM to about 33 μM.

Those skilled in the art will recognize that various modifications, additions, and substitutions to the illustrative examples set forth herein can be made without departing from the spirit of the invention and are, therefore, considered within the scope of the invention.

Citation of any reference throughout this application is not to be construed as an admission that such reference is prior art to the present application.

1. A compound selected from compounds of Formula Ia and pharmaceutically acceptable salts, solvates, and hydrates thereof: 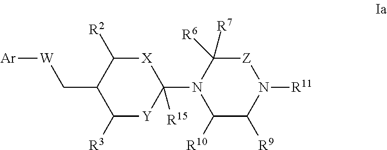
wherein:
Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl;
W is selected from: O and NR¹; or W is absent;
R¹ is selected from: H and C₁-C₆ alkyl;
R² and R³ are each H; or R² and R³ together form —CH₂—CH₂—;
X is selected from: —CH₂—CH₂—, CHR⁴, C(O), CHF, and CF₂; or X is absent;
Y is CHR⁵; or Y is absent;
R⁴ and R⁵ are each H; or R⁴ and R⁵ together form —CH₂—CH₂—;
Z is selected from: —CH₂—CH₂—, CHR⁸, and C(O);
R⁶, R⁹ and R¹⁰ are each H, and R⁷ and R⁸ are each independently selected from: H and C₁-C₆ alkyl; or R⁸, R⁹ and R¹⁰ are each H, and R⁶ and R⁷ together form —CH₂—CH₂—; or R⁶, R⁸ and R⁹ are each H, and R⁷ and R¹⁰ together form —CH₂—CH₂, or a bond; or R⁶, R⁷ and R¹⁰ are each H, and R⁸ and R⁹ together form —CH₂—CH₂; or R⁶, R⁷ and R⁹ are each H, and R⁸ and R¹⁰ together form —CH₂—CH₂—; or R⁶, R⁷ and R⁸ are each H, and R⁹ and R¹⁰ together form —CH₂—;
R¹¹ is selected from: C₁-C₆ alkoxycarbonyl, C₁-C₆ alkylsulfonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, heteroaryl-C₁-C₆ alkylene, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen; and
R¹⁵ is selected from: H and cyano.

2. A compound selected from compounds of Formula I and pharmaceutically acceptable salts, solvates, and hydrates thereof: 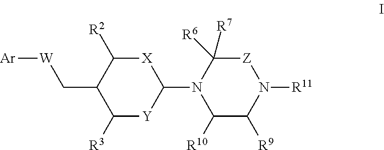
wherein:
Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl;
W is selected from: O and NR¹; or W is absent;
R¹ is selected from: H and C₁-C₆ alkyl;
R² and R³ are each H; or R² and R³ together form —CH₂—CH₂—;
X is selected from: —CH₂—CH₂— and CHR⁴; or X is absent;
Y is CHR⁵; or Y is absent;
R⁴ and R⁵ are each H; or R⁴ and R⁵ together form —CH₂—CH₂—;
Z is selected from: —CH₂—CH₂— and CHR⁸;
R⁶, R⁹ and R¹⁰ are each H, and R⁷ and R⁸ are each independently selected from: H and C₁-C₆ alkyl; or R⁸, R⁹ and R¹⁰ are each H, and R⁶ and R⁷ together form —CH₂—CH₂—; or R⁶, R⁸ and R⁹ are each H, and R⁷ and R¹⁰ together form —CH₂—CH₂; or R⁶, R⁷ and R¹⁰ are each H, and R⁸ and R⁹ together form —CH₂—CH₂; or R⁶, R⁷ and R⁹ are each H, and R⁸ and R¹⁰ together form —CH₂—CH₂—; and
R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

3. The compound according to claim 1, wherein Ar is selected from: aryl and heteroaryl; each optionally substituted with one or more substituents selected from: chloro, cyano, dimethylcarbamoyl, cyclopropylsulfonyl, fluoro, imidazolyl, methyl, methylpyrazolyl, methylsulfonyl, pyrrolyl, tetrazolyl, triazolyl, and trifluoromethylthio.

4. The compound according to claim 1, wherein Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl.

5. The compound according to claim 1, wherein Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, 6-cyano-2-methylpyrimidin-4-yl, and pyridin-4-yl.

6. The compound according to claim 1, wherein W is O.

7. The compound according to claim 1, wherein R² and R³ are each H

8. The compound according to claim 1, wherein X is CH₂.

9. The compound according to claim 1, wherein X is CHF or CF₂.

10. The compound according to claim 1, wherein Y is CH₂.

11. The compound according to claim 1, wherein Z is CHR⁸.

12. The compound according to claim 1, wherein R⁶, R⁷, R⁸, R⁹ and R¹⁰ are each H.

13. The compound according to claim 1, wherein:
R⁶, R⁸, R⁹ and R¹⁰ are each H; and
R⁷ is methyl;
or
R⁶, R⁷, R⁹ and R¹⁰ are each H; and
R⁸ is methyl;
or
R⁸, R⁹ and R¹⁰ are each H; and
R⁶ and R⁷ together form —CH₂—CH₂—;
or
R⁶, R⁸ and R⁹ are each H; and
R⁷ and R¹⁰ together form —CH₂—CH₂—;
or
R⁶, R⁷ and R¹⁰ are each H; and
R⁸ and R⁹ together form —CH₂—CH₂—.

14. The compound according to claim 1, wherein:
R⁶, R⁷ and R⁸ are each H;
and R⁹ and R¹⁰ together form —CH₂—;
or
R⁶, R⁸ and R⁹ are each H;
and R⁷ and R¹⁰ together form a bond.

15. The compound according to claim 1, wherein R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: chloro, cyclopropyl, difluoromethyl, ethyl, fluoro, 2-fluoropropan-2-yl, isopropyl, methoxy, methyl, and trifluoromethyl.

16. The compound according to claim 1, wherein R¹¹ is selected from: (1-(trifluoromethyl)cyclobutoxy)carbonyl, (1-(trifluoromethyl)cyclobutyl)methyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (3-(trifluoromethyl)oxetan-3-yloxy)carbonyl, (3,3,4,4,4-pentafluorobutan-2-yloxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(difluoromethyl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, 5-isopropyl-1,2,4-oxadiazol-3-yl, 5-methoxypyrimidin-2-yl, cyclobutylthiocarbonyl, cyclopropylthiocarbonyl, isopropylthiocarbonyl, and tert-butoxycarbonyl.

17. The compound according to claim 1, wherein R¹¹ is selected from: C₁-C₆ alkoxycarbonyl, C₁-C₆ alkylsulfonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, heteroaryl-C₁-C₆ alkylene, and heterocyclyloxycarbonyl; each optionally substituted with one or more substituents selected from: chloro, cyclopropyl, ethyl, fluoro, 2-fluoropropan-2-yl, isopropyl, methyl, and trifluoromethyl.

18. The compound according to claim 1, wherein R¹¹ is selected from: (1-(trifluoromethyl)cyclobutoxy)carbonyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (2,2,2-trifluoroethyl)sulfonyl, (3-(trifluoromethyl)oxetan-3-yloxy)carbonyl, 2,2,3,3,3-pentafluoropropoxy)carbonyl, 3-(2-fluoropropan-2-yl)-1,2,4-oxadiazol-5-yl, 3-(trifluoromethyl)-1,2,4-oxadiazol-5-yl, 3-(trifluoromethyl)-1,2,4-oxadiazol-5-ylmethyl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, cyclobutylthiocarbonyl, cyclopropylthiocarbonyl, and tert-butoxycarbonyl.

19. The compound according to claim 1, selected from compounds of Formula XVII and pharmaceutically acceptable salts, solvates, and hydrates thereof: 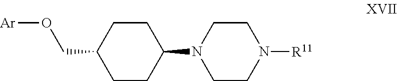
wherein:
Ar is selected from: aryl and heteroaryl; each optionally substituted with one or more substituents selected from: C₁-C₆ alkyl, C₁-C₄ alkylcarboxamide, C₁-C₆ alkylsulfinyl, C₁-C₆ alkylsulfonyl, C₁-C₆ alkylthio, carboxamide, cyano, C₃-C₇ cycloalkylsulfinyl, C₃-C₇ cycloalkylsulfonyl, C₃-C₇ cycloalkylthio, C₂-C₈ dialkylcarboxamide, C₁-C₆ haloalkylthio, halogen, heteroaryl, and C₁-C₆ alkylheteroaryl; and
R¹¹ is selected from: C₁-C₆ alkoxycarbonyl, C₁-C₆ alkylthiocarbonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, and heterocyclyloxycarbonyl; each optionally substituted with one or more substituents selected from: C₁-C₆ alkoxy, C₁-C₆ alkyl, C₃-C₇ cycloalkyl, C₁-C₆ haloalkyl, and halogen.

20. The compound according to claim 1, selected from compounds of Formula XVII and pharmaceutically acceptable salts, solvates, and hydrates thereof: 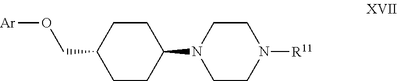
wherein:
Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(dimethylcarbamoyl)-3-methylphenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, and 6-cyano-2-methylpyrimidin-4-yl; and
R¹¹ is selected from: (1-(trifluoromethyl)cyclobutoxy)carbonyl, (1-(trifluoromethyl)cyclobutyl)methyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (3-(trifluoromethyl)oxetan-3-yloxy)carbonyl, (3,3,4,4,4-pentafluorobutan-2-yloxy)carbonyl, (trifluoromethyl)cyclopropanecarbonyl, 2,2,3,3,3-pentafluoropropoxy)carbonyl, 3-(2-fluoropropan-2-yl)-1,2,4-oxadiazol-5-yl, 3-(difluoromethyl)-1,2,4-oxadiazol-5-yl, 3-(trifluoromethyl)-1,2,4-oxadiazol-5-yl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, 5-isopropyl-1,2,4-oxadiazol-3-yl, 5-methoxypyrimidin-2-yl, cyclobutylthiocarbonyl, cyclopropylthiocarbonyl, isopropylthiocarbonyl, and tert-butoxycarbonyl.

21. The compound according to claim 1, selected from compounds of Formula XIX and pharmaceutically acceptable salts, solvates, and hydrates thereof: 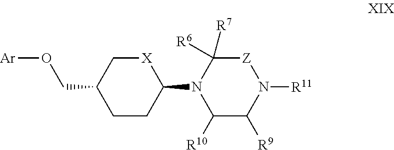
wherein:
Ar is selected from: aryl and heteroaryl; each optionally substituted with one or more substituents selected from: C₁-C₆ alkyl, C₁-C₆ alkylsulfonyl, cyano, C₃-C₇ cycloalkylsulfonyl, C₂-C₈ dialkylcarboxamide, C₁-C₆ haloalkylthio, halogen, heteroaryl, and C₁-C₆ alkylheteroaryl;
X is selected from: —CH₂—, C(O), CHF, and CF₂
R⁶, R⁸, R⁹ and R¹⁰ are each H; and R⁷ is C₁-C₆ alkyl; or R⁶, R⁷, R⁹ and R¹⁰ are each H; and R⁸ is C₁-C₆ alkyl; or R⁸, R⁹ and R¹⁰ are each H; and R⁶ and R⁷ together form —CH₂—CH₂—; or R⁶, R⁸ and R⁹ are each H; and R⁷ and R¹⁰ together form —CH₂—CH₂—; or R⁶, R⁷ and R¹⁰ are each H; and R⁸ and R⁹ together form —CH₂—CH₂—; or R⁶, R⁷ and R⁸ are each H; and R⁹ and R¹⁰ together form —CH₂—; or R⁶, R⁸ and R⁹ are each H; and R⁷ and R¹⁰ together form a bond; and
R¹¹ is selected from: C₁-C₆ alkoxycarbonyl, C₁-C₆ alkylsulfonyl, C₄-C₁₃ cycloalkylalkyl, C₃-C₇ cycloalkoxycarbonyl, C₃-C₇ cycloalkylcarbonyl, C₃-C₇ cycloalkythiocarbonyl, heteroaryl, heteroaryl-C₁-C₆ alkylene, and heterocyclyloxycarbonyl; each optionally substituted with one or more substituents selected from: chloro, cyclopropyl, ethyl, fluoro, 2-fluoropropan-2-yl, isopropyl, methyl, and trifluoromethyl

22. The compound according to claim 1, selected from compounds of Formula XIX and pharmaceutically acceptable salts, solvates, and hydrates thereof: 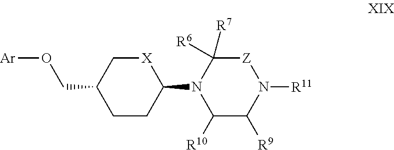
wherein:
Ar is selected from: 2-chloro-4-(methylsulfonyl)phenyl, 2-fluoro-4-(1H-1,2,3-triazol-1-yl)phenyl, 2-fluoro-4-(1H-1,2,4-triazol-1-yl)phenyl, 2-fluoro-4-(1H-imidazol-1-yl)phenyl, 2-fluoro-4-(1H-pyrrol-1-yl)phenyl, 2-fluoro-4-(1H-tetrazol-1-yl)phenyl, 2-fluoro-4-(2H-1,2,3-triazol-2-yl)phenyl, 2-fluoro-4-(4H-1,2,4-triazol-4-yl)phenyl, 2-fluoro-4-(4-methyl-1H-pyrazol-1-yl)phenyl, 2-fluoro-4-(methylsulfonyl)phenyl, 2-methyl-6-(methylsulfonyl)pyridin-3-yl, 3-chloro-4-(methylsulfonyl)phenyl, 3-cyanopyridin-4-yl, 4-(1H-1,2,4-triazol-1-yl)phenyl, 4-(1H-tetrazol-1-yl)phenyl, 4-(cyclopropylsulfonyl)-2-fluorophenyl, 4-(dimethylcarbamoyl)-2-fluorophenyl, 4-(methylsulfonyl)phenyl, 4-(trifluoromethylthio)phenyl, 4-cyanophenyl, 5-(methylsulfonyl)pyrazin-2-yl, 5-(methylsulfonyl)pyridin-2-yl, 6-cyano-2-methylpyrimidin-4-yl, and pyridin-4-yl;
X is selected from: —CH₂—, C(O), CHF, and CF₂
R⁶, R⁸, R⁹ and R¹⁰ are each H; and R⁷ is methyl; or R⁶, R⁷, R⁹ and R¹⁰ are each H; and R⁸ is methyl; or R⁸, R⁹ and R¹⁰ are each H; and R⁶ and R⁷ together form —CH₂—CH₂—; or R⁶, R⁸ and R⁹ are each H; and R⁷ and R¹⁰ together form —CH₂—CH₂—; or R⁶, R⁷ and R¹⁰ are each H; and R⁸ and R⁹ together form —CH₂—CH₂—; or R⁶, R⁷ and R⁸ are each H; and R⁹ and R¹⁰ together form —CH₂—; or R⁶, R⁸ and R⁹ are each H; and R⁷ and R¹⁰ together form a bond; and
R¹¹ is selected from: (1-(trifluoromethyl)cyclobutoxy)carbonyl, (1-(trifluoromethyl)cyclopropyl)carbonyl, (1-(trifluoromethyl)cyclopropyl)methyl, (1,1,1,3,3,3-hexafluoropropan-2-yloxy)carbonyl, (1,1,1-trifluoro-2-methylpropan-2-yloxy)carbonyl, (1,1,1-trifluoropropan-2-yloxy)carbonyl, (1-fluoro-2-methylpropan-2-yloxy)carbonyl, (1-fluoropropan-2-yloxy)carbonyl, (1-methylcyclobutoxy)carbonyl, (1-methylcyclopropoxy)carbonyl, (2,2,2-trifluoroethoxy)carbonyl, (2,2,2-trifluoroethyl)sulfonyl, (3-(trifluoromethyl)oxetan-3-yloxy)carbonyl, 2,2,3,3,3-pentafluoropropoxy)carbonyl, 3-(2-fluoropropan-2-yl)-1,2,4-oxadiazol-5-yl, 3-(trifluoromethyl)-1,2,4-oxadiazol-5-yl, 3-(trifluoromethyl)-1,2,4-oxadiazol-5-ylmethyl, 3-cyclopropyl-1,2,4-oxadiazol-5-yl, 3-isopropyl-1,2,4-oxadiazol-5-yl, 5-(2-fluoropropan-2-yl)-1,2,4-oxadiazol-3-yl, 5-(trifluoromethyl)-1,2,4-oxadiazol-3-yl, 5-(trifluoromethyl)pyrimidin-2-yl, 5-chloropyrimidin-2-yl, 5-ethylpyrimidin-2-yl, cyclobutylthiocarbonyl, cyclopropylthiocarbonyl, and tert-butoxycarbonyl.

23. The compound according to claim 1, selected from the following compounds and pharmaceutically acceptable salts, solvates, and hydrates thereof:
tert-butyl 4-((1r,4r)-4-((2-fluoro-4-(methylsulfonyl)phenoxy)methyl)cyclohexyl)piperazine-1-carboxylate;
5-ethyl-2-(4-((1r,4r)-4-((2-fluoro-4-(methylsulfonyl)phenoxy)methyl)cyclohexyl)piperazin-1-yl)pyrimidine;
2-(4-((1r,4r)-4-((2-fluoro-4-(methylsulfonyl)phenoxy)methyl)cyclohexyl)piperazin-1-yl)-5-(trifluoromethyl)pyrimidine;
isopropyl 4-((1r,4r)-4-((4-(methylsulfonyl)phenoxy)methyl)cyclohexyl)piperazine-1-carboxylate;
5-ethyl-2-(4-((1r,4r)-4-((4-(methylsulfonyl)phenoxy)methyl)cyclohexyl)piperazin-1-yl)pyrimidine;
3-isopropyl-5-(4-((1r,4r)-4-((4-(methylsulfonyl)phenoxy)methyl)cyclohexyl)piperazin-1-yl)-1,2,4-oxadiazole;
5-ethyl-2-(4-((1r,4r)-4-((5-(methylsulfonyl)pyrazin-2-yloxy)methyl)cyclohexyl)piperazin-1-yl)pyrimidine;
tert-butyl 4-((1s,4s)-4-((2-methyl-6-(methylsulfonyl)pyridin-3-yloxy)methyl)cyclohexyl)piperazine-1-carboxylate;
tert-butyl 4-((1r,4r)-4-((2-methyl-6-(methylsulfonyl)pyridin-3-yloxy)methyl)cyclohexyl)piperazine-1-carboxylate;
5-ethyl-2-(4-((1r,4r)-4-((2-methyl-6-(methylsulfonyl)pyridin-3-yloxy)methyl)cyclohexyl)piperazin-1-yl)pyrimidine;
3-(2-fluoropropan-2-yl)-5-(4-((1r,4r)-4-((4-(methylsulfonyl)phenoxy)methyl)cyclohexyl)piperazin-1-yl)-1,2,4-oxadiazole;
2-(4-((1r,4r)-4-((2-methyl-6-(methylsulfonyl)pyridin-3-yloxy)methyl)cyclohexyl)piperazin-1-yl)-5-(trifluoromethyl)pyrimidine;
1-methylcyclopropyl 4-((1r,4r)-4-((2-fluoro-4-(methylsulfonyl)phenoxy)methyl)cyclohexyl)piperazine-1-carboxylate;
tert-butyl 4-((1r,4r)-4-((4-(1H-1,2,4-triazol-1-yl)phenoxy)methyl)cyclohexyl)piperazine-1-carboxylate;
2-(4-((1r,4r)-4-((4-(1H-1,2,4-triazol-1-yl)phenoxy)methyl)cyclohexyl)piperazin-1-yl)-5-(trifluoromethyl)pyrimidine;
1,1,1,3,3,3-hexafluoropropan-2-yl 4-((1r,4r)-4-((4-(1H-1,2,4-triazol-1-yl)phenoxy)methyl)cyclohexyl)piperazine-1-carboxylate;
5-chloro-2-(4-((1r,4r)-4-((2-fluoro-4-(methylsulfonyl)phenoxy)methyl)cyclohexyl)piperazin-1-yl)pyrimidine;
5-(4-((1r,4r)-4-((2-fluoro-4-(methylsulfonyl)phenoxy)methyl)cyclohexyl)piperazin-1-yl)-3-(2-fluoropropan-2-yl)-1,2,4-oxadiazole;
3-(4-((1r,4r)-4-((2-fluoro-4-(methylsulfonyl)phenoxy)methyl)cyclohexyl)piperazin-1-yl)-5-(2-fluoropropan-2-yl)-1,2,4-oxadiazole;
tert-butyl 4-((1r,4r)-4-((4-(1H-tetrazol-1-yl)phenoxy)methyl)cyclohexyl)piperazine-1-carboxylate;
2-(4-((1r,4r)-4-((4-(1H-tetrazol-1-yl)phenoxy)methyl)cyclohexyl)piperazin-1-yl)-5-(trifluoromethyl)pyrimidine;
tert-butyl 4-((1r,4r)-4-((4-cyanophenoxy)methyl)cyclohexyl)piperazine-1-carboxylate;
4-(((1r,4r)-4-(4-(5-(trifluoromethyl)pyrimidin-2-yl)piperazin-1-yl)cyclohexyl)methoxy)benzonitrile;
2-((S)-4-((1r,4S)-4-((2-fluoro-4-(methylsulfonyl)phenoxy)methyl)cyclohexyl)-3-methylpiperazin-1-yl)-5-(trifluoromethyl)pyrimidine;
5-((S)-((1r,4S)-4-((2-fluoro-4-(methylsulfonyl)phenoxy)methyl)cyclohexyl)-3-methylpiperazin-1-yl)-3-(2-fluoropropan-2-yl)-1,2,4-oxadiazole;
2-((S)-3-methyl-4-((1r,4S)-4-((2-methyl-6-(methylsulfonyl)pyridin-3-yloxy)methyl)cyclohexyl)piperazin-1-yl)-5-(trifluoromethyl)pyrimidine;
2-((R)-4-((1r,4R)-4-((2-fluoro-4-(methylsulfonyl)phenoxy)methyl)cyclohexyl)-3-methylpiperazin-1-yl)-5-(trifluoromethyl)pyrimidine;
(R)-1-fluoropropan-2-yl 4-((1r,4R)-4-((2-fluoro-4-(methylsulfonyl)phenoxy)methyl)cyclohexyl)piperazine-1-carboxylate;
(S)-1-fluoropropan-2-yl 4-((1r,4S)-4-((2-fluoro-4-(methylsulfonyl)phenoxy)methyl)cyclohexyl)piperazine-1-carboxylate;
1-fluoro-2-methylpropan-2-yl 4-((1r,4S)-4-((2-fluoro-4-(methylsulfonyl)phenoxy)methyl)cyclohexyl)piperazine-1-carboxylate;
5-chloro-2-((R)-4-((1r,4R)-4-((2-fluoro-4-(methylsulfonyl)phenoxy)methyl)cyclohexyl)-3-methylpiperazin-1-yl)pyrimidine;
(R)—((R)-1,1,1-trifluoropropan-2-yl) 4-((1r,4R)-4-((2-fluoro-4-(methylsulfonyl)phenoxy)methyl)cyclohexyl)-3-methylpiperazine-1-carboxylate;
tert-butyl 4-((1r,4r)-4-((2-fluoro-4-(methylsulfonyl)phenylamino)methyl)cyclohexyl)piperazine-1-carboxylate;
tert-butyl 4-((1r,4s)-4-((4-(trifluoromethylthio)phenoxy)methyl)cyclohexyl)piperazine-1-carboxylate;
tert-butyl 4-((1s,4s)-4-((2-fluoro-4-(methylsulfonyl)phenylamino)methyl)cyclohexyl)piperazine-1-carboxylate;
(4-((1r,4r)-4-((2-fluoro-4-(methylsulfonyl)phenoxy)methyl)cyclohexyl)piperazin-1-yl)(1-(trifluoromethyl)cyclopropyl)methanone;
1-methylcyclobutyl 4-((1r,4r)-4-((2-fluoro-4-(methylsulfonyl)phenoxy)methyl)cyclohexyl)piperazine-1-carboxylate;
tert-butyl 4-((1r,4r)-4-((4-(dimethylcarbamoyl)-2-fluorophenoxy)methyl)cyclohexyl)piperazine-1-carboxylate;
1-((1r,4r)-4-((2-fluoro-4-(methylsulfonyl)phenoxy)methyl)cyclohexyl)-4-((1-(trifluoromethyl)cyclopropyl)methyl)piperazine;
3-cyclopropyl-5-(4-((1r,4r)-4-((2-fluoro-4-(methylsulfonyl)phenoxy)methyl)cyclohexyl)piperazin-1-yl)-1,2,4-oxadiazole;
tert-butyl 4-((1r,4r)-4-((2-fluoro-4-(4H-1,2,4-triazol-4-yl)phenoxy)methyl)cyclohexyl)piperazine-1-carboxylate;
1,1,1,3,3,3-hexafluoropropan-2-yl 4-((1r,4r)-4-((2-fluoro-4-(methylsulfonyl)phenoxy)methyl)cyclohexyl)piperazine-1-carboxylate;
tert-butyl 4-((1r,4r)-4-((2-fluoro-4-(2H-1,2,3-triazol-2-yl)phenoxy)methyl)cyclohexyl)piperazine-1-carboxylate;
(R)-1,1,1-trifluoropropan-2-yl 4-((1r,4R)-4-((2-fluoro-4-(1H-tetrazol-1-yl)phenoxy)methyl)cyclohexyl)piperazine-1-carboxylate;
5-chloro-2-(4-((1r,4r)-4-((2-fluoro-4-(1H-tetrazol-1-yl)phenoxy)methyl)cyclohexyl)piperazin-1-yl)pyrimidine;
2,2,2-trifluoroethyl 4-((1r,4r)-4-((2-fluoro-4-(methylsulfonyl)phenoxy)methyl)cyclohexyl)piperazine-1-carboxylate;
tert-butyl 4-((1r,4r)-4-((6-cyano-2-methylpyrimidin-4-yloxy)methyl)cyclohexyl)piperazine-1-carboxylate;
2,2,3,3,3-pentafluoropropyl 4-((1r,4r)-4-((2-fluoro-4-(methylsulfonyl)phenoxy)methyl)cyclohexyl)piperazine-1-carboxylate;
tert-butyl 4-((1r,4r)-4-((2-fluoro-4-(1H-tetrazol-1-yl)phenoxy)methyl)cyclohexyl)piperazine-1-carboxylate;
(R)-1,1,1-trifluoropropan-2-yl 4-((1r,4R)-4-((2-fluoro-4-(methylsulfonyl)phenoxy)methyl)cyclohexyl)piperazine-1-carboxylate;
(S)-1,1,1-trifluoropropan-2-yl 4-((1r,45)-4-((2-fluoro-4-(methylsulfonyl)phenoxy)methyl)cyclohexyl)piperazine-1-carboxylate;
tert-butyl 4-((1r,4r)-4-((2-fluoro-4-(1H-imidazol-1-yl)phenoxy)methyl)cyclohexyl)piperazine-1-carboxylate;
1,1,1-trifluoro-2-methylpropan-2-yl 4-((1r,4r)-4-((2-fluoro-4-(methylsulfonyl)phenoxy)methyl)cyclohexyl)piperazine-1-carboxylate;
S-cyclopropyl 4-((1r,4r)-4-((2-fluoro-4-(methylsulfonyl)phenoxy)methyl)cyclohexyl)piperazine-1-carbothioate;
S-cyclobutyl 4-((1r,4r)-4-((2-fluoro-4-(methylsulfonyl)phenoxy)methyl)cyclohexyl)piperazine-1-carbothioate;
tert-butyl 4-((1r,4r)-4-((2-fluoro-4-(1H-1,2,3-triazol-1-yl)phenoxy)methyl)cyclohexyl)piperazine-1-carboxylate;
tert-butyl 4-((1r,4r)-4-((2-fluoro-4-(1H-1,2,4-triazol-1-yl)phenoxy)methyl)cyclohexyl)piperazine-1-carboxylate;
tert-butyl 4-((1r,4r)-4-((2-fluoro-4-(1H-pyrrol-1-yl)phenoxy)methyl)cyclohexyl)piperazine-1-carboxylate;
tert-butyl 4-((1r,4r)-4-((2-fluoro-4-(4-methyl-1H-pyrazol-1-yl)phenoxy)methyl)cyclohexyl)piperazine-1-carboxylate;
tert-butyl 4-((1r,4r)-4-((4-(methylsulfonyl)pyridin-2-yloxy)methyl)cyclohexyl)piperazine-1-carboxylate;
tert-butyl 4-((1r,4r)-4-((4-(cyclopropylsulfonyl)-2-fluorophenoxy)methyl)cyclohexyl)piperazine-1-carboxylate;
tert-butyl 4-((1r,4r)-4-((2-chloro-4-(methylsulfonyl)phenoxy)methyl)cyclohexyl)piperazine-1-carboxylate;
tert-butyl 4-((1r,4r)-4-((3-chloro-4-(methylsulfonyl)phenoxy)methyl)cyclohexyl)piperazine-1-carboxylate;
5-(4-((1r,4r)-4-((2-fluoro-4-(methylsulfonyl)phenoxy)methyl)cyclohexyl)piperazin-1-yl)-3-(trifluoromethyl)-1,2,4-oxadiazole;
3-(4-((1r,4r)-4-((2-fluoro-4-(methylsulfonyl)phenoxy)methyl)cyclohexyl)piperazin-1-yl)-5-(trifluoromethyl)-1,2,4-oxadiazole; and
1-(trifluoromethyl)cyclobutyl 4-((1r,4r)-4-((2-fluoro-4-(methylsulfonyl) phenoxy)methyl)cyclohexyl)piperazine-1-carboxylate.

24. The compound according to claim 1, selected from the following compounds and pharmaceutically acceptable salts, solvates, and hydrates thereof:
tert-butyl 4-((1r,4r)-4-((3-cyanopyridin-4-yloxy)methyl)cyclohexyl)piperazine-1-carboxylate;
tert-butyl 4-((1r,4r)-4-((pyridin-4-yloxy)methyl)cyclohexyl)piperazine-1-carboxylate;
(S)-tert-butyl 4-((1r,45)-4-((2-fluoro-4-(methylsulfonyl)phenoxy) methyl)cyclohexyl)-2-methyl piperazine-1-carboxylate;
(R)-tert-butyl 4-((1r,4R)-4-((2-fluoro-4-(methylsulfonyl)phenoxy) methyl)cyclohexyl)-2-methyl piperazine-1-carboxylate;
1-((1r,4r)-4-((2-fluoro-4-(methylsulfonyl)phenoxy)methyl)cyclohexyl)-4-(2,2,2-trifluoroethylsulfonyl)piperazine;
4-(((1r,4r)-4-(4-(5-(trifluoromethyl)pyrimidin-2-yl)piperazin-1-yl)cyclohexyl)methoxy)nicotinonitrile;
3-(trifluoromethyl)oxetan-3-yl 4-((1r,4r)-4-((2-fluoro-4-(methylsulfonyl)phenoxy)methyl)cyclohexyl)piperazine-1-carboxylate;
5-((4-((1r,4r)-4-((2-fluoro-4-(methylsulfonyl)phenoxy) methyl)cyclohexyl)piperazin-1-yl)methyl)-3-(trifluoromethyl)-1,2,4-oxadiazole;
(S)-3-(trifluoromethyl)oxetan-3-yl 4-((1r,45)-4-((2-fluoro-4-(methylsulfonyl) phenoxy)methyl)cyclohexyl)-2-methylpiperazine-1-carboxylate;
tert-butyl 4-((1r,4r)-1-cyano-4-((2-fluoro-4-(methylsulfonyl)phenoxy) methyl)cyclohexyl)piperazine-1-carboxylate;
tert-butyl 3-((1r,4r)-4-((2-fluoro-4-(methylsulfonyl) phenoxy)methyl)cyclohexyl)-3,8-diazabicyclo[3.2.1]octane-8-carboxylate;
5-((S)-4-((1r,45)-4-((2-fluoro-4-(methylsulfonyl)phenoxy)methyl)cyclohexyl)-2-methylpiperazin-1-yl)-3-(trifluoromethyl)-1,2,4-oxadiazole;
1-(trifluoromethyl)cyclobutyl 8-((1r,4r)-4-((2-fluoro-4-(methylsulfonyl) phenoxy)methyl)cyclohexyl)-3,8-diazabicyclo[3.2.1]octane-3-carboxylate;
tert-butyl 4-((1r,4r)-4-((2-fluoro-4-(methylsulfonyl) phenoxy)methyl)cyclohexyl)-2-oxopiperazine-1-carboxylate;
tert-butyl 4-((1R,2S,4R)-2-fluoro-4-((2-fluoro-4-(methylsulfonyl)phenoxy) methyl)cyclohexyl)piperazine-1-carboxylate;
tert-butyl 4-((1R,2R,4R)-2-fluoro-4-((2-fluoro-4-(methylsulfonyl)phenoxy) methyl)cyclohexyl)piperazine-1-carboxylate;
tert-butyl 4-((1R,4R)-2-fluoro-4-((2-fluoro-4-(methylsulfonyl)phenoxy) methyl)cyclohexyl)piperazine-1-carboxylate;
tert-butyl 5-((1r,4r)-4-((2-fluoro-4-(methylsulfonyl)phenoxy) methyl)cyclohexyl)-2,5-diazabicyclo[4.1.0]heptane-2-carboxylate;
tert-butyl 4-((1R,4R)-4-((2-fluoro-4-(methylsulfonyl)phenoxy)methyl)-2-oxocyclohexyl)piperazine-1-carboxylate;
((1R,6S)-tert-butyl 5-((1r,4S)-4-((2-fluoro-4-(methylsulfonyl)phenoxy) methyl)cyclohexyl)-2,5-diazabicyclo[4.1.0]heptane-2-carboxylate;
(1S,6R)-tert-butyl 5-((1r,4R)-4-((2-fluoro-4-(methylsulfonyl)phenoxy) methyl)cyclohexyl)-2,5-diazabicyclo[4.1.0]heptane-2-carboxylate;
tert-butyl 4-((1R,4R)-2,2-difluoro-4-((2-fluoro-4-(methylsulfonyl)phenoxy) methyl)cyclohexyl)piperazine-1-carboxylate;
tert-butyl 4-((1r,4r)-4-((2-fluoro-4-(methylsulfonyl) phenoxy)methyl)cyclohexyl)-4,7-diazaspiro[2.5]octane-7-carboxylate;
3-((1S,6R)-5-((1r,4R)-4-((2-fluoro-4-(methylsulfonyl)phenoxy) methyl)cyclohexyl)-2,5-diazabicyclo[4.1.0]heptan-2-yl)-5-(trifluoromethyl)-1,2,4-oxadiazole;
tert-butyl 6-((1r,4r)-4-((2-fluoro-4-(methylsulfonyl) phenoxy)methyl)cyclohexyl)-3,6-diazabicyclo[3.1.0]hexane-3-carboxylate.

25. A composition comprising a compound according to claim 1.

26. A composition comprising a compound according to claim 1 and a pharmaceutically acceptable carrier.

27. A method for preparing a composition comprising the step of admixing a compound according to claim 1 and a pharmaceutically acceptable carrier.

28. A composition comprising a compound according to claim 1 and a second pharmaceutical agent.

29. A method for preparing a composition comprising the step of admixing a compound according to claim 1 and a second pharmaceutical agent.

30. A pharmaceutical product selected from: a pharmaceutical composition, a formulation, a dosage form, a combined preparation, a twin pack, and a kit; comprising a compound according to claim 1 and a second pharmaceutical agent.

31. A method for increasing the secretion of an incretin in an individual, or for increasing a blood incretin level in an individual, comprising administering to said individual in need thereof, a therapeutically effective amount of a compound according to claim 1.

32. A method for the treatment of a disorder selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity; in an individual; comprising administering to said individual in need thereof, a therapeutically effective amount of a compound according to claim 1.

33-42. (canceled)

43. The method according to claim 32; wherein said incretin is GLP-1.

44. The method according to claim 32; wherein said incretin is GIP.

45. The method according to claim 32; wherein said incretin is PYY.

46. The method according to claim 32; wherein said disorder is a condition characterized by low bone mass selected from: osteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine, and loss of height.

47. The method according to claim 32; wherein said disorder is a neurological disorder selected from: stroke and Parkinsonism.

48. The method according to claim 32; wherein said disorder is a metabolic-related disorder selected from: diabetes, type 1 diabetes, type 2 diabetes, inadequate glucose tolerance, impaired glucose tolerance, insulin resistance, hyperglycemia, hyperlipidemia, hypertriglyceridemia, hypercholesterolemia, dyslipidemia, atherosclerosis, stroke, syndrome X, hypertension, pancreatic beta-cell insufficiency, enteroendocrine cell insufficiency, glucosuria, metabolic acidosis, cataracts, diabetic nephropathy, diabetic neuropathy, peripheral neuropathy, diabetic coronary artery disease, diabetic cerebrovascular disease, diabetic peripheral vascular disease, diabetic retinopathy, metabolic syndrome, a condition related to diabetes, myocardial infarction, learning impairment, memory impairment, a neurodegenerative disorder, a condition ameliorated by increasing a blood GLP-1 level in an individual with a neurodegenerative disorder, excitotoxic brain damage caused by severe epileptic seizures, Alzheimer's disease, Parkinson's disease, Huntington's disease, prion-associated disease, stroke, motor-neuron disease, traumatic brain injury, spinal cord injury, and obesity.

49. The method according to claim 32; wherein said disorder is type 2 diabetes.

50. The composition according to claim 28 wherein said second pharmaceutical agent is selected from: an inhibitor of DPP-IV, a biguanide, an alpha-glucosidase inhibitor, an insulin analogue, a sulfonylurea, a SGLT2 inhibitor, a meglitinide, a thiazolidinedione, and an anti-diabetic peptide analogue.

51. The composition according to claim 28; wherein said second pharmaceutical agent is an inhibitor of DPP-IV selected from the following inhibitors of DPP-IV and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;
8-[3 (R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-yl boronic acid;
4(S)-fluoro-1-[2-[(1R,3 S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl) ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5 difluoropiperidin-2-one;
(R)-2-((6-(3-aminopiperidin-1-yl)-3-methyl-2,4-dioxo-3,4-dihydropyrimidin-1 (2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo [2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5 dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro [1,2,4]triazolo[4,3-a]pyrazin-7(8H)-yl]carbonyl}-6-(2,4,5-trifluorophenyl)cyclohex-3-en-1-amine.
